(19) United States
(12) Patent Application Publication
(10) Pub. No.: US 2026/0098061 A1
(43) Pub. Date: Apr. 9, 2026

(54) EPHA2-BINDING PENDING PEPTIDE AND COMPOSITION COMPRISING SAME

(71) Applicant: PeptiDream Inc., Kawasaki-shi (JP)

(72) Inventors: Takanori Aoki, Kanagawa (JP); Takeru Ehara, Kawasaki (JP); Sora Enya, Kanagawa (JP); Makoto Jitsuoka, Kanagawa (JP); Kouki Morimoto, Kawasaki (JP); Shunichi Nakano, Kanagawa (JP); Takayuki Nagasawa, Kawasaki (JP); Masaki Ohuchi, Kanagawa (JP); Hayato Yanagida, Kawasaki (JP); Zaid Amso, San Diego, CA (US); Rongjun He, San Diego, CA (US); Derek Cole, San Diego, CA (US)

(21) Appl. No.: 19/115,394

(22) PCT Filed: Sep. 28, 2023
(86) PCT No.: PCT/US2023/075427

§ 371 Date: Mar. 26, 2025

Related U.S. Application Data
(60) Provisional application No. 63/411,222, filed on Sep. 29, 2022.

(51) Int. Cl. C07K 7/64; A61K 47/64

(52) U.S. Cl. CPC C07K 7/64; A61K 47/64

(57) ABSTRACT

The present technology generally relates to peptides that bind to the Eph receptor A2 (EphA2), to peptides that bind to the EphA2, and to compositions comprising such peptides.

### RELATED APPLICATIONS

This application claims priority to U.S. Provisional Application No. 63/411,222, filed on Sep. 29, 2022. The entire contents of the foregoing application, including any drawings and sequence listing, are expressly incorporated herein by reference.

### JOINT RESEARCH AGREEMENT

Subject matter disclosed herein was developed, and the claimed invention was made by, or on behalf of, one or more parties to a Joint Research Agreement (JRA), within the meaning of 35 U.S.C. § 100 (h) and 37 C.F.R. § 1.9 (e), that was in effect on or before the effective filing date of the claimed invention. Said one or more parties to the JRA consist of PeptiDream, Inc. (Kanagawa, Japan) and RayzeBio, Inc. (San Diego, CA, U.S.A.). The claimed invention was made as a result of activities undertaken within the scope of said Joint Research Agreement.

### TECHNICAL FIELD

The present technology relates to peptides that bind to the Eph receptor tyrosine kinase A2 (EphA2) and to compositions comprising such peptides. The invention also includes conjugates comprising said peptides, conjugated to one or more effector and/or functional groups, to any substance such as pharmaceutical compositions, comprising said peptide ligands and the substance conjugates and to the use of said peptide ligands and substance conjugates in preventing, suppressing or treating a disease or disorder characterized by either overexpression or decreased expression of EphA2 in diseased tissue, such as in a tumor when EphA2 is overexpressed.

### BACKGROUND INFORMATION

Eph receptor A2 (Ephrin type-A receptor 2; EphA2) is a member of a receptor type-Tyrosine kinase. The ephrin A, which is ligand for the EphA2, is a membrane protein so that cell-cell interaction is known to be necessary to transmit a signal via EphA2. It is known that the EphA2-related signal plays important roll in a cell proliferation or differentiation. Moreover, the EphA2 is known as one of the important cancer-related protein; EphA2 is overexpressed in some of human tumor cells, and the decrease of EphA2 expression level using anti-EphA2 antibody leads to the proliferation of cancer cells (JP2010246546). Thus, antibody that binds to EphA2 is expected to be a pharmaceutical component for cancers. Also, it is expected that Antibody-Drug conjugate which includes anti-EphA2 antibody and anti-cancer drug will be a pharmaceutical component for cancers such as Medimmune (MedImmune LLC). However, such Antibody-Drug Conjugate (ADC) has not yet reached the pharmaceutical market.

Recently, Drug-conjugated peptide (PDC), is known to be a solution. Because of the characteristic of peptides (insert PD's scientific paper), peptides, especially cyclic peptides will cover ADC's weakness and be an alternative pharmaceutical component to ADC.

Thus, it is possible to be a pharmaceutical component for disease related to the overexpression or decreased expression of EphA2. Additionally, use a peptide that binds/has avidity to EphA2 (EphA2-binding peptide) to target and transport subjects having pharmacological actions to the EphA2, such as low molecular weight compounds, middle molecular weight compounds, high molecular weight compounds, peptides, proteins, antibodies, and nucleic acids.

Using a EphA2-binding peptide, the distribution and amount of EphA2 expression may be confirmed, for example, by measuring the binding of a fluorescent-labeled or tagged peptide to EphA2. Furthermore, the affinity of a ligand for the EphA2, or for different species EphA2s may be determined through the use of EphA2-binding peptides.

Therefore, novel EphA2-binding peptides and compositions comprising the EphA2-binding peptide are both useful and desired.

### SUMMARY OF TECHNOLOGY

The invention described herein provides, inter alia, a peptide (e.g., a cyclic peptide) that binds to EphA2, in particular to human EphA2; a linker-attached peptide thereof; a conjugate thereof; a kit thereof (e.g., a kit for use in a method of diagnosing disease or disorder characterized by overexpression or decreased expression of EphA2 by determination of the expression level of EphA2); a composition (e.g., pharmaceutical composition) comprising such EphA2-binding peptide or conjugate thereof; and methods of use thereof.

Specific, non-limiting, and illustrative aspects and embodiments of the invention described herein are provided herein below as numbered embodiments. As used herein, “a (cyclic) peptide” means “a peptide, such as a cyclic peptide.”

1. A (cyclic) peptide that has avidity for ephrin type-A receptor 2 (EphA2), wherein the peptide comprises an amino acid sequence including deletion, substitution, and/or addition of one or several (e.g., 1-6) amino acids in the amino acid sequence of SEQ ID NO: 1:

(SEQ ID NO: 1)
da-MeF-N-L-Hgl-MeF-W1Me-V-W1Me-T-E-C

or a pharmaceutically acceptable salt thereof, wherein the (cyclic) peptide consists of 10 to 12 amino acid residues.

2. The (cyclic) peptide of embodiment 1, wherein 1-5 amino acids selected the group consisting of the 3^{rd} N, 4^{th} L, 6^{th} MeF, 10^{th} T and 11^{th} E of SEQ ID NO: 1, is/are deleted, optionally without additional addition and/or substitution.

3. The (cyclic) peptide of embodiment 1 or 2, wherein one to several (e.g., 1, 2, 3, 4 or 5) amino acids are added.

4. The (cyclic) peptide of any one of embodiments 1 to 3, wherein one or more amino acid residues selected from the 2^{nd} MeF, 6^{th} MeF, 8^{th} V and 11^{th} E are substituted.

5. The (cyclic) peptide of any one of embodiments 1 to 4, wherein the peptide comprises an amino acid sequence with deletion of 2 or less amino acids in the amino acid SEQ ID NO: 1, optionally without additional addition and/or substitution.

6. The (cyclic) peptide of embodiment 5, wherein 1-2 amino acids selected from the group consisting of the 10^{th} T and 11^{th} E of SEQ ID NO: 1 is/are deleted, optionally without additional addition and/or substitution.

7. A (cyclic) peptide that has avidity for ephrin type-A receptor 2 (EphA2), wherein the peptide comprises an amino acid sequence of Formula (I), or a pharmaceutically acceptable salt thereof,

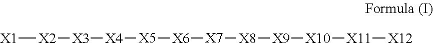

wherein,
   X1 is an amino acid;
   X2 is an amino acid comprising an aromatic ring, an N-methylated amino acid thereof, or a variant thereof;
   X3 is a hydrophilic amino acid (e.g. N, Q, Cit, K or a variant thereof), glycine (G), Alanine (A) or a variant thereof (e.g., da, 2-Aminoisobutyric acid (Aib);
   X4 is a hydrophobic amino acid (e.g., leucine (L)), a hydrophilic amino acid (e.g., citrulline (Cit)), or a variant thereof;
   X5 is a hydrophilic amino acid, or a variant thereof;
   X6 is a hydrophilic amino acid, an amino acid comprising an aromatic ring, or an N-methylated amino acid thereof;
   X7 is an amino acid comprising an aromatic ring (e.g., W, F, or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof.

8. The (cyclic) peptide of embodiment 7, wherein X3 is a hydrophilic amino acid.

9. The (cyclic) peptide of embodiment 8, wherein X3 is an amino acid comprising an electrically charged side chain (e.g., K or a variant thereof), an amino acid comprising a polar uncharged side chain (e.g., Q, Cit, N, or a variant thereof), or G, A or variant thereof.

10. The (cyclic) peptide of any one of embodiments 7-9, wherein X4 is a hydrophobic amino acid.

11. The (cyclic) peptide of embodiment 10, wherein X4 is an amino acid comprising a hydrophobic side chain (e.g., L), an amino acid comprising a polar uncharged side chain (e.g., Cit or a variant thereof).

12. The (cyclic) peptide of any one of embodiments 7-11, wherein X5 is a hydrophilic amino acid.

13. The (cyclic) peptide of embodiment 12, wherein X5 is an amino acid comprising an electrically charged side chain (e.g., E, Hgl, D, or a variant thereof), or an amino acid comprising a polar uncharged side chain (e.g., Q, Cit, Hgn, N, or a variant thereof).

14. The (cyclic) peptide of any one of embodiments 7-13, wherein X6 is a hydrophilic amino acid.

15. The (cyclic) peptide of embodiment 14, wherein X6 is an amino acid comprising an electrically charged side chain (e.g., E, Hgl, D, or a variant thereof), or an amino acid comprising a polar uncharged side chain (e.g., Q, Cit, Hgn, N, or variant).

16. The (cyclic) peptide of any one of embodiments 7-15, wherein X11 is a hydrophilic amino acid.

17. The (cyclic) peptide of embodiment 16, wherein X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof).

18. The (cyclic) peptide of any one of embodiments 1-17, wherein the peptide has an amino acid sequence of Formula (I), or a pharmaceutically acceptable salt thereof,

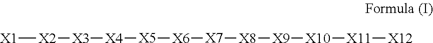

X1 is an amino acid;
X2 is F, or a variant thereof that substitutes the unsubstituted phenyl ring of F with:
   (i) a phenyl ring substituted by 1 or 2 substituents each independently selected from —OH, —CN, —C₁₋₃ alkyl (e.g., —CH₃), or
   (ii) a 6-membered heteroaryl ring optionally substituted by 1 or 2 substituents each independently selected from —OH, —CN, —C₁₋₃ alkyl (e.g., —CH₃), wherein the F or the structural variant thereof is optionally N-methylated;
X3 is a hydrophilic amino acid (e.g. N, Q, Cit, K or a variant thereof), G, Aib, Hgn, Ala, or a variant thereof (e.g., da);
X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
X5 is an amino acid (e.g., a hydrophilic amino acid; Dab, Dap, R, E or a variant thereof; or an amino acid with a functional side chain (e.g., not glycine);
X6 is an N-methylated amino acid thereof;
X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F);
X8 is an amino acid with —H on the alpha-amino group;
X9 is W or Y or a variant thereof; (e.g., W or a variant thereof);
X10 is absent, or a polar amino acid (e.g., T or a variant thereof);
X11 is absent, or an amino acid (e.g., a hydrophilic amino acid; Dab, Dap, R, E or a variant thereof; or an amino acid with a functional side chain (e.g., not glycine)); and
X12 is C or a variant thereof.

19. The (cyclic) peptide of any one of embodiments 1 to 18, wherein the peptide has an amino acid sequence of Formula (Ia), or a pharmaceutically acceptable salt thereof,

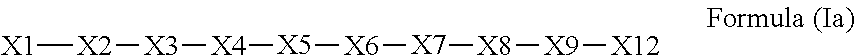

wherein,
   X1 is an amino acid (e.g., D-amino acid);
   X2 is an amino acid comprising an aromatic ring, an N-methylated amino acid thereof, or a variant thereof;
   X3 is a hydrophilic amino acid (e.g., N, Q, Cit, K or a variant thereof), G, A, or a variant thereof (e.g., da, Alb);
   X4 is a hydrophobic amino acid, or a hydrophilic amino acid (e.g., Cit or a variant thereof);
   X5 is a hydrophilic amino acid (e.g., Dab, Dap, R, E, Q, D, K), or a variant thereof);
   X6 is a hydrophilic amino acid, an amino acid comprising an aromatic ring (e.g., W, or F, or a variant thereof), or an N-methylated amino acid thereof;
   X7 is an amino acid comprising an aromatic ring (e.g., W, F, or a variant thereof);
   X8 is a hydrophobic amino acid, a hydrophilic amino acid, or an N-methylated amino acid;
   X9 is an amino acid comprising an aromatic ring (e.g., W, F or a variant thereof); and
   X12 is C or a variant thereof.

20. The (cyclic) peptide of any one of embodiments 1 to 18, wherein the peptide has an amino acid sequence according to Formula (I), or a pharmaceutically acceptable salt thereof,

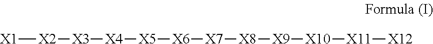

wherein,
   X1 is an amino acid (e.g., D-amino acid);
   X2 is an amino acid comprising an aromatic ring, an N-methylated amino acid thereof, or a variant thereof;
   X3 is a hydrophilic amino acid (e.g., N, Q, Cit, K or a variant thereof), G. A, or a variant thereof (e.g., da, Alb);
   X4 is a hydrophobic amino acid, or a hydrophilic amino acid (e.g., Cit or a variant thereof);
   X5 is a hydrophilic amino acid (e.g., Dab, Dap, R, E, Q, D, K), or a variant thereof);
   X6 is a hydrophilic amino acid, an amino acid comprising an aromatic ring (e.g., W, or F, or a variant thereof), or an N-methylated amino acid thereof;
   X7 is an amino acid comprising an aromatic ring (e.g., W, F, or a variant thereof);
   X8 is a hydrophobic amino acid, a hydrophilic amino acid, or an N-methylated amino acid;
   X9 is an amino acid comprising an aromatic ring (e.g., W, F or a variant thereof);
   X10 is a hydrophilic amino acid (e.g., T, S, N, Q, K, Cit, or a variant thereof);
   X11 is a hydrophilic amino acid; and
   X12 is C or a variant thereof.

21. The (cyclic) peptide of any one of embodiments 1 to 20, wherein
X1 is an amino acid (e.g., D-amino acid);
X2 is F, Y, W, a variant thereof, or an N-methylated amino acid thereof;
X3 is N, Q, Cit, G, Alb, K, A, or a variant thereof;
X4 is G, A, Cit, or a variant thereof (e.g., G substituted with straight or branched C₁₋₅ alkyl, G substituted with C₃₋₇ cycloalkyl, or A substituted with C₃₋₇ cycloalkyl);
X5 is a hydrophilic L-amino acid, wherein the L-amino acid comprises a functional group selected from —NH₂, —C(O)OH, —NHC(NH)NH₂, —NHC(O)NH₂, —C(O)NH₂, and —NHC(O)CH₃;
X6 is a hydrophilic amino acid, F, Y, W, N-methylated amino acid thereof, or a variant thereof, wherein the hydrophilic amino acid comprises a functional group selected from —C(O)OH, —C(O)NH₂, and —NHC(O)CH₃;
X7 is F, W, or a variant thereof;
X8 is G substituted with one or two straight or branched C₁₋₅ alkyl, G substituted with C₃₋₇ cycloalkyl, A substituted with C₃₋₇ cycloalkyl, or a hydrophilic L-amino acid wherein the hydrophilic L-amino acid comprises —NH₂, one or more —OH, —C(O)OH, —NHC(NH)NH₂, —NHC(O)NH₂, —C(O)NH₂, or —NHC(O)CH₃; or the hydrophilic amino acid comprises a zwitterion;
X9 is F, W, or a variant thereof;
X10 is absent, Q, S, K, Cit, N, T, or a variant thereof (e.g., Q, S, K, Cit, N, or T optionally substituted with straight or branched C₁₋₅ alkyl) or an L-amino acid comprising —NHC(NH)NH₂, —NHC(O)NH₂, —C(O)NH₂, or —NHC(O)CH₃;
X11 is absent, E, Q, R, Cit, K, D, or N, or a variant thereof; and
X12 is C or a variant thereof.

22. The (cyclic) peptide of any one of embodiments 1 to 21, wherein the peptide has an amino acid sequence according to Formula (I), or a pharmaceutically acceptable salt thereof,

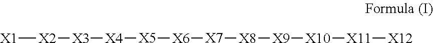

wherein,
   X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Alb, G, Norvaline, Norleucine, d4PyCON, or dhAla;
   X2 is MeF, Me3Py, MeF3CON, MeF3F, Me4Py, or MeY(Me);
   X3 is absent, N, Q, Cit, G, Aib, Hgn, hCit, norCit, LysAc, OrnAc, Ala, or da;
   X4 is L, Cbg, Chg, Cba, Cha, Ahx, Dahp, Cit, I, V, Norleucine, or Norvaline;
   X5 is Hgl, Hgn, Dab, Dap, DabAc, DapAc, R, hArg, E, or D;
   X6 is absent, MeF, MeE, Me3Py, Me4Py, MeF4F, MeF4F, MeF4C, or MeY;
   X7 is W1Me, W1Me7Cl, W1Me7N, W, F, 7-AzaTrp, W7Me, W1Et, W1Me7Br, W1Me7OMe, or W1Me6O7Cl;
   X8 is V, KCOpipzaa, N, Cit, Qglucamine, hCit, K, KAc, Aib, Alb, DapAc, OrnAc, A, T, alT, Norleucine, Norvaline, Hgl, E, Hgn, Q, I, or L;
   X9 is W1Me, W1Me7Cl, W1Me7N, F23dMe, W1Et, W7Me, W, F, or 7-AzaTrp;
   X10 is absent, T, Q, S, Hgn, Alpha-methylserine, hSer, hThr, N, OrnAc, LysAc, Cit, or hCit;
   X11 is absent, E, Hgn, R, hArg, Cit, hCit, Hgl, Orn, D, N, Q, DapAc, OrnAc, DabAc, norCit; and
   X12 is C, hCys, CdMe, C3RMe, C3SMe, Selenocysteine, dc, or Penicillamine.

23. The (cyclic) peptide of any one of embodiments 18-22, wherein
X7 is W1Me or a variant thereof; and
X9 is W1Me or a variant thereof.

24. The (cyclic) peptide of any one of embodiments 18-23, wherein
X7 is W1Me, W1MeCl, W1MeBr, Nal1, Nal2, W1Et, 3Bzf, 3Bzt, F23dC, W1Me7N, or F23dMe;
X8 is V, KCOpipzaa, N, Cit, hCit, KAc, DapAc, OrAc, A, T, alT, Aib, Alb, Qglucamine, Hgl, Q, E, Hgn, or K; and
X9 is W1Me, Nal1, W1Et, Nal21N, 3Bzf, 3Bzt, Nal18N, F23dMe, or F23dC.

25. The (cyclic) peptide of any one of embodiments 1 to 17, wherein the peptide comprises an amino acid sequence according to Formula (I), or a pharmaceutically acceptable salt thereof,

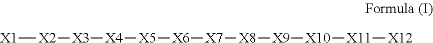

wherein,
   X1 is any amino acid,
   X2 is an amino acid having an aromatic ring or a variant thereof,
   X3 is N,
   X4 is a hydrophobic amino acid or a variant thereof;
   X5 is a hydrophilic amino acid or a variant thereof;
   X6 is a hydrophilic amino acid or amino acid having aromatic ring;
   X7 is W or a variant thereof;
   X8 is V or hydrophilic amino acid or a variant thereof,
   X9 is W or a variant thereof;
   X10 is T or a variant thereof;
   X11 is a hydrophilic amino acid;
   X12 is C or a variant thereof (such as C).

26. The (cyclic) peptide of any one of embodiments 1 to 17, wherein the peptide has an amino acid sequence according to Formula (Ia), or a pharmaceutically acceptable salt thereof,

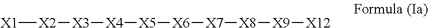

wherein,
   X1 is any amino acid;
   X2 is an amino acid having an aromatic ring or a variant thereof;
   X3 is N or a variant thereof;
   X4 is a hydrophobic amino or a variant thereof,
   X5 is a hydrophilic amino acid or a variant thereof;
   X6 is a hydrophilic amino acid or amino acid having aromatic ring;
   X7 is W or a variant thereof;
   X8 is a hydrophilic amino acid or a variant thereof,
   X9 is W or a variant thereof; and
   X12 is C or a variant thereof.

27. A (cyclic) peptide that has avidity for ephrin type-A receptor 2 (EphA2), wherein the peptide consists of a sequence of Formula (I),

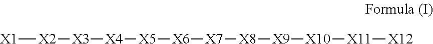

or a pharmaceutically acceptable salt thereof,
wherein
   each of X1, X2, X3, X4, X5, X6, and X8 is independently an amino acid;
   X7 is W1Me or a variant thereof;
   X9 is W1Me or a variant thereof;
   each of X10 and X11 is independently absent or an amino acid; and
   X12 is cysteine (C) or a variant thereof; and,
   optionally, a linker that connects the peptide with a payload molecule.

28. The (cyclic) peptide of any one of embodiments 7-27, wherein the variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆ alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl.

29. The (cyclic) peptide of embodiment 28, wherein the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, and C₁-C₆ alkyl.

30. The (cyclic) peptide of any one of embodiments 7-29, wherein the variant is selected from amino acids that have the similar hydrophilicity or hydrophobicity compared to the reference amino acid.

31. The (cyclic) peptide of any one of embodiments 7-29, wherein the variant is selected from amino acids that have the same functional group as the reference amino acid, and wherein the variant has a different length of a side chain compared to the reference amino acid.

32. The (cyclic) peptide of any one of embodiments 7-31, wherein the variant has a molecular weight that does not vary for more than 14, 28, 30, 45 or 60 g/mol compared to the reference amino acid.

33. A (cyclic) peptide having avidity for ephrin type-A receptor 2 (EphA2), wherein the peptide has an amino acid sequence of Formula (I),

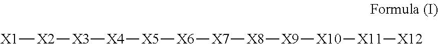

wherein,
   X1 is any D- or L-amino acid;
   X2 has a structure of

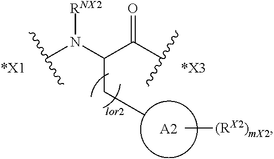

wherein
   ring A2 is phenyl or a 6-membered heteroaryl (e.g., heteroaryl having 1 or 2 N);
   R^{X2} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx2 is 0, 1, 2, or 3;
   mx2 is 0, 1, 2, 3 or 4;
   R^{NX2} is H, C₁-C₆ alkyl, or C₁-C₆ haloalkyl;
   *X1 indicates the point of attachment to X1; and,
   *X3 indicates the point of attachment to X3;
X3 has a structure of

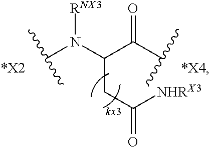

wherein
   kx3 is 0, 1, 2, or 3;
   NX3 is H, C₁-C₆alkyl, or C₁-C₆haloalkyl;
   RX3 is H, C₁-C₆alkyl, C₁-C₆haloalkyl, C₁-C₆hydroxyalkyl, C₁-C₆aminoalkyl, or C₁-C₆heteroalkyl;
   *X2 indicates the point of attachment to X2; and,
   *X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of

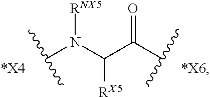

wherein:
   R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
   R^{X5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
   provided that at least one of R^{NX5} and R^{X5} comprises a moiety selected from —OH, —NH₂, and —NH— (e.g., —NH—C(═NH)—NH₂, —CO—NH₂, —NH₂, —COOH, —C(OH)—C₀₋₆ alkyl, —NH—CO—C₁₋₆ alkyl);
   *X4 indicates the point of attachment to X4; and,
   *X6 indicates the point of attachment to X6;
X6 is

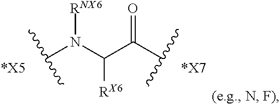

wherein
   R^{NX6} is H, C₁-C₆alkyl, or C₁-C₆haloalkyl;
   R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{XA};
   *X5 indicates the point of attachment to X5; and,
   *X7 indicates the point of attachment to X7:
X7 has a structure of

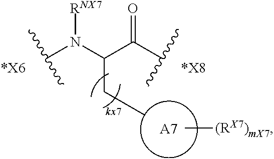

wherein
   R^{NX7} is H, C₁-C₆alkyl, or C₁-C₆haloalkyl;
   ring A7 is an aryl or heteroaryl;
   R^{X7} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx7 is 0, 1, 2, or 3;
   mx7 is 0, 1, 2, 3, 4 or 5;
   *X6 indicates the point of attachment to X6; and,
   *X8 indicates the point of attachment to X8;
X8 is an L-amino acid with —H on the alpha-amino group;
X9 has a structure of

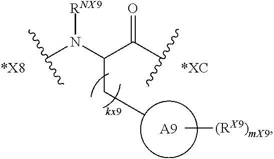

wherein
   R^{NX9} is H, C₁-C₆alkyl, or C₁-C₆haloalkyl;
   ring A9 is an aryl or heteroaryl;
   R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx9 is 0, 1, 2, or 3;
   mx9 is 0, 1, 2, 3, 4, or 5;
   *X8 indicates the point of attachment to X8; and,
   *XC indicates the point of attachment to (i) X10 or (i) when X10 and X11 are absent, X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
   each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
   each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
   each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
   or R^{c} and R^{d} are taken together with the atom to which they are attached to form a heterocycloalkyl optionally substituted with one or more R; and
   each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
   optionally, the peptide is linked to a payload molecule via a linker.

34. The (cyclic) peptide of embodiment 33, wherein ring A7 is a 6-membered aryl or heteroaryl, or a 9- or 10-membered bicyclic aryl or heteroaryl, wherein the 6-, 9- or 10-membered heteroaryl has one heteroatom selected from N, O, and S.

35. The (cyclic) peptide of embodiment 33 or 34, wherein R^{NX7} is H.

36. The (cyclic) peptide of any one of embodiments 33 to 35, wherein each R^{X7} is independently selected from —CH₃, -ethyl, —Cl, and —F, and mx7 is 0, 1, or 2.

37. The (cyclic) peptide of embodiment 33, wherein X7 is W1Me, Nal1, Nal2, W1Et, Nal21N, 3Bzf, 3Bzt, Nal15N, Nal14N, Nal24N, Nal28N, F23dMe, F23dC, W1Me7N, or W1Me7Cl.

38. The (cyclic) peptide of embodiment 37, wherein X7 is W1Me, F23dMe or W1Me7Cl.

39. The (cyclic) peptide of any one of embodiments 33 to 38, wherein X9 is

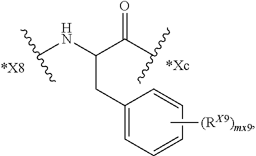

each R^{X9} is independently selected from —OH, CN, NH₂, C₁-C₃alkyl, —Cl, —F, —Br, —CONH₂, and —SO₂F.

40. The (cyclic) peptide of any one of embodiments 33 to 39, wherein

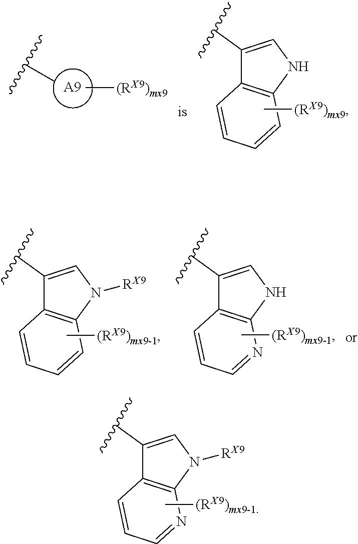

41. The (cyclic) peptide of any one of embodiments 33 to 40, wherein R^{X9} is each independently halogen, —CN, —NO₂, —OH, —OR^{a}, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

42. The (cyclic) peptide of any one of embodiments 33 to 38, wherein X9 is W1Me, W, Nal1, W1Et, Nal21N, 3Bzf, 3Bzt, Nal14N, Nal18N, F23dMe, F23dC, or W1Et.

43. The (cyclic) peptide of embodiment 42, wherein X9 is W1Me or F23dMe.

44. The (cyclic) peptide of any one of embodiments 33 to 43, wherein ring A2 is a 6-membered heteroaryl containing 1 or 2 N.

45. The (cyclic) peptide of any one of embodiments 33 to 44, wherein R^{X5} is C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

46. The (cyclic) peptide of any one of embodiments 27-33, wherein
X7 is W1Me, W1MeCl, W1MeBr, Nal1, Nal2, W1Et, 3Bzf, 3Bzt, F23dC, W1Me7N, or F23dMe;
X8 is V, KCOpipzaa, Hse, N, Cit, hCit, KAc, DapAc, OrnAc, T, alT, Aib, Alb, Qglucamine, Hgl, E, Hgn, MeF, 3Py6NH2, W1Me, A, Q, or K; and
X9 is W1Me, Nal1, W1Et, Nal21N, 3Bzf, 3Bzt, Nal18N, F23dMe, or F23dC.

47. The (cyclic) peptide of embodiment 24 or 46, wherein
X7 is W1Me;
X8 is V; and,
X9 is W1Me.

48. The (cyclic) peptide of any one of embodiments 1-47, wherein the peptide or the pharmaceutically accepted salt thereof has a cyclic structure, wherein the first amino acid (or X1) is covalently linked to the last amino acid (or X12).

49. The (cyclic) peptide of any one of embodiments 1-48, wherein the peptide or the pharmaceutically accepted salt thereof has a cyclic structure having an amino acid in the first residue X1 and a cysteine residue or a variant thereof, and wherein the amino acid in X1 and the cysteine residue or variant thereof form a covalent bond.

50. The (cyclic) peptide of any one of embodiments 1-49, wherein the peptide has a monocyclic structure.

51. The (cyclic) peptide of embodiment 50, wherein the amino acid X1 and a cysteine or a variant thereof form a covalent bond.

52. The (cyclic) peptide of any one of embodiments 1-51, wherein the peptide has a structure of Formula (I-1),

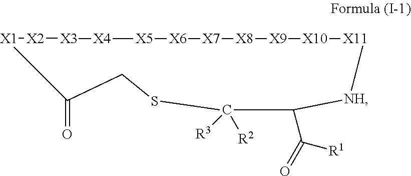

wherein
   R¹ is selected from the group consisting of NH₂ and OH;
   R² is selected from the group consisting of H or C₁₋₃ alkyl;
   R³ is selected from the group consisting of H or C₁₋₃ alkyl;
   wherein X1 to X11 have the definitions described in Formula (I).

53. The (cyclic) peptide of embodiment 52, or a pharmaceutically acceptable salt thereof, wherein the peptide of Formula (I-1) has a structure of Formula (I-2),

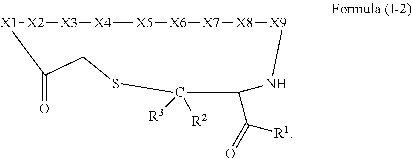

54. The (cyclic) peptide of any one of embodiments 1-53, wherein the peptide or the salt thereof comprises an amino acid sequence that is at least 95% identical to a sequence selected from SEQ ID NOs: 1-171, or a sequence with up to 1, 2, 3, 4, or 5 substitutions by a conserved variant compared to any one of the sequences selected from SEQ ID NOs: 1-171.

55. The (cyclic) peptide of any one of embodiments 1-54, wherein the peptide or the salt thereof (a) consists of an amino acid sequence selected from SEQ ID NOs: 1-171; or (b) is not SEQ ID NO: 1.

56. The (cyclic) peptide of embodiment 55, wherein the peptide consists of an amino acid sequence selected from SEQ ID NOs: 1-122, 159-163, and 165-171, and the peptide has a cyclic structure having a cysteine residue or a variant thereof at the 12^{th} residue, and wherein the amino acid at X1 (e.g., a chloroacetylated amino acid) and the cysteine residue or a variant thereof at the 12 h residue form a covalent bond (e.g., by reacting a chloroacetyl group in the amino acid of X1 with the cysteine residue or a variant thereof).

57. The (cyclic) peptide of embodiment 55, wherein the peptide consists of an amino acid sequence selected from SEQ ID NOs: 123-149 and 164, and the peptide has a cyclic structure having a cysteine residue or a variant thereof at the 100 residue, and wherein the amino acid at X1 (e.g., a chloroacetylated amino acid) and the cysteine residue or a variant thereof at the 10 h residue form a covalent bond.

58. The (cyclic) peptide of any one of embodiments 1-57, wherein the peptide has a binding affinity to a human EphA2 of at most 100 nM as determined by K_{d} in surface plasmon resonance (SPR) analysis.

59. The (cyclic) peptide of embodiment 58, wherein the peptide has a binding affinity to a human EphA2 of at most 1 nM as determined by K_{d} in surface plasmon resonance (SPR) analysis.

60. The (cyclic) peptide of any one of embodiments 1-59, wherein the peptide binds to a ligand-binding domain (LBD) domain of the EphA2.

61. The (cyclic) peptide of any one of embodiments 1-60, wherein the peptide interacts with a human EphA2 at one or more amino acid residues selected from Asp53, Met55, Asn57, Met59, Met66, Thr101, Arg103, Phe156, Glu157, Arg159, Val161, Val189, and Ala190.

62. The (cyclic) peptide of any one of embodiments 1-61, wherein the peptide interacts with a human EphA2 at Asp53 and Glu157.

63. The (cyclic) peptide of any one of embodiments 1-62, wherein the peptide has a plasma half-life (T_{1/2}) of at least 50, 100, 150, 200, 250, 300, 350, 400, 450, or 500 minutes as determined in vitro in human plasma at 37° C.

64. The (cyclic) peptide of embodiment 63, wherein the peptide has a plasma half-life (T_{1/2}) of at least 250 minutes as determined in vitro in human plasma at 37° C.

65. A (cyclic) peptide of any one of embodiments 1-64, covalently linked to a linker that connects the peptide to a payload molecule.

66. The (cyclic) peptide of embodiment 65, wherein the linker is attached to the peptide via a non-terminal amino acid residue of the peptide.

67. The (cyclic) peptide of embodiment 66, wherein the linker is attached to the 5^{th} amino acid residue or X5.

68. The (cyclic) peptide of embodiment 66, wherein the linker is attached to the 8^{th} amino acid residue or X8.

69. The (cyclic) peptide of embodiment 66, wherein the linker is attached to the 11^{th} amino acid residue or X11.

70. The (cyclic) peptide of any one of embodiments 65 to 69, wherein linker is attached to a lysine of the peptide.

71. The (cyclic) peptide of any one of embodiments 65 to 70, wherein the linker is attached to the peptide via the N terminus of the peptide.

72. The (cyclic) peptide of any one of embodiments 65 to 70, wherein the linker is attached to the peptide via the C terminus of the peptide.

73. The (cyclic) peptide of any one of embodiments 65 to 72, wherein the linker is a bond.

74. The (cyclic) peptide of any one of embodiments 65 to 72, wherein the linker comprises 3 to 30 intervening atoms between the payload molecule and the peptide.

75. The (cyclic) peptide of any one of embodiments 65 to 72, wherein the linker comprises 6 to 18 intervening atoms between the payload molecule and the peptide.

76. The (cyclic) peptide of embodiment 74 or 75, wherein the intervening atoms comprise 1 to 6 nitrogen and 0 to 4 oxygen.

77. The (cyclic) peptide of any one of embodiments 65-72 and 74-76, wherein the linker comprises one or more amino acid residues.

78. The (cyclic) peptide of embodiment 77, wherein the linker comprises one or more amino acids chosen from a lysine residue, an alanine residue, or a phenylalanine residue.

79. The (cyclic) peptide of any one of embodiments 65-72 and 74-78, wherein the linker comprises one or more structures selected from AEEA, AEEP, AEEEP, and AEEEEP.

80. The (cyclic) peptide of any one of embodiments 65-72, wherein the linker has a structure of Formula (II-1)

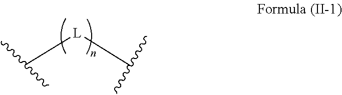

wherein each L is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂—, or —N(R^{L})₂—(C₁-C₃₀ alkylene)-; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20.

81. The (cyclic) peptide of embodiment 80, wherein the linker comprises a structure of Formula (II-1a),

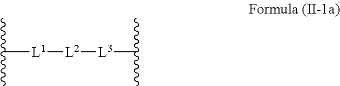

wherein each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}, —NR^{L}S(═O)₂—, —S(═O)₂NR^{L}, —C(═O)NR^{L}S(═O)₂—, or —S(═O)₂NR^{L}C(═O)—; and
L² is absent, substituted or unsubstituted C₁-C₃₀ alkylene, or substituted or unsubstituted C₁-C₃₀ heteroalkylene.

82. The (cyclic) peptide of embodiment 81, wherein L¹ is —NH—.

83. The (cyclic) peptide of embodiment 81 or 82, wherein L² is substituted or unsubstituted C₁-C₃₀ alkylene, or substituted or unsubstituted C₁-C₃₀ heteroalkylene.

84. The (cyclic) peptide of embodiment 81 or 82, wherein L² is substituted or unsubstituted C₁-C₁₈ alkylene, or substituted or unsubstituted C₁-C₁₈ heteroalkylene.

85. The (cyclic) peptide of any one of embodiments 81 to 84, wherein L² is optionally substituted with one or more substituents selected from —OH, —SH, oxo, amino, C₁-C₆ alkyl, C₁-C₈ hydroxyalkyl, C₁-C₆ haloalkyl, C₁-C₆ aminoalkyl, —C(═O)OR^{L}, —OC(═O)R^{L}—, —OC(═O)OR^{L}—, —C(═O)N(R^{L})₂, —NR^{L}C(═O)R^{L}, —OC(═O)N(R^{L})₂, and —NR^{L}C(═O)OR^{L}; and the C₁-C₆ alkyl is further optionally substituted with one or more substituents chosen from —OH, —SH, oxo, amino, C₆-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

86. The (cyclic) peptide of any one of embodiments 81-85, wherein L³ is —NH—.

87. The (cyclic) peptide of embodiment 81, wherein the linker has a structure of

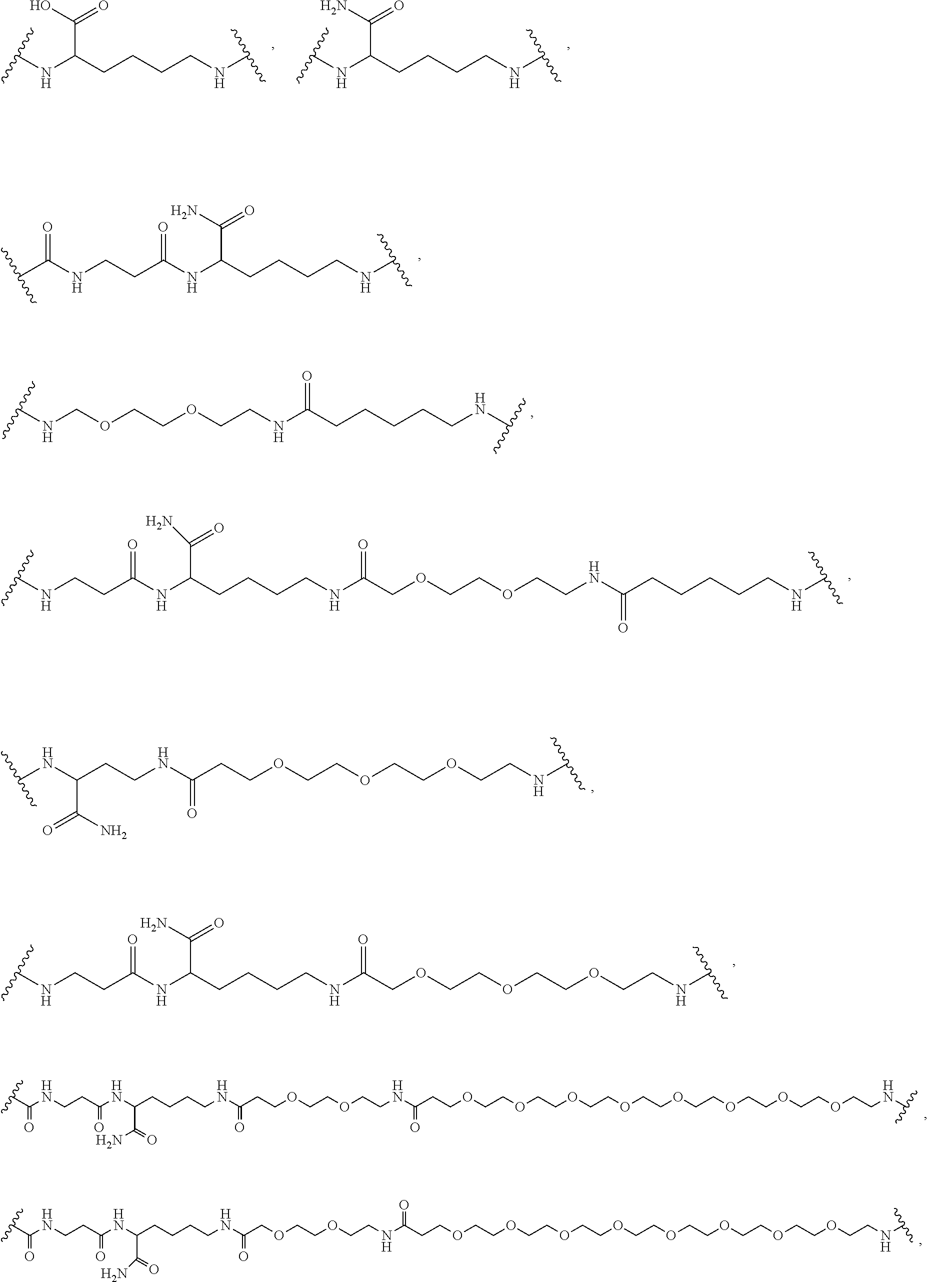

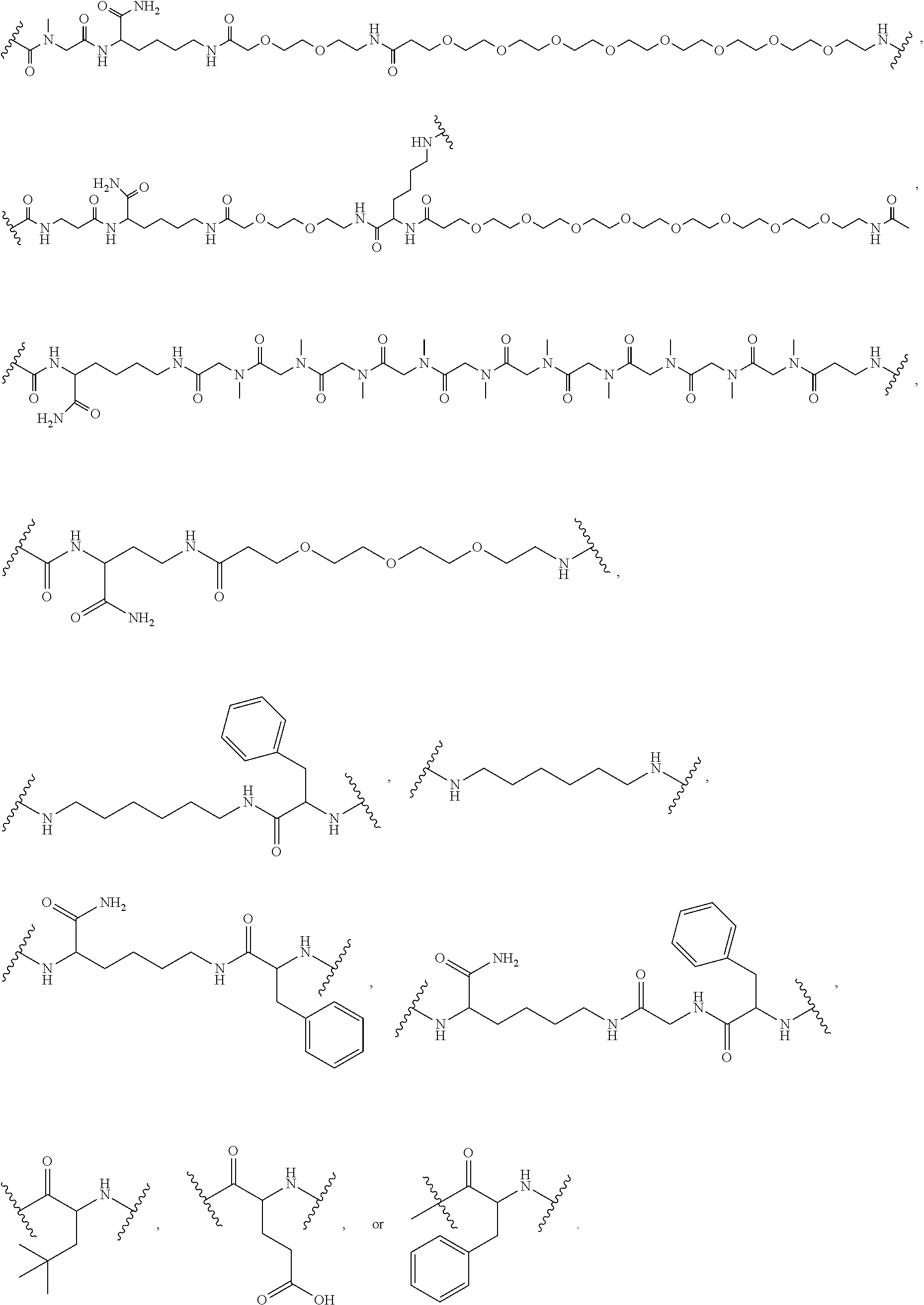

88. The (cyclic) peptide of embodiment 81, wherein the linker has a structure of

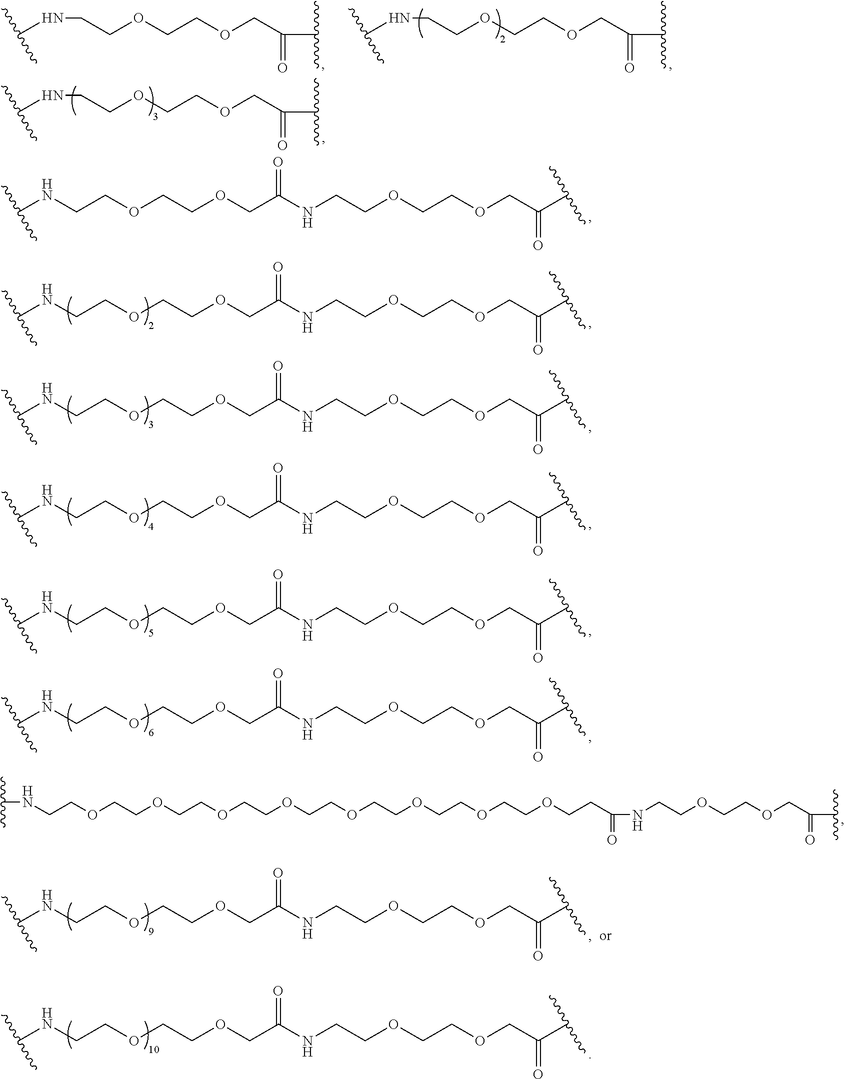

89. The (cyclic) peptide of any one of embodiments 1-88, wherein the peptide is a peptide of Formula (I) and wherein, when the peptide is bound to the human EphA2, amino acid residue X7 is located less than 10 Å from the Phe156 of the human EphA2.

90. The (cyclic) peptide of embodiment 89, wherein amino acid residue X7 is located less than 6 Å from the Phe156.

91. The (cyclic) peptide of embodiment 89, wherein amino acid residue X7 is located less than 4 Å from the Phe156.

92. The (cyclic) peptide of any one of embodiments 1-91, wherein the peptide is a peptide of Formula (I) and wherein, when the peptide is bound to the human EphA2, amino acid residue X9 is located less than 10 Å from the Phe156 of the human EphA2.

93. The (cyclic) peptide of embodiment 92, wherein amino acid residue X9 is located less than 6 Å from the Phe156.

94. The (cyclic) peptide of embodiment 93, wherein amino acid residue X9 is located less than 4 Å from the Phe156.

95. The (cyclic) peptide of any one of embodiments 1-94, wherein the peptide is a peptide of Formula (I) and wherein, when the peptide is bound to the human EphA2, amino acid residue X8 is located less than 10 Å from the Phe156 of the human EphA2.

96. The (cyclic) peptide of any one of embodiments 89 to 95, wherein the human EphA2 comprises a sequence of SEQ ID NO: 276 or SEQ ID NO: 277.

97. A (cyclic) peptide that has avidity for ephrin type-A receptor 2 (EphA2), and competes for binding to a human EphA2 with a peptide that has an amino acid sequence including deletion, substitution, or addition of one or several amino acids in the amino acid of SEQ ID NO: 1:

(SEQ ID NO: 1)
da-MeF-N-L-Hgl-MeF-W1Me-V-W1Me-T-E-C

or a pharmaceutically acceptable salt thereof.

98. A (cyclic) peptide that has avidity for ephrin type-A receptor 2 (EphA2), wherein the peptide competes for binding to a human EphA2 with a peptide that has a structure of Formula (I), or a pharmaceutically acceptable salt thereof,

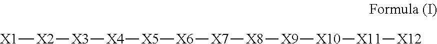

wherein,
   X1 is an amino acid;
   X2 is an amino acid comprising an aromatic ring, an N-methylated amino acid thereof, or a variant thereof;
   X3 is a hydrophilic amino acid (e.g. N, Q, Cit, K or a variant thereof), glycine (G), Alanine (A) or a variant thereof (e.g., da, 2-Aminoisobutyric acid (Aib));
   X4 is a hydrophobic amino acid (e.g., leucine (L)), a hydrophilic amino acid (e.g., citrulline (Cit), or a variant thereof;
   X5 is a hydrophilic amino acid, or a variant thereof;
   X6 is a hydrophilic amino acid, an amino acid comprising an aromatic ring, or an N-methylated amino acid thereof;
   X7 is an amino acid comprising an aromatic ring (e.g., W, F. or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof.

99. A (cyclic) peptide that has avidity for ephrin type-A receptor 2 (EphA2), wherein the peptide consists of a sequence of Formula (I).

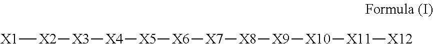

or a pharmaceutically acceptable salt thereof,
   wherein
   each of X1, X2, X3, X4, X5, X6, and X8 is independently an amino acid;
   X7 is W1Me or a variant thereof;
   X9 is W1Me or a variant thereof;
   each of X10 and X11 is independently absent or an amino acid; and
   X12 is cysteine (C) or a variant thereof; and,
   wherein the peptide is optionally linked to a payload molecule through a linker.

100. The (cyclic) peptide of any one of embodiments 97 to 99, wherein the peptide competes for binding to a human EphA2 at one or more amino acid residues selected from Asp53, Met55, Asn57, Met59, Met66, Thr101, Arg103, Phe156, Glu157, Arg159, Val161, Val189, and Ala190.

101. The (cyclic) peptide of embodiment 100, wherein the peptide competes for binding to human EphA2 at one or more amino acid residues selected from Asp53, Phe156, and Glu157.

102. The (cyclic) peptide of any one of embodiments 97 to 101, wherein the human EphA2 comprises a sequence of SEQ ID NO: 276 or SEQ ID NO: 277.

103. A pharmaceutical composition comprising the peptide or salt thereof according to any one of embodiments 1-102, and a pharmaceutically acceptable excipient or carrier.

104. A conjugate comprising the peptide or salt thereof according to any one of the preceding embodiments and a substance, wherein the substance is selected from the group consisting of: a nucleotide, a small molecule, a medium sized molecule (e.g., with a M.W. of about 1,000-2,500 Da), a large sized molecule (e.g., with a M.W. of >2,500 Da), a polymer compound, a protein, a peptide, a tag, a biological fragment, a carrier including pharmaceutical compound, or a combination thereof.

105. A method of treating a disease or disorder characterized by overexpression of EphA2, comprising administering to the subject the peptide or salt thereof according to any one of embodiments 1-102, the conjugate of embodiment 103, or the pharmaceutical composition of embodiment 104.

106. The method of embodiment 105, wherein the disease or disorder is cancer.

107. The method of embodiment 106, wherein the cancer is selected from glioblastoma, prostate cancer, lung cancer, breast cancer, gastric cancer, ovarian cancer, gladder cancer, colon cancer, esophageal cancer, multiple myeloma and fibrosarcoma.

108. The method of claim 106, wherein the cancer is non-small cell lung carcinomas (NSCLC).

109. The method of embodiment 106, wherein the cancer is triple negative breast cancer.

110. A kit, tester, or composition for determining the expression level of EphA2 in a sample, wherein the kit, tester, or composition comprises the peptide or salt thereof according to any one of embodiments 1-102, the conjugate of embodiment 103, or the pharmaceutical composition of embodiment 104.

111. The kit, tester, or composition of embodiment 110, adapted for use in a method of diagnosing disease or disorder characterized by an overexpression or a decreased expression of EphA2.

112. The kit, tester, or composition of embodiment 110 or 111, wherein the sample is from a subject having a disease or disorder characterized by an overexpression or a decreased expression of EphA2.

113. Use of the peptide or salt thereof according to any one of the preceding claims in the manufacture of a medicament for diagnosing and/or treating a disease or disorder characterized by an overexpression or a decreased expression of EphA2.

114. The peptide or salt thereof according to any one of the preceding embodiments, for use in diagnosing and/or treating a disease or disorder characterized by an overexpression or a decreased expression of EphA2.

In some embodiments, the peptide of the present technology is an isolated peptide.

In some embodiments, the peptide of the present technology is a purified peptide.

In all aspects of this disclosure, however, the substance or payload molecule excludes any radioactive materials. Examples of the substance that are excluded are: radioisotope, radiopharmaceutical, or any compound having radioactive component. In all aspects of this disclosure, the substance further excludes any chelators for radioisotope conjugation, regardless of whether the chelator is connected to the peptide directly or via a linker. Accordingly, a complex, conjugate or PDC described herein does not encompass any compound containing a chelator for radioisotope conjugation, and does not encompass a radioisotope.

Since the peptide of the present technology has the ability to bind to the EphA2, it is possible for the peptide to target and transport compounds having pharmacological actions to the EphA2, such as low molecular weight compounds, middle molecular weight compounds, high molecular weight compounds, peptides, proteins, antibodies, and nucleic acids.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 illustrates exemplary PDC of the present disclosure, wherein [symbol] represents the linker, and the peptide covalently connected to the payload represented by rounded square shown in the circle.

FIG. 2 illustrates general synthetic scheme A of the macrocyclic peptide I of this invention.

FIG. 3 illustrates general synthetic scheme B of the macrocyclic peptide II of this invention.

FIG. 4 illustrates general synthetic scheme C of the macrocyclic peptide Ill of this invention.

FIG. 5 illustrates synthetic scheme of PDC_EphA2-00007196-C004 (SEQ ID NO: 224).

FIG. 6 illustrates synthetic scheme of PDC_EphA2-00007196-C010 (SEQ ID NO: 231). FIG. 6 discloses “bA-MeG-MeG-MeG-MeG-MeG-MeG-MeG-MeG-MeG-MeG” as SEQ ID NO: 279.

### DETAILED DESCRIPTION

It should be understood that both the general descriptions and the detailed description below are merely illustrative and descriptive and do not limit the present technology of the present application. In the present specification, the use of the singular form includes the plural form unless otherwise specified. In the present specification, the use of “or (or)” means “and/or (and/or)” unless otherwise stated. Furthermore, terms such as “element” or “component” encompass both an element and a component including one unit and an element and a component including two or more subunits unless when otherwise specified.

The headings used in the present specification are for structural purposes only and must not be construed as limiting the subject matter described. All of the documents or parts of the documents cited in the present application including but not limited to patents, patent applications, articles, books, and papers are expressly incorporated by reference in part or entirety from among the documents discussed in the present specification.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) are to be construed in an open, inclusive sense, that is, as “including, but not limited to.” The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amino” refers to the —NH₂ radical.

“Cyano” refers to the —CN radical.

“Nitro” refers to the —NO₂ radical.

“Oxo” refers to the ═O radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Hydroxyamino” refers to the —NH—OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂—NO₂, or —C≡CH. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula-N(Rₐ)₂ where Rₐ is an alkyl radical as defined, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl; —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle [1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl; heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

As used herein, a “heteroalkylene” refers to divalent heteroalkyl group. Examples of such heteroalkylene are, for example, —CH₂—O—CH₂—, —CH₂—N(alkyl)-CH₂—, —CH₂—N(aryl)-CH₂—, —OCH—CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one hetero ring atom, e.g., a heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1˜4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease.

In certain embodiments, “treating” includes the concepts of “alleviating,” which refers to lessening the frequency of occurrence or recurrence, or the severity, of any symptoms or other ill effects related to a disorder and/or the associated side effects. In certain embodiments, the term “treating” also encompasses the concept of “managing” which refers to reducing the severity of a particular disease or disorder in a patient or delaying its recurrence, e.g., lengthening the period of remission in a patient who had suffered from the disease.

In certain embodiments, the term “prevent” or “preventing” as related to a disease or disorder can refer to a compound that in a statistical sample, reduces the occurrences of the disorder or condition in the treated sample relative to an untreated control sample, or delays the onset or reduces the severity of one or more symptoms of the disorder or condition relative to the untreated control sample.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

For the purpose of the disclosure, one event of “substitution” of an amino acid or an amino sequence is not considered two separate events of one deletion plus one addition. Thus, for the avoidance of doubt, as an example, a sequence change of “up to two deletion, substitution and/or addition” includes one deletion and one substitution, one deletion and one addition (at a different position), one substitution and one addition, one deletion only, one substitution only, one addition only, two deletions, two substitutions, two additions, etc. The deletion, addition, or substitution position may be at one or both ends of the peptide, or in the middle of the peptide.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH (alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitutions, i.e. replacement of one hydrogen up to replacement of all hydrogens by substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

The term “peptide” as used herein refers to a compound that includes two or more amino acids. A peptide described herein can comprise one or more unnatural amino acids. The term “peptide” also encompasses peptide mimetics. In the present disclosure, the term “amino acid” is used in its broadest meaning and it embraces not only natural amino acids but also derivatives thereof and artificial amino acids. For example, the term “amino acid” encompasses unnatural amino acids.

As used herein, the term “unnatural amino acid” refers to an amino acid other than the 20 canonical amino acids. The 20 canonical amino acids refer to alanine (ala or A), arginine (arg or R), asparagine (asn or N), aspartic acid (asp or D), cysteine (cys or C), glutamine (gin or Q), glutamic acid (glu or E), glycine (gly or G), histidine (his or H), isoleucine (ile or 1), leucine (leu or L), lysine (lys or K), methionine (met or M), phenylalanine (phe or F), proline (pro or P), serine (ser or S), threonine (thr or T), tryptophan (trp or W), tyrosine (tyr or Y), and valine (val or V).

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain. For example, polypeptide chains can be linked by one or more disulfide bonds or associated by other means.

The term “peptide mimetic” or “mimetic” refers to biologically active compounds that mimic the biological activity of a peptide or a protein but are no longer entirely peptidic in chemical nature, e.g., they can contain non-peptide bonds (that are, bonds other than amide bonds between amino acids). As used herein, the term peptide mimetic is used in a broader sense to include molecules that are no longer completely peptidic in nature, such as pseudo-peptides, semi-peptides and peptoids. Whether completely or partially non-peptide, peptide mimetics described herein can provide a spatial arrangement of reactive chemical moieties that closely resemble the three-dimensional arrangement of active groups in the subject amino acid sequence or subject molecule on which the peptide mimetic is based. As a result of this similar active-site geometry, the peptide mimetic can have effects on biological systems that are similar to the biological activity of the subject entity.

In some embodiments, the peptide mimetics are substantially similar in both three-dimensional shape and biological activity to the subject amino acid sequence or subject molecule on which the peptide mimetic is based. An example is described in the paper “Tritiated D-ala1-Peptide T Binding”, Smith C. S. et al., Drug Development Res., 15, pp. 371-379 (1988). A second method is altering cyclic structure for stability, such as N to C interchain imides and lactams (Ede et al. in Smith and Rivier (Eds.) “Peptides: Chemistry and Biology”, Escom, Leiden (1991), pp. 268-270). An example of this is provided in conformationally restricted thymopentin-like compounds, such as those disclosed in U.S. Pat. No. 4,457,489. A third method is to substitute peptide bonds in the subject entity by pseudopeptide bonds that confer resistance to proteolysis.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ (or C₁₋ₓ) includes C₁-C₂, C₁-C₃ . . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

The term “cyclized” or “cyclization” as used herein means that two amino acids apart from each other by at least one amino acid bind directly or bind indirectly to each other in one peptide to form a cyclic structure in the molecule. In some cases, the two amino acids bind via a linker or the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

The term “therapeutically effective amount” as used herein to refer to an amount effective at the dosage to achieve the desired therapeutic result. A therapeutically effective amount of a composition may vary depending on factors such as the individual's condition (e.g., age, sex, and weight), the conjugate, and the method of administration (e.g., oral or parenteral).

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination. For example, a conjugate of this disclosure can comprise any peptide ligand described herein (e.g., a peptide ligand of Formula (I), (I-1), (I-2), (I-3), (I-4), (I-5), (Ia), (Ib), or (Ic), or Table 1), any payload molecule described herein, optionally a linker described herein (e.g., a linker of Formula (II-1), (II-1a), (II-1b), or (II-2), and optionally a payload molecule described herein. For another example, a peptide of Formula (I) (or any other Formulae such as (III-1) and (III-2) can comprise X1 to X12 amino acids as described herein, and any combinations of the embodiments of amino acids are encompassed by this disclosure (even though, in some cases, they are described in the context of separate embodiments).

Unless special definitions are given, the terminology used in relation to analytical chemistry, synthetic organic chemistry, and medical chemistry and pharmaceutical chemistry described in the present specification, as well as their procedures and techniques, are well known and commonly used in the field of the present art. Standard techniques may be used for chemical synthesis and chemical analysis. Those defined from among such techniques and procedures can be found in, for example, “K. J. Jensen, P. T. Shelton, S. L. Pedersen, Peptide Synthesis and Applications, 2nd Edition, Springer, 2013” and the like, and these are incorporated into the present specification by reference for all purposes. All patents, applications, published applications, and other publications, and other data referred to throughout the entire disclosure, when permitted, are incorporated into the present specification by reference.

### Abbreviations:

Unless otherwise stated in the present specification, the following abbreviations are used according to the following meanings:
Alloc allyloxycarbonyl
aq. aqueous
Biotin-OSu Biotin N-hydroxysuccinimide ester (CAS 35013-72-0)
Boc tert-butyloxycarbonyl
ClAcOH chloroacetic acid
ClAcOSu N-succinimidyl 2-chloroacetate (CAS 27243-15-8)
DCM dichloromethane (CAS 75-09-2)
DIC N,N′-diisopropylcarbodiimide (CAS 693-13-0)
DIPEA, DIEA N,N-diisopropylethylamine (CAS 7087-68-5)
DMF N,N-dimethylformamide (CAS 68-12-2)
DODT 2,2′-(ethylenedioxy)diethanethiol (CAS 14970-87-7)
EDCI-HCl N-(3-dimethylaminopropyl)-N′-ethylcarbodiimide hydrochloride (CAS 25952-53-8)
eq equivalent
Et ethyl
Et3N, TEA triethylamine (CAS 121-44-8)
Fmoc 9-fluorenylmethoxycarbonyl
hr hour
HATU 1-[bis(dimethylamino)methylene]-1H-1,2,3-triazolo[4,5-b]pyridinium 3-oxide hexafluorophosphate (CAS 148893-10-1)
HOSu N-hydroxysuccinimide (CAS 6066-82-6)
iPrOH/IPA isopropanol
M molar
min minutes
NHS N-hydroxysuccinimide (CAS 6066-82-6)
NMP N-methylpyrrolidone (CAS 872-50-4)
Pd(PPh3)4 tetrakis(triphenylphosphine)palladium(0) (CAS: 14221-01-3)
Ph phenyl
rpm rotations per minute
rt room temperature
SPPS solid phase peptide synthesis
Su succinimidyl
SulfoCy5 sulfo Cyanine5
tert tertiary
TFA trifluoroacetic acid (CAS 76-05-1)
TIS triisopropylsilane (CAS 6485-79-6)
TR retention time
Trt trityl

### Peptide:

In one aspect, the disclosure relates to a peptide (e.g., a binding peptide) that has avidity for ephrin type-A receptor 2 (EphA2). The EphA2 can be a mammalian EphA2. The EphA2 can be a human EphA2. The EphA2 can be a wild-type or mutated EphA2. In some embodiments, the conjugate of the disclosure comprises two or more peptides, which can be the same or different. The peptide can be linear or cyclic. In some embodiments, the peptide is monocyclic. The peptide can comprise any suitable number of amino acid residues. In some embodiments, the peptide comprises from 5 to 50, 6 to 40, 7 to 30, 8 to 25, 12 to 25, or 9 to 20 amino acid residues. In some embodiments, the peptide comprises from 5 to 14 amino acid residues. In some embodiments, the peptide comprises from 7 to 12 amino acid residues. In some embodiments, the peptide comprises from 8 to 12 amino acid residues. In some embodiments, the peptide comprises from 8 to 10 amino acid residues. In some embodiments, the peptide comprises from 7 to 13 amino acid residues. In some embodiments, the peptide comprises from 12 to 15 amino acid residues. In some embodiments, the peptide comprises from 13 to 14 amino acid residues. In some embodiments, the peptide comprises 6 amino acid residues. In some embodiments, the peptide comprises 7 amino acid residues. In some embodiments, the peptide comprises 8 amino acid residues. In some embodiments, the peptide comprises 9 amino acid residues. In some embodiments, the peptide comprises 10 amino acid residues. In some embodiments, the peptide comprises 11 amino acid residues. In some embodiments, the peptide comprises 12 amino acid residues. In some embodiments, the peptide comprises 13 amino acid residues. In some embodiments, the peptide comprises 14 amino acid residues. In some embodiments, the peptide comprises 15 amino acid residues. In some embodiments, the peptide comprises 16 amino acid residues. In some embodiments, the peptide consists of 6 amino acid residues. In some embodiments, the peptide consists of 7 amino acid residues. In some embodiments, the peptide consists of 8 amino acid residues. In some embodiments, the peptide consists of 9 amino acid residues. In some embodiments, the peptide consists of 10 amino acid residues. In some embodiments, the peptide consists of 11 amino acid residues. In some embodiments, the peptide consists of 12 amino acid residues. In some embodiments, the peptide consists of 13 amino acid residues. In some embodiments, the peptide consists of 14 amino acid residues. In some embodiments, the peptide consists of 15 amino acid residues. In some embodiments, the peptide consists of 16 amino acid residues. In some embodiments, the conjugate comprises a monocyclic peptide of 6, 7, 8, 9, 10, 11, 12, 13, 14, or 15 amino acid residues. A peptide described herein can be a binding peptide that binds to EphA2. In some embodiments, the binding peptide consists of 6 to 20 amino acid residues. In some embodiments, the binding peptide consists of 7 to 12 amino acid residues. In some embodiments, the binding peptide consists of 10 to 12 amino acid residues. In some embodiments, the binding peptide consists of 8 to 12 amino acid residues. In some embodiments, the binding peptide is monocyclic. In some embodiments, the peptide of the present technology is an isolated peptide. In some embodiments, the peptide of the present technology is a purified peptide.

In one aspect, described herein is a peptide (e.g., a cyclic peptide) that has avidity for ephrin type-A receptor 2 (EphA2), wherein the peptide comprises an amino acid sequence including deletion, substitution, and/or addition of one or several (e.g., 1-6) amino acids in the amino acid sequence of SEQ ID NO: 1:

(SEQ ID NO: 1)
da-MeF-N-L-Hgl-MeF-W1Me-V-W1Me-T-E-C

or a pharmaceutically acceptable salt thereof. Optionally, the (cyclic) peptide consists of 10 to 12 amino acid residues.

In some embodiments, the (cyclic) peptide consists of 10 to 12 amino acid residues.

In some embodiments, the peptide comprises an amino acid sequence including a total of at most 6 deletion, substitution, and/or addition of one or several amino acids in the amino acid of SEQ ID NO: 1. In some embodiments, the peptide comprises an amino acid sequence including a total of at most 5 deletion, substitution, and/or addition of one or several amino acids in the amino acid of SEQ ID NO: 1. In some embodiments, the peptide comprises an amino acid sequence including a total of at most 4 deletion, substitution, and/or addition of one or several amino acids in the amino acid of SEQ ID NO: 1. In some embodiments, the peptide comprises an amino acid sequence including a total of at most 3 deletion, substitution, and/or addition of one or several amino acids in the amino acid of SEQ ID NO: 1. In some embodiments, the peptide comprises an amino acid sequence including a total of at most 2 deletion, substitution, and/or addition of one or several amino acids in the amino acid of SEQ ID NO: 1. In some embodiments, the peptide comprises an amino acid sequence including a total of at most 1 deletion, substitution, and/or addition of one or several amino acids in the amino acid of SEQ ID NO: 1. In some embodiments, the amino acid substitution is a conservative amino acid substitution. The deletion, addition, or substitution position may be at one or both ends of the peptide, or in the middle of the peptide.

In some embodiments, the peptide comprises an amino acid sequence wherein 1-5 amino acids selected the group consisting of the 3^{rd} N, 4^{th} L, 5^{th} Hgl, 6^{th} MeF, 10^{th} T and 11^{th} E of SEQ ID NO: 1, is deleted in the peptide. In some embodiments, the peptide comprises an amino acid sequence wherein 1, 2, 3, 4 or 5 amino acids selected the group consisting of the 3^{rd} N, 4^{th} L, 5^{th} Hgl, 6^{th} MeF, 10^{th} T and 11^{th} E of SEQ ID NO: 1, is deleted in the peptide. In some embodiments, the 3^{rd} N is deleted. In some embodiments, the 4^{th} L is deleted. In some embodiments, the 5^{th} Hgl is deleted. In some embodiments, the 6^{th} MeF is deleted. In some embodiments, the 11^{th} E is deleted. In some embodiments, the peptide comprises an amino acid sequence wherein 1-5 amino acids selected from the group consisting of amino acids at the 3^{rd}, 4^{th}, 5^{th}, 6^{th}, 10^{th}, and 11^{th} position of SEQ ID NO: 1, is deleted in the peptide. In some embodiments, the peptide comprises an amino acid sequence wherein 1, 2, 3, 4 or 5 amino acids selected the group consisting of amino acids at the 3^{rd}, 4^{th}, 5^{th}, 6^{th}, 10^{th}, and 11^{th} position of SEQ ID NO: 1, is deleted in the peptide. In some embodiments, the 3^{rd} amino acid is deleted. In some embodiments, the 4^{th} amino acid is deleted. In some embodiments, the 5^{th} amino acid is deleted. In some embodiments, the 6^{th} amino acid is deleted. In some embodiments, the 10^{th} amino acid is deleted. In some embodiments, the 11^{th} amino acid is deleted. In certain embodiments, the peptide has deletions of 1-5 amino acids of SEQ ID NO: 1, and no additional residue addition. In certain embodiments, the peptide has deletions of 1-5 amino acids of SEQ ID NO: 1, and no additional residue substitutions. In certain embodiments, the peptide has deletions of 1-5 amino acids of SEQ ID NO: 1, and no additional residue addition or substitution. In certain embodiments, the peptide has deletions of 1-5 amino acid residues of SEQ ID NO: 1, and no residue addition. In certain embodiments, the peptide has deletions of 1-5 amino acid residues of SEQ ID NO: 1, and no residue substitution. In certain embodiments, the peptide has deletions of 1-5 amino acid residues of SEQ ID NO: 1, and no residue addition and substitution.

In one aspect, described herein is a peptide (e.g., cyclic peptide) that has avidity for ephrin type-A receptor 2 (EphA2), wherein the peptide has an amino acid sequence according to Formula (I), or a pharmaceutically acceptable salt thereof,

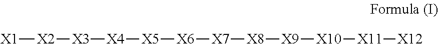

X1 is an amino acid;
X2 is an amino acid comprising an aromatic ring, an N-methylated amino acid thereof, or a variant thereof;
X3 is a hydrophilic amino acid (e.g. N, Q, Cit, K or a variant thereof), glycine (G), Alanine (A) or a variant thereof (e.g., da, 2-Aminoisobutyric acid (Aib);
X4 is a hydrophobic amino acid (e.g., leucine (L), a hydrophilic amino acid (e.g., citrulline (Cit), or a variant thereof;
X5 is a hydrophilic amino acid, or a variant thereof;
X6 is a hydrophilic amino acid, an amino acid comprising an aromatic ring, or an N-methylated amino acid thereof;
X7 is an amino acid comprising an aromatic ring (e.g., W, F, or a variant thereof);
X8 is a hydrophobic amino acid, a hydrophilic amino acid, an N-methylated amino acid, or a variant thereof;
X9 is an amino acid comprising an aromatic ring (e.g., W or a variant thereof);
X10 is absent or a hydrophilic amino acid (e.g., Threonine (T) or a variant thereof);
X11 is absent or a hydrophilic amino acid; and
X12 is cysteine (C) or a variant thereof.

In certain embodiments, X3 is a hydrophilic amino acid. In certain embodiments, X3 is an amino acid comprising an electrically charged side chain (e.g., K or a variant thereof), an amino acid comprising a polar uncharged side chain (e.g., Q, Cit, N, or a variant thereof), or G, A or variant thereof. In certain embodiments, X4 is a hydrophobic amino acid. In certain embodiments, X4 is an amino acid comprising a hydrophobic side chain (e.g., L), an amino acid comprising a polar uncharged side chain (e.g., Cit or a variant thereof). In certain embodiments, X5 is a hydrophilic amino acid. In certain embodiments, X5 is an amino acid comprising an electrically charged side chain (e.g., E, Hgl, D, or a variant thereof), or an amino acid comprising a polar uncharged side chain (e.g., Q, Cit, Hgn, N, or a variant thereof). In certain embodiments, X6 is a hydrophilic amino acid. In certain embodiments, X6 is an amino acid comprising an electrically charged side chain (e.g., E, Hgl, D, or a variant thereof), or an amino acid comprising a polar uncharged side chain (e.g., Q, Cit, Hgn, N, or variant). In certain embodiments, X11 is a hydrophilic amino acid. In certain embodiments, X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof).

In one aspect, described herein is a peptide that has avidity for ephrin type-A receptor 2 (EphA2), wherein the peptide has an amino acid sequence according to Formula (I), or a pharmaceutically acceptable salt thereof,

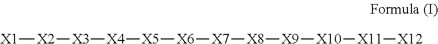

wherein,
X1 is an amino acid;
X2 is F, or a variant thereof that substitutes the unsubstituted phenyl ring of F with
   (i) a phenyl ring substituted by 1 or 2 substituents each independently selected from —OH, —CN, —C₁₋₃ alkyl (e.g., —CH₃), or
   (ii) a 6-membered heteroaryl ring optionally substituted by 1 or 2 substituents each independently selected from —OH, —CN, —C₁₋₃ alkyl (e.g., —CH₃),
   wherein the F or the structural variant thereof is optionally N-methylated;
X3 is a hydrophilic amino acid (e.g. N, Q, Cit, K or a variant thereof), G, Alb, Hgn, Ala, or a variant thereof (e.g., da);
X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
X5 is an amino acid (e.g., a hydrophilic amino acid; or an amino acid with a functional side chain, e.g., not glycine);
X6 is an N-methylated amino acid thereof;
X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F);
X8 is an amino acid with —H on the alpha-amino group;
X9 is W or Y or a variant thereof; (e.g., W or a variant thereof);
X10 is absent, or a polar amino acid (e.g., T or a variant thereof);
X11 is absent, or an amino acid (e.g., a hydrophilic amino acid; Dab, Dap, R. E or a variant thereof; or an amino acid with a functional side chain (e.g., not glycine)); and
X12 is C or a variant thereof.

In some embodiments of Formula (I), both X10 and X11 are present. In some embodiments of Formula (I); both X10 and X11 are absent.

In some embodiments, described herein is a peptide (e.g., a cyclic peptide) of Formula (I), or a pharmaceutically acceptable salt thereof, wherein
X1 is an amino acid (e.g., D-amino acid);
X2 is F or a variant thereof, Y or a variant thereof, or W or a variant thereof, or N-methylated amino acid thereof;
X3 is absent, N, Q, Cit or a variant thereof, G, Aib, Hgn, K or a variant thereof, Ala, or da;
X4 is absent, G substituted with straight or branched Cis alkyl, A substituted with C₃₋₇ cycloalkyl, or Cit or variant thereof;
X5 is absent, a hydrophilic amino acid, or an amino acid with a functional side chain (e.g., Dab, Dap, R, E), wherein the hydrophilic amino acid comprises an L-amino acid comprising —NH₂, —C(O)OH, —NHC(NH)NH₂, —NHC(O)NH₂, —C(O)NH₂, or —NHC(O)CH₃;
X6 is absent, a hydrophilic amino acid, F or a variant thereof, Y or a variant thereof, W or a variant thereof, or N-methylated amino acid thereof, wherein the hydrophilic amino acid comprises a substituent selected from the group consisting of —C(O)OH, —C(O)NH₂, and —NHC(O)CH₃;
X7 is F or a variant thereof, or W or a variant thereof;
X8 is G substituted with one or two straight or branched C₁₋₅ alkyl, G substituted with C₃₋₇ cycloalkyl, A substituted with C₃₋₇ cycloalkyl, or a hydrophilic amino acid wherein the hydrophilic amino acid comprises an L-amino acid comprising —NH₂, one or more —OH, —C(O)OH, —NHC(NH)NH₂, —NHC(O)NH₂, —C(O)NH₂, —NHC(O)CH₃; or the hydrophilic amino acid comprises a zwitterion;
X9 is F or a variant thereof, or W or a variant thereof;
X10 is absent, Q, Hgn, S or variant thereof, T or variant thereof (e.g., T optionally substituted with straight or branched C₁₋₅ alkyl), K or a variant thereof, Cit or a variant thereof, or an L-amino acid substituted with —NHC(NH)NH₂, —NHC(O)NH₂, —C(O)NH₂, or —NHC(O)CH₃;
X11 is absent, E, Hgn, R or a variant thereof, Cit or a variant thereof, Hgl, K or a variant thereof, D, N, or Q; and
X12 is C or a variant thereof.

In some embodiments of a peptide of Formula (I), or a pharmaceutically acceptable salt thereof, wherein:
X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, or dhAla;
X2 is MeF, Me3Py, MeF3CON, MeF3F, Me4Py, MeY(Me), or N-methylated amino acid thereof;
X3 is absent, N, Q, Cit, G, Alb, Hgn, hCit, norCit, LysAc, OrnAc, Ala, or da;
X4 is L, Cbg, Chg, Cba, Cha, Ahx, Dahp, Cit, I, V, Norleucine, or Norvaline;
X5 is Hgl, Hgn, Dab, Dap, DabAc, DapAc, R, hArg, E, or D;
X6 is absent, MeF, MeE, Me3Py, Me4Py, MeF4F, MeF4F, MeF4C, or MeY;
X7 is W1Me, W1Me7Cl, W1Me7N, W, F, 7-AzaTrp, W7Me, or W1Et;
X8 is V, KCOpipzaa, Cit, Qglucamine, hCit, Aib, Norleucine, or Norvaline;
X9 is W1Me, W1Me7Cl, W1Me7N, F23dMe, W1Et, W7Me, W, F, or 7-AzaTrp;
X10 is absent, T, Q, S, Hgn, Alpha-methylserine, hSer, hThr, N, OrnAc, LysAc, Cit, or hCit;
X11 is absent, E, Hgn, R, hArg, Cit, hCit, Hgl, Orn, D, N, Q, DapAc, OrnAc, DabAc, norCit; and
X12 is C, hCys, CdMe, C3RMe, C3SMe, Selenocysteine, dc, or Penicillamine.

In some embodiments, of a peptide of Formula (I), or a pharmaceutically acceptable salt thereof,
X7 is W1Me, or a variant thereof; and
X9 is W1Me, or a variant thereof.

In some embodiments of a peptide of Formula (I), or a pharmaceutically acceptable salt thereof, wherein:
X7 is W1Me, W1MeCl, W1MeBr, Nal1, Nal2, W1Et, 3Bzf, 3Bzt, F23dC, W1Me7N, or F23dMe;
X8 is V, KCOpipzaa, N, Cit, hCit, KAc, DapAc, OrnAc, A, T, alT, Alb, Alb, Qglucamine, Hgl, Q, E, Hgn, or K; and
X9 is W1Me, Nal1, W1Et, Nal21N, 3Bzf, 3Bzt, Nal18N, F23dMe, or F23dC.

In some embodiments of a peptide of Formula (I), or a pharmaceutically acceptable salt thereof, wherein:
X7 is W1Me;
X8 is V; and,
X9 is W1Me.

In one aspect, described herein is a peptide (e.g., a cyclic peptide) that has avidity for ephrin type-A receptor 2 (EphA2), wherein the peptide has an amino acid sequence according to Formula (I), or a pharmaceutically acceptable salt thereof,

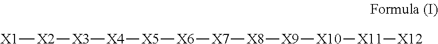

wherein,
X1 is any amino acid (e.g., D-amino acid);
X2 is an amino acid comprising an aromatic ring or a substitution thereof, N-methylated amino acid, or a substitution thereof;
X3 is absent, N or a substitution thereof;
X4 is absent, any hydrophobic amino acid or a substitution thereof;
X5 is absent, a hydrophilic amino acid or a substitution thereof, or an amino acid with a functional side chain (e.g., Dab, Dap, K);
X6 is absent, a hydrophilic amino acid or amino acid having aromatic ring, N-methylated amino acid thereof, or a substitution thereof;
X7 is W or a substitution thereof;
X8 is V, hydrophilic amino acid or a substitution thereof, an N-methylated amino acid, or an amino acid with a functional side chain;
X9 is W or a substitution thereof;
X10 is absent, T or a substitution thereof;
X11 is absent, any hydrophilic amino acid, or an amino acid with a functional side chain; and
X12 is C or a substitution thereof.

In some embodiments, described herein is a peptide (e.g., a cyclic peptide) that has an amino acid sequence according to Formula (I), or a pharmaceutically acceptable salt thereof, wherein
X1 is any amino acid (e.g., D-amino acid);
X2 is an amino acid comprising an aromatic ring or a variant thereof, or N-methylated amino acid thereof;
X3 is absent, N or a variant thereof;
X4 is absent, any hydrophobic amino acid or a variant thereof;
X5 is absent, a hydrophilic amino acid or a variant thereof, or an amino acid with a functional side chain (e.g., Dab, Dap, K);
X6 is absent, a hydrophilic amino acid or amino acid having aromatic ring, or N-methylated amino acid thereof;
X7 is W or a variant thereof;
X8 is V, hydrophilic amino acid or a variant thereof, an N-methylated amino acid, or an amino acid with a functional side chain;
X9 is W or a variant thereof;
X10 is absent, T or a variant thereof;
X11 is absent, any hydrophilic amino acid, or an amino acid with a functional side chain; and
X12 is C or a variant thereof.

In some embodiments, described herein is the peptide of Formula (I), or a pharmaceutically acceptable salt thereof, wherein
X1 is any amino acid;
X2 is an amino acid comprising an aromatic ring, or N-methylated amino acid thereof;
X3 is absent, a hydrophilic amino acid (e.g. N, Q, Cit, K or a variant thereof), G, Aib, Hgn, or Ala or a variant thereof (e.g., da);
X4 is absent, a hydrophobic amino acid, or a hydrophilic amino acid (e.g., Cit or a variant thereof);
X5 is absent, a hydrophilic amino acid, or an amino acid with a functional side chain;
X6 is absent, a hydrophilic amino acid, or an or amino acid having aromatic ring, or N-methylated amino acid thereof;
X7 is an amino acid comprising an aromatic ring (e.g., W or a variant thereof);
X8 is a hydrophobic amino acid, a hydrophilic amino acid, an N-methylated amino acid, or an amino acid with a functional side chain;
X9 is an amino acid comprising an aromatic ring (e.g., W or a variant thereof);
X10 is absent, or a polar amino acid (e.g., T or a variant thereof);
X11 is absent, a hydrophilic amino acid, or an amino acid with a functional side chain; and
X12 is C or a variant thereof.

In some embodiments, described herein is a peptide (e.g., a cyclic peptide) of Formula (I), or a pharmaceutically acceptable salt thereof, wherein
X1 is an amino acid (e.g., D-amino acid);
X2 is an amino acid comprising an aromatic ring, or N-methylated amino acid thereof;
X3 is absent, a hydrophilic amino acid (e.g. N, Q, Cit, K or a variant thereof), G, Aib, Hgn, or Ala or a variant thereof (e.g., da);
X4 is a hydrophobic amino acid, or a hydrophilic amino acid (e.g., Cit or a variant thereof);
X5 is a hydrophilic amino acid (e.g., Dab, Dap, R, E or a variant thereof);
X6 is absent, a hydrophilic amino acid, an amino acid having aromatic ring (e.g., W), or N-methylated amino acid thereof;
X7 is an amino acid comprising an aromatic ring (e.g., W or a variant thereof);
X8 is a hydrophobic amino acid, a hydrophilic amino acid, or an N-methylated amino acid;
X9 is an amino acid comprising an aromatic ring (e.g., W or a variant thereof);
X10 is absent, or a hydrophilic amino acid (e.g., T or a variant thereof);
X11 is absent, or a hydrophilic amino acid; and
X12 is C or a variant thereof.

In some embodiments, described herein is a peptide (e.g., a cyclic peptide) of Formula (I), or a pharmaceutically acceptable salt thereof,

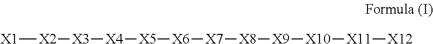

wherein,
   X1 is any amino acid,
   X2 is an amino acid having an aromatic ring or a variant thereof,
   X3 is N,
   X4 is a hydrophobic amino acid or a variant thereof;
   X5 is a hydrophilic amino acid or a variant thereof;
   X6 is a hydrophilic amino acid or amino acid having aromatic ring;
   X7 is W or a variant thereof;
   X8 is V or hydrophilic amino acid or a variant thereof,
   X9 is W or a variant thereof;
   X10 is T or a variant thereof;
   X11 is a hydrophilic amino acid;
   X12 is C or a variant thereof (such as C).

In some embodiments, described herein is a peptide (e.g., a cyclic peptide) of Formula (Ia), or a pharmaceutically acceptable salt thereof,

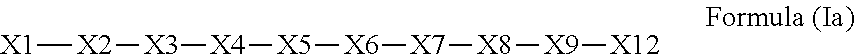

wherein,
   X1 is any amino acid;
   X2 is an amino acid having an aromatic ring or a variant thereof;
   X3 is N or a variant thereof;
   X4 is a hydrophobic amino or a variant thereof,
   X5 is a hydrophilic amino acid or a variant thereof;
   X6 is a hydrophilic amino acid or amino acid having aromatic ring;
   X7 is W or a variant thereof;
   X8 is a hydrophilic amino acid or a variant thereof,
   X9 is W or a variant thereof; and
   X12 is C or a variant thereof.

In some embodiments, described herein is a (cyclic) peptide that has avidity for ephrin type-A receptor 2 (EphA2), wherein the peptide consists of a sequence of Formula (I),

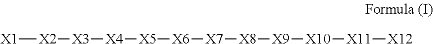

or a pharmaceutically acceptable salt thereof,
wherein
each of X1, X2, X3, X4, X5, X6, and X8 is independently an amino acid;
X7 is W1Me or a variant thereof;
X9 is W1Me or a variant thereof;
each of X10 and X11 is independently absent or an amino acid; and
X12 is cysteine (C) or a variant thereof.

In some embodiments, the peptide of Formula (I), or a pharmaceutically acceptable salt thereof,

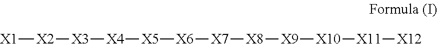

wherein,
X1 is any amino acid;
X2 is an amino acid comprising an aromatic ring or a variant thereof, or N-methylated amino acid thereof;
X3 is absent, N or a variant thereof;
X4 is any hydrophobic amino acid or a variant thereof;
X5 is a hydrophilic amino acid or a variant thereof;
X6 is absent, a hydrophilic amino acid or amino acid having aromatic ring, or N-methylated amino acid thereof;
X7 is W or a variant thereof;
X8 is V, hydrophilic amino acid or a variant thereof, or an N-methylated amino acid;
X9 is W or a variant thereof;
X10 is absent, T or a variant thereof;
X11 is absent, any hydrophilic amino acid; and
X12 is C or a variant thereof.

In one aspect, described herein is a linker-added peptide or conjugate comprising:
(a) a (cyclic) peptide that has avidity for ephrin type-A receptor 2 (EphA2), wherein the peptide has an amino acid sequence of Formula (I),

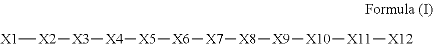

wherein,
X1 is any D- or L-amino acid;
X2 has a structure of

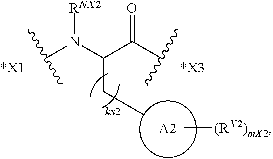

wherein
   ring A2 is phenyl or a 6-membered heteroaryl (e.g., heteroaryl having 1 or 2 N);
   R^{X2} is each independently halogen, —CN, —NO₂, —OH, —OR^{a}, —OC(═O)R^{a}, —OC(═O)OR^{a}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx2 is 0, 1, 2, or 3;
   mx2 is 0, 1, 2, 3 or 4;
   R^{NX2} is H, C₁-C₆alkyl, or C₁-C₆haloalkyl;
   *X1 indicates the point of attachment to X1; and,
   *X3 indicates the point of attachment to X3;
X3 has a structure of

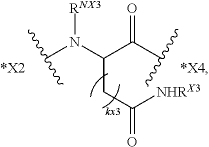

wherein
   kx3 is 0, 1, 2, or 3;
   R^{NX3} is H, C₁-C₆alkyl, or C₁-C₆haloalkyl;
   R^{X3} is H, C₁-C₆alkyl, C₁-C₆haloalkyl, C₁-C₆hydroxyalkyl, C₁-C₆aminoalkyl, or C₁-C₆heteroalkyl;
   *X2 indicates the point of attachment to X2; and,
   *X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of

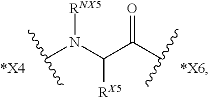

wherein:
   R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
   R^{X5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
   provided that at least one of R^{NX5} and R^{X5} comprises a moiety selected from —OH, —NH₂, and —NH— (e.g., —NH—C(═NH)—NH₂, —CO—NH₂, —NH₂, —COOH, —C(OH)—C₀₋₆ alkyl, —NH—CO—C₁₋₆ alkyl);
   *X4 indicates the point of attachment to X4; and,
   *X6 indicates the point of attachment to X6;
X6 is

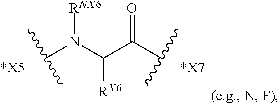

wherein
   R^{NX6} is H, C₁-C₆alkyl, or C₁-C₆haloalkyl;
   R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{XA};
   *X5 indicates the point of attachment to X5; and,
   *X7 indicates the point of attachment to X7;
X7 has a structure of

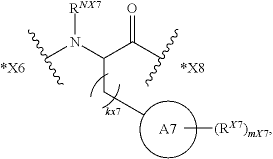

wherein
   R^{NX7} is H, C₁-C₆alkyl, or C₁-C₆haloalkyl;
   ring A7 is an aryl or heteroaryl;
   R^{X7} is each independently halogen, —CN, —NO₂, —OH, —OR^{a}, —OC(═O)R^{a}, —OC(═O)OR^{b}, —OC(═O)NR^{c}R^{d}, —SH, SF₆, —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx7 is 0, 1, 2, or 3;
   mx7 is 0, 1, 2, 3, 4 or 5;
   *X6 indicates the point of attachment to X6; and,
   *X8 indicates the point of attachment to X8;
X8 is an L-amino acid with —H on the alpha-amino group;
X9 has a structure of

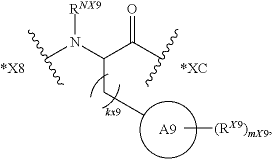

wherein
   R^{NX9} is H, C₁-C₆alkyl, or C₁-C₆haloalkyl;
   ring A9 is an aryl or heteroaryl;
   R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx9 is 0, 1, 2, or 3;
   mx9 is 0, 1, 2, 3, 4, or 5;
   *X8 indicates the point of attachment to X8; and,
   *XC indicates the point of attachment to (i) X10 or (i) when X10 and X11 are absent, X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl). C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;

or R^{c} and R^{d} are taken together with the atom to which they are attached to form a heterocycloalkyl optionally substituted with one or more R; and
each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and,

(b) optionally, a linker that connects the peptide to a payload molecule.

In some embodiments, ring A7 is a 6-membered aryl or heteroaryl. In some embodiments, ring A7 is a 9- or 10-membered bicyclic aryl or heteroaryl. In some embodiments, the 6-, 9- or 10-membered heteroaryl has one heteroatom selected from N, O, and S. In some embodiments, R^{NX7} is H. In some embodiments, each R^{X7} is independently selected from —CH₃, -ethyl, —Cl, and —F, and mx7 is 0, 1, or 2.

In some embodiments, X7 is W1Me, Nal1, Nal2, W1Et, Nal21N, 3Bzf, 3Bzt, Nal15N, Nal14N, Nal24N, Nal28N, F23dMe, F23dC, W1Me7N, or W1Me7Cl. In some embodiments, X7 is W1Me, F23dMe or W1Me7Cl.

In some embodiments, X9 is

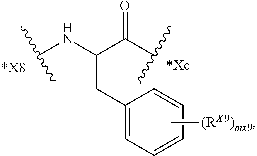

wherein each R^{X9} is independently selected from —OH, CN, NH₂, C₁-C₃alkyl, —Cl, —F, —Br, —CONH₂, and —SO₂F.

In some embodiments,

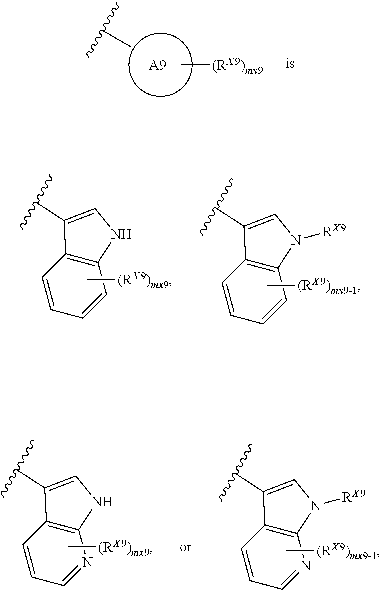

In some embodiments, each of R^{X9} is independently halogen, —CN, —NO₂, —OH, —OR^{a}, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments, X9 is W1Me, W, Nal1, W1Et, Nal21N, 3Bzf, 3Bzt, Nal14N, Nal18N, F23dMe, F23dC, or W1Et. In some embodiments, X9 is W1Me or F23dMe.

In some embodiments, ring A2 is a 6-membered heteroaryl containing 1 or 2 N.

In some embodiments, R^{X5} is C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments, X7 is W1Me, W1MeCl, W1MeBr, Nal1, Nal2, W1Et, 3Bzf, 3Bzt, F23dC, W1Me7N, or F23dMe; X8 is V, KCOpipzaa, Hse, N, Cit, hCit, KAc, DapAc, OrAc, T, alT, Aib, Alb, Qglucamine, Hgl, E, Hgn, MeF, 3Py6NH2, W1Me, A, Q, or K; and X9 is W1Me, Nal1, W1Et, Nal21N, 3Bzf, 3Bzt, Nal18N, F23dMe, or F23dC.

In some embodiments, X7 is W1Me; X8 is V; and, X9 is W1Me.

In some embodiments of Formulae (I), (I-1), (I-2), (I-3), (I-4), (I-5), (Ia), (Ib), (Ic), (III-1) and (III-2), X1 is any amino acid (e.g., D-amino acid). In some embodiments, X1 is any one of the canonical amino acids. In some embodiments, X1 is an unnatural amino acid. In some embodiments, X1 is alanine (A). In some embodiments, X1 is D-alanine. In some embodiments, X1 is df3CON. In some embodiments, X1 is dkCOpipzaa. In some embodiments, X1 is dahp. In some embodiments, X1 is F. In some embodiments, X1 is an amino acid selected from Tables 5A to 5F. In some embodiments, the payload molecule or linker is attached to X1

In some embodiments of Formulae (I), (I-1), (I-2), (I-3), (I-4), (I-5), (Ia), (Ib), (Ic), (III-1) and (III-2), X1 is any amino acid. In some embodiments, X1 is an amino acid (e.g., a D-amino acid). In some embodiments, X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, or dhAla. X1 is da. X1 is df3CON. X1 is dkCOpipzaa. X1 is dahp. X1 is dDab-NH₂-Ph3-SO₂F. X1 is dDap-NH₂-Ph3-SO₂F. X1 is dCit. X1 is Aib. X1 is G. X1 is Norvaline. X1 is Norleucine. X1 is dhAla. In some embodiments, X1 is F.

In some embodiments of Formulae (I), (I-1), (I-2), (I-3), (I-4), (I-5), (Ia), (Ib), (Ic), (III-1) and (III-2), X2 is a canonical amino acid. In some embodiments, X2 is an unnatural amino acid. In some embodiments, X2 is an aromatic amino acid or a variant thereof. In some embodiments, X2 is V. In some embodiments, X2 is an N-methylated amino acid or a variant thereof. In some embodiments, X2 is an N-alkylated amino acid or a variant thereof. In some embodiments, X2 is an amino acid comprising an aryl group. In some embodiments, X2 is an amino acid comprising an optionally substituted phenyl group. In some embodiments, X2 is an amino acid comprising an optionally substituted naphthyl group. In some embodiments, X2 is an amino acid comprising a heteroaryl group. In some embodiments, X2 is an amino acid comprising an optionally substituted monocyclic heteroaryl group. In some embodiments, X2 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₃alkyl, C₁-C₆ alkoxyl, and C₁-C₆ haloalkyl. In some embodiments, X2 is F, or a variant thereof that substitutes the unsubstituted phenyl ring of F with (i) a phenyl ring substituted by 1 or 2 substituents each independently selected from —OH, —CN, —C₁₋₃ alkyl, or (ii) a 6-membered heteroaryl ring optionally substituted by 1 or 2 substituents each independently selected from —OH, —CN, —C₁₋₃ alkyl, wherein the F or the structural variant thereof is optionally N-methylated. In some embodiments, X2 is Me3Py. In some embodiments, X2 is In some embodiments, X2 is MeF. In some embodiments, X2 is MeF3H. In some embodiments, X2 is MeF3CN. In some embodiments, X2 is MeF3H. In some embodiments, X2 is Me4Py2NH₂. In some embodiments, X2 is 4Py2NH2. In some embodiments, X2 is 4Py. In some embodiments, X2 is Me3Py. In some embodiments, X2 is an amino acid substituted with an aryl or heteroaryl. In some embodiments, X2 is histidine (H). In some embodiments, X2 is phenylalanine, tryptophan, tyrosine, or a variant thereof. In some embodiments, X2 is phenylalanine or a variant thereof. In some embodiments, X2 is tryptophan or a variant thereof. In some embodiments, X2 is W1Me. In some embodiments, X2 is tyrosine or a variant thereof. In some embodiments, X2 is absent. In some embodiments, the payload molecule or linker is attached to X2. In some embodiments of Formulae (I), (I-1), (I-2), (I-3), (I-4), (I-5), (III-1), (Ia), (Ib), (Ic), and (III-2), X2 is an amino acid comprising an aromatic ring, or N-methylated amino acid thereof. In some embodiments, X2 is N-methylated amino acid. In some embodiments, X2 is an amino acid comprising an aromatic ring. In some embodiments, X2 is an N-methylated amino acid comprising an aromatic ring. In some embodiments, X2 is F or a variant thereof, Y or a variant thereof, or W or a variant thereof, or N-methylated amino acid thereof. In some embodiments, X2 is F or a variant thereof. In some embodiments, X2 is N-methyl F or a variant thereof. In some embodiments, X2 is Y or a variant thereof. In some embodiments, X2 is N-methyl Y or a variant thereof. In some embodiments, X2 is W or a variant thereof. In some embodiments, X2 is N-methyl W or a variant thereof. In some embodiments, X2 is MeF, Me3Py, MeF3CON, MeF3F, Me4Py, or MeY(Me). In some embodiments, X2 is MeF. In some embodiments, X2 is Me3Py. In some embodiments, X2 is MeF3CON. In some embodiments, X2 is MeF3F. In some embodiments, X2 is Me4Py. In some embodiments, X2 is MeY. In some embodiments, X2 is MeY(Me).

In some embodiments of Formulae (I), (I-1), (I-2), (I-3), (I-4), (I-5), (Ia), (III-1) and (III-2), X3 is a canonical amino acid. In some embodiments, X3 is an unnatural amino acid. In some embodiments, X3 is asparagine (N). In some embodiments, X3 is a substitute of asparagine. In some embodiments, X3 is absent. In some embodiments of Formulae (I), (I-1), (I-2), (I-3), (I-4), (I-5), (III-1), (Ia) and (III-2), X3 is absent. In some embodiments of Formulae (I), (I-1), (I-2), (I-3), (I-4), (I-5), (III-1), (Ia) and (III-2), X3 is a hydrophilic amino acid (e.g. N, Hgn, Q, Cit, K or a variant thereof), glycine (G), Alanine (A) or a variant thereof (e.g., da, 2-Aminoisobutyric acid (Aib). In some embodiments, X3 is a hydrophilic amino acid. In some embodiments, X3 is an amino acid comprising —OH, —NH₂, —C(O)OH, —NHC(═NH)NH₂, —NHC(O)NH₂, —C(O)NH₂, or —NHC(O)CH₃ group. In some embodiments, X3 has an electrically charged side chain. In some embodiments, X3 has a positively charged side chain. In some embodiments, X3 has a negatively charged side chain. In some embodiments of Formulas (I), (I-1), (I-2), (I-3), (I-4), (I-5), (III-1), (Ia) and (III-2), X3 is an amino acid comprising an electrically charged side chain (e.g., Kor a variant thereof), an amino acid comprising a polar uncharged side chain (e.g., Q, Cit, N, or a variant thereof), or G, A or variant thereof. In some embodiments, X3 is an amino acid comprising an electrically charged side chain. In some embodiments, X3 is an amino acid comprising a polar uncharged side chain. In some embodiments, X3 has zwitterionic (e.g., KCOpipzaa) side chain. In some embodiments, X3 is zwitterionic. In some embodiments, X3 comprises a —OH, —COOH, —NH— or NH₂ moiety. In some embodiments, X3 comprises —OH, —C(O)OH, —NHC(═NH)NH₂, —NHC(O)NH₂, —C(O)NH₂, or —NHC(O)CH₃. In some embodiments, X3 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X3 is absent, a hydrophilic amino acid (e.g. N, Q, Hgn, Cit, K or a variant thereof), G, Ala, or a variant thereof (e.g., da, ib). In some embodiments, X3 is N, Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments X3 is absent, N, Q, Cit or a variant thereof, G, Aib, Hgn, K or a variant thereof, or Ala or a variant thereof (e.g., da). In some embodiments, X3 is absent, N, Q, Cit, G, Alb, Hgn, hCit, norCit, LysAc, OrnAc, Ala, or da. In some embodiments, X3 is N or a variant thereof. In some embodiments, X3 is N. In some embodiments, X3 is Q or a variant thereof. In some embodiments, X3 is Q. In some embodiments, X3 is Cit or a variant thereof. In some embodiments, X3 is Cit, hCit, or norCit. In some embodiments, X3 is Cit. In some embodiments, X3 is hCit. In some embodiments, X3 is norCit. In some embodiments, X3 is K or a substitution there of. In some embodiments, X3 is K, LysAc, or OrnAc. In some embodiments. X3 is K. In some embodiments, X3 is LysAc. In some embodiments, X3 is OrnAc. In some embodiments, X3 is G or a variant thereof. In some embodiments, X3 is G. In some embodiments, X3 is Hgn. In some embodiments, X3 is Aib. In some embodiments, X3 is Ala or a variant thereof. In some embodiments, X3 is Ala or da. In some embodiments, X3 is Ala. In some embodiments, X3 is da. In some embodiments, X3 is absent. In some embodiments, the payload molecule or linker is attached to X3. In some embodiments, X1 is directly bound to X3.

In some embodiments of Formulae (I), (I-1), (I-2), (I-3), (I-4), (I-5), (Ia), (Ib), (III-1) and (III-2), X4 is a hydrophobic amino acid or a variant thereof. In some embodiments, X4 is an unnatural amino acid. In some embodiments, X4 is a canonical amino acid. In some embodiments, X4 is leucine. In some embodiments, X4 comprises 4 or more carbon atoms in a side chain comprising a linear, branched, or cyclic carbon chain. In some embodiments, X4 comprises 4 or more contiguous carbon atoms in a side chain. In some embodiments, X4 comprises an ethylene, propylene, or butylene group in a side chain. In some embodiments, X4 is Cbg. In some embodiments, X4 is absent. In some embodiments, X4 is selected from glycine (G), methionine (M), alanine (A), valine (V), leucine (L), isoleucine (I), proline (P), phenylalanine (F), cysteine (C), substitutes thereof. In some embodiments of Formulas (I), (I-1), (I-2), (I-3), (I-4), (I-5), (Ia), (Ib), (III-1) and (III-2), X4 is an amino acid comprising a hydrophobic side chain (e.g., L), an amino acid comprising a polar uncharged side chain (e.g., Cit or a variant thereof). In some embodiments, X4 is an amino acid comprising a hydrophobic side chain. In some embodiments, X4 is an amino acid comprising a polar uncharged side chain. In some embodiments of Formulae (I), (I-1), (I-2), (I-3), (I-4), (I-5), (Ia), (Ib), (III-1) and (III-2), X4 is absent, a hydrophobic amino acid, or a hydrophilic amino acid (e.g., Cit or a variant thereof). In some embodiments, X4 is absent, G substituted with straight or branched C₁₋₅ alkyl, A substituted with C₃₋₇ cycloalkyl, or Cit or variant thereof. In some embodiments, X4 is absent, L, Cbg, Chg, Cba, Cha, Ahx, Dahp, citrulline (Cit), I, V, Norleucine, or Norvaline. In some embodiments, X4 is absent. In some embodiments, X4 is a hydrophobic amino acid. In some embodiments, X4 is Leu, Hcit, Cbg, Chg, or Cba. In some embodiments, X4 is Leu, Cbg, Chg or Cba. In some embodiments, X4 is G substituted with straight or branched C₁₋₅ alkyl. In some embodiments, X4 is G substituted with methyl, ethyl, propyl, isopropyl, butyl, isobutyl, pentyl, or isopentyl. In some embodiments, X4 A substituted with C₃₋₇ cycloalkyl. In some embodiments, X4 is A substituted with cyclopropyl. In some embodiments, X4 is A substituted with cyclobutyl. In some embodiments, X4 is A substituted with cyclopentyl. In some embodiments, X4 is A substituted with cyclohexyl. In some embodiments, X4 is A substituted with cycloheptyl. In some embodiments, X4 is L, Cbg, Chg, Cba, Cha, Ahx, Dahp, I, V, Norleucine, or Norvaline. In some embodiments, X4 is L. In some embodiments, X4 is Cbg. In some embodiments, X4 is Chg. In some embodiments, X4 is Cba. In some embodiments, X4 is Cha. In some embodiments, X4 is Ahx. In some embodiments, X4 is Dahp. In some embodiments, X4 is I. In some embodiments, X4 is V. In some embodiments, X4 is Norleucine. In some embodiments, X4 is Norvaline. In some embodiments, X4 is a hydrophilic amino acid. In some embodiments, X4 is Cit or a variant thereof. In some embodiments, X4 is Cit. In some embodiments, X4 is optionally N-methylated. In some embodiments, the payload molecule or linker is attached to X4. In some embodiments, X1 is directly bound to X4. In some embodiments of Formulae (I), (I-1), (I-2), (I-3), (I-4), (I-5), (Ia), (Ib), (III-1) and (III-2), X4 is a hydrophilic amino acid. In some embodiments, X4 is an amino acid comprising —OH, —NH₂, —C(O)OH, —NHC(═NH)NH₂, —NHC(O)NH₂, —C(O)NH₂, or —NHC(O)CH₃ group. In some embodiments, X4 has an electrically charged side chain. In some embodiments, X4 has a positively charged side chain. In some embodiments, X4 has a negatively charged side chain. In some embodiments, X4 is zwitterionic. In some embodiments, X4 comprises a —OH, —COOH, —NH— or NH₂ moiety. In some embodiments, X4 comprises —OH, —C(O)OH, —NHC(═NH)NH₂, —NHC(O)NH₂, —C(O)NH₂, or —NHC(O)CH₃. In some embodiments, X4 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments of Formulae (I), (I-1), (I-2), (I-3), (I-4), (I-5), (Ia), (Ib), (III-1) and (III-2), X4 is a hydrophobic amino acid. In some embodiments, X4 comprises at least 4 contiguous carbon atoms, either linear or branched. In some embodiments, X4 comprises at least 5 contiguous carbon atoms, either linear or branched. In some embodiments, X4 comprises a propylene moiety in the side chain. In some embodiments, X4 comprises a butylene moiety in the side chain.

In some embodiments of Formulas (I), (I-1), (I-2), (I-3), (I-4), (I-5), (Ia), (Ib), (III-1) and (III-2), X5 is a hydrophilic amino acid or a variant thereof. In some embodiments, X5 is a hydrophilic amino acid. In some embodiments, X5 is an unnatural amino acid. In some embodiments, X5 is a positively charged amino acid. In some embodiments, X5 is a negatively charged amino acid. In some embodiments, X5 is not charged. In some embodiments, X5 is a canonical amino acid. In some embodiments, X5 is Ala or a variant thereof. In some embodiments, X5 is N, Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X5 is Hgn, N, Qglucamine, KCOpipzaa, Hgl, Nmm, Ndm, KCOpipzaa, K, S, T, or E. In some embodiments, X5 is Hgn. In some embodiments, X5 is asparagine (N). In some embodiments, X5 is Qglucamine. In some embodiments, X5 is Hgl. In some embodiments, X5 is Nmm. In some embodiments, X5 is Ndm. In some embodiments, X5 is KCOpipzaa. In some embodiments, X5 is Dab. In some embodiments, X5 is S. In some embodiments, X5 is K. In some embodiments, X5 is absent. In some embodiments of Formulas (I), (I-1), (I-2), (I-3), (I-4), (I-5), (Ia), (Ib), (III-1) and (III-2), X5 is an amino acid comprising an electrically charged side chain (e.g., E, Hgl, D, or a variant thereof), or an amino acid comprising a polar uncharged side chain (e.g., Q, Cit, Hgn, N, or a variant thereof). In some embodiments, X5 is an amino acid comprising an electrically charged side chain. In some embodiments, X5 is an amino acid comprising a polar uncharged side chain. In some embodiments of Formulas (I), (I-1), (I-2), (I-3), (I-4), (I-5), (III-1), (Ia), (Ib), and (III-2), X5 is absent, a hydrophilic amino acid, or a variant thereof. In some embodiments, X5 is absent, a hydrophilic amino acid, or an amino acid with a functional side chain (e.g., Dab, Dap, R, E), wherein the hydrophilic amino acid comprises an L-amino acid comprising —NH₂, —C(O)OH, —NHC(NH)NH₂, —NHC(O)NH₂, —C(O)NH₂, or —NHC(O)CH₃. In some embodiments, X5 is absent, Hgl, Hgn, Dab, Dap, DabAc, DapAc, R, hArg, E, or D. In some embodiments, X5 is absent. In some embodiments, X5 is a hydrophilic amino acid. In some embodiments, X5 is an amino acid comprising —NH₂, —C(O)OH, —NHC(NH)NH₂, —NHC(O)NH₂, —C(O)NH₂, or —NHC(O)CH₃. In some embodiments, X5 is an L-amino acid comprising —NH₂, —C(O)OH, —NHC(NH)NH₂, —NHC(O)NH₂, —C(O)NH₂, or —NHC(O)CH₃. In some embodiments, X5 is Hgl. In some embodiments, X5 is Hgn. In some embodiments, X5 is Dab. In some embodiments, X5 is Dap. In some embodiments, X5 is DabAc. In some embodiments, X5 is DapAc. In some embodiments, X5 is R or a variant thereof. In some embodiments, X5 is R or hArg. In some embodiments, X5 is R. In some embodiments, X5 is hArg. In some embodiments, X5 is E. In some embodiments, X5 is hCit. In some embodiments, X5 is G. In some embodiments, X5 is D. In some embodiments, the linker is attached to X5. In some embodiments, X1 is directly bound to X5.

In some embodiments of Formulae (I), (I-1), (I-2), (I-3), (I-4), (I-5), (Ia), (Ic), (III-1) and (III-2), X6 is any amino acid. In some embodiments, X6 is a canonical amino acid. In some embodiments, X6 is an unnatural amino acid. In some embodiments, X6 is hydrophilic amino acid or amino acid having aromatic ring, or N-methylated amino acid thereof, or a substitute thereof. In some embodiments, X6 is an amino acid having aromatic ring or a substitute thereof. In some embodiments, X6 is an amino acid comprising an aryl group. In some embodiments, X6 is an amino acid comprising an optionally substituted phenyl group. In some embodiments, X6 is an amino acid comprising an optionally substituted naphthyl group. In some embodiments, X6 is an amino acid comprising a heteroaryl group. In some embodiments, X6 is an amino acid comprising an optionally substituted monocyclic heteroaryl group. In some embodiments, X6 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X6 is N-methylated amino acid. In some embodiments, X6 is hydrophilic amino acid or a substitute thereof. In some embodiments, X6 is an amino acid having aromatic ring or a substitute thereof. In some embodiments, X6 is an N-methylated amino acid or a substitute thereof. In some embodiments, X6 is MeE. In some embodiments, X6 is N. In some embodiments, X6 is MeN. In some embodiments, X6 is Me3Py. In some embodiments, X6 is MeF. In some embodiments, X6 is Qglucamine. In some embodiments, X6 is MeF4C. In some embodiments, X6 is absent. In some embodiments of Formulas (I), (I-1), (I-2), (I-3), (I-4), (I-5), (Ia), (Ic), (III-1) and (III-2), X6 is an amino acid comprising an electrically charged side chain (e.g., E, Hgl, D, or a variant thereof), or an amino acid comprising a polar uncharged side chain (e.g., Q, Cit, Hgn, N, or variant). In some embodiments, X6 is an amino acid comprising an electrically charged side chain. In some embodiments, X6 is an amino acid comprising a polar uncharged side chain. In some embodiments of Formulae (I), (I-1), (I-2), (I-3), (I-4), (I-5), (III-1), (Ia), (Ic), and (III-2), X6 is absent, a hydrophilic amino acid, an amino acid comprising an aromatic ring, or N-methylated amino acid thereof. In some embodiments of Formulae (I), (I-1), (I-2), (I-3), (I-4), (I-5), (Ia), (Ib), (III-1) and (III-2), X6 is a hydrophilic amino acid. In some embodiments, X6 is an amino acid comprising —OH, —NH₂, —C(O)OH, —NHC(═NH)NH₂, —NHC(O)NH₂, —C(O)NH₂, or —NHC(O)CH₃ group. In some embodiments, X6 has an electrically charged side chain. In some embodiments, X6 has a positively charged side chain. In some embodiments, X6 has a negatively charged side chain. In some embodiments, X6 is zwitterionic. In some embodiments, X6 comprises a —OH, —COOH, —NH— or NH₂ moiety. In some embodiments, X6 comprises —OH, —C(O)OH, —NHC(═NH)NH₂, —NHC(O)NH₂, —C(O)NH₂, or —NHC(O)CH₃. In some embodiments, X6 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X6 is absent, a hydrophilic amino acid, F or a variant thereof, Y or a variant thereof, W or a variant thereof, or N-methylated amino acid thereof, wherein the hydrophilic amino acid comprises a substituent selected from the group consisting of —C(O)OH, —C(O)NH₂, and —NHC(O)CH₃. In some embodiments, X6 is absent, MeF, MeE, Me3Py, Me4Py, MeF4F, MeF4C, or MeY. In some embodiments, X6 is MeE, MeN, Me3Py, MeF, MeF4C, or N. In some embodiments, X6 is absent. In some embodiments, X6 is a hydrophilic amino acid. In some embodiments, X6 is an amino acid comprising —NH₂, —C(O)OH, —NHC(NH)NH₂, —NHC(O)NH₂, —C(O)NH₂, or —NHC(O)CH₃. In some embodiments, X6 is E or N-methylated amino acid thereof. In some embodiments, X6 is E. In some embodiments, X6 is MeE. In some embodiments, X6 is an amino acid comprising an aromatic ring or N-methylated amino acid thereof. In some embodiments, X6 is an amino acid comprising an optionally substituted phenyl. In some embodiments, X6 is an amino acid comprising an optionally substituted heteroaryl. In some embodiments, X6 is F or a variant thereof, or N-methylated amino acid thereof. In some embodiments, X6 is F, MeF, Me3Py, Me4Py, MeF4F, or MeF4C. In some embodiments, X6 is F. In some embodiments, X6 is MeF. In some embodiments, X6 is Me3Py. In some embodiments, X6 is Me4Py. In some embodiments, X6 is MeF4F. In some embodiments, X6 is MeF4C. In some embodiments, X6 is Y or a variant thereof, or N-methylated amino acid thereof. In some embodiments, X6 is Y or MeY. In some embodiments, X6 is Y. In some embodiments, X6 is MeY. In some embodiments, the payload molecule or linker is attached to X6. In some embodiments, X1 is directly bound to X6.

In some embodiments of Formulae (I), (I-1), (I-2), (I-3), (I-4), (I-5), (Ia), (Ib), (Ic), (III-1) and (III-2), X7 is W or a variant thereof. In some embodiments, X7 is a canonical amino acid. In some embodiments, X7 is an unnatural amino acid. In some embodiments, X7 is W1Me. In some embodiments, X7 is W1Me7Cl. In some embodiments, X7 is W1Me7N. In some embodiments, X7 is absent. In some embodiments, X7 is an amino acid having aromatic ring or a substitute thereof. In some embodiments, X7 is an amino acid comprising an aryl group. In some embodiments, X7 is an amino acid comprising an optionally substituted phenyl group. In some embodiments, X7 is an amino acid comprising an optionally substituted naphthyl group. In some embodiments, X7 is an amino acid comprising a heteroaryl group. In some embodiments, X7 is an amino acid comprising an optionally substituted monocyclic heteroaryl group. In some embodiments, X7 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X7 is W, Y, or a variant thereof (such as an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F). In some embodiments of Formulae (I), (I-1), (I-2), (I-3), (I-4), (I-5), (Ia), (Ib), (Ic), (III-1) and (III-2), X7 is an amino acid comprising an aromatic ring. In some embodiments, X7 is an amino acid comprising an aromatic ring (e.g., W or a variant thereof). In some embodiments, X7 is F or a variant thereof, or W or a variant thereof. In some embodiments, X7 is W1Me, W1Me7Cl, W1Me7N, W, F, 7-AzaTrp, W7Me, or W1Et. In some embodiments, X7 is F or a variant thereof. In some embodiments, X7 is F. In some embodiments, X7 is W or a variant thereof. In some embodiments, X7 is Nal1, Nal2, W1Et, Nal21N, 3Bzf, 3Bzt, Nal15N, Nal14N, Nal24N, Nal28N, F23dC, W1Me, W1Me7Cl, or W1Me7N. In some embodiments, X7 is W1Me, W1Me7Cl, W1Me7N, W, 7-AzaTrp, W7Me, or W1Et. In some embodiments, X7 is W1Me, W1Me7Cl, or F23dMe. In some embodiments, X7 is W1Me, W1Me7Cl, Nal1, Nal2, W1Et, Nal21N, 3Bzf, 3Bzt, Nal15N, Nal14N, Nal24N, Nal28N, F23dC, or W1Me7N. In some embodiments, X7 is W1Me, W1Me7Cl, or W1Me7N. In some embodiments, X7 is W1Me. In some embodiments, X7 is W1Me7Cl. In some embodiments, X7 is W1Me7N. In some embodiments, X7 is W. In some embodiments, X7 is 7-AzaTrp. In some embodiments, X7 is W7Me. In some embodiments, the payload molecule or linker is attached to X7. In some embodiments, X1 is directly bound to X7.

In some embodiments of Formulae (I), (I-1), (I-2), (I-3), (I-4), (I-5), (Ia), (Ib), (Ic), (III-1) and (III-2), X8 is any amino acid. In some embodiments, X8 is any one of the canonical amino acids. In some embodiments, X8 is an unnatural amino acid. In some embodiments, X8 is V, hydrophilic amino acid, an N-methylated amino acid, or a substitute thereof. In some embodiments, X8 is V. In some embodiments, X8 is phenylalanine, tryptophan, tyrosine, or a variant thereof. In some embodiments, X8 is phenylalanine or a variant thereof. In some embodiments, X8 is tryptophan or a variant thereof. In some embodiments, X8 is W1Me. In some embodiments, X8 is tyrosine or a variant thereof. In some embodiments, X8 is N-methylated amino acid or a substitute thereof. In some embodiments, X8 is N-alkylated amino acid or a substitute thereof. In some embodiments, X8 is KCOpipzaa. In some embodiments, X8 is K. In some embodiments, X8 is valine (V). In some embodiments, X8 is Qglucamine. In some embodiments, X8 is Cit. In some embodiments, X8 is hCit. In some embodiments, X8 is absent. In some embodiments of Formulae (I), (I-1), (I-2), (I-3), (I-4), (I-5), (Ia), (Ib), (Ic), (III-1) and (III-2), X8 is a hydrophobic amino acid, a hydrophilic amino acid, an N-methylated amino acid, or an amino acid with a functional side chain. In some embodiments; X8 is G substituted with one or two straight or branched C₁₋₅ alkyl, A substituted with C₃₋₇ cycloalkyl, or a hydrophilic amino acid wherein the hydrophilic amino acid comprises an L-amino acid comprising —NH₂, one or more —OH, —C(O)OH, —NHC(NH)NH₂, —NHC(O)NH₂, —C(O)NH₂, —NHC(O)CH₃; or the hydrophilic amino acid comprises a zwitterion. In some embodiments, X8 is V, A, E, N, K, Qglucamine, KCOpipzaa, Q, Hse, N, Cit, Hcit, Kac, DapAc, OrnAc, T, alT, Aib, Alb, or 3Py6NH2. In some embodiments, X8 is A, E, N, K, Qglucamine, KCOpipzaa, Q, Hse, N, Cit, Hcit, Kac, DapAc, OrnAc, T, alT, Aib, Alb, or 3Py6NH2. In some embodiments, X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L. In certain embodiments, X8 is KCOpipzaa, V, Qglucamine, Cit, Hcit, K, or 3Py6NH2. In certain embodiments, X8 is KCOpipzaa, Qglucamine, Cit, Hcit, K, or 3Py6NH2. In some embodiments, X8 is V, KCOpipzaa, Cit, Qglucamine, hCit, Aib, Alb, Norleucine, or Norvaline. In some embodiments, X8 is KCOpipzaa, Cit, Qglucamine, hCit, Aib, Alb, Norleucine, or Norvaline. In some embodiments, X8 is KCOpipzaa, N, Cit, hCit, KAc, DapAc, OrnAc, A, T, alT, Aib, Alb, Qglucamine, Hgl, Q, E, Hgn, or K. In some embodiments, X8 is a hydrophobic amino acid. In some embodiments, X8 is G substituted with straight or branched C₁₋₅ alkyl. In some embodiments, X8 is G substituted with one or more substituents selected from methyl, ethyl, propyl, isopropyl, butyl, isobutyl, pentyl, and isopentyl. In some embodiments, X8 A substituted with C₃₋₇ cycloalkyl. In some embodiments, X8 is A substituted with cyclopropyl. In some embodiments, X8 is A substituted with cyclobutyl. In some embodiments, X8 is A substituted with cyclopentyl. In some embodiments, X8 is A substituted with cyclohexyl. In some embodiments, X8 is A substituted with cycloheptyl. In some embodiments, X8 is V, Aib, Alb, Norleucine, or Norvaline. In some embodiments, X8 is Aib, Alb, Norleucine, or Norvaline. In some embodiments, X8 is V. In some embodiments, X8 is Aib. In some embodiments, X8 is Alb. In some embodiments, X8 is Norleucine. In some embodiments, X8 is Norvaline. In some embodiments, X8 is a hydrophilic amino acid. In some embodiments, X8 is an amino acid comprising —NH₂, one or more —OH, —C(O)OH, —NHC(NH)NH₂, —NHC(O)NH₂, —C(O)NH₂, or —NHC(O)CH₃. In some embodiments, X8 is an L-amino acid comprising —NH₂, one or more —OH, —C(O)OH, —NHC(NH)NH₂, —NHC(O)NH₂, —C(O)NH₂, or —NHC(O)CH₃. In some embodiments, X8 is an amino acid comprising a zwitterion. In some embodiments, X8 is Cit or a variant thereof. In some embodiments, X8 is Cit or hCit. In some embodiments, X8 is KCOpipzaa. In some embodiments, X8 is Qglucamine. In some embodiments, the payload molecule or linker is attached to X8. In some embodiments, X1 is directly bound to X8.

In some embodiments of Formulae (I), (I-1), (I-2), (I-3), (I-4), (I-5), (Ia), (Ib), (Ic), (III-1) and (III-2), X9 is W or a variant thereof. In some embodiments, X9 is a canonical amino acid. In some embodiments, X9 is an unnatural amino acid. In some embodiments, X9 is W1Me, W1Me7Cl, F23dMe, Nal1, Nal2, W1Et, Nal21N, 3Bzf, 3Bzt, Nal15N, Nal14N, Nal24N, Nal28N, F23dC, or W1Me7N. In some embodiments, X9 is W1Me or F23dMe. In some embodiments, X9 is W1Me. In some embodiments, X9 is W1Me7Cl. In some embodiments, X9 is W1Me7N. In some embodiments, X9 is absent. In some embodiments, X9 is F23dMe. In some embodiments, X9 an amino acid having aromatic ring or a substitute thereof. In some embodiments of Formulae (I), (I-1), (I-2), (I-3), (I-4), (I-5), (Ia), (Ib), (Ic), (III-1) and (III-2), X9 is an amino acid comprising an aromatic ring. In some embodiments, X9 is an amino acid comprising an aryl group. In some embodiments, X9 is an amino acid comprising an optionally substituted phenyl group. In some embodiments, X9 is an amino acid comprising an optionally substituted naphthyl group. In some embodiments, X9 is an amino acid comprising a heteroaryl group. In some embodiments, X9 is an amino acid comprising an optionally substituted monocyclic heteroaryl group. In some embodiments, X9 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X9 is W, Y, or a variant thereof (such as an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH3, -ethyl, —Cl, and —F). In some embodiments, X9 is an amino acid comprising an aromatic ring (e.g., W or a variant thereof). In some embodiments, X9 is F or a variant thereof, or W or a variant thereof. In some embodiments, X9 is W1Me, W1Me7Cl, W1Me7N, F23dMe, W1Et, W7Me, W, F, or 7-AzaTrp. In some embodiments, X9 is F or a variant thereof. In some embodiments, X9 is F or F23dMe. In some embodiments, X9 is F. In some embodiments, X9 is F23dMe. In some embodiments, X9 is W or a variant thereof. In some embodiments, X9 is W1Me, W1Me7Cl, W1Me7N, W, 7-AzaTrp, W7Me, or W1Et. In some embodiments, X9 is W1Me or F23dMe. In some embodiments, X9 is W1Me. In some embodiments, X9 is W1Me7Cl. In some embodiments, X9 is W1Me7N. In some embodiments, X9 is W. In some embodiments, X9 is 7-AzaTrp. In some embodiments, X9 is W7Me. In some embodiments, X9 is W1Et. In some embodiments, the payload molecule or linker is attached to X9. In some embodiments, X1 is directly bound to X9.

In some embodiments of Formulae (I), (I-1), (I-2), (I-3), (I-4), (I-5), (III-1) and (III-2), X10 is absent, T or a variant thereof. In some embodiments, X10 is a canonical amino acid. In some embodiments, X10 is an unnatural amino acid. In some embodiments, X10 is threonine (T). In some embodiments, X10 is absent. In some embodiments of Formulae (I), (I-1), (I-2), (I-3), (I-4), (I-5), (III-1) and (III-2), X10 is absent, or a polar amino acid (e.g., T or a variant thereof). In some embodiments, X10 is absent, Q, Hgn, S or a variant thereof, T or variant thereof optionally substituted with straight or branched C₁₋₅ alkyl, K or a variant thereof, Cit or a variant thereof, or an L-amino acid substituted with —NHC(NH)NH₂—NHC(O)NH₂, —C(O)NH₂, or —NHC(O)CH₃. In some embodiments, X10 is absent, T, Q, S, Hgn, Alpha-methylserine, hSer, hThr, N, OrnAc, LysAc, Cit, or hCit. In some embodiments, X10 is absent. In some embodiments, X10 is a polar amino acid. In some embodiments, X10 is Q. In some embodiments, X10 is Hgn. In some embodiments, X10 is S or a variant thereof. In some embodiments. X10 is S, Alpha-methylserine, or hSer. In some embodiments, X10 is S. In some embodiments, X10 is Alpha-methylserine. In some embodiments, X10 is hSer. In some embodiments, X10 is T or a variant thereof optionally substituted with straight or branched Cis alkyl. In some embodiments, X10 is T or hThr. In some embodiments, X10 is T. In some embodiments, X10 is hThr. In some embodiments, X10 is T substituted with methyl, ethyl, propyl, isopropyl, butyl, isobutyl, pentyl, or isopentyl. In some embodiments, X10 is N. In some embodiments, X10 is K or a variant thereof. In some embodiments, X10 is K, OrnAc, or LysAc. In some embodiments, X10 is K. In some embodiments, X10 is OrnAc. In some embodiments, X10 is LysAc. In some embodiments, X10 is Cit or a variant thereof. In some embodiments, X10 is Cit or hCit. In some embodiments, X10 is Cit. In some embodiments, X10 is hCit. In some embodiments, the payload molecule or linker is attached to X10. In some embodiments, X1 is directly bound to X10.

In some embodiments of Formulae (I), (I-1), (I-2), (I-3), (I-4), (I-5), (III-1) and (III-2), X11 is absent, a hydrophilic amino acid, or a substitute thereof. In some embodiments, X11 is serine, threonine, tyrosine, asparagine, glutamine, or a substitute thereof. In some embodiments, X11 is a canonical amino acid. In some embodiments, X11 is an unnatural amino acid. In some embodiments, X11 is Hgn. In some embodiments, X11 is K. In some embodiments, X11 is glutamate. In some embodiments, X11 is hArg. In some embodiments, X11 is hCit. In some embodiments, X11 is Nmm. In some embodiments, X11 is Ndm. In some embodiments, X11 is Har. In some embodiments, X11 is R. In some embodiments, X11 is Har. In some embodiments, X11 is Arg (R). In some embodiments, X11 is Cit. In some embodiments, X11 is asparagine. In some embodiments, X11 is absent. In some embodiments of Formulae (I), (I-1), (I-2), (I-3), (I-4), (I-5), (III-1) and (III-2), X11 is absent, a hydrophilic amino acid, or an amino acid with a functional side chain. In some embodiments, X11 is a hydrophilic amino acid. In some embodiments of Formulas (I), (I-1), (I-2), (I-3), (I-4), (I-5), (III-1) and (III-2), X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof). In some embodiments, X11 is an amino acid comprising an electrically charged side chain. In some embodiments, X11 is an amino acid comprising a polar uncharged side chain. In some embodiments, X11 is an amino acid comprising —OH, —NH₂, —C(O)OH, —NHC(═NH)NH₂, —NHC(O)NH₂, —C(O)NH₂, or —NHC(O)CH₃ group. In some embodiments, X11 has an electrically charged side chain. In some embodiments, X11 has a positively charged side chain. In some embodiments, X11 has a negatively charged side chain. In some embodiments, X11 is zwitterionic. In some embodiments, X11 comprises a —OH, —COOH, —NH— or NH₂ moiety. In some embodiments, X11 comprises —OH, —C(O)OH, —NHC(═NH)NH₂, —NHC(O)NH₂, —C(O)NH₂, or —NHC(O)CH₃. In some embodiments, X11 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X11 is absent, E, Hgn, R or a variant thereof, Cit or a variant thereof, Hgl, K or a variant thereof, D, N, or Q. In some embodiments, X11 is absent, E, Hgn, R, hArg, Cit, hCit, Hgl, Orn, D. N, Q, DapAc, OrnAc, DabAc, or norCit. In some embodiments, X11 is absent, arginine (R), asparagine (N), aspartate (D), glutamine (Q), lysine (K), or an unnatural hydrophilic amino acid. In some embodiments, X11 is absent, Hgn, R, hArg, Cit, hCit, Hgl, Orn, D, N, Q, DapAc, OrnAc, DabAc, or norCit. In some embodiments, X11 is Hgn, R, hArg, Cit, hCit, Hgl, Orn, D, N, Q, DapAc, OrnAc, DabAc, or norCit. In some embodiments, X11 is Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X11 is Q, K, G, S, T, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X11 is Hgn, N, R, Har, Nmm, Ndm, E, or K. In some embodiments, X11 is absent. In some embodiments, X11 is a hydrophilic amino acid. In some embodiments, X11 is E. In some embodiments, X11 is Hgn. In some embodiments, X11 is R or a variant thereof. In some embodiments, X11 is R or hArg. In some embodiments, X11 is R. In some embodiments, X11 is hARg. In some embodiments, X11 is Cit or a variant thereof. In some embodiments, X11 is Cit, hCit, or norCit. In some embodiments, X11 is Cit. In some embodiments, X11 is hCit. In some embodiments, X11 is norCit. In some embodiments, X11 is Hgl. In some embodiments, X11 is K or a variant thereof. In some embodiments, X11 is K, Om, OrnAc, DabAc, or DapAc. In some embodiments, X11 is K. In some embodiments, X11 is Orn. In some embodiments, X11 is OrnAc. In some embodiments, X11 is DabAc. In some embodiments, X11 is DapAc. In some embodiments, X11 is D, N or Q. In some embodiments, X11 is D. In some embodiments, X11 is N. In some embodiments, X11 is Q. In some embodiments, the payload molecule or linker is attached to X11. In some embodiments, X1 is directly bound to X11.

In some embodiments of Formulae (I), (I-5), (Ia), (Ib), (Ic), and (III-2), X12 is C or a variant thereof. In some embodiments, X12 is a canonical amino acid. In some embodiments, X12 is an unnatural amino acid. In some embodiments, X12 is cysteine. In some embodiments, X12 is a substitute of cysteine. In some embodiments, X12 is homocysteine. In some embodiments, X12 is CdMe. In some embodiments, X12 is C3SMe. In some embodiments, X12 is C3RMe. In some embodiments, the payload molecule or linker is attached to X12. In some embodiments of Formulae (I), (I-5), (Ia), (Ib), (Ic), and (III-2), X12 is C or a variant thereof. In some embodiments, X12 is X12 is C, hCys, CdMe, C3RMe, C3SMe, Selenocysteine, do, or Penicillamine. In some embodiments, X12 is C. In some embodiments, X12 is hCys. In some embodiments, X12 is CdMe. In some embodiments, X12 is C3RMe. In some embodiments, X12 is C3SMe. In some embodiments, X12 is Selenocysteine. In some embodiments, X12 is do. In some embodiments, X12 is Penicillamine. In some embodiments, the payload molecule or linker is attached to X12. In some embodiments, X1 is directly bound to X12.

In some embodiments, the peptide or the pharmaceutically accepted salt thereof has a cyclic structure, wherein the first amino acid (or X1) is covalently linked to the last amino acid (or X12).

In some embodiments, the peptide or the pharmaceutically accepted salt thereof has a cyclic structure having an amino acid (e.g., a chloroacetylated amino acid) in the first residue X1 and a cysteine residue or a variant thereof, and wherein the amino acid (e.g., the chloroacetylated amino acid) in X1 and the cysteine residue or variant thereof form a covalent bond.

In some embodiments, the peptide has a monocyclic structure. In certain embodiments, the amino acid X1 and a cysteine or a variant thereof form a covalent bond.

In some embodiments, the peptide of Formula (I) has a structure of Formula (I-1), or a pharmaceutically acceptable salt thereof,

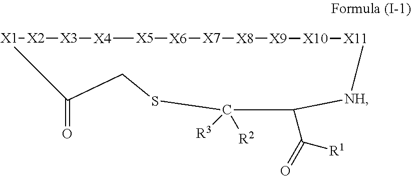

wherein
   R¹ is selected from the group consisting of NH₂ and OH;
   R² is selected from the group consisting of H or C₁₋₃ alkyl;
   R³ is selected from the group consisting of H or C₁₋₃ alkyl;
   wherein the attachment point to the payload molecule or the linker is not shown, and wherein X1-X11 are described in Formula (I).

In some embodiments, the peptide of Formula (I-1) has a structure of Formula (I-2), or a pharmaceutically acceptable salt thereof,

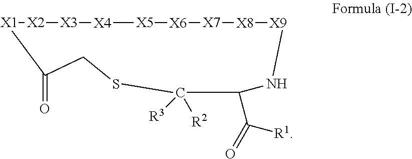

In some embodiments, the peptide of Formula (I-1) has a structure of Formula (I-3), or a pharmaceutically acceptable salt thereof,

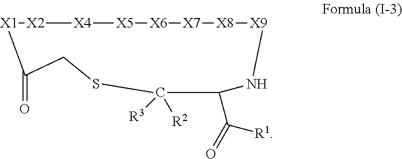

In some embodiments, the peptide of Formula (I-1) has a structure of Formula (I-4), or a pharmaceutically acceptable salt thereof,

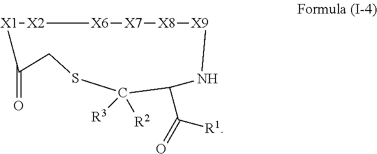

In some embodiments of Formula (I-1), (1-2), (1-3) or (1-4), R¹ is OH. In some embodiments of Formula (I-1), (I-2), (I-3) or (I-4), R¹ is NH₂.

In some embodiments of Formula (I-1), (I-2), (I-3) or (I-4), R² is H. In some embodiments of Formula (I-1), (I-2), (I-3) or (I-4), R² is C₁₋₃ alkyl. In some embodiments of Formula (I-1), (I-2), (I-3) or (I-4), R² is methyl.

In some embodiments of Formula (I-1), (I-2), (I-3) or (I-4), R³ is H. In some embodiments of Formula (I-1), (I-2), (I-3) or (I-4), R³ is C₁₋₃ alkyl. In some embodiments of Formula (I-1), (I-2), (I-3) or (I-4), R³ is methyl.

In some embodiments, the peptide of Formula (I) has a structure of Formula (I-5), or a pharmaceutically acceptable salt thereof,

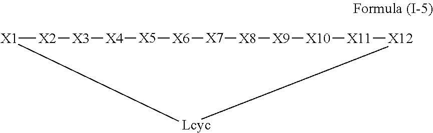

wherein X1-X12 have the definition described above and Lcyc is a ring closing group that covalently connecting X1 with X12.

In some embodiments, the Lcyc is a group selected from Table 4B. In some embodiments, the Lcyc is formed by reacting the first and the second functional groups in Table 4C.

In some embodiments, the peptide of Formula (I) or a pharmaceutically acceptable salt thereof,

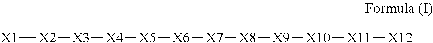

wherein,
X1 is any amino acid (e.g., D-amino acid);
X2 is an amino acid comprising an aromatic ring or a variant thereof, or N-methylated amino acid thereof;
X3 is N or a variant thereof;
X4 is any hydrophobic amino acid or a variant thereof;
X5 is a hydrophilic amino acid or a variant thereof;
X6 is a hydrophilic amino acid or amino acid having aromatic ring, or N-methylated amino acid thereof;
X7 is W or a variant thereof;
X8 is V or hydrophilic amino acid or a variant thereof;
X9 is W or a variant thereof;
X10 is T or a variant thereof;
X11 is any hydrophilic amino acid; and
X12 is C or a variant thereof.

In some embodiments of Formula (I), wherein,
X1 is D-amino acid (such as da, df3CON, dahp, or dkCOpipzaa);
X2 is N-methylated phenylalanine or a variant thereof (such as Me3Py, MeF, MeF3H, or MeF3CN);
X3 is N;
X4 is a hydrophobic amino acid or N-methylated amino acid (such as leucine, Cbg, or Chg);
X5 is a Hgn, asparagine (N), 2,4-Diaminobutyric Acid (Dab), Qglucamine, KCOpipzaa, Hgl, Nmm, Ndm, or lysine (K);
X6 is asparagine (N) or N-methylated glutamic acid (E), N-methylated asparagine, N-methylated phenylalanine (F) or substitutions thereof (such as Qglucamine, MeE, MeN, Me3Py, MeF, MeF4C, or N);
X7 is W1Me, W1Me7Cl, or W1Me7N;
X8 is KCOpipzaa, V, Qglucamine, Cit, Hcit, or K;
X9 is W1Me or F23dMe;
X10 is T;
X11 is hArg, hCit, Citrulline (Cit), A Hgn, asparagine (N), Arginine (R), Har, Nmm, Ndm, Glutamic Acid (E), lysine (K); and
X12 is cysteine.

In some embodiments, an amino acid of Formula (I) has a sequence of Formula (Ia), or a pharmaceutically acceptable salt thereof,

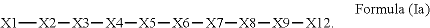

In some embodiments, an amino acid of Formula (I) has a sequence of Formula (Ib), or a pharmaceutically acceptable salt thereof,

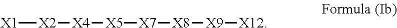

In some embodiments, an amino acid of Formula (I) has a sequence of Formula (Ic), or a pharmaceutically acceptable salt thereof,

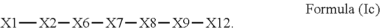

In some embodiments, a herein described peptide has an amino acid sequence according to Formula (Ia), or a pharmaceutically acceptable salt thereof.

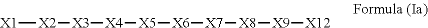

wherein,
   X1 is any amino acid (e.g., a D-amino acid);
   X2 is an amino acid comprising an aromatic ring or a variant thereof, or an N-methylated amino acid thereof;
   X3 is N or a variant thereof;
   X4 is any hydrophobic amino or a variant thereof,
   X5 is a hydrophilic amino acid or a variant thereof;
   X6 is a hydrophilic amino acid or amino acid having aromatic ring, or an N-methylated amino acid thereof;
   X7 is W or a variant thereof;
   X8 is any hydrophilic amino acid or a variant thereof;
   X9 is W or a variant thereof; and
   X12 is C or a variant thereof.

In some embodiments, a herein described peptide has an amino acid sequence according to Formula (Ib), or a pharmaceutically acceptable salt thereof,

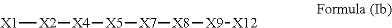

wherein,
X1 is any amino acid (e.g., D-amino acid);
X2 is an amino acid comprising an aromatic ring or a variant thereof, or N-methylated amino acid thereof;
X4 is any hydrophobic amino or a variant thereof,
X5 is a hydrophilic amino acid or a variant thereof;
X7 is W or a variant thereof;
X8 is an N-methylated amino acid;
X9 is W or a variant thereof; and
X12 is C or a variant thereof.

In some embodiments, a herein described peptide has an amino acid sequence according to Formula (Ic), or a pharmaceutically acceptable salt thereof,

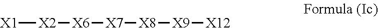

wherein,
X1 is any amino acid (e.g., D-amino acid);
X2 is an amino acid comprising an aromatic ring or a variant thereof, or N-methylated amino acid thereof;
X6 is an N-methyl amino acid;
X7 is W or a variant thereof;
X8 is an N-methyl amino acid;
X9 is W or a variant thereof; and
X12 is C or a variant thereof.

In some embodiments, the peptide of Formula (I), (Ia), (Ib), and/or (Ic) are monocyclic. In some embodiments, the amino acid in X1 and the cysteine or the substitution of cysteine are bound.

In some embodiments, the peptide or the salt thereof comprises an amino acid sequence that is at least 95% identical to a sequence selected from SEQ ID NOs: 1-171, or a sequence with up to 1, 2, 3, 4, or 5 substitutions by a conserved variant compared to any one of the sequences selected from SEQ ID NOs: 1-171.

In some embodiments, the peptide or the salt thereof consists of an amino acid sequence selected from SEQ ID NOs: 1-171.

In some embodiments, the peptide consists of an amino acid sequence selected from SEQ ID NOs: 1-122, 159-163, and 165-171, and the peptide has a cyclic structure having a cysteine residue or a variant thereof at the 12^{th} residue, and wherein the amino acid at X1 (e.g., a chloroacetylated amino acid) and the cysteine residue or a variant thereof at the 12^{th} residue form a covalent bond (e.g., by reacting a chloroacetyl group in the amino acid of X1 with the cysteine residue or a variant thereof).

In some embodiments, the peptide consists of an amino acid sequence selected from SEQ ID NOs: 123-149 and 164, and the peptide has a cyclic structure having a cysteine residue or a variant thereof at 10^{th} residue, and wherein the amino acid at X1 (e.g., a chloroacetylated amino acid) and the cysteine residue or a variant thereof at 10^{th} residue form a covalent bond.

In some embodiments, the peptide has a binding affinity to a human EphA2 of at most 100 nM as determined by K_{d} in surface plasmon resonance (SPR) analysis.

In some embodiments, the peptide has a binding affinity to a human EphA2 of at most 1 nM as determined by K_{d} in surface plasmon resonance (SPR) analysis.

In some embodiments, a peptide of the present disclosure binds to a ligand-binding domain (LBD) of human EphA2.

In some embodiments, a peptide of the present disclosure has good contact with Asp53 and/or Glu157 of the human EphA2, according to SEQ ID NO: 276. In some embodiments, a peptide of the present disclosure interacts with Asp53 and/or Glu157 of the human EphA2, according to SEQ ID NO: 276. In some embodiments, a peptide of the present disclosure interacts with Asp53 and/or Glu157 of the human EphA2, according to SEQ ID NO: 277. The interaction can be the formation of one or more hydrogen bonds, Van der Waals interactions, dipole-dipole interactions, or pi-pi stacking interactions.

In some embodiments, a peptide of the present disclosure interacts with human EphA2 at one or more residues selected from Asp53, Met55, Asn57, Met59, Met66, Thr101, Arg103, Phe156, Glu157, Arg159, Val161, Val189, and Ala190. In some embodiments, a peptide of the present disclosure binds to Asp53 and Glu157 of the human EphA2. In some embodiments, amino acid residue X5 of Formula (I) interact with Glu157 of a human EphA2. In some embodiments, amino acid residue X6 of Formula (I) interacts with Arg159 of a human EphA2. In some embodiments, amino acid residue X7 of Formula (I) interacts with one or more of Phe156, Thr101, Asn57, Val161, Met59, Ala190, and Met66 of a human EphA2. In some embodiments, amino acid residue X9 of Formula (I) interacts with one or more of Phe156, Arg103, and Val189. In some embodiments, amino acid residue X11 of Formula (I) interact with Asp53 of a human EphA2. In some embodiments, amino acid residue X7 of Formula (I) forms a pi-pi stacking interaction with Phe156 of a human EphA2 of the human EphA2. In some embodiments, amino acid residue X9 of Formula (I) forms a pi-pi stacking interaction with Phe156 of a human EphA2. In some embodiments, amino acid residue X2 of Formula (I) interacts with the backbone carbonyl of C70 of human EphA2 protein via intermolecular aromatic H-bond interactions.

In some embodiments, when a peptide of Formula (I), or a conjugate comprising the peptide, is bound to a human EphA2, amino acid residue X2 of Formula (I) is located less than 15 Å from the C70 of the human EphA2. In some embodiments, X2 is located less than 10λ from the C70. In some embodiments, X2 is located less than 6 Å from the C70. In some embodiments, X2 is located less than 4 Å from the C70.

In some embodiments, when a peptide of Formula (I), or a conjugate comprising the peptide, is bound to a human EphA2, amino acid residue X7 of Formula (I) is located less than 10 Å from the Phe156 of the human EphA2. In some embodiments, X7 is located less than 6 Å from the Phe156. In some embodiments, X7 is located less than 4 Å from the Phe156.

In some embodiments, when a peptide of Formula (I), or a conjugate comprising the peptide, is bound to a human EphA2, amino acid residue X7 of Formula (I) is located less than 20 Å from the Thr101 of the human EphA2. In some embodiments, X7 is located less than 15 Å from the Thr101. In some embodiments, X7 is located less than 10 Å from the Thr101. In some embodiments, X7 is located less than 6 Å from the Thr101. In some embodiments, X7 is located less than 4 Å from the Thr101.

In some embodiments, when a peptide of Formula (I), or a conjugate comprising the peptide, is bound to a human EphA2, amino acid residue X7 of Formula (I) is located less than 20 Å from the Asn57 of the human EphA2. In some embodiments, X7 is located less than 15 Å from the Asn57. In some embodiments, X7 is located less than 10 Å from the Asn57. In some embodiments, X7 is located less than 6 Å from the Asn57. In some embodiments, X7 is located less than 4 Å from the Asn57.

In some embodiments, when a peptide of Formula (I) is, or a conjugate comprising the peptide, bound to a human EphA2, amino acid residue X7 of Formula (I) is located less than 20 Å from the Val161 of the human EphA2. In some embodiments, X7 is located less than 15 Å from the Val161. In some embodiments, X7 is located less than 10 Å from the Val161. In some embodiments, X7 is located less than 6 Å from the Val161. In some embodiments, X7 is located less than 4 Å from the Val161.

In some embodiments, when a peptide of Formula (I), or a conjugate comprising the peptide, is bound to a human EphA2, amino acid residue X7 of Formula (I) is located less than 20 Å from the Met59 of the human EphA2. In some embodiments, X7 is located less than 15 Å from the Met59. In some embodiments, X7 is located less than 10 Å from the Met59. In some embodiments, X7 is located less than 6 Å from the Met59. In some embodiments, X7 is located less than 4 Å from the Met59.

In some embodiments, when a peptide of Formula (I), or a conjugate comprising the peptide, is bound to a human EphA2, amino acid residue X7 of Formula (I) is located less than 20 Å from the Ala190 of the human EphA2. In some embodiments, X7 is located less than 15 Å from the Ala190. In some embodiments, X7 is located less than 10 Å from the Ala190. In some embodiments, X7 is located less than 6 Å from the Ala190. In some embodiments, X7 is located less than 4 Å from the Ala190.

In some embodiments, when a peptide of Formula (I), or a conjugate comprising the peptide, is bound to a human EphA2, amino acid residue X7 of Formula (I) is located less than 20 Å from the Met66 of the human EphA2. In some embodiments, X7 is located less than 15 Å from the Met66. In some embodiments, X7 is located less than 10 Å from the Met66. In some embodiments, X7 is located less than 6 Å from the Met66. In some embodiments, X7 is located less than 4 Å from the Met66.

In some embodiments, when a peptide of Formula (I), or a conjugate comprising the peptide, is bound to a human EphA2, amino acid residue X9 of Formula (I) is located less than 10 Å from the Phe156 of the human EphA2. In some embodiments, X9 is located less than 6 Å from the Phe156. In some embodiments, X9 is located less than 4 Å from the Phe156.

In some embodiments, when a peptide of Formula (I), or a conjugate comprising the peptide, is bound to a human EphA2, amino acid residue X9 of Formula (I) is located less than 15 Å from the Asn3 of the human EphA2. In some embodiments, X9 is located less than 10 Å from the Asn3. In some embodiments, X9 is located less than 6 Å from the Asn3. In some embodiments, X9 is located less than 4 Å from the Asn3.

In some embodiments, when a peptide of Formula (I), or a conjugate comprising the peptide, is bound to a human EphA2, amino acid residue X9 of Formula (I) is located less than 15 Å from the Arg103 of the human EphA2. In some embodiments, X9 is located less than 10 Å from the Arg103. In some embodiments, X9 is located less than 6 Å from the Arg103. In some embodiments, X9 is located less than 4 Å from the Arg103.

In some embodiments, when a peptide of Formula (I), or a conjugate comprising the peptide, is bound to a human EphA2, amino acid residue X9 of Formula (I) is located less than 15 Å from the Val189 of the human EphA2. In some embodiments, X9 is located less than 10 Å from the Val189. In some embodiments, X9 is located less than 6 Å from the Val189. In some embodiments, X9 is located less than 4 Å from the Val189.

In some embodiments, when a peptide of Formula (I), or a conjugate comprising the peptide, is bound to a human EphA2, amino acid residue X8 of Formula (I) is located less than 10 Å from the Phe156 of the human EphA2. In some embodiments, X8 is located less than 6 Å from the Phe156. In some embodiments, X8 is located less than 4 Å from the Phe156.

In some embodiments, when a peptide of Formula (I), or a conjugate comprising the peptide, is bound to a human EphA2, amino acid residue X2 of Formula (I) is located less than 15 Å from the C70 of the human EphA2. In some embodiments, X2 is located less than 10 Å from the C70. In some embodiments, X2 is located less than 7 Å from the C70. In some embodiments, X2 is located less than 4 Å from the C70.

In some embodiments, when a peptide of Formula (I), or a conjugate comprising the peptide, is bound to a human EphA2, amino acid residue X7 of Formula (I) is located less than 10 Å from the Phe156 of the human EphA2. In some embodiments, X7 is located less than 6 Å from the Phe156. In some embodiments, X7 is located less than 3 Å from the Phe156.

In some embodiments, when a peptide of Formula (I), or a conjugate comprising the peptide, is bound to a human EphA2, amino acid residue X9 of Formula (I) is located less than 20 Å from the Thr101 of the human EphA2. In some embodiments, X9 is located less than 15 Å from the Thr101. In some embodiments, X9 is located less than 10 Å from the Thr101. In some embodiments, X9 is located less than 6 Å from the Thr101. In some embodiments, X9 is located less than 5 Å from the Thr101.

In some embodiments, when a peptide of Formula (I), or a conjugate comprising the peptide, is bound to a human EphA2, amino acid residue X8 of Formula (I) is located less than 20 Å from the Asn57 of the human EphA2. In some embodiments, X8 is located less than 15 Å from the Asn57. In some embodiments, X8 is located less than 10 Å from the Asn57. In some embodiments, X8 is located less than 6 Å from the Asn57. In some embodiments, X8 is located less than 4 Å from the Asn57.

In some embodiments, when a peptide of Formula (I) is, or a conjugate comprising the peptide, bound to a human EphA2, amino acid residue X7 of Formula (I) is located less than 20 Å from the Val161 of the human EphA2. In some embodiments, X7 is located less than 15 Å from the Val161. In some embodiments, X7 is located less than 11 Å from the Val161. In some embodiments, X7 is located less than 6 Å from the Val161. In some embodiments, X7 is located less than 5 Å from the Val161.

In some embodiments, when a peptide of Formula (I), or a conjugate comprising the peptide, is bound to a human EphA2, amino acid residue X7 of Formula (I) is located less than 20 Å from the Met59 of the human EphA2. In some embodiments, X7 is located less than 15 Å from the Met59. In some embodiments, X7 is located less than 11 Å from the Met59. In some embodiments, X7 is located less than 6 Å from the Met59. In some embodiments, X7 is located less than 4 Å from the Met59.

In some embodiments, when a peptide of Formula (I), or a conjugate comprising the peptide, is bound to a human EphA2, amino acid residue X7 of Formula (I) is located less than 20 Å from the Ala190 of the human EphA2. In some embodiments, X7 is located less than 15 Å from the Ala190. In some embodiments, X7 is located less than 11 Å from the Ala190. In some embodiments, X7 is located less than 6 Å from the Ala190. In some embodiments, X7 is located less than 4 Å from the Ala190.

In some embodiments, when a peptide of Formula (I), or a conjugate comprising the peptide, is bound to a human EphA2, amino acid residue X7 of Formula (I) is located less than 20 Å from the Met66 of the human EphA2. In some embodiments, X7 is located less than 15 Å from the Met66. In some embodiments, X7 is located less than 10 Å from the Met66. In some embodiments, X7 is located less than 6 Å from the Met66. In some embodiments, X7 is located less than 4 Å from the Met66.

In some embodiments, when a peptide of Formula (I), or a conjugate comprising the peptide, is bound to a human EphA2, amino acid residue X2 of Formula (I) is located less than 15 Å from the Arg103 of the human EphA2. In some embodiments, X2 is located less than 10 Å from the Arg103. In some embodiments, X2 is located less than 6 Å from the Arg103. In some embodiments, X2 is located less than 4 Å from the Arg103.

In some embodiments, when a peptide of Formula (I), or a conjugate comprising the peptide, is bound to a human EphA2, amino acid residue X9 of Formula (I) is located less than 15 Å from the Val189 of the human EphA2. In some embodiments, X9 is located less than 10 Å from the Val189. In some embodiments, X9 is located less than 6 Å from the Val189. In some embodiments, X9 is located less than 4 Å from the Val189.

In certain embodiments, the peptide has a plasma half-life (T_{1/2}) of at least 50, 100, 150, 200, 250, 300, 350, 400, 450, or 500 minutes as determined in vitro in human plasma at 37° C. In certain embodiments, the peptide has a plasma half-life (T_{1/2}) of at least 250 minutes as determined in vitro in human plasma at 37° C.

In some embodiments, a conjugate of the present disclosure has a structure of Formula (III-1),

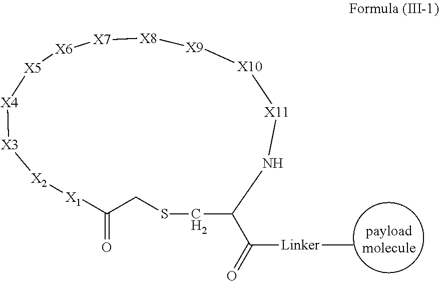

wherein -Linker- represents the linker.

In some embodiments, a conjugate comprising a cyclic peptide of formula (I) has a structure of Formula (III-2),

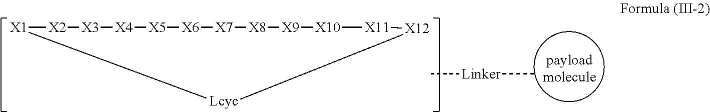

wherein
   X1-X12 have the definition described above and Lcyc is a ring closing group that covalently connecting X1 with X12; and
   -Linker- represents the linker.

In some embodiments, the Lcyc is a group selected from Table 4B. In some embodiments, the Lcyc is formed by reacting the first and the second functional groups in Table 4C. In some embodiments, the Lcyc is —C(═O)—CH₂—. In some embodiments, the Loyd is —C(═O)—CH₂—, which is formed by reacting with a chloroacetylated (or bromoacetylated) amino acid with a cysteine. In some embodiments, the Lcyc is —C(═O)—CH₂—S—, which is formed by reacting with a chloroacetylated (or bromoacetylated) amino acid with an amino acid comprising a SH group.

In some embodiments, a peptide disclosed herein or a pharmaceutically accepted salt thereof has a cyclic structure having an amino acid (e.g., a chloroacetylated amino acid) in the first residue X1 and a cysteine residue or a variant thereof, and wherein the amino acid (e.g., the chloroacetylated amino acid) in X1 and the cysteine residue or a variant thereof are bound. In some embodiments, a peptide disclosed herein or a pharmaceutically accepted salt thereof has a cyclic structure having an amino acid (e.g., a chloroacetylated amino acid) in the first residue X1 and a cysteine residue or a variant thereof, and wherein the amino acid (e.g., the chloroacetylated amino acid) in X1 and the cysteine residue or a variant thereof form a covalent bond. In some embodiments, a peptide disclosed herein or a pharmaceutically accepted salt thereof has a cyclic structure having a bromoacetylated amino acid in the first residue X1 and a cysteine residue or a variant thereof, and wherein the bromoacetylated amino acid in X1 and the cysteine residue or a variant thereof form a covalent bond.

In some embodiments, the peptide consists of an amino acid sequence selected from SEQ ID NOs: 1-122, 159-163, and 165-171, and the peptide has a cyclic structure having a cysteine residue or a variant thereof at the 12th residue (X12). In some embodiments, the peptide consists of an amino acid sequence selected from SEQ ID NOs: 1-122, 159-163, and 165-171, and the peptide has a cyclic structure having a cysteine residue or a variant thereof at the 12^{th} residue (X12), and wherein the chloroacetylated amino acid and the cysteine residue or a variant thereof at 12^{th} residue form a covalent bond. In some embodiments, the chloroacetyl group can be replaced with a bromoacetyl group.

In some embodiments, the peptide consists of an amino acid sequence selected from SEQ ID NOs: 123-149 and 164, and the peptide has a cyclic structure having a cysteine residue or a variant thereof at the 10^{th} residue (X10). In some embodiments, the peptide consists of an amino acid sequence selected from SEQ ID NOs: 123-149 and 164, and the peptide has a cyclic structure having a cysteine residue or a variant thereof at the 10^{th} residue (X10), and wherein the amino acid at X1 (e.g., a chloroacetylated amino acid) and the cysteine residue or a variant thereof at the 10^{th} residue form a covalent bond. In some embodiments, the chloroacetyl group can be replaced with a bromoacetyl group.

In some embodiments, the peptide consists of an amino acid sequence selected from SEQ ID NOs: 150-157, and the peptide has a cyclic structure having a cysteine residue or a variant thereof at the 8^{th} residue (X8). In some embodiments, the peptide consists of an amino acid sequence selected from SEQ ID NOs: 150-157, and the peptide has a cyclic structure having a chloroacetylated amino acid and a cysteine residue or a variant thereof at the 8^{th} residue (X8), and wherein the chloroacetylated amino acid and the cysteine residue or a variant thereof at 8^{th} residue form a covalent bond. In some embodiments, the chloroacetyl group can be replaced with a bromoacetyl group.

In some embodiments, the peptide consists of an amino acid sequence selected from SEQ ID NO: 158, and the peptide has a cyclic structure having a cysteine residue or a variant thereof at the 7^{th} residue (X7). In some embodiments, the peptide consists of an amino acid sequence selected from SEQ ID NO: 158, and the peptide has a cyclic structure having a chloroacetylated amino acid and a cysteine residue or a variant thereof at the 7^{th} residue (X7), and wherein the chloroacetylated amino acid and the cysteine residue or a variant thereof at 7^{th} residue form a covalent bond. In some embodiments, the chloroacetyl group can be replaced with a bromoacetyl group.

In some embodiments, a peptide disclosed herein or a pharmaceutically salt thereof has a cyclic structure having the first amino acid covalently linked to the last amino acid.

In some embodiments, the peptide or the pharmaceutically accepted salt thereof has a cyclic structure having a chloroacetylated amino acid in X1 and a cysteine or substituted cysteine residue, and wherein the chloroacetylated amino acid in X1 and the cysteine or substituted cysteine are bound. In some embodiments, the peptide consists of an amino acid sequence selected from SEQ ID NOs: 1-171. In some embodiments, the peptide consists of an amino acid sequence selected from SEQ ID NOs: 1-171, and the peptide has a cyclic structure. In some embodiments, the peptide consists of an amino acid sequence selected from SEQ ID NOs: 1-171, and the peptide has a cyclic structure having a chloroacetylated amino acid and a cysteine or substituted cysteine residue at C-terminus, and wherein the chloroacetylated amino acid and the cysteine or substituted cysteine at C-terminus are bound. In some embodiments, the peptide has a cyclic structure having a chloroacetylated amino acid and; (i) a cysteine or substituted cysteine residue at the 12^{th} residue, and wherein the chloroacetylated amino acid and the cysteine or substituted cysteine at the 12^{th} residue are bound; or (ii) a cysteine or substituted cysteine residue at the 10^{th} residue, and wherein the chloroacetylated amino acid and the cysteine or substituted cysteine at the 10^{th} residue are bound. In some embodiments, the chloroacetyl group can be replaced with a bromoacetyl group.

For example, a cyclic peptide of formula (I) can have a structure as illustrated below

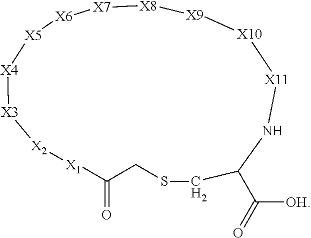

For example, a cyclic peptide of formula (I) can have a structure as illustrated below

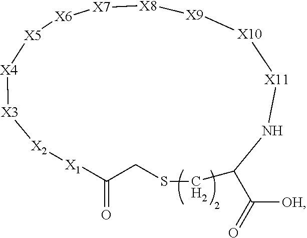

In some embodiments, a conjugate comprising a cyclic peptide of formula (I) has a structure of

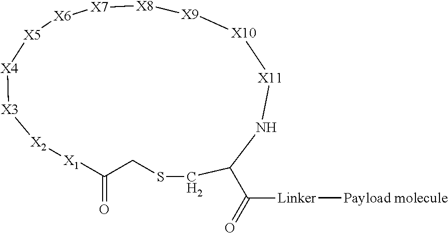

In some embodiments, a conjugate of the present disclosure has a structure of

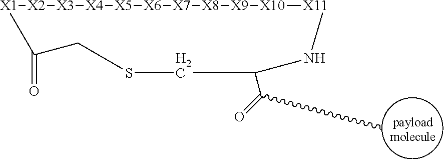

wherein [symbol] represents the linker.

In some embodiments, the peptide or the salt thereof comprises an amino acid sequence that is at least 95% identical to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158. In some embodiments, the peptide or the salt thereof comprises an amino acid sequence that is at least 80%, 85%, 90%, 95%, or 98% identical to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158. In some embodiments, the peptide or the salt thereof consists of an amino acid sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158. In some embodiments, the peptide or the salt thereof comprises an amino acid sequence that has at most 1, 2, 3, 4, or 5 amino acid residues that are different compared to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158. In some embodiments, the peptide or the salt thereof comprises an amino acid sequence that has at most 1, 2, 3, 4, or 5 additions, deletions and/or substitutions (including conservative substitutions) to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158. In some embodiments, the peptide or the salt thereof comprises an amino acid sequence that has at most 1 addition, deletion, or substitutions (including conservative substitutions) to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID NO: 158.

Exemplary peptides of the present disclosure include the peptides described in Table 1. In some embodiments, the peptides of Table 1 have a —C(═O)-halogen group attached to the N-terminus. In some embodiments, the peptides of Table 1 have a —C(═O)—CH₂-halogen group attached to the N-terminus. In some embodiments, the peptides of Table 1 have a —C(═O)-halogen group attached at residue position 1 (e.g., X1). In some embodiments, the peptides of Table 1 have a —C(═O)—CH₂-halogen group attached at residue position 1 (e.g., X1). In some embodiments, the peptides of Table 1 have a —C(═O)—Cl group attached to the N-terminus. In some embodiments, the peptides of Table 1 have a —C(═O)—CH₂—Cl group attached to the N-terminus. In some embodiments, the peptides of Table 1 have a —C(═O)—Cl group attached at residue position 1 (e.g., X1). In some embodiments, the peptides of Table 1 have a —C(═O)—CH₂—Cl group attached at residue position 1 (e.g., X1). In some embodiments, the peptides of Table 1 have a —C(═O)—Br group attached at residue position 1 (e.g., X1). In some embodiments, the peptides of Table 1 have a —C(═O)—CH₂—Br group attached at residue position 1 (e.g., X1).

In some embodiments, the conjugates of the disclosure have a —C(═O)-halogen group attached to the N-terminus. In some embodiments, the conjugates of the disclosure have a —C(═O)—CH₂-halogen group attached to the N-terminus. In some embodiments, the conjugates of the disclosure have a —C(═O)-halogen group attached at residue position 1 (e.g., X1). In some embodiments, the conjugates of the disclosure have a —C(═O)—CH₂-halogen group attached at residue position 1 (e.g., X1). In some embodiments, the conjugates of the disclosure have a —C(═O)—Cl group attached to the N-terminus. In some embodiments, the conjugates of the disclosure have a —C(═O)—CH₂—Cl group attached to the N-terminus. In some embodiments, the conjugates of the disclosure have a —C(═O)—Cl group attached at residue position 1 (e.g., X1). In some embodiments, the conjugates of the disclosure have a —C(═O)—CH₂—Cl group attached at residue position 1 (e.g., X1). In some embodiments, the conjugates of the disclosure have a —C(═O)—Br group attached at residue position 1 (e.g., X1). In some embodiments, the conjugates of the disclosure have a —C(═O)—CH₂—Br group attached at residue position 1 (e.g., X1). In some embodiments, the peptides in the conjugates of the disclosure are monocyclic.

In some embodiments, the peptides of the conjugates described herein are monocyclic peptides, wherein the —C(═O)—Cl at residue position 1 (e.g., X1) forms a bond with the cysteine at residue position 12 (e.g., X12). In some embodiments, the peptides of the conjugates described herein are monocyclic peptides, wherein the —C(═O)—CH₂—Cl at residue position 1 (e.g., X1) forms a bond with the cysteine at residue position 12 (e.g., X12). In some embodiments, the peptides in the conjugates described herein are monocyclic peptides with 12 amino acid residues forming the ring.

In some embodiments, the peptides of conjugates described herein are monocyclic peptides, wherein the —C(═O)—Cl at residue position 1 (e.g., X1) forms a bond with the cysteine at residue position 10 (e.g., X10). In some embodiments, the peptides in the conjugates described herein are monocyclic peptides with 10 amino acid residues forming the ring.

In one aspect, described herein is a peptide that has avidity for ephrin type-A receptor 2 (EphA2), wherein the peptide competes for binding to human EphA2 with a peptide that has an amino acid sequence including deletion, substitution, and/or addition of one or several amino acids in the amino acid of SEQ ID NO: 1:

(SEQ ID NO: 1)
da-MeF-N-L-Hgl-MeF-W1Me-V-W1Me-T-E-C

or a pharmaceutically acceptable salt thereof.

In one aspect, described herein is a peptide that has avidity for ephrin type-A receptor 2 (EphA2), wherein the peptide competes for binding to human EphA2 with a peptide that has a structure of Formula (I) as described herein (e.g., Formula (I-1) and Formula (I-2), or a pharmaceutically acceptable salt thereof.

In some embodiments, the peptide competes for binding to human EphA2 at one or more amino acid residues selected from Asp53, Met55, Asn57, Met59, Met66, Thr101, Arg103, Phe156, Glu157, Arg159, Val161, Val189, and Ala190. In some embodiments, the peptide competes for binding to human EphA2 at one or more amino acid residues selected from Asp53, Phe156, and Glu157. In some embodiments, the peptide competes for binding to human EphA2 at Asp53, Glu157, or both.

The structures of exemplary unnatural amino acids that are present in Table 1 can be found in Table 3.

As described in Table 1 or other tables, abbreviations have the following meanings:
Lower case di means D-amino acids, e.g., dF refers to d-phenylalanine;
Me refers to a methyl group. e.g., MeG represents N-Methyl-Glycine;
Ala or A refer to alanine;
Arg or R refer to arginine;
Asn or N refer to asparagine;
Asp or D refer to aspartic acid;
Cys or C refer to cysteine;
Gin or Q refer to glutamine;
Gly or G refer to glycine;
His or H refer to histidine;
Ile or I refer to isoleucine;
Leu or L refer to leucine;
Lys or K refer to lysine;
Met or M refer to methionine;
Phe or F refer to phenylalanine;
Pro or P refer to proline;
Ser or S refer to serine;
Thr or T refer to threonine;
Trp or W refer to tryptophan;
Tyr or Y refer to tyrosine;
Val or V refer to valine;

Unless otherwise stated in the present specification, the following abbreviations for non-natural amino acids are used according to the following meanings:
Ahp 2-aminoheptanoic acid;
Alb 2-amino-3-ureidopropanoic acid, such as(S)-2-amino-3-ureidopropanoic acid (CAS No. 1483-07-4);
Da or da 2-aminopropanoic acid, such as (2R)-2-aminopropanoic acid;
dkCOpipzaa 2-amino-6-{[4-(carboxymethyl)piperazine-1-carbonyl]amino}hexanoic acid, such as (2R)-2-amino-6-{[4-(carboxymethyl)piperazine-1-carbonyl]amino}hexanoic acid

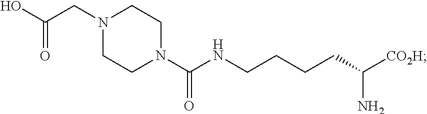 [SMILES: N[C@H](CCCCNC(=O)N1CCN(CC(=O)O)CC1)C(=O)O]

Dahp 2-aminoheptanoic acid, such as (2R)-2-aminoheptanoic acid

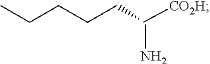

df3CON 2-amino-3-(3-carbamoylphenyl) propanoic acid, such as (2R)-2-amino-3-(3-carbamoylphenyl) propanoic acid (CAS No. 1217637-40-5)

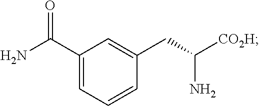 [SMILES: NC(=O)c1cccc(C[C@@H](N)C(=O)O)c1]

MeF 2-(methylamino)-3-phenylpropanoic acid, such as (2S)-2-(methylamino)-3-phenylpropanoic acid;
Me3Py 2-(methylamino)-3-(pyridin-3-yl) propanoic acid, such as (2S)-2-(methylamino)-3-(pyridin-3-yl)propanoic acid (CAS No. 1979173-93-7)

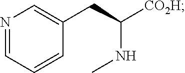

Nal1 1-naphthylalanine;
4Py 2-amino-3-(pyridin-4-yl) propanoic acid, such as (2S)-2-amino-3-(pyridin-4-yl) propanoic acid (CAS No. 169555-95-7)

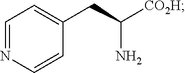 [SMILES: N[C@@H](Cc1ccncc1)C(=O)O]

MeHph 2-(methylamino)-4-phenylbutanoic acid, such as (2S)-2-(methylamino)-4-phenylbutanoic acid (CAS No. 1065076-30-3);
W7N 2-amino-3-{1H-pyrrolo[2,3-b]pyridin-3-yl}propanoic acid, such as (2S)-2-amino-3-{1H-pyrrolo[2,3-b]pyridin-3-yl}propanoic acid (CAS No. 737007-45-3)

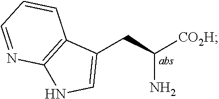 [SMILES: N[C@@H](Cc1c[nH]c2ncccc12)C(=O)O]

QPh 2-amino-4-(phenylcarbamoyl) butanoic acid, such as (2S)-2-amino-4-(phenylcarbamoyl)butanoic acid (CAS No. 198134-12-2);
MeF3CN 3-(3-cyanophenyl)-2-(methylamino) propanoic acid, such as (2S)-3-(3-cyanophenyl)-2-(methylamino)propanoic acid (CAS No. 2642331-80-2)

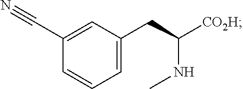

MeF3H 3-(3-hydroxyphenyl)-2-(methylamino) propanoic acid, such as (2S)-3-(3-hydroxyphenyl)-2-(methylamino)propanoic acid

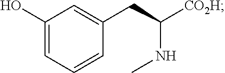

alT 2-amino-3-hydroxybutanoic acid, such as (2S,3S)-2-amino-3-hydroxybutanoic acid;
W1Me 2-amino-3-(1-methyl-1H-indol-3-yl)propanoic acid, such as (2S)-2-amino-3-(1-methyl-1H-indol-3-yl)propanoic acid (CAS No. 1334509-86-2)

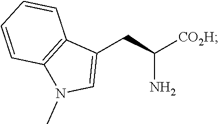 [SMILES: Cn1cc(C[C@H](N)C(=O)O)c2ccccc21]

tma 2-amino-4,4-dimethylpentanoic acid, such as (R)-2-amino-4,4-dimethylpentanoic acid

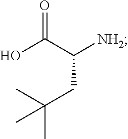

Cbg 2-amino-2-cyclobutylacetic acid, such as(S)-2-amino-2-cyclobutylacetic acid (CAS No. 1391630-31-1)

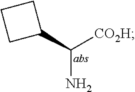

Chg 2-amino-2-cyclohexylacetic acid, such as (2S)-2-amino-2-cyclohexylacetic acid (CAS No. 161321-36-4)

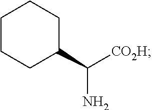 [SMILES: N[C@H](C(=O)O)C1CCCCC1]

Cba 2-amino-3-cyclobutylpropanoic acid, such as (2S)-2-amino-3-cyclobutylpropanoic acid (CAS No. 478183-62-9)

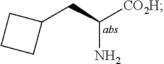

KCOpipzaa 2-amino-6-{[4-(carboxymethyl)piperazine-1-carbonyl]amino}hexanoic acid, such as (2S)-2-amino-6-{[4-(carboxymethyl)piperazine-1-carbonyl]amino}hexanoic acid

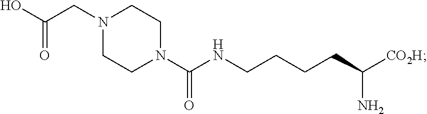 [SMILES: N[C@@H](CCCCNC(=O)N1CCN(CC(=O)O)CC1)C(=O)O]

Hgn 2-amino-5-carbamoylpentanoic acid, such as (2S)-2-amino-5-carbamoylpentanoic acid (CAS No. 1263046-43-0)

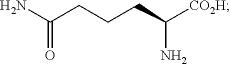 [SMILES: NC(=O)CCC[C@H](N)C(=O)O]

Hph homophenylalanine;
Nmm 2-amino-3-(methylcarbamoyl)propanoic acid, such as (2S)-2-amino-3-(methylcarbamoyl)propanoic acid (CAS No. 149204-93-3)

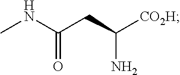

Ndm 2-amino-3-(dimethylcarbamoyl) propanoic acid, such as (2S)-2-amino-3-(dimethylcarbamoyl) propanoic acid (CAS No. 138585-02-1)

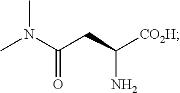

Hcit or hCit 2-amino-6-(carbamoylamino) hexanoic acid, such as (2S)-2-amino-6-(carbamoylamino)hexanoic acid (CAS No. 201485-17-8)

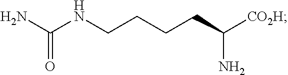 [SMILES: NC(=O)NCCCC[C@H](N)C(=O)O]

Qglucamine 2-amino-4-{[(2S,3R,4R,5R)-2,3,4,5,6 pentahydroxyhexyl]carbamoyl}butanoic acid, such as (2S)-2-amino-4-{[(2S,3R,4R,5R)-2,3,4,5,6 pentahydroxyhexyl]carbamoyl}butanoic acid

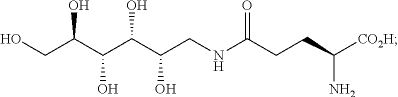 [SMILES: N[C@@H](CCC(=O)NC[C@H](O)[C@@H](O)[C@H](O)[C@H](O)CO)C(=O)O]

mBph 3-phenylphenylalanine;
MeE 2-(methylamino)pentanedioic acid, such as (2S)-2-(methylamino)pentanedioic acid;
MeN 3-carbamoyl-2-(methylamino)propanoic acid, such as (2S)-3-carbamoyl-2-(methylamino)propanoic acid;
MeF4C 3-(4-chlorophenyl)-2-(methylamino) propanoic acid, such as (2S)-3-(4-chlorophenyl)-2-(methylamino) propanoic acid (CAS No. 1217779-77-5);
Hph 2-amino-4-phenylbutanoic acid, such as (2S)-2-amino-4-phenylbutanoic acid;
W1Me7N 2-amino-3-{1-methyl-1H-pyrrolo[2,3-b]pyridin-3-yl}propanoic acid, such as (2S)-2-amino-3-{1-methyl-1H-pyrrolo[2,3-b]pyridin-3-yl}propanoic acid (CAS No. 1813528-10-7)

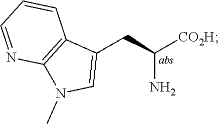 [SMILES: Cn1cc(C[C@H](N)C(=O)O)c2cccnc21]

W1Me7Cl 2-amino-3-(7-chloro-1-methyl-1H-indol-3-yl)propanoic acid, such as (2S)-2-amino-3-(7-chloro-1-methyl-1H-indol-3-yl) propanoic acid

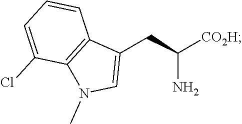 [SMILES: Cn1cc(C[C@H](N)C(=O)O)c2cccc(Cl)c21]

W6C 6-chlorotryptophan;
3Py6NH2 2-amino-3-(6-aminopyridin-3-yl)propanoic acid, such as (2S)-2-amino-3-(6-aminopyridin-3-yl)propanoic acid

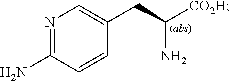 [SMILES: Nc1ccc(C[C@H](N)C(=O)O)cn1]

Cit 2-amino-5-(carbamoylamino)pentanoic acid, such as (2S)-2-amino-5-(carbamoylamino)pentanoic acid

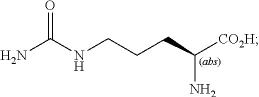 [SMILES: NC(=O)NCCC[C@H](N)C(=O)O]

F23dMe 2-amino-3-(2,3-dimethylphenyl) propanoic acid, such as (2S)-2-amino-3-(2,3-dimethylphenyl)propanoic acid (CAS No. 1270295-08-3)

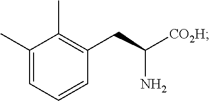

F3C 3-chlorophenylalanine;
Har 2-amino-6-carbamimidamidohexanoic acid, such as (2S)-2-amino-6-carbamimidamidohexanoic acid (CAS No. 776277-76-0);
bA 3-aminopropanoic acid;
Kac or KAc (2S)-2-amino-6-acetamidohexanoic acid (CAS No. 159766-56-0);
dkAc (2R)-2-amino-6-acetamidohexanoic acid (CAS No. 320410-22-8);
CdMe (R)-2-amino-3-mercapto-3-methylbutanoic acid;
C3SMe (2R,3S)-2-amino-3-mercaptobutanoic acid;
C3RMe (2R,3R)-2-amino-3-mercaptobutanoic acid;
4Py2NH2 (S)-2-amino-3-(2-aminopyridin-4-yl)propanoic acid;
Hgl (S)-2-aminohexanedioic acid.

TABLE 1
Exemplary peptide sequences with avidity to EphA2

| | SEQ | | | | | | | | | | | | | | | |
|---|---|---|---|---|---|---|---|---|---|---|---|---|---|---|---|---|
| | ID | Peptide Sequence (from residue position 1 to residue position 14, if present) | | | | | | | | | | | | | | |
| Peptide Name | NO: | 1 | 2 | 3 | 4 | 5 | 6 | 7 | 8 | 9 | 10 | 11 | 12 | 13 | 14 | 15 |
| PDC_EphA2-00001417 | 1 | da | MeF | N | L | Hgl | MeF | W1Me | V | W1Me | T | E | C | NH₂ | | |
| PDC_EphA2-00008082 | 183 | da | MeF | N | L | Hgl | MeF | W1Me | V | W1Me | T | E | C | bA | dkAc | NH₂ |
| PDC_EphA2-00008083 | 184 | da | Me3Py | N | L | Hgl | MeF | W1Me | V | W1Me | T | E | C | bA | dkAc | NH₂ |
| PDC_EphA2-00008084 | 185 | dkCOpipzaa | Me3Py | N | L | Hgl | MeF | W1Me | V | W1Me | T | E | C | bA | dkAc | NH₂ |
| PDC_EphA2-00008085 | 186 | dkCOpipzaa | Me3Py | N | L | KCOpipzaa | MeF | W1Me | V | W1Me | T | E | C | bA | dkAc | NH₂ |
| PDC_EphA2-00008086 | 187 | dkCOpipzaa | Me3Py | N | L | KCOpipzaa | MeF | W1Me | V | W1Me | T | Hgn | C | bA | dkAc | NH₂ |
| PDC_EphA2-00008087 | 188 | da | Me3Py | N | L | Hgl | MeF | W1Me | KCOpipzaa | W1Me | T | E | C | bA | dkAc | NH₂ |
| PDC_EphA2-00008088 | 189 | da | Me3Py | N | L | KCOpipzaa | MeF | W1Me | KCOpipzaa | W1Me | T | E | C | bA | dkAc | NH₂ |
| PDC_EphA2-00008089 | 190 | da | Me3Py | N | L | KCOpipzaa | MeF | W1Me | KCOpipzaa | W1Me | T | E | C | KAc | NH₂ | |
| PDC_EphA2-00008090 | 191 | da | Me3Py | N | L | KCOpipzaa | MeF | W1Me | KCOpipzaa | W1Me | T | E | C | dkAc | NH₂ | |
| PDC_EphA2-00008091 | 192 | da | Me3Py | N | L | Hgl | MeF | W1Me | KCOpipzaa | W1Me | T | Hgn | C | bA | dkAc | NH₂ |
| PDC_EphA2-00008092 | 193 | da | Me3Py | N | L | KCOpipzaa | MeF | W1Me | KCOpipzaa | W1Me | T | Hgn | C | bA | dkAc | NH₂ |
| PDC_EphA2-00008093 | 194 | da | Me3Py | N | L | Hgn | MeF | W1Me | KCOpipzaa | W1Me | T | Hgn | C | bA | dkAc | NH₂ |
| PDC_EphA2-00008094 | 195 | da | Me3Py | N | L | Hgl | MeE | W1Me | KCOpipzaa | W1Me | T | Hgn | C | bA | dkAc | NH₂ |
| PDC_EphA2-00008095 | 196 | da | Me3Py | N | L | KCOpipzaa | MeE | W1Me | KCOpipzaa | W1Me | T | Hgn | C | bA | dkAc | NH₂ |
| PDC_EphA2-00007196 | 197 | da | Me3Py | N | L | Hgn | MeE | W1Me | KCOpipzaa | W1Me | T | Hgn | C | bA | dkAc | NH₂ |
| PDC_EphA2-00007196-C002 | 200 | da | Me3Py | N | L | Hgn | MeE | W1Me | KCOpipzaa | W1Me | T | Hgn | C | dk | NH₂ | |
| PDC_EphA2-00008097 | 198 | A | Me3Py | N | L | Hgl | MeF | W1Me | KCOpipzaa | W1Me | T | E | C | bA | dkAc | NH₂ |
| PDC_EphA2-00009998 | 2 | da | Me3Py | N | L | Hgn | MeF | W1Me | KCOpipzaa | W1Me | T | Hgn | C | NH₂ | | |
| PDC_EphA2-00009999 | 3 | da | Me3Py | N | Cbg | Hgn | MeF | W1Me | KCOpipzaa | W1Me | T | Hgn | C | NH₂ | | |
| PDC_EphA2-00010000 | 4 | da | Me3Py | N | Cbg | Hgn | N | W1Me | KCOpipzaa | W1Me | T | Hgn | C | NH₂ | | |
| PDC_EphA2-00010001 | 5 | da | Me3Py | N | Cbg | Hgn | MeN | W1Me | KCOpipzaa | W1Me | T | Hgn | C | NH₂ | | |
| PDC_EphA2-00010002 | 6 | da | Me3Py | N | Cbg | Hgn | MeF | W1Me7N | KCOpipzaa | W1Me | T | Hgn | C | NH₂ | | |
| PDC_EphA2-00010003 | 7 | da | Me3Py | N | Cbg | Hgn | MeF | W1Me | KCOpipzaa | F23dMe | T | Hgn | C | NH₂ | | |
| PDC_EphA2-00010004 | 8 | da | Me3Py | N | Cbg | Hgn | MeF | W1Me | KCOpipzaa | W1Me | T | N | C | NH₂ | | |
| PDC_EphA2-00010005 | 9 | da | Me3Py | N | Chg | Hgn | MeF | W1Me | KCOpipzaa | W1Me | T | Hgn | C | NH₂ | | |
| PDC_EphA2-00010006 | 10 | da | Me3Py | N | L | Hgl | MeE | W1Me | KCOpipzaa | W1Me | T | Hgn | C | NH₂ | | |
| PDC_EphA2-00010007 | 11 | da | Me3Py | N | Cbg | Hgl | MeE | W1Me | KCOpipzaa | W1Me | T | Hgn | C | NH₂ | | |
| PDC_EphA2-00010008 | 12 | da | Me3Py | N | L | Hgn | MeE | W1Me | KCOpipzaa | W1Me | T | Hgn | C | NH₂ | | |
| PDC_EphA2-00010009 | 13 | da | Me3Py | N | Cbg | Hgn | MeE | W1Me | KCOpipzaa | W1Me | T | Hgn | C | NH₂ | | |
| PDC_EphA2-00010010 | 14 | da | Me3Py | N | Cbg | Hgn | N | W1Me | V | W1Me | T | Hgn | C | NH₂ | | |
| PDC_EphA2-00010011 | 15 | da | Me3Py | N | Cbg | Hgn | MeN | W1Me | V | W1Me | T | Hgn | C | NH₂ | | |
| PDC_EphA2-00010012 | 16 | da | Me3Py | N | Cbg | Hgn | Me3Py | W1Me | V | W1Me | T | Hgn | C | NH₂ | | |
| PDC_EphA2-00013693 | 17 | da | MeF | N | Cbg | Hgn | MeN | W1Me | V | W1Me | T | Hgn | C | NH₂ | | |
| PDC_EphA2-00010011-C003 | 180 | da | Me3Py | N | Cbg | Hgn | MeN | W1Me | V | W1Me | T | Hgn | C | bA | dk | NH₂ |
| PDC_EphA2-00010012-C003 | 181 | da | Me3Py | N | Cbg | Hgn | Me3Py | W1Me | V | W1Me | T | Hgn | C | bA | dk | NH₂ |
| PDC_EphA2-00013693-C003 | 182 | da | MeF | N | Cbg | Hgn | MeN | W1Me | V | W1Me | T | Hgn | C | bA | dk | NH₂ |
| PDC_EphA2-00013703 | 18 | da | MeF | N | Cbg | Hgn | MEN | W1Me | Qglucamine | W1Me | T | Hgn | C | NH₂ | | |
| PDC_EphA2-00013704 | 19 | da | MeF | N | Cbg | Hgn | Me3Py | W1Me | Qglucamine | W1Me | T | Hgn | C | NH₂ | | |
| PDC_EphA2-00013705 | 20 | da | Me3Py | N | Cbg | Hgn | Me3Py | W1Me | Qglucamine | W1Me | T | Hgn | C | NH₂ | | |
| PDC_EphA2-00008093-C002 | 199 | da | Me3Py | N | L | Hgn | MeF | W1Me | KCOpipzaa | W1Me | T | Hgn | C | dk | NH₂ | |
| PDC_EphA2_00007196-C002 | 200 | da | Me3Py | N | L | Hgn | MeE | W1Me | KCOpipzaa | W1Me | T | Hgn | C | dk | NH₂ | |
| PDC_EphA2-00010011-C002 | 201 | da | Me3Py | N | Cbg | Hgn | MeN | W1Me | V | W1Me | T | Hgn | C | dk | NH₂ | |
| PDC_EphA2-00010012-C002 | 202 | da | Me3Py | N | Cbg | Hgn | Me3Py | W1Me | V | W1Me | T | Hgn | C | dk | NH₂ | |
| PDC_EphA2-00013693-C002 | 203 | da | MeF | N | Cbg | Hgn | MeN | W1Me | V | W1Me | T | Hgn | C | dk | NH₂ | |
| PDC_EphA2-00013917 | 21 | da | MeF | N | Cbg | Hgn | MeF | W1Me | QGlucamine | W1Me | T | Hgn | C | NH₂ | | |
| PDC_EphA2-00018526 | 22 | dahp | Me3Py | N | L | Hgl | MeF | W1Me | KCOpipzaa | W1Me | T | Hgn | C | NH₂ | | |
| PDC_EphA2-00018527 | 23 | da | Me3Py | N | L | Hgl | MeF4C | W1Me | KCOpipzaa | W1Me | T | Hgn | C | NH₂ | | |
| PDC_EphA2-00018528 | 24 | da | Me3Py | N | L | Hgl | MeF | W1Me | KCOpipzaa | W1Me | T | Nmm | C | NH₂ | | |
| PDC_EphA2-00018529 | 25 | da | Me3Py | N | L | Hgl | MeF | W1Me | KCOpipzaa | W1Me | T | Ndm | C | NH₂ | | |
| PDC_EphA2-00018530 | 26 | dahp | Me3Py | N | L | Hgn | MeE | W1Me | KCOpipzaa | W1Me | T | Hgn | C | NH₂ | | |
| PDC_EphA2-00018531 | 27 | da | Me3Py | N | L | Nmm | MeE | W1Me | KCOpipzaa | W1Me | T | Hgn | C | NH₂ | | |
| PDC_EphA2-00018532 | 28 | da | Me3Py | N | L | Ndm | MeE | W1Me | KCOpipzaa | W1Me | T | Hgn | C | NH₂ | | |
| PDC_EphA2-00018533 | 29 | da | Me3Py | N | L | Hgn | MeE | W1Me | KCOpipzaa | W1Me | T | Nmm | C | NH₂ | | |
| PDC_EphA2-00018534 | 30 | da | Me3Py | N | L | Hgn | MeE | W1Me | KCOpipzaa | W1Me | T | Ndm | C | NH₂ | | |
| PDC_EphA2-00018535 | 31 | dahp | Me3Py | N | Chg | Hgn | MeF | W1Me | KCOpipzaa | W1Me | T | Hgn | C | NH₂ | | |
| PDC_EphA2-00018536 | 32 | da | Me3Py | N | Chg | Nmm | MeF | W1Me | KCOpipzaa | W1Me | T | Hgn | C | NH₂ | | |
| PDC_EphA2-00018537 | 33 | da | Me3Py | N | Chg | Ndm | MeF | W1Me | KCOpipzaa | W1Me | T | Hgn | C | NH₂ | | |
| PDC_EphA2-00018538 | 34 | da | Me3Py | N | Chg | Hgn | MeF4C | W1Me | KCOpipzaa | W1Me | T | Hgn | C | NH₂ | | |
| PDC_EphA2-00018539 | 35 | da | Me3Py | N | Chg | Hgn | MeF | W1Me | KCOpipzaa | W1Me | T | Nmm | C | NH₂ | | |
| PDC_EphA2-00018540 | 36 | da | Me3Py | N | Chg | Hgn | MeF | W1Me | KCOpipzaa | W1Me | T | Ndm | C | NH₂ | | |
| PDC_EphA2-00019421 | 37 | df3CON | MeF | N | Cbg | Hgn | MeN | W1Me7Cl | V | W1Me | T | Hgn | C | NH₂ | | |
| PDC_EphA2-00019422 | 38 | df3CON | MeF | N | L | Hgn | MeN | W1Me7Cl | V | W1Me | T | Hgn | C | NH₂ | | |
| PDC_EphA2-00019423 | 39 | df3CON | MeF | N | L | Hgn | MeN | W1Me | V | W1Me | T | Hgn | C | NH₂ | | |
| PDC_EphA2-00019424 | 40 | df3CON | Me3Py | N | Cbg | Hgn | MeN | W1Me7Cl | V | W1Me | T | Hgn | C | NH₂ | | |
| PDC_EphA2-00019425 | 41 | df3CON | MeF | N | Cbg | Hgn | MeN | W1Me7Cl | KCOpipzaa | W1Me | T | Hgn | C | NH₂ | | |
| PDC_EphA2-00019426 | 42 | df3CON | MeF | N | Cbg | Hgn | MeN | W1Me7Cl | Hcit | W1Me | T | Hgn | C | NH₂ | | |
| PDC_EphA2-00019427 | 43 | df3CON | MeF | N | Cbg | Hgn | MeE | W1Me7Cl | Hcit | W1Me | T | Har | C | NH₂ | | |
| PDC_EphA2-00019428 | 44 | df3CON | Me3Py | N | Cbg | Hgn | MeN | W1Me7Cl | Hcit | W1Me | T | R | C | NH₂ | | |
| PDC_EphA2-00019429 | 45 | df3CON | Me3Py | N | Cbg | Hgn | MeN | W1Me7Cl | Hcit | W1Me | T | Har | C | NH₂ | | |
| PDC_EphA2-00019430 | 46 | df3CON | Me3Py | N | Cbg | Hgn | MeN | W1Me7Cl | KCOpipzaa | W1Me | T | Har | C | NH₂ | | |
| PDC_EphA2-00019431 | 47 | df3CON | MeF | N | Cbg | Hgn | MeF | W1Me7Cl | Qglucamine | W1Me | T | Hgn | C | NH₂ | | |
| PDC_EphA2-00019432 | 48 | df3CON | MeF | N | Cbg | Hgn | MeF | W1Me7Cl | KCOpipzaa | W1Me | T | Hgn | C | NH₂ | | |
| PDC_EphA2-00019433 | 49 | df3CON | Me3Py | N | Cbg | Hgn | MeF | W1Me7Cl | Qglucamine | W1Me | T | Hgn | C | NH₂ | | |
| PDC_EphA2-00019434 | 50 | df3CON | Me3Py | N | Cbg | Hgn | MeF | W1Me7Cl | KCOpipzaa | W1Me | T | Hgn | C | NH₂ | | |
| PDC_EphA2-00019435 | 51 | df3CON | MeF | N | Cbg | Hgn | Me3Py | W1Me7Cl | Qglucamine | W1Me | T | Hgn | C | NH₂ | | |
| PDC_EphA2-00019436 | 52 | df3CON | MeF | N | Cbg | Hgn | Me3Py | W1Me7Cl | KCOpipzaa | W1Me | T | Hgn | C | NH₂ | | |
| PDC_EphA2-00019437 | 53 | df3CON | Me3Py | N | Cbg | Hgn | Me3Py | W1Me7Cl | Qglucamine | W1Me | T | Hgn | C | NH₂ | | |
| PDC_EphA2-00019438 | 54 | df3CON | Me3Py | N | Cbg | Hgn | Me3Py | W1Me7Cl | KCOpipzaa | W1Me | T | Hgn | C | NH₂ | | |
| PDC_EphA2-00019439 | 55 | df3CON | Me3Py | N | Cbg | Hgn | MeE | W1Me7Cl | Qglucamine | W1Me | T | Hgn | C | NH₂ | | |
| PDC_EphA2-00019440 | 56 | df3CON | Me3Py | N | Cbg | Hgn | MeE | W1Me7Cl | KCOpipzaa | W1Me | T | Hgn | C | NH₂ | | |
| PDC_EphA2-00019441 | 57 | df3CON | Me3Py | A | Cbg | KCOpipzaa | MeF | W1Me7Cl | KCOpipzaa | W1Me | T | Hgn | C | NH₂ | | |
| PDC_EphA2-00019442 | 58 | df3CON | Me3Py | N | Cbg | KCOpipzaa | MeF | W1Me7Cl | Qglucamine | W1Me | T | Hgn | C | NH₂ | | |
| PDC_EphA2-00019443 | 171, 273 | df3CON | Me3Py | N | Cbg | Hgn | MeE | W1Me7Cl | KCOpipzaa | W1Me | T | hArg | C | | | |
| PDC_EphA2-00019437-C002 | 204 | df3CON | Me3Py | N | Cbg | Hgn | Me3Py | W1Me7Cl | Qglucamine | W1Me | T | Hgn | C | dk | NH₂ | |
| PDC_EphA2-00022593-C002 | 205 | df3CON | Me3Py | A | Cbg | Hgn | MeN | W1Me7Cl | Qglucamine | W1Me | T | Hgn | C | dk | NH₂ | |
| PDC_EphA2-00022594-C002 | 206 | df3CON | Me3Py | N | Cbg | Qglucamine | MeN | W1Me7Cl | Qglucamine | W1Me | T | Hgn | C | dk | NH₂ | |
| PDC_EphA2-00022595-C002 | 207 | df3CON | Me3Py | N | Cbg | Hgn | MeN | W1Me | Qglucamine | W1Me | T | Hgn | C | dk | NH₂ | |
| PDC_EphA2-00020708 | 150 | F | 4Py2NH2 | L | Hcit | W1Me | MeF | W1Me | C | NH₂ | | | | | | |
| PDC_EphA2-00022601 | 246 | F | 4Py2NH2 | L | Hcit | W1Me | MeF | W1Me | C | G | NH₂ | | | | | |
| PDC_EphA2-00022602 | 247 | F | 4Py2NH2 | L | Hcit | W1Me | MeF | W1Me | C | KAc | NH₂ | | | | | |
| PDC_EphA2-00022603 | 248 | F | 4Py2NH2 | L | Hcit | W1Me | MeF | W1Me | C | dkAc | NH₂ | | | | | |
| PDC_EphA2-00022417 | 123 | F | H | N | L | S | MeF | W1Me | Hcit | W1Me | C | NH₂ | | | | |
| PDC_EphA2-00022606 | 232 | F | H | N | L | S | MeF | W1Me | Hcit | W1Me | C | G | NH₂ | | | |
| PDC_EphA2-00022607 | 233 | F | H | N | L | S | MeF | W1Me | Hcit | W1Me | C | KAc | NH₂ | | | |
| PDC_EphA2-00022608 | 234 | F | H | N | L | S | MeF | W1Me | Hcit | W1Me | C | dkAc | NH₂ | | | |
| PDC_EphA2-00019440-C002 | 208 | df3CON | Me3Py | N | Cbg | Hgn | MeE | W1Me7Cl | KCOpipzaa | W1Me | T | Hgn | C | dk | NH₂ | |
| PDC_EphA2-00022612-C002 | 209 | df3CON | MeF3CN | N | Cbg | Hgn | MeE | W1Me7Cl | KCOpipzaa | W1Me | T | Hgn | C | dk | NH₂ | |
| PDC_EphA2-00022613-C002 | 210 | df3CON | MeF3H | N | Cbg | Hgn | MeE | W1Me7Cl | KCOpipzaa | W1Me | T | Hgn | C | dk | NH₂ | |
| PDC_EphA2-00022614-C002 | 211 | df3CON | MeF3CN | N | Cbg | Hgn | Me3Py | W1Me7Cl | Qglucamine | W1Me | T | Hgn | C | dk | NH₂ | |
| PDC_EphA2-00022615-C002 | 212 | df3CON | MeF3CN | N | Cbg | Hgn | MeN | W1Me7Cl | Qglucamine | W1Me | T | Hgn | C | dk | NH₂ | |
| PDC_EphA2-00013703-C002 | 213 | da | MeF | N | Cbg | Hgn | MeN | W1Me | Qglucamine | W1Me | T | Hgn | C | dk | NH₂ | |
| PDC_EphA2-00013704-C002 | 214 | da | MeF | N | Cbg | Hgn | Me3Py | W1Me | Qglucamine | W1Me | T | Hgn | C | dk | NH₂ | |
| PDC_EphA2-00013705-C002 | 215 | da | Me3Py | N | Cbg | Hgn | Me3Py | W1Me | Qglucamine | W1Me | T | Hgn | C | dk | NH₂ | |
| PDC_EphA2-00019498-L092 | 251 | da | 4Py2NH2 | MeF | W1Me | MeF | W1Me | C | G | NH₂ | | | | | | |
| PDC_EphA2-00020295-L092 | 249 | F | 4Py | L | G | W1Me | MeF | W1Me | C | G | NH₂ | | | | | |
| PDC_EphA2-00021407-L092 | 235 | da | MeF | N | Cba | T | N | W1Me | Hcit | W1Me | C | G | NH₂ | | | |
| PDC_EphA2-00022057-L092 | 236 | F | 4Py | N | L | E | MeF | W1Me | 3Py6NH2 | W1Me | C | G | NH₂ | | | |
| PDC_EphA2-00022062-L092 | 272 | F | W1Me | W1Me | W1Me | S | 3Py6NH2 | MeF | E | C | G | NH₂ | | | | |
| PDC_EphA2-00020708-C002 | 250 | F | 4Py2NH2 | L | Hcit | W1Me | MeF | W1Me | C | dk | NH₂ | | | | | |
| PDC_EphA2-00022417-C002 | 237 | F | H | N | L | S | MeF | W1Me | Hcit | W1Me | C | dk | NH₂ | | | |
| PDC_EphA2-00026424 | 59 | df3CON | Me3Py | N | L | N | Me3Py | W1Me7Cl | Qglucamine | W1Me | T | N | C | NH₂ | | |
| PDC_EphA2-00026425 | 60 | df3CON | Me3Py | N | L | N | MeN | W1Me7Cl | Qglucamine | W1Me | T | N | C | NH₂ | | |
| PDC_EphA2-00026426 | 61 | df3CON | Me3Py | N | L | N | MeN | W1Me7Cl | Cit | W1Me | T | N | C | NH₂ | | |
| PDC_EphA2-00026424-C002 | 216 | df3CON | Me3Py | N | L | N | Me3Py | W1Me7Cl | Qglucamine | W1Me | T | N | C | dk | NH₂ | |
| PDC_EphA2-00026425-C002 | 217 | df3CON | Me3Py | N | L | N | MeN | W1Me7Cl | Qglucamine | W1Me | T | N | C | dk | NH₂ | |
| PDC_EphA2-00026426-C002 | 218 | df3CON | Me3Py | N | L | N | MeN | W1Me7Cl | Cit | W1Me | T | N | C | dk | NH₂ | |
| PDC_EphA2-00026479 | 151 | F | 4Py2NH2 | L | MeA | W1Me | MeF | W1Me | C | NH₂ | | | | | | |
| PDC_EphA2-00026481 | 152 | F | 4Py2NH2 | L | MeHcit | W1Me | MeF | W1Me | C | NH₂ | | | | | | |
| PDC_EphA2-0002652 | 62 | da | Me3Py | N | L | K | MeE | W1Me7Cl | KCOpipzaa | W1Me | T | Hgn | C | NH₂ | | |
| PDC_EphA2-00026522 | 63 | da | Me3Py | N | L | Hgn | MeE | W1Me7Cl | K | W1Me | T | Hgn | C | NH₂ | | |
| PDC_EphA2-00026523 | 64 | da | Me3Py | N | L | Hgn | MeE | W1Me7Cl | KCOpipzaa | W1Me | T | K | C | NH₂ | | |
| PDC_EphA2-00026524 | 65 | df3CON | Me3Py | N | L | K | MeN | W1Me7Cl | Qglucamine | W1Me | T | Hgn | C | NH₂ | | |
| PDC_EphA2-00026525 | 66 | df3CON | Me3Py | N | L | Hgn | MeN | W1Me7Cl | K | W1Me | T | Hgn | C | NH₂ | | |
| PDC_EphA2-00026526 | 67 | df3CON | Me3Py | N | L | Hgn | MeN | W1Me7Cl | Qglucamine | W1Me | T | K | C | NH₂ | | |
| PDC_EphA2-00026527 | 68 | df3CON | Me3Py | N | L | K | MeE | W1Me7Cl | KCOpipzaa | W1Me | T | Hgn | C | NH₂ | | |
| PDC_EphA2-00026528 | 69 | df3CON | Me3Py | N | L | Hgn | MeE | W1Me7Cl | K | W1Me | T | Hgn | C | NH₂ | | |
| PDC_EphA2-00026529 | 70 | df3CON | Me3Py | N | L | Hgn | MeE | W1Me7Cl | KCOpipzaa | W1Me | T | K | C | NH₂ | | |
| PDC_EphA2-00026600 | 124 | df3CON | Me3Py | N | L | S | MeF | W1Me7Cl | Hcit | W1Me | C | NH₂ | | | | |
| PDC_EphA2-00026601 | 125 | df3CON | Me3Py | N | L | S | Me3Py | W1Me7Cl | Hcit | W1Me | C | NH₂ | | | | |
| PDC_EphA2-00026602 | 126 | df3CON | Me3Py | N | Cbg | S | Me3Py | W1Me7Cl | Hcit | W1Me | C | NH₂ | | | | |
| PDC_EphA2-00026603 | 127 | df3CON | Me3Py | N | Cbg | Qglucamine | Me3Py | W1Me7Cl | KCOpipzaa | W1Me | C | NH₂ | | | | |
| PDC_EphA2-00026604 | 128 | df3CON | Me3Py | N | Cbg | Qglucamine | Me3Py | W1Me7Cl | Qglucamine | W1Me | C | NH₂ | | | | |
| PDC_EphA2-00026605 | 129 | df3CON | Me4Py2NH2 | N | Cbg | Qglucamine | Me3Py | W1Me7Cl | KCOpipzaa | W1Me | C | NH₂ | | | | |
| PDC_EphA2-00026606 | 130 | df3CON | Me3Py | N | Cbg | Hgn | MeE | W1Me7Cl | KCOpipzaa | W1Me | C | NH₂ | | | | |
| PDC_EphA2-00026607 | 131 | df3CON | Me3Py | N | Cbg | Qglucamine | MeE | W1Me7Cl | Hcit | W1Me | C | NH₂ | | | | |
| PDC_EphA2-00026608 | 132 | df3CON | MeF3H | N | Cbg | Hgn | MeE | W1Me7Cl | KCOpipzaa | W1Me | C | NH₂ | | | | |
| PDC_EphA2-00026609 | 133 | df3CON | Me3Py | N | Cbg | Hgn | MeE | W1Me7Cl | KCOpipzaa | W1Me | CdMe | NH₂ | | | | |
| PDC_EphA2-00026610 | 134 | df3CON | Me3Py | N | Cbg | Hgn | MeE | W1Me7Cl | KCOpipzaa | W1Me | C3SMe | NH₂ | | | | |
| PDC_EphA2-00026611 | 135 | df3CON | Me3Py | N | Cbg | Hgn | MeE | W1Me7Cl | KCOpipzaa | W1Me | C3RMe | NH₂ | | | | |
| PDC_EphA2-00026600-C001 | 238 | df3CON | Me3Py | N | L | S | MeF | W1Me7Cl | Hcit | W1Me | C | K | NH₂ | | | |
| PDC_EphA2-00026601-C001 | 239 | df3CON | Me3Py | N | L | S | Me3Py | W1Me7Cl | Hcit | W1Me | C | K | NH₂ | | | |
| PDC_EphA2-00026602-0001 | 240 | df3CON | Me3Py | N | Cbg | S | Me3Py | W1Me7Cl | Hcit | W1Me | C | K | NH₂ | | | |
| PDC_EphA2-00026603-C001 | 241 | df3CON | Me3Py | N | Cbg | Qglucamine | Me3Py | W1Me7Cl | KCOpipzaa | W1Me | C | K | NH₂ | | | |
| PDC_EphA2-00026604-C001 | 242 | df3CON | Me3Py | N | Cbg | Qglucamine | Me3Py | W1Me7Cl | Qglucamine | W1Me | C | K | NH₂ | | | |
| PDC_EphA2-00026606-C001 | 243 | df3CON | Me3Py | N | Cbg | Hgn | MeE | W1Me7Cl | KCOpipzaa | W1Me | C | K | NH₂ | | | |
| PDC_EphA2-00026607-C001 | 244 | df3CON | Me3Py | N | Cbg | Qglucamine | MeE | W1Me7Cl | Hcit | W1Me | C | K | NH₂ | | | |
| PDC_EphA2-00026608-C001 | 245 | df3CON | MeF3H | N | Cbg | Hgn | MeE | W1Me7Cl | KCOpipzaa | W1Me | C | K | NH₂ | | | |
| PDC_EphA2-00026624 | 219 | da | Me3Py | N | L | Hgn | MeE | W1Me | KCOpipzaa | W1Me | T | Hgn | C | dk | —OH | |
| PDC_EphA2-00026625 | 220 | da | MeF | N | Cbg | Hgn | MeN | W1Me | V | W1Me | T | Hgn | C | dk | —OH | |
| PDC_EphA2-00026626 | 221 | df3CON | Me3Py | N | Cbg | Hgn | Me3Py | W1Me7Cl | Qglucamine | W1Me | T | Hgn | C | dk | —OH | |
| PDC_EphA2-00026627 | 222 | df3CON | MeF3H | N | Cbg | Hgn | MeE | W1Me7Cl | KCOpipzaa | W1Me | T | Hgn | C | dk | —OH | |
| PDC_EphA2-00027090 | 254 | da | Me3Py | N | Cbg | K | MeE | W1Me7Cl | KCOpipzaa | W1Me | T | Hgn | C | de | —OH | |
| PDC_EphA2-00027091 | 255 | df3CON | Me3Py | N | Cbg | K | MeE | W1Me7Cl | KCOpipzaa | W1Me | T | Hgn | C | de | —OH | |
| PDC_EphA2-00027092 | 256 | da | Me3Py | N | Cbg | K | MeE | W1Me7Cl | KCOpipzaa | W1Me | T | Hgn | C | de | NH₂ | |
| PDC_EphA2-00027094-C002 | 252 | df3CON | Me3Py | N | Cbg | Hgn | MeE | W1Me7N | KCOpipzaa | W1Me | T | Hgn | C | dk | NH₂ | |
| PDC_EphA2-00027095-C002 | 253 | da | Me3Py | N | Cbg | Hgn | MeE | W1Me7N | KCOpipzaa | W1Me | T | Hgn | C | dk | NH₂ | |
| PDC_EphA2-00008010-C002 | 267 | da | Me3Py | N | L | Hgn | MeE | W1Me | KCOpipzaa | W1Me | T | hArg | C | dk | NH₂ | |

The molecular weight of the described peptide can vary. In some embodiments, the peptide has a molecular weight of about 0.1 to about 25 kDa. In some embodiments, the peptide has a molecular weight of about 0.2 to about 20 kDa, about 0.5 to about 15 kDa, about 0.75 to about 10 kDa, about 0.5 to about 10 kDa, about 0.5 to about 5 kDa, about 0.5 to about 2.5 kDa, about 0.5 to about 2 kDa, about 0.5 to about 1.5 kDa, about 0.5 to about 1 kDa, about 1 to about 10 kDa, about 1 to about 5 kDa, about 1 to about 2.5 kDa, about 1 to about 2 kDa, about 1 to about 1.5 kDa, about 1 to about 1.25 kDa, or about 0.5 to about 1.25 kDa. In some embodiments, the peptide has a molecular weight of about 0.5 to 5 kDa. In some embodiments, the peptide has a molecular weight of about 0.5 to 2 kDa. In some embodiments, the peptide has a molecular weight of about 0.75 to 1.75 kDa. In some embodiments, the peptide has a molecular weight of about 1 to 1.5 kDa. In some embodiments, the peptide is monocyclic.

A peptide described herein can be cyclized (i.e., macrocyclized). Cyclization can be achieved less ideally via a single disulfide bond, or more ideally via a peptide bond, alkyl bond, alkenyl bond, ester bond, thioester bond, ether bond, thioether bond, phosphate ether bond, azo bond, C—S—C bond, C—N—C bond, C═N—C bond, C═N—O bond, amide bond, lactam bridge, carbamoyl bond, urea bond, thiourea bond, amine bond, thioamide bond, or the like, but not limited to them. In some embodiments, the peptide is a cyclic peptide that is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via an oxime cyclization reaction. A cyclization of a peptide sometimes stabilizes the peptide structure and thereby enhance affinity for a target. The cyclization can occur between the N- and C-terminus, or it can occur between a terminal amino acid and a non-terminal amino acid. In some embodiments, the cyclization occurs between two non-terminal amino acids. In some embodiments, the peptide is cyclized via oxime cyclization. In some embodiments, the peptide is cyclized between cysteine and haloacyl. In some embodiments, the peptide comprises a haloacetyl group (e.g., chloroacetyl or bromoacetyl) at the N-terminus. In some embodiments, the peptide comprises a haloacetyl group (e.g., chloroacetyl or bromoacetyl) at the C-terminus. In some embodiments, the peptide comprises a Cys at the C-terminus. In some embodiments, the peptide comprises a Cys at the N-terminus. In some embodiments, the cyclization occurs via a thioether bond between Cys and a haloacetyl group. In some embodiments, the cyclization occurs between the N-terminus and the C-terminus of the peptide.

As amino acids for macrocyclization, for example, an amino acid having the following functional group A and an amino acid having a corresponding functional group B can be used (see Table 4A). Either the functional group A or the functional group B may be placed on the N-terminal side. The amino acid having the functional group A and the amino acid having the functional group B can each be an N-terminal amino acid or C-terminal amino acid or a non-terminal amino acid. In some embodiments, an amino acid having the functional group A is placed at the N-terminus. In some embodiments, an amino acid having the functional group A is placed at the C-terminus. In some embodiments, an amino acid having the functional group A is placed at a non-terminal amino acid. In some embodiments, an amino acid having the functional group B is placed at the N-terminus. In some embodiments, an amino acid having the functional group B is placed at the C-terminus. In some embodiments, an amino acid having the functional group B is placed at a non-terminal amino acid.

In some embodiments, as the amino acid (I-A), for example, a chloroacetylated amino acid can be used. Examples of the chloroacetylated amino acids include N-chloroacetyl-L-alanine, N-chloroacetyl-L-phenylalanine, N-chloroacetyl-L-tyrosine, N-chloroacetyl-L-tryptophan, N-3-(2-chloroacetamido)benzoyl-L-phenylalanine, N-3-(2-chloroacetamido)benzoyl-L-tyrosine, N-3-(2-chloroacetamido)benzoyl-L-tryptophan, β-N-chloroacetyl-L-diaminopropanoic acid, γ-N-chloroacetyl-L-diaminobutyric acid, σ-N-chloroacetyl-L-ornithine, ε—N-chloroacetyl-L-lysine, N-3-chloromethylbenzoyl-L-tyrosine, and N-3-chloromethylbenzoyl-L-tryptophane and D-amino acid derivatives corresponding thereto (for example, N-Chloroacetyl-D-alanine, N-Chloroacetyl-D-phenylalanine, N-Chloroacetyl-D-tyrosine, and N-Chloroacetyl-D-tryptophan).

TABLE 4A
Functional groups for cyclization

| | Functional group A | Functional group B |
|---|---|---|
| (I) | 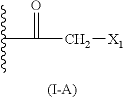 | 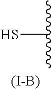 [SMILES: *S ; "(I-B)"] |
| | X₁ is a halogen such as Cl, Br or I | |
| (II) | 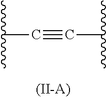 [SMILES: *C#C*] | 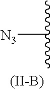 [SMILES: *N=[N+]=[N-] ; "(II-B)"] |
| (III) | 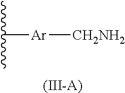 Ar is substituted or unsubstituted aryl or heteroaryl | 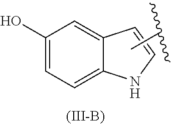 |
| (IV) | 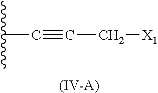 | 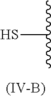 [SMILES: *S ; "(IV-B)"] |
| | X₁ is a halogen such as Cl, Br or I | |
| (V) | 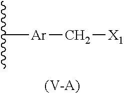 | 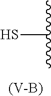 [SMILES: *S ; "(V-B)"] |
| | X₁ is a halogen such as Cl, Br or I; | |
| | Ar is substituted or unsubstituted | |
| | aryl or heteroaryl | |
| (VI) | 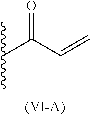 | 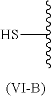 [SMILES: *S ; "(VI-B)"] |
| (VII) | 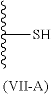 | 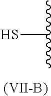 |
| (VIII) | 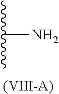 | 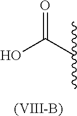 |
| (IX) | 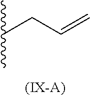 | 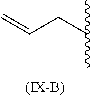 |

Examples of the amino acid (I-B) include, but are not limited to, cysteine, homocysteine, mercaptonorvaline, mercaptonorleucine, 2-amino-7-mercaptoheptanoic acid, 2-amino-8-mercaptooctanoic acid, and amino acids obtained by protecting the SH group of these amino acids and then eliminating the protecting group, and D-amino acid derivatives corresponding thereto.

The cyclization method can be carried out, for example, according to the method described in Kawakami, T. et al., Nature Chemical Biology 5, 888-890 (2009); Yamagishi, Y. et al., ChemBioChem 10, 1469-1472 (2009); Sako, Y. et al., Journal of American Chemical Society 130, 7932-7934 (2008); or WO2008/117833.

In some embodiments, for example, the amino acid (II-A) is selected from propargylglycine, homopropargylglycine, 2-amino-6-heptynoic acid, 2-amino-7-octynoic acid, and 2-amino-8-nonynoic acid can be used. In addition, 4-pentynoylated or 5-hexynoylated amino acids can also be used. Examples of the 4-pentynoylated amino acids include N-(4-pentenoyl)-L-alanine, N-(4-pentenoyl)-L-phenylalanine, N-(4-pentenoyl)-L-tyrosine, N-(4-pentenoyl)-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-(4-pentenoyl)-L-diaminopropanoic acid, γ-N-(4-pentenoyl)-L-diaminobutyric acid, σ-N-(4-pentenoyl)-L-ornithine, and ε—N-(4-pentenoyl)-L-lysine, and D-amino acid derivatives corresponding thereto.

In some embodiments, for example, the amino acid (II-B) is selected from azidoalanine, 2-amino-4-azidobutanoic acid, azidoptonorvaline, azidonorleucine, 2-amino-7-azidoheptanoic acid, and 2-amino-8-azidooctanoic acid can be used. In addition, azidoacetylated or 3-azidopentanoylated amino acids can also be used. Examples of the azidoacetylated amino acids include N-azidoacetyl-L-alanine, N-azidoacetyl-L-phenylalanine, N-azidoacetyl-L-tyrosine, N-azidoacetyl-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-azidoacetyl-L-diaminopropanoic acid, γ-N-azidoacetyl-L-diaminobutyric acid, α-N-azidoacetyl-L-ornithine, and ε—N-azidoacetyl-L-lysine, and D-amino acid derivatives corresponding thereto.

The cyclization method can be performed, for example, according to the method described in Sako, Y. et al., Journal of American Chemical Society 130, 7932-7934 (2008) or WO2008/117833.

Examples of amino acid (III-A) include, but are not limited to, N-(4-aminomethyl-benzoyl)-phenylalanine (AMBF) and 4-3-aminomethyltyrosine.

Examples of the amino acid (III-B) include, but are not limited to, 5-hydroxytryptophan (WoH). The cyclization method can be performed, for example, according to the method described in Yamagishi, Y. et al., ChemBioChem 10, 1469-1472 (2009) or WO2008/117833.

Examples of the amino acid (IV-A) include, but are not limited to, 2-amino-6-chloro-hexynoic acid, 2-amino-7-chloro-heptynoic acid, and 2-amino-8-chloro-octynoic acid.

Examples of the amino acid (IV-B) include, but are not limited to, cysteine, homocysteine, mercaptonorvaline, mercaptonorleucine, 2-amino-7-mercaptoheptanoic acid, and 2-amino-8-mercaptooctanoic acid, amino acids obtained by protecting the SH group of these amino acids and then eliminating the protecting group, and D-amino acid derivatives corresponding thereto. The cyclization method can be performed, for example, according to the method described in WO2012/074129.

Examples of the amino acid (V-A) include, but are not limited to, N-3-chloromethylbenzoyl-L-phenylalanine, N-3-chloromethylbenzoyl-L-tyrosine, and N-3-chloromethylbenzoyl-L-tryptophane.

Examples of the amino acid (V-B) include, but are not limited to, cysteine, homocysteine, mercaptonorvaline, mercaptonorleucine, 2-amino-7-mercaptoheptanoic acid, and 2-amino-8-mercaptooctanoic acid, and amino acids obtained by protecting the SH group of these amino acids and then eliminating the protecting group, and D-amino acid derivatives corresponding thereto.

The amino acids I-A to V-A and I-B to V-B can be introduced into the peptide in a known manner by chemical synthesis or translation and synthesis described herein. In some embodiments, the cyclization reaction comprises forming a thioether bond using an amino acid comprising a sulfanyl group, e.g., cysteine, homocysteine, mercaptonorvaline, mercaptovaline, mercaptonorleucine, 2-amino-7-mercaptoheptanoic acid, and 2-amino-8-mercaptooctanoic acid.

A peptide described herein can comprise one or more negatively charged amino acids and/or one or more positively charged amino acids. Positively charged amino acids include, for example, lysine, arginine, histidine, and amino acids that contain additional amine groups. Positively charged amino acids can comprise a heteroaryl substitution such as pyridine, imidazole, pyrazole, or triazole that has one or more ring nitrogen atoms. Negatively charged amino acids include, for example, amino acids that contain an additional carboxylic acid group such as glutamic acid or the like.

In some embodiments, a cyclic peptide of Formula (I), Formula (I-1), Formula (I-2), Formula (Ia), Formula (Ib), or Formula (Ic) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. In some embodiments, a cyclic peptide of Formula (I), Formula (I-1), Formula (I-2), Formula (Ia), Formula (Ib), or Formula (Ic) has a net charge of at most-4. In some embodiments, the cyclic peptide has a net charge of −4. In some embodiments, a cyclic peptide of Formula (I), Formula (I-1), Formula (I-2), Formula (Ia), Formula (Ib), or Formula (Ic) has a net charge of at least +2. In some embodiments, the cyclic peptide has a net charge of +2. In some embodiments, the cyclic peptide has a net charge of +3. The net charge can be determined by aggregating the charge of each of the X1 to X12 amino acids (or each of the amino acid in the peptide). For example, aspartic acid (D) and glutamic acid (E) each has a charge of −1, lysine (K), arginine (R) and histidine (H) each has a charge of +1, and the rest of the canonical amino acids each has a charge of 0.

In some embodiments, a (cyclic) peptide of formula (I) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. The net charge can be determined by aggregating the charge of each of the amino acids of the (cyclic) peptide.

In some embodiments, a (cyclic) peptide described herein (e.g., a (cyclic) peptide of Formula (I), Formula (1-1), Formula (I-2), Formula (Ia), Formula (Ib), or Formula (Ic) is configured to bind to EphA2 with a prescribed affinity, for example, measured as Plasma Protein Albumin Binding (PPB) percentage. The % bound can be determined by HSA-HPLC method (measurement of drug protein binding by immobilized human serum albumin-HPLC). PPB can be determined in vitro by HPLC (e.g., Example B3) or by other suitable means known in the art. In some embodiments, 1% to 99% of the cyclic peptide binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the cyclic peptide binds to HSA in vitro as determined by HPLC. In some embodiments, about 10% to about 95% of the cyclic peptide binds to HSA in vitro (i.e., PPB of about 10% to about 95%). In some embodiments, about 20% to about 90% of the cyclic peptide binds to HSA in vitro. In some embodiments, about 20% to about 60% of the cyclic peptide binds to HSA in vitro. In some embodiments, about 40% to about 95% of the cyclic peptide binds to HSA in vitro. In some embodiments, about 40% to about 80% of the cyclic peptide binds to HSA in vitro. In some embodiments, about 40% to about 60% of the cyclic peptide binds to HSA in vitro. In some embodiments, about 60% to about 99% of the cyclic peptide binds to HSA in vitro. In some embodiments, about 60% to about 95% of the cyclic peptide binds to HSA in vitro. In some embodiments, about 60% to about 80% of the cyclic peptide binds to HSA in vitro. In some embodiments, about 60% to about 70% of the cyclic peptide binds to HSA in vitro. In some embodiments, about 40% to about 50% of the cyclic peptide binds to HSA in vitro. In some embodiments, about 50% to about 60% of the cyclic peptide binds to HSA in vitro. In some embodiments, about 70% to about 80% of the cyclic peptide binds to HSA in vitro. In some embodiments, about 80% to about 99% of the cyclic peptide binds to HSA in vitro. In some embodiments, about 80% to about 85% of the cyclic peptide binds to HSA in vitro.

In some embodiments, a conjugate described herein (e.g., a conjugate comprising a (cyclic) peptide of Formula (I), Formula (I-1), Formula (I-2), Formula (Ia), Formula (Ib), or Formula (Ic) is configured to bind to a plasma protein with a prescribed affinity, for example, measured as Plasma Protein Albumin Binding (PPB) percentage. PPB can be determined in vitro by HPLC (e.g., Example B3) or by other suitable means known in the art. In some embodiments, 1% to 99% of the conjugate binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the conjugate binds to HSA in vitro as determined by HPLC. In some embodiments, about 10% to about 95% of the conjugate binds to HSA in vitro (i.e., PPB of about 10% to about 95%). In some embodiments, about 20% to about 90% of the conjugate binds to HSA in vitro. In some embodiments, about 20% to about 60% of the conjugate binds to HSA in vitro. In some embodiments, about 40% to about 95% of the conjugate binds to HSA in vitro. In some embodiments, about 40% to about 80% of the conjugate binds to HSA in vitro. In some embodiments, about 40% to about 60% of the conjugate binds to HSA in vitro. In some embodiments, about 60% to about 99% of the conjugate binds to HSA in vitro. In some embodiments, about 60% to about 95% of the conjugate binds to HSA in vitro. In some embodiments, about 60% to about 80% of the conjugate binds to HSA in vitro. In some embodiments, about 60% to about 70% of the conjugate binds to HSA in vitro. In some embodiments, about 40% to about 50% of the conjugate binds to HSA in vitro. In some embodiments, about 50% to about 60% of the conjugate binds to HSA in vitro. In some embodiments, about 70% to about 80% of the conjugate binds to HSA in vitro. In some embodiments, about 80% to about 99% of the conjugate binds to HSA in vitro. In some embodiments, about 80% to about 85% of the conjugate binds to HSA in vitro.

In some embodiments, a (cyclic) peptide of Formula (I), Formula (I-1), Formula (I-2), Formula (Ia), Formula (Ib), or Formula (Ic) does not contain any S—S bond.

In some embodiments, a peptide of the present disclosure can be cyclized by forming a group as illustrated in Table 4B.

TABLE 4B
Ring Closing Groups (m and n are independently
0 or an integer from 1 to 6.)

—C(═O)—CH₂—
—C(═O)—CH₂—S—
—C(═O)—CH₂—S—CH₂—
—C(═O)—CH₂—S—CH₂—CH₂—
—(CH₂)ₘ—NH—CO—(CH₂)ₙ—
—(CH₂)ₘ—CO—NH—(CH₂)ₙ—
—(CH₂)ₘ—S—(CH₂)ₙ—
—(CH₂)ₘ—CH═CH—(CH₂)ₙ—
—(CH₂)ₘ—NH—(CH₂)ₙ—
—(CH₂)ₘ—S—CH₂-benzene-CH₂—S—(CH₂)ₙ—
—(CH₂)ₘ-triazole-(CH₂)ₙ—
—(CH₂)ₘ-succinimide-S—(CH₂)ₙ—
—C(═O)—CH₂—NH—CH₂—
—C(═O)—CH₂—O—CH₂—
—C(═O)—CH₂—CH₂—S—
—(CH₂)ₘ—S—S—(CH₂)ₙ—
—(CH₂)ₘ—C(═O)—NH—(CH₂)ₙ—
—(CH₂)ₘ—CH₂—CH₂—(CH₂)ₙ—

In some embodiments, m is 0 and n is 0. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, a peptide of the present disclosure, e.g., peptides of Formulae (I), (Ia), (Ib) and (Ic), can be cyclized by reacting a first functional group with a second functional group, see Table 4C. In some embodiments, the first functional group is located at the N-terminus. In some embodiments, the first functional group is located at a non-terminal amino acid. In some embodiments, the second functional group is located at the C-terminus. In some embodiments, the second functional group is located at a non-terminal amino acid.

TABLE 4C
Formation of Ring Closing Groups

| First Functional group | Second Functional group (or amino acid) (e.g., at |
|---|---|
| (e.g., at N-terminus) | C-terminus or at a non-terminal amino acid) |
| —C(═O)—CH₂Cl | Cysteine, homocysteine, lysine, homolysine, |
| | ornithine, diaminobutric acid, serine, homoserine, |
| | threonine, homothreonine |
| —(CH₂)ₙ—NH₂ | Aspartic acid, glutamic acid, homoglutamic acid |
| —(CH₂)ₙ—CO₂H | Lysine, homolysine, ornithine, diaminobutric |
| | acid, |
| —(CH₂)ₙ—Br | Cysteine, homocysteine |
| —(CH₂)ₙ—CH═CH₂ | Allyl-glycine, homoallyl-glycine |
| —(CH₂)ₙ—NH₂ | Aspartate-4-semialdehyde, glutamate-5- |
| | semialdehyde |
| —(CH₂)ₙ—SH | Cysteine, homocysteine conjugated with 1,2- or |
| | 1,3- or 1,4-bis-(bromomethyl)benzene |
| —(CH₂)n-alkyne | XaaC is an amino acid with a side chain with |
| | azide |
| —(CH₂)ₙ—N₃ | Alkynyl-glycine, homoalkynyl-glycine |
| —(CH₂)ₙ-maleimide | Cysteine, homocysteine |

In some embodiments, a conjugate comprising any one of peptide of Table 1 may further comprise amino acid residues at the N and/or C terminus of the peptide, which is not part of the cyclic structure. In some embodiments, the conjugate further comprises a linker.

A peptide described herein can be a peptide mimetic. For example, the peptide can comprise non-peptide bonds and it can comprise one or more unnatural amino acids. Unless stated otherwise, each of the amino acid in a peptide described herein (except the natural amino acid glycine) can independently be in its D or L form. Both D and L forms are encompassed by the present disclosure.

In the present disclosure, the term amino acid embraces derivatives of amino acids. The derivatives include, for example, amino acids obtained by modifying a natural amino acid constituting a protein produced by cellular DNA-encoded biological matter. Examples of such non-natural amino acids include hydroxyproline and hydroxylysine, which are amino acids having a hydroxyl group introduced therein, and diaminopropionic acid, which is an amino acid having an amino group introduced therein.

A peptide described herein can comprise an N-substituted amino acid. In some embodiments, the N-substituted amino acid is a derivative of tryptophan, phenylalanine, tyrosine, arginine, histidine, isoleucine, leucine, lysine, or valine. In some embodiments, the N-substitution is an N-alkyl, such as N-methyl and N-ethyl. In some embodiments, the N-substitution is N-methyl. In some embodiments, the N-substitution is an N-aryl, such as N-phenyl or N-biphenyl. In some embodiments, the N-substitution is an N-heteroaryl such as N-pyridyl. In some embodiments, the N-substituted amino acid is at the N-terminus of the peptide. In some embodiments, the N-substituted amino acid is a non-terminal amino acid.

In some embodiments, peptides described herein comprise one or more amino acids in Tables 5A to 5F.

TABLE 5A
Exemplary Amino Acids at N or C-terminus

| | |
|---|---|
| N-Chloroacetyl-L-alanine | Acetyl-L-alanine |
| N-Chloroacetyl-L-phenylalanine | Acetyl-L-phenylalanine |
| N-Chloroacetyl-L-phenylalanine | Acetyl-L-tyrosine |
| N-Chloroacetyl-L-tyrosine | Acetyl-L-tryptophan |
| N-Chloroacetyl-L-tryptophan | Acetyl-D-alanine |
| N-Chloroacetyl-D-alanine | Acetyl-D-phenylalanine |
| N-Chloroacetyl-D-phenylalanine | Acetyl-D-tyrosine |
| N-Chloroacetyl-D-tyrosine | Acetyl-D-tryptophan |
| N-Chloroacetyl-D-tryptophan | N-3-chloromethylbenzoyl-L-tyrosine |
| | N-3-chloromethylbenzoyl-L-tryptophan |

TABLE 5B
Exemplary Amino Acids That Crosslink With A Peptide

Nγ-(2-chloroacetyl)-α,γ-diaminobutylic acid
Nγ-(2-chloroacetyl)-α,γ-diaminopropanoic acid

TABLE 5C
D-amino Acids

D-Serine
D-Phenylalanine
D-Tyrosine
D-Tryptophan

TABLE 5D
Exemplary N-alkylamino Acids

N-alkyl-Glycine
N-alkyl-Alanine
N-alkyl-Phenylalanine
N-alkyl-Tyrosine
N-alkyl-Serine
N-alkyl-Histidine
N-alkyl-Tryptophan

Exemplary alkyl groups for Table 5D include methyl, ethyl, and propyl groups.

TABLE 5E
Exemplary Peptoid Blocks

N-ethyl-Glycine
N-n-propyl-Glycine
N-n-butyl-Glycine
N-n-pentyl-Glycine
N-n-hexyl-Glycine
N-n-heptyl-Glycine
N-n-octyl-Glycine
N-isopentyl-Glycine
N-(2-phenylethyl)-Glycine
N-(3-phenylpropyl)-Glycine
N-[2-(p-hydroxyphenyl)ethyl]-Glycine

TABLE 5F
Exemplary Unnatural Amino Acids

p-biphenylalanine
p-trifluoromethylphenylalanine
p-azidophenylalanine
p-biotinyl-aminophenylalanine
e-N-Biotinyl-lysine
e-N-Acetyl-lysine
L-Citrulline
L-5-Hydroxytryptphan
L-1,2,3,4,-Tetrahydroisoquinoline-3-carboxylic acid
Aminoisobutyric acid
N-methyl-aminoisobutyric acid
N-methyl-Phenylglycine

Amino acids used in the disclosed peptides can be substituted with similar amino acids. In some embodiments, an amino acid can be substituted with another amino acid with similar hydrophobicity. In some embodiments, an amino acid can be substituted with another amino acid with similar hydrophilicity. In some embodiments, an amino acid can be substituted with another amino acid with similar size. In some embodiments, an amino acid can be substituted with another amino acid with similar charge. In some embodiment, an amino acid can be substituted with another amino acid with a similar functional group. In some embodiments, an amino acid can be substituted with another amino acid with the same functional group.

In some embodiments, an amino acid described herein can be replaced with a variant thereof. Examples of an amino acid substitution or variant include derivatives having an amine, amide, ester, or carboxyl group as the C-terminus and/or N-terminus thereof. Additional examples of amino acid/peptide variants include those obtained by modification such as phosphorylation, alkylation (e.g., methylation), acetylation, adenylylation, ADP-ribosylation, or glycosylation and fused protein obtained by fusion with another peptide or protein. These variants can be prepared by those skilled in the art in a known manner or a method based thereon. An amino acid variant further encompasses the amino acids that have the same functional groups but with different lengths of the side chain (e.g., LysAc vs. OmnAc and cysteine vs. homocysteine). An amino acid variant further encompasses amino acids with a different aromatic moiety compared to the canonical amino acid (e.g., the indole in tryptophan vs the 7-azaindole in 7-AzaTrp; the phenyl in phenylalanine vs the pyridine in 4Py). An amino acid variant further encompasses amino acids with optional substituents, i.e., optionally substituted amino acid. In some embodiments, the optionally substituted amino acid is optionally substituted with one or more substituents independently selected from halogen, hydroxyl, cyano, amino, amide, nitro, ureido, C₁-C₆ alkyl, C₁-C₆ alkoxy, C₆-C₁₀ aryl, C₃-C₆ cycloalkyl, 6-10 membered heterocycloalkyl, and 6-10 membered heteroaryl. In some embodiments, the optionally substituted amino acid is optionally substituted with one or more substituents independently selected from halogen, —CN, —NH₂, —NH (alkyl), —N(alkyl)₂, oxo, —OH, —CO₂H, —CO₂alkyl, —C(═O)NH₂, —C(═O)NH (alkyl), —C(═O)N(alkyl)₂, —S(═O)₂NH₂, —S(═O)₂NH (alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), SF₅, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, a variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl.

In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, and C₁-C₆ alkyl. In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, and C₁-C₆ alkyl. In some embodiments, the variant is selected from amino acids having one or two substituents based on the amino acid, and wherein the substituents are independently selected from C₁-C₆ alkyl.

In some embodiments, a variant of an amino acid is selected from amino acids that have the similar hydrophilicity or hydrophobicity compared to the amino acid. Thus, in some embodiments, a positively charged amino acid can be a variant of another positively charged amino acid. In some embodiments, a negatively charged amino acid can be a variant of another negatively charged amino acid. In some embodiments, a zwitterionic amino acid can be a variant of another zwitterionic amino acid.

In some embodiments, a hydrophilic amino acid has an electrically charged side chain. In some embodiments, a hydrophilic amino acid has a positive charge. In some embodiments, a hydrophilic amino acid has a negative charge. In some embodiments, a hydrophilic amino acid is zwitterionic (e.g., KCOpipzaa). In some embodiments, a hydrophilic amino acid comprises a —OH, COOH, —NH— or NH₂ moiety. In some embodiments, a hydrophilic amino acid comprises —OH, —C(O)OH, —NHC(═NH)NH₂, —NHC(O)NH₂, —C(O)NH₂, or —NHC(O)CH₃. In some embodiments, a hydrophilic amino acid comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂; —C₀₋₆ alkylene-COOH, or —NH—CO—C₁₋₆ alkyl.

In some embodiments, a hydrophobic amino acid is not charged. In some embodiments, a hydrophobic amino acid contains at least 2 contiguous carbon atoms. In some embodiments, a hydrophobic amino acid comprises at least 3 contiguous carbon atoms, either linear or branched. In some embodiments, a hydrophobic amino acid comprises at least 4 contiguous carbon atoms, either linear or branched. In some embodiments, a hydrophobic amino acid comprises at least 5 contiguous carbon atoms, either linear or branched. In some embodiments, a hydrophobic amino acid comprises an ethylene moiety in the side chain. In some embodiments, a hydrophobic amino acid comprises a propylene moiety in the side chain. In some embodiments, a hydrophobic amino acid comprises a butylene moiety in the side chain. In some embodiments, a hydrophobic amino acid comprises phenyl moiety. In some embodiments, a hydrophobic amino acid comprises a heteroaryl moiety. In some embodiments, a hydrophobic amino acid is Trp, Tyr, Phe, or derivatives thereof.

In some embodiments, a variant of an amino acid is selected from amino acids that have the same functional group as the amino acid, and wherein the variant has a different length of a side chain compared to the amino acid. In some embodiments, a variant of an amino acid is selected from amino acids that have the same charge compared to the amino acid. In some embodiments, a variant of an amino acid is selected from amino acids that have the same polarity compared to the amino acid. In some embodiments, an amino acid comprising an aromatic group can be a variant of another amino acid having an aromatic group. In some embodiments, an amino acid comprising a phenyl can be a variant of another amino acid having a phenyl. In some embodiments, an amino acid comprising a heteroaryl can be a variant of another amino acid having a heteroaryl. Amino acids having an aromatic group include, but are not limited to, F, W, Me3Py, MeF, MeF3H, MeFCN, MeF4F, MeF3F, MeFCON, F23dMe, df3CON, W1Me, W1Me7Cl, W1Me7N, W1Et, 7-AzaTrp, W1Me7Br, W1Me7OMe, W1Me6O7Cl, d4PyCON, W7Me, dDab-NH₂-Ph3-SO₂F, dDap-NH₂-Ph3-SO₂F, dDap-NH₂-Ph4-SO₂F, MeF4C, 4Py, 3Py6NH2, 4Py2NH2, and Me4Py. In some embodiments, an amino acid comprising a cycloalkyl group can be a variant of another amino acid having a cycloalkyl group. In some embodiments, an amino acid comprising a heterocycloalkyl group can be a variant of another amino acid having a heterocycloalkyl group.

In some embodiments, a variant of an amino acid is selected from amino acids that have similar polarity and/or charge with the amino acid. For example, in some embodiments, a polar, uncharged amino acid can be a variant of another polar, uncharged amino acid (e.g., Hgn, Q, S, T, Qglucamine),

In some embodiments, a variant of an amino acid has the same number of hydrogen donor as the amino acid. In some embodiments, a variant of an amino acid has the same number of hydrogen acceptor as the amino acid.

In some embodiments, the variant has a molecular weight that does not vary for more than 14, 28, 30, 45 or 60 g/mol compared to the amino acid. In some embodiments, the variant has a molecular weight that does not vary for more than 14 g/mol compared to the amino acid. In some embodiments, the variant has a molecular weight that does not vary for more than 50 g/mol compared to the amino acid. In some embodiments, the variant has a molecular weight that does not vary for more than 28 g/mol compared to the amino acid.

An amino acid variant further encompasses amino acids wherein a functional group is substituted with another functional group having similar properties, e.g., a cysteine can be substituted with a homocysteine. In some embodiments, an aryl functional group can be substituted with an aryl or heteroaryl group. In some embodiments, a heteroaryl functional group can be substituted with an aryl or heteroaryl group. In some embodiments, an amino functional group can be substituted with a NH (alkyl) group.

As used herein, the expression “conservative amino acid substitution” refers to a substitution of functionally equivalent or similar amino acids. A conservative amino acid substitution in a peptide brings about a static change to the amino acid sequence of the peptide. For example, one or two or more amino acids having similar polarity act functionally equivalent to each other and bring about a static change in the amino acid sequence of the peptide. In general, a substitution within a certain group may be considered conservative regarding structure and function. However, as is clear to a person having ordinary skill in the art, the role played by a defined amino acid residue may be determined by its implication in the three-dimensional structure of the molecule containing the amino acid. For example, a cysteine residue in an oxidized-type (disulfide) form may have a lower polarity than that of a reduced-type (thiol) form. The long aliphatic part of the arginine side chain may constitute structurally and functionally important features. Furthermore, the side chain (tryptophan, tyrosine, phenylalanine) including an aromatic ring may contribute to ion-aromatic interaction or cation-pi interaction. In such a case, even if the amino acids having these side chains are substituted for amino acids belonging to the acidic or non-polar groups, they may be structurally and functionally conservative. There is a possibility that residues such as proline, glycine, cysteine (disulfide foam) have a direct effect on the three-dimensional structure of the main chain and often may not be substituted without structural distortion.

Conservative amino acid substitution, as shown below, includes specific substitution based on the similarity of side chains (for example, substitutions are described in Lehninger, Biochemistry, Revised 2nd Edition, published in 1975, pp. 73 to 75: L. Lehninger, Biochemistry, 2nd edition, pp. 73 to 75, Worth Publisher, New York (1975), incorporated herein by reference, and typical substitution.

Hydrophobic amino acids include amino acids that exhibit hydrophobicity, including alanine (also referred to as “Ala” or simply “A”), glycine (also referred to as “Gly” or simply “G”), valine (also referred to as “Val” or simply “V”), leucine (also referred to as “Leu” or simply “L”), isoleucine (also referred to as “Ile” or simply “I”), proline (also referred to as “Pro” or simply “P”), phenylalanine (also referred to as “Phe” or simply “F”), tryptophan (also referred to as Trp” or simply “W”), tyrosine (also referred to as “Tyr” or simply “Y”), and methionine (also referred to as “Met” or simply “M”).

Exemplary hydrophobic amino acids may be further divided into the following groups:
Aliphatic amino acids: Amino acids having a fatty acid or hydrogen in the side chain, including e.g., Ala, Gly, Val, Ile, and Leu.
Aliphatic/branched-chain amino acids: Amino acids having a branched fatty acid in the side chain, including e.g., Val, Ile, and Leu.
Aromatic amino acids: Amino acids having an aromatic ring in the side chain, including e.g., Trp, Tyr, and Phe.

In some embodiments, a hydrophobic amino acid has 4 or more carbon atoms in a side chain (a linear, branched, or cyclic carbon side chain), e.g., Leu, Hcit, Cbg, Chg, or Cba, each of which is optionally N-methylated.

Hydrophilic amino acids include amino acids that exhibit hydrophilicity, including e.g., serine (also referred to as “Ser” or simply “S”), threonine (also referred to as “Thr” or simply “T”), cysteine (also referred to as “Cys” or simply “C”), asparagine (also referred to as “Asn” or simply “N”), glutamine (also referred to as “Gln” or simply “Q”), aspartic acid (also referred to as “Asp” or simply “D”), glutamic acid (also referred to as “Glu” or simply “E”), lysine (also referred to as “Lys” or simply “K”), arginine (also referred to as “Arg” or simply “R”), and histidine (also referred to as “His” or “H”).

Exemplary hydrophilic amino acids may be further divided into the following groups:
Acidic amino acids: Amino acids whose side chains exhibit acidity, including Asp and Glu.
Basic amino acids: Amino acids whose side chains exhibit basicity, including Lys, Arg, and His.
Neutral amino acids: Amino acids whose side chains exhibit neutrality, including Ser, Thr, Asn, Gln, and Cys.

Exemplary hydrophilic amino acids include, for example, N, Q, K, G, S, T, E, Aib, Hcit, Cit, Hgn, KCOpipzaa, Har, Nmm, Ndm, Ala, Hgl, 3Py6NH2, or a variant thereof (including D-amino acid such as da and variations such as Qglucamine, which has gulucamine composition added to the NH₂ terminus of its side chain).

In some embodiments, a peptide described herein comprises an amino acid that affects the direction of the main chain, e.g., Gly and Pro. In some embodiments, a peptide described herein comprises a sulfur-containing amino acid, e.g., Cys and Met. In some embodiments, a peptide described herein comprises an amino acid that comprises an aromatic ring, which can be optionally substituted. Amino acids comprising an aromatic ring include, e.g., F (Phe; phenylalanine), Y (Tyr; tyrosine), W (Trp; tryptophan).

In some embodiments, W or a variant thereof can be W, an amino acid having a heteroatom in the indole ring of W in the side chain, an amino acid in which the hydrogen of NH in the indole ring of W is substituted, or an amino acids having a substituent in the benzene ring of W, or the like.

In some embodiments, F or a variant thereof can be F (phenylalanine), an amino acid wherein (i) the phenyl ring of F is substituted with 1 or 2 substituents each independently selected from —OH, —CN, —Cia alkyl, such as —CH₃; (ii) a 6-membered heteroaryl ring optionally substituted by 1 or 2 substituents each independently selected from —OH, —CN, —C₁₋₃ alkyl, such as —CH₃; or (iii-1) having a heteroatom in the phenyl ring of F in the side chain; (iii-2) a derivative amino acid of F in which a 6-membered heteroaryl ring in the side chain is substituted; or the like. In some aspect, F or a variant thereof is optionally N-methylated.

In some embodiments, W, Y or a variant thereof can be W, Y, an amino acid having either a 6-membered aryl or heteroaryl, or a 9- or 10-membered bi-cyclic aryl or heteroaryl linked to the alpha-carbon through a carbon (e.g., a methylene group). In some embodiments, the 6-, 9-, and 10-membered heteroaryl has one heteroatom (e.g., N), and wherein the 6-, 9-, and 10-membered aryl or heteroaryl is optionally substituted with 1 or 2 substituents independently selected from -methyl, -ethyl, —Cl, and —F. In certain embodiments, W or Y or a variant thereof is W1Me, W1Me7Cl, or F23dMe, Nal1, Nal2, W1Et, Nal21N, 3Bzf, 3Bzt, Nal15N, Nal14N, Nal24N, Nal28N, F23dC, or W1Me7N. In some embodiments, a variant of W is W1Me. In some embodiments, a variant of W is W1Me7Cl. In some embodiments, a variant of Y is F23dMe.

In some embodiments, an amino acid described herein is N-alkylated.

In some embodiments, an amino acid described herein is not N-alkylated (e.g., an amino acid with —H on the alpha-amino group). In certain embodiments, such amino acid is A, E, N, K, Qglucamine, KCOpipzaa, Q, Hse, Cit, Hcit, KAc, DapAc, OrnAc, T, alT, Aib, or 3Py6NH2, more preferably, V, Qglucamine, Cit, Hcit, K, or 3Py6NH2.

Examples of the amino acids include natural protein L-amino acids, unnatural amino acids, and chemically synthesized compounds having properties known in the art as characteristics of an amino acid. Examples of the unnatural amino acids include, but not limited to, α,α-disubstituted amino acids (such as α-methylalanine), N-alkyl-α-amino acids, D-amino acids, β-amino acids, and α-hydroxy acids, each having a backbone structure different from that of natural amino acids; amino acids (such as norleucine and homohistidine) having a side-chain structure different from that of natural amino acids; amino acids (such as “homo” amino acids, homophenylalanine, and homohistidine) having extra methylene in the side chain thereof; and amino acids (such as cysteic acid) obtained by substituting a carboxylic acid functional amino group in the side chain thereof by a sulfonic acid group.

In some embodiments, an amino acid described herein is N-alkylated. In some embodiments, an amino acid described herein is not N-alkylated (e.g., an amino acid with —H on the alpha-amino group). In certain embodiments, such amino acid is A, E, N, K, Qglucamine, KCOpipzaa, Q, Hse, Cit, Hcit, KAc, DapAc, OrnAc, T, alT, Aib, or 3Py6NH2, more preferably, V, Qglucamine, Cit, Hcit, K, or 3Py6NH2.

The peptides described herein can comprise one or more unnatural amino acids. Unnatural amino acids include, but are not limited to, (1) amino acids corresponding to an amino acid residue on a polypeptide subjected to modification after expression (ex. phosphorylated tyrosine, acetylated lysine, or farnesylated cysteine), (2) amino acids that cannot be used in expression on a ribosome but occur naturally, and (3) artificial amino acids that do not occur naturally (unnatural amino acids). Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, and azido-lysine (AzK). In some embodiments, the unnatural amino acid is an unnatural analogue of a tyrosine amino acid; an unnatural analogue of a glutamine amino acid; an unnatural analogue of a phenylalanine amino acid; an unnatural analogue of an alanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid; or a combination thereof. In some embodiments, the unnatural amino acid is an amino acid with a photoactivatable cross-linker; a spin-labeled amino acid; a fluorescent amino acid; a metal binding amino acid; a metal-containing amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α,α-disubstituted amino acid; a β-amino acid; a cyclic amino acid other than proline or histidine, or an aromatic amino acid other than phenylalanine, tyrosine or tryptophan.

Unnatural amino acids include, for example, N-alkyl amino acids in which a natural amino acid described above is N-alkylated, e.g., those modified with lower alkyl groups (for example, of C1 to C5, C1 to C3, and C1) in which the nitrogen forming a peptide bond is branched or not branched. Exemplary N-alkyl amino acids include, e.g., N-ethyl amino acid, N-butyl amino acid, and N-methyl amino acid. Also included are amino acids to which a functional group is further added to the side chain of a natural amino acid or substituted for another functional group (for example, an amino acid having a substitution or an addition in a part such as an arylene group, an alkylene group, or the like of the side chain; an amino acid wherein the arylene group or the alkyl group of the side chain has an increased C-number; an amino acid having a substitution in the aromatic ring of the side chain; a heterocyclic or condensed cyclic amino acid; or the like). Exemplary N-alkyl amino acids further include, e.g., N-alkyllysine and N-methyllysine. Exemplary N-alkyl amino acids further include, e.g., N-methyllysine in which an albumin binder is bound.

In a non-limiting manner, unnatural amino acids include, but are not limited to N-methyl amino acids, da, kCOpipzaa, dahp, df3CON, 4Py, W7N, QPh, alT, W1Me, Cbg, Chg, Cba, Hgl, Hgn, Nmm, Ndm, Hcit, Qglucamine, Hph, W1Me7N, W1Me7Cl, 3Py6NH2, Cit, F23dMe, Har, bA, Kac, dkAc, MeF, Me3Py, MeHph, MeF3CN, MeF3H, MeE, MeN, MeF4C, Nal1, Nal2, W1Et, Nal21N, 3Bzf, 3Bzt, Nal15N, Nal14N, Nal24N, Nal28N, F23dMe, F23dC, W1Me7N, W1Me7Cl, Hse, DapAc, OrnAc, Alb and the like. Note that D-amino acids such as da may be classified as D-amino acids, but they may also be classified according to the properties of their side chains, and N-methyl amino acids may be classified as N-alkyl amino acids and may also be classified according to the property of the side chain.

In some embodiments, the unnatural amino acids incorporated into the peptides include one or more of: 1) a ketone functional group (as found in para or meta acetyl-phenylalanine) that can be specifically reacted with hydrazines, hydroxylamines and their derivatives (Addition of the keto functional group to the genetic code of Escherichia coli. Wang L, Zhang Z, Brock A, Schultz P G. Proc Natl Acad Sci USA. 2003 Jan. 7; 100 (1): 56-61; Bioorg Med Chem Lett. 2006 Oct. 15; 16 (20): 5356-9. Genetic introduction of a diketone-containing amino acid into proteins. Zeng H, Xie J, Schultz P G), 2) azides (as found in p-azido-phenylalanine) that can be reacted with alkynes via copper catalyzed “click chemistry” or strain promoted (3+2) cycloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124 (31): 9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1; 125 (39): 11782-3), or azides that can be reacted with aryl phosphines, via a Staudinger ligation (Selective Staudinger modification of proteins containing p-azidophenylalanine. Tsao M L, Tian F, Schultz P G. Chembiochem. 2005 December; 6 (12): 2147-9), to form the corresponding amides, 3) alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15 (5): 1521-4), and 4) boronic acids (boronates) than can be specifically reacted with compounds containing more than one appropriately spaced hydroxyl group or undergo palladium mediated coupling with halogenated compounds (Angew Chem Int Ed Engl. 2008; 47 (43): 8220-3. A genetically encoded boronate-containing amino acid, Brustad E, Bushey M L, Lee J W, Groff D, Liu W, Schultz P G).

The peptide of the present disclosure embraces various derivatives thereof. Examples of the derivatives include derivatives having an amide, ester, or carboxyl group as the C-terminus and/or N-terminus thereof. Additional examples of the derivatives of the peptide include those obtained by modification such as phosphorylation, methylation, acetylation, adenylylation, ADP-ribosylation, or glycosylation and fused protein obtained by fusion with another peptide or protein. These derivatives can be prepared by those skilled in the art in a known manner or a method based thereon.

In some embodiments, the peptide described herein comprises a basic amino acid. Examples of the basic amino acid include arginine, lysine, citrulline, ornithine, creatine, histidine, diaminobutanoic acid, and diaminopropionic acid.

In some embodiments, provided herein is a peptide having 90% or more sequence identity to any of sequences disclosed herein. In some embodiments, the sequence identity is at least 95% or 99%.

In some embodiments, the peptide is bicyclic or polycyclic. In some embodiments, a conjugate described herein comprises a bicyclic peptide. Exemplary bicyclic peptides include the bicyclic targeting peptides of BT5528, BT1718, and BT8009. Exemplary bicyclic peptides are described in US20180200378, U.S. Pat. Nos. 10,441,663, 8,680,022B2, US20180280525, and US20200215199, each of which is hereby incorporated by reference in its entirety. In some cases, when a peptide is cyclized, protease resistance is improved, metabolic stability is improved, and restrictions are also added to conformational change, so that rigidity is increased and membrane permeability and affinity for the target protein is improved.

In some embodiments, the peptide of the present disclosure has a cyclic structure in which a chloroacetylated amino acid and a cysteine residue present in the peptide are bound. In one aspect, the peptide has a cyclic structure in which an N-terminal amino acid and a cysteine residue present in the peptide are bound. In some embodiments, the peptide has a cyclic structure in which an N-terminal amino acid and the thirteenth cysteine residue present in the peptide are bound. In some embodiments, the peptide has a cyclic structure in which a chloroacetylated N-terminal amino acid and the 12th cysteine residue present in the peptide are bound. “Chloroacetylation” may be replaced with “haloacetylation” using another halogen. Furthermore, “acetylation” may be “acylation” using an acyl group other than an acetyl group.

In some embodiments, the peptide is a lasso peptide. Lasso peptides can be synthetic or naturally produced by bacteria, and they possess a distinctive threaded lariat fold that offers a 3D array of functionality for engaging biological targets. This lasso structure can enable beneficial properties such as affinity, stability and potent biological activities. Suitable lasso structure can be designed by algorithms. Exemplary lasso peptides are provided in Hegemann, J. D., et al., Lasso Peptides: An Intriguing Class of Bacterial Natural Products, Acc. Chem. Res., 2015, 48, 1909-1919; Tietz, J. I., et al., A new genome-mining tool redefines the lasso peptide biosynthetic landscape, Nature Chem Bio, 2017, 13, 470-478; DiCaprio, A. J., et al., Enzymatic Reconstitution and Biosynthetic Investigation of the Lasso Peptide Fusilassin, J. Am. Chem. Soc., 2019, 141, 290-297; Al Toma, R. S., et al., Site-Directed and Global Incorporation of Orthogonal and Isostructural Noncanonical Amino Acids into the Ribosomal Lasso Peptide Capistruin, ChemBioChem, 2015, 16, 503-509.

Further exemplary peptides include BMS-753493, Somatostatins, Octreotide, Octreotate, Lanreotide, Pasireotide, JR-11, L-779,976, BIM-23120, Satoreotide, depreotide, 18F-KYNDRLPLYISNP (SEQ ID NO: 274), CalX-P1, and FAP-2286.

The peptide of the present disclosure embraces salts thereof. As the salts of the peptide, salts with physiologically acceptable base or acid are used. Examples include addition salts with an inorganic acid (such as hydrochloric acid, hydrobromic acid, hydroiodic acid, sulfuric acid, or phosphoric acid), addition salts with an organic acid (such as p-toluenesulfonic acid, methanesulfonic acid, oxalic acid, p-bromophenylsulfonic acid, carboxylic acid, succinic acid, citric acid, benzoic acid, or acetic acid), inorganic bases (such as ammonium hydroxide, alkali or alkaline earth metal hydroxide, carbonate, or bicarbonate), and an amino acid.

Moreover, a linker may be further added to the (cyclic) peptide. Examples of the linker include the foregoing amino acid linker (peptide linker), a chemical linker, a fatty acid linker, a nucleic acid linker, a sugar chain linker, or the like, or it may be a complex, for example, a chemical linker, a peptide linker, or the like. Examples of the chemical linker include a PEG (polyethylene glycol) linker. For example, the PEG linker may comprise between 1 to 24 ethylene glycol units. Furthermore, the linker may be a fatty acid linker containing a divalent chemical moiety derived from a fatty acid. The linker includes at least one amino acid, and, for example, a glycine-rich peptide such as a peptide having a sequence [Gly-Gly-Gly-Gly-Ser] n (in the formula, n is 1, 2, 3, 4, 5, or 6 (SEQ ID NO: 275)) such as that according to U.S. Pat. No. 7,271,149, incorporated by reference herein, or a serine-rich peptide linker according to U.S. Pat. No. 5,525,491, incorporated by reference herein, may be used. In a non-limiting manner, there are some cases where a physical property (for example, solubility) of the peptide may be changed by the addition of a linker. In one aspect, the amino acid linker includes an amino acid sequence according to any one of SEQ ID NOs: 1 to 171.

The linker may be added at any position. For example, it may be bound to Cys positioned on the C-terminal side or may be bound to an amino acid comprised in the cyclic peptide. In some instances, it is bound to Cys or variant thereof positioned on the C-terminal side. In this case, linker is added to the —COOH on the Cys residue. It may be possible to add one to several amino acid to the C-terminus of such Cys residue and then the liker is added to its terminus; for example, Gly is added to the C-terminus of Cys within the cyclic structure peptide, then the —COOH of the Gly is bound to linker such as PEG linker or amino acid linker. In other instances, linker is added to the side chain on amino acid, preferably Lys, within the cyclic peptide. In this case, for example, linker is added to the side chain of Lys at X5, X8 or X10.

### EphA2-Binding Peptide and Peptide Having EphA2 Antagonistic Activity

EPH receptor A2 (ephrin type-A receptor 2) is a protein that in humans is encoded by the EPHA2 gene. EphA2 may be upregulated in multiple cancers, often correlating with disease progression, metastasis and poor prognosis e.g., in solid tumors such as breast, lung, gastric, pancreatic, prostate, liver and glioblastoma.

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) can control cell positioning and tissue organization. Functional and biochemical Eph responses can occur at higher ligand oligomerization states.

Among other patterning functions, various Ephs and ephrins have been shown to play a role in vascular development. Knockout of EphB4 and ephrin-B2 can result in a lack of the ability to remodel capillary beds into blood vessels and embryonic lethality. Persistent expression of some Eph receptors and ephrins has also been observed in newly-formed, adult micro-vessels (Brantley-Sieders et al. (2004) Curr Pharm Des 10, 3431-42). The de-regulated re-emergence of some ephrins and their receptors in adults may contribute to tumor invasion, metastasis and neo-angiogenesis. Furthermore, some Eph family members may be over-expressed on tumor cells from a variety of human tumors (Booth et al. (2002) Nat Med 8, 1360-1).

In some embodiments, the peptide of the present technology binds to EphA2. In some implementations of these embodiments, the peptide has EphA2 antagonistic activity. In some instances, the peptide binds to human EphA2 (hEphA2) and has hEphA2 antagonistic activity.

As used herein, the term “EphA2” refers to any form of EphA2 and a variant thereof for retaining at least a part of the activity of EphA2. The EphA2 includes all the native sequences of EphA2 in mammals such as, for example, humans, dogs, cats, horses, and cows, unless otherwise specifically described as human EphA2 (hEphA2). One exemplification of EphA2 is hEphA2 (Gene ID: 1969), which is human EphA2 and is a protein having an amino acid sequence (SEQ ID NO: 276, Isoform 1, P29317-1).

(SEQ ID NO: 276)
MELQAARACFALLWGCALAAAAAAQGKEVVLLDFAAAGGELGWLT
HPYGKGWDLMQNIMNDMPIYMYSVCNVMSGDQDNWLRTNWVYRGE
AERIFIELKFTVRDCNSFPGGASSCKETFNLYYAESDLDYGTNFQ
KRLFTKIDTIAPDEITVSSDFEARHVKLNVEERSVGPLTRKGFYL
AFQDIGACVALLSVRVYYKKCPELLQGLAHFPETIAGSDAPSLAT
VAGTCVDHAVVPPGGEEPRMHCAVDGEWLVPIGQCLCQAGYEKVE
DACQACSPGFFKFEASESPCLECPEHTLPSPEGATSCECEEGFFR
APQDPASMPCTRPPSAPHYLTAVGMGAKVELRWTPPQDSGGREDI
VYSVTCEQCWPESGECGPCEASVRYSEPPHGLTRTSVTVSDLEPH
MNYTFTVEARNGVSGLVTSRSFRTASVSINQTEPPKVRLEGRSTT
SLSVSWSIPPPQQSRVWKYEVTYRKKGDSNSYNVRRTEGFSVTLD
DLAPDTTYLVQVQALTQEGQGAGSKVHEFQTLSPEGSGNLAVIGG
VAVGVVLLLVLAGVGFFIHRRRKNQRARQSPEDVYFSKSEQLKPL
KTYVDPHTYEDPNQAVLKFTTEIHPSCVTRQKVIGAGEFGEVYKG
MLKTSSGKKEVPVAIKTLKAGYTEKQRVDFLGEAGIMGQFSHHNI
IRLEGVISKYKPMMIITEYMENGALDKFLREKDGEFSVLQLVGML
RGIAAGMKYLANMNYVHRDLAARNILVNSNLVCKVSDFGLSRVLE
DDPEATYTTSGGKIPIRWTAPEAISYRKFTSASDVWSFGIVMWEV
MTYGERPYWELSNHEVMKAINDGFRLPTPMDCPSAIYQLMMQCWQ
QERARRPKFADIVSILDKLIRAPDSLKTLADFDPRVSIRLPSTSG
SEGVPFRTVSEWLESIKMQQYTEHFMAAGYTAIEKVVQMTNDDIK
RIGVRLPGHQKRIAYSLLGLKDQVNTVGIPI.

Another isoform of Human EphA2 can have a sequence according to the following SEQ ID NO: 277 (Isoform 2, P29317-2):

(SEQ ID NO: 277)
MELQAARACFALLWGCALAAAAAAQGKEWLLDFAAAGGELGWLTH
PYGKGWDLMQNIMNDMPIYMYSVCNVMSGDQDNWLRTNWVYRGEA
ERIFIELKFTVRDCNSFPGGASSCKETFNLYYAESDLDYGTNFQK
RLFTKIDTIAPDEITVSSDFEARHVKLNVEERSVGPLTRKGFYLA
FQDIGACVALLSVRVYYKKCPELLQGLAHFPETIAGSDAPSLATV
AGTCVDHAVVPPGGEEPRMHCAVDGEWLVPIGQCLCQAGYEKVED
ACQACSPGFFKFEASESPCLECPEHTLPSPEGATSCECEEGFFRA
PQDPASMPCTRPPSAPHYLTAVGMGAKVELRWTPPQDSGGREDIV
YSVTCEQCWPESGECGPCEASVRYSEPPHGLTRTSVTVSDLEPHM
NYTFTVEARNGVSGLVTSRSFRTASVSINQTEPPKVRLEGRSTTS
LSVSWSIPPPQQSRVWKYEVTYRKKVTPRGAGLALAGPTAGDRLV
T

As used herein, the expression “has avidity for EphA2” or “binds to EphA2” indicates having the activity of binding to EphA2. Binding site of the peptide of the present invention on the EphA2 is not limited, the peptide can bind to anywhere on the EphA2 protein. Binding to EphA2 may be measured by any method for measuring known intermolecular binding. In a non-limiting manner, for example, this may be determined by competitive binding assays such as surface plasmon resonance (SPR) assays, scatter analysis and/or radioimmunoassays (RIA), enzyme immunoassays (EIA), and sandwich and competitive assays, and in any suitable manner which is known, including different variants of the examples given that are known in the technical field.

In some embodiments, the peptide of this invention competes for binding to hEphA2 at one or more amino acid residues selected from Asp53, Met55, Asn57, Met59, Met66, Thr101, Arg103, Phe156, Glu157, Arg159, Val161, Val189, and Ala190. In some embodiments, the peptide competes for binding to human EphA2 at one or more amino acid residues selected from Asp53, Phe156, and Glu157. In some embodiments, the peptide competes for binding to human EphA2 at Asp53, Glu157, or both.

In one aspect, the binding affinity of the peptide of the present technology is at most 100 nM as determined by Kd in surface plasmon resonance (SPR) analysis. In some implementations, the Kd of the peptides of the present technology is 100 nM ore less, 50 nM or less, 30 nM or less, 20 nM or less, 10 nM or less, 5 nM or less, 4 nM or less, 3 nM or less, 2 nM or less, 1 nM or less, 0.9 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 0.01 nM or less.

In some embodiments, a peptide described herein has a binding affinity to a human EphA2 of at most 1, 5, 10, 50, 100, 200, 500, 1000, 5000 or 10,000 nM as determined by Kd in surface plasmon resonance (SPR) analysis. In some embodiments, a peptide described herein has a binding affinity to a human EphA2 of at most 100 nM as determined by Kd in surface plasmon resonance (SPR) analysis. In some embodiments, a peptide described herein has a binding affinity to a human EphA2 of at most 1 nM as determined by Kd in surface plasmon resonance (SPR) analysis. In some embodiments, a peptide described herein has a binding affinity to a human EphA2 of at most 2 nM as determined by Kd in surface plasmon resonance (SPR) analysis. In some embodiments, a peptide described herein has a binding affinity to a human EphA2 of at most 5 nM as determined by Kd in surface plasmon resonance (SPR) analysis. In some embodiments, a peptide described herein has a binding affinity to a human EphA2 of at most 10 nM as determined by Kd in surface plasmon resonance (SPR) analysis.

In some embodiments, a conjugate described herein has a binding affinity to a human EphA2 of at most 1, 5, 10, 50, 100, 200, 500, 1000, 5000 or 10,000 nM as determined by Kd in surface plasmon resonance (SPR) analysis. In some embodiments, a conjugate described herein has a binding affinity to a human EphA2 of at most 100 nM as determined by Kd in surface plasmon resonance (SPR) analysis. In some embodiments, a conjugate described herein has a binding affinity to a human EphA2 of at most 1 nM as determined by Kd in surface plasmon resonance (SPR) analysis. In some embodiments, a conjugate described herein has a binding affinity to a human EphA2 of at most 2 nM as determined by Kd in surface plasmon resonance (SPR) analysis. In some embodiments, a conjugate described herein has a binding affinity to a human EphA2 of at most 5 nM as determined by Kd in surface plasmon resonance (SPR) analysis. In some embodiments, a conjugate described herein has a binding affinity to a human EphA2 of at most 10 nM as determined by Kd in surface plasmon resonance (SPR) analysis.

In one aspect, the binding affinity of the peptide or conjugate of the present disclosure is at most 100 nM as determined by Kd in surface plasmon resonance (SPR) analysis. In some implementations, the Kd of the peptide or conjugate of the present disclosure is 100 nM ore less, 50 nM or less, 30 nM or less, 20 nM or less, 10 nM or less, 5 nM or less, 4 nM or less, 3 nM or less, 2 nM or less, 1 nM or less, 0.9 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 0.01 nM or less.

### Unnatural Amino Acids

In certain embodiments, the peptides and conjugates described herein comprises one or more unnatural amino acids that are not any one of the 20 canonical amino acids found in proteins. Representative unnatural amino acids that can be incorporated into the peptides and conjugates described herein are provided in the table below.

TABLE 3
Structures of exemplary unnatural amino acids
that can be incorporated into a peptide/conjugate described herein

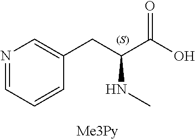

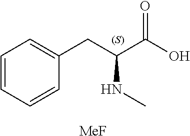

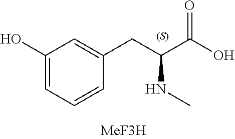

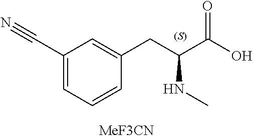

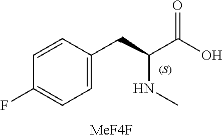

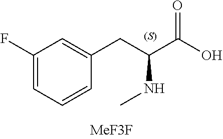

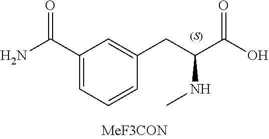

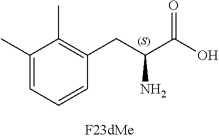

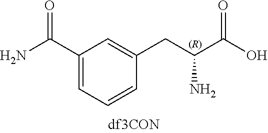

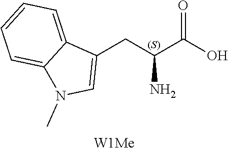

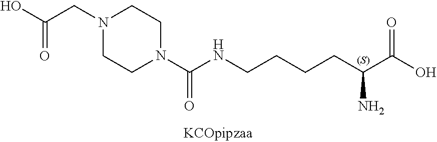

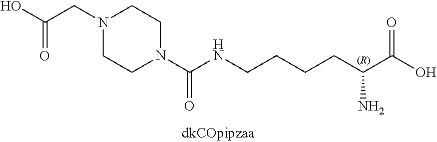

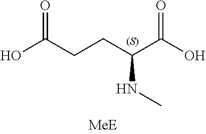

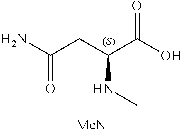

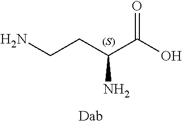

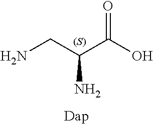

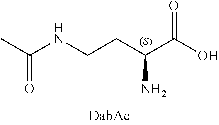

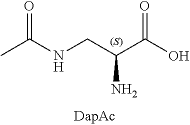

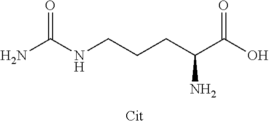 [SMILES: NC(=O)NCCC[C@H](N)C(=O)O]

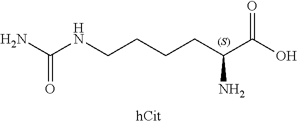

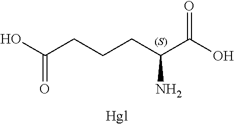

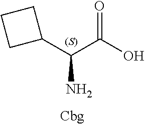

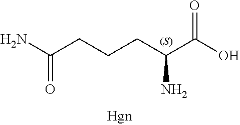

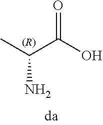

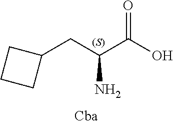

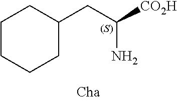

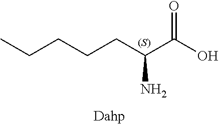

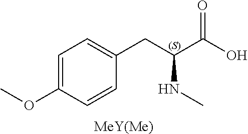

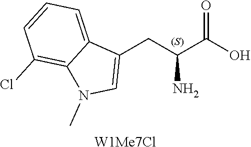

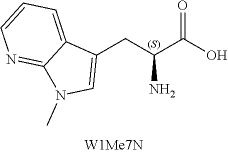

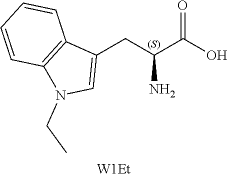

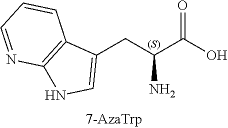

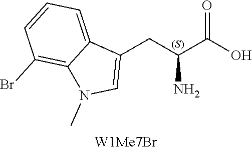

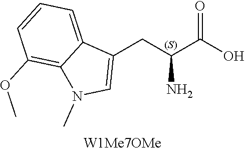

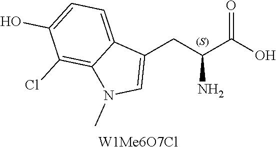

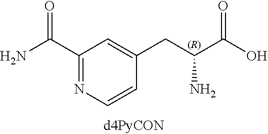

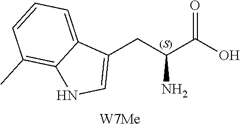

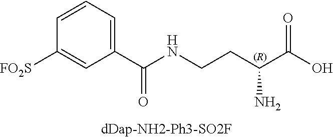

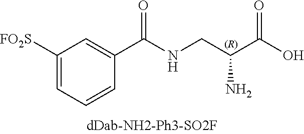

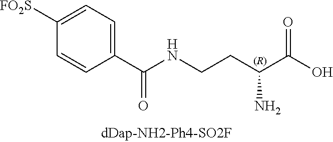

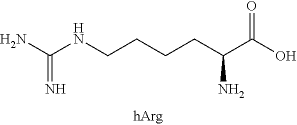 [SMILES: N=C(N)NCCCC[C@H](N)C(=O)O]

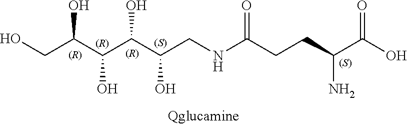

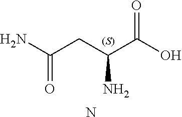

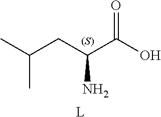

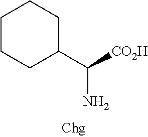 [SMILES: N[C@H](C(=O)O)C1CCCCC1]

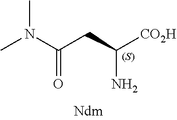

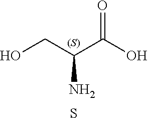

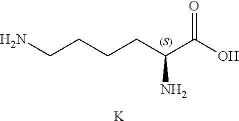

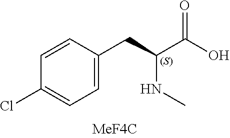

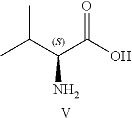

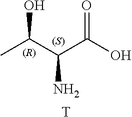

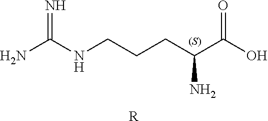

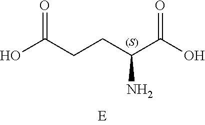

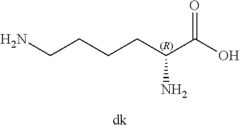

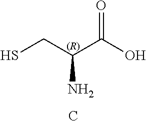

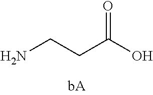

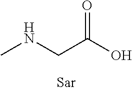

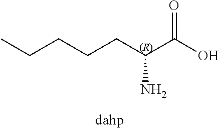

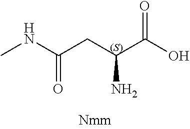

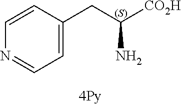

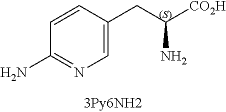

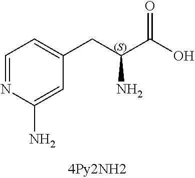

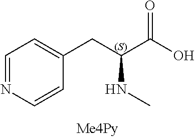

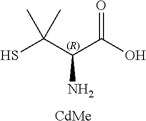

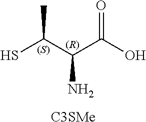

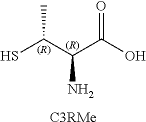

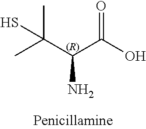

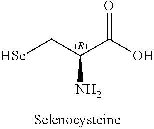

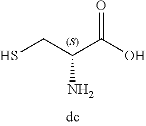

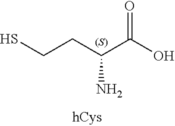

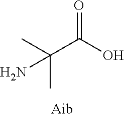

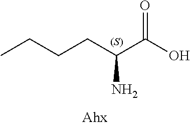

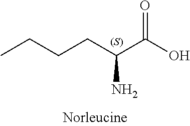

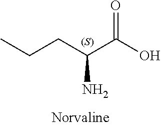

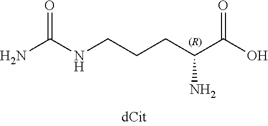

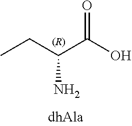

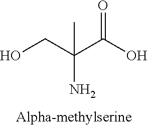

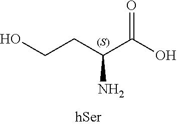

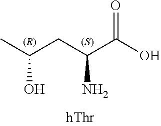

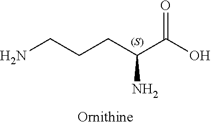

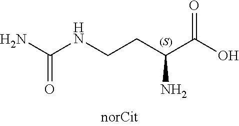

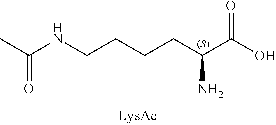

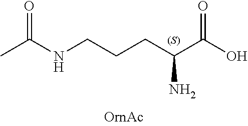

### Linkers & Peptide-Linkers

A peptide described herein may be linked to one or more linkers, before such peptide-linker intermediate is further linked to a payload molecule to form the conjugate described herein. Thus, a conjugate described herein can comprise one or more linkers. In some embodiments, the linker covalently attaches the peptide with the payload molecule in the conjugate. In some other embodiments, the peptide attaches directly to the payload molecule without a linker. In some embodiments, the present disclosure describes linkers that function as a spacer.

A linker can comprise a number of intervening atoms (on a linear chain, excluding pendant groups or substituents) between the payload molecule and the binding peptide described herein, thereby creating a distance between the payload molecule and the binding peptide. In some embodiments, a linker comprises 10-100 intervening atoms between the payload molecule and the binding peptide. In some embodiments, a linker comprises 2-60 intervening atoms between the payload molecule and the binding peptide. In some embodiments, a linker comprises 2 to 20, 2 to 50, 5 to 15, 5 to 25, 10 to 40, 30 to 60, or 10 to 20 intervening atoms between the payload molecule and the binding peptide. In some embodiments, a linker comprises 3 to 30 intervening atoms between the payload molecule and the binding peptide. In some embodiments, a linker comprises 5 to 25 intervening atoms between the payload molecule and the binding peptide. In some embodiments, a linker comprises 6 to 18 intervening atoms between the payload molecule and the binding peptide. In some embodiments, a linker comprises 10 to 20 intervening atoms between the payload molecule and the binding peptide. The intervening atoms can comprise 1 or more carbons, and optionally one or more heteroatoms such as O and N. In some embodiments, the intervening atoms comprise 2 to 20, 2 to 50, 5 to 15, 5 to 25, 10 to 40, 30 to 60, or 10 to 20 carbons. In some embodiments, the intervening atoms comprise 0, 1, 2, 3, 4, 5, or 6 nitrogen. In some embodiments, the intervening atoms comprise 0, 1, 2, 3, 4, 5, 6, 7 or 8 oxygen. In some embodiments, the intervening atoms comprise 1 to 6 nitrogen and 0 to 4 oxygen.

A linker can comprise one or more amino acid residues. In some embodiments, the linker comprises 1 to 3, 1 to 5, 1 to 10, 5 to 10, or 5 to 20 amino acid residues. In some embodiments, the linker comprises 1, 2, 3, 4, 5, 6, 7, 8, 9, or 10 amino acid residues. In some embodiments, the linker comprises 1 to 5 amino acid residues. For example, the linker can comprise one or more lysine (K) residues such as K, KK, or KKK sequences. In some embodiments, the linker comprises a lysine or a derivative thereof. In some embodiments, the linker comprises a lysine. In some embodiments, one or more amino acids of the linker are unnatural amino acids. In some embodiments, the linker comprises a lysine residue, an alanine residue, or both. In certain embodiments, the linker comprises one or more amino acids chosen from a lysine residue, an alanine residue, or a phenylalanine residue. In some embodiments, the linker comprises a lysine residue. In some embodiments, the linker comprises an alanine residue.

A herein-described linker can attach to the N-terminus of the peptide, the C-terminus of the peptide, or a non-terminal amino acid of the peptide, or it can attach to the peptide through a combination of the above. In some embodiments, the linker is attached to the peptide via its N-terminus. In some embodiments, the linker is attached to the peptide via a cysteine residue at the C-terminus. In some embodiments, the linker is attached to the peptide via a cysteine residue at the N-terminus. In some embodiments, the linker is attached to the peptide via its C-terminus. In some embodiments, the linker is attached to the peptide via a non-terminal amino acid. The linker can be bonded to the peptide, the payload molecule, or both, for example, through a chemically reactive group. Exemplary chemically reactive groups include, but are not limited to, a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound to the peptide can be a natural or unnatural amino acid of the peptide and/or it can be introduced into the peptide, e.g., by DNA recombinant technology (e.g., by introducing a cysteine or protease cleavage site in the amino acid sequence) or by protein biochemistry (e.g., reduction, pH adjustment or proteolysis). Exemplary methods for attaching the linker includes carbodiimide reaction, reactions using bifunctional agents such as dialdehydes or imidoesters, Schiff base reaction, Suzuki-Miyaura cross-coupling reactions, Isothiocyanates as coupling agents, and click chemistry.

The linker can have a prescribed length thereby linking the payload molecule and the peptide while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 278), proline-rich sequences, and regions rich in double and/or triple bonds.

The linker can be cleavable, e.g., under physiological conditions, e.g., under intracellular conditions, such that cleavage of the linker releases the payload molecule in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises an amino acid sequence, such as a combination of amino acid sequence and a flexible and/or rigid region, as exemplified in Table 6B, shown in the “Linker” column. For example, PDC_EphA2-00010011-C003 includes a linker comprising an amino acid residue, bA-dk. In another example, PDC_EphA2-00001417-C004 includes a linker comprising a combination of amino acid residues and PEG, KA-dk-(PEG8c-PEG2c).

In some embodiments, the linker can comprise an amino acid sequence, or a combination of amino acid sequence and flexible and/or rigid region, such as those provided in Table 13 (see the “Linker/payload” column). For example, in Table 13, Biotin, SulfoCys5 are shown as payloads. PDC_EphA2-00010011-C003 includes the linker comprising amino acid residue, bA-dk.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀. In some embodiments, the linker comprises a structure selected from:

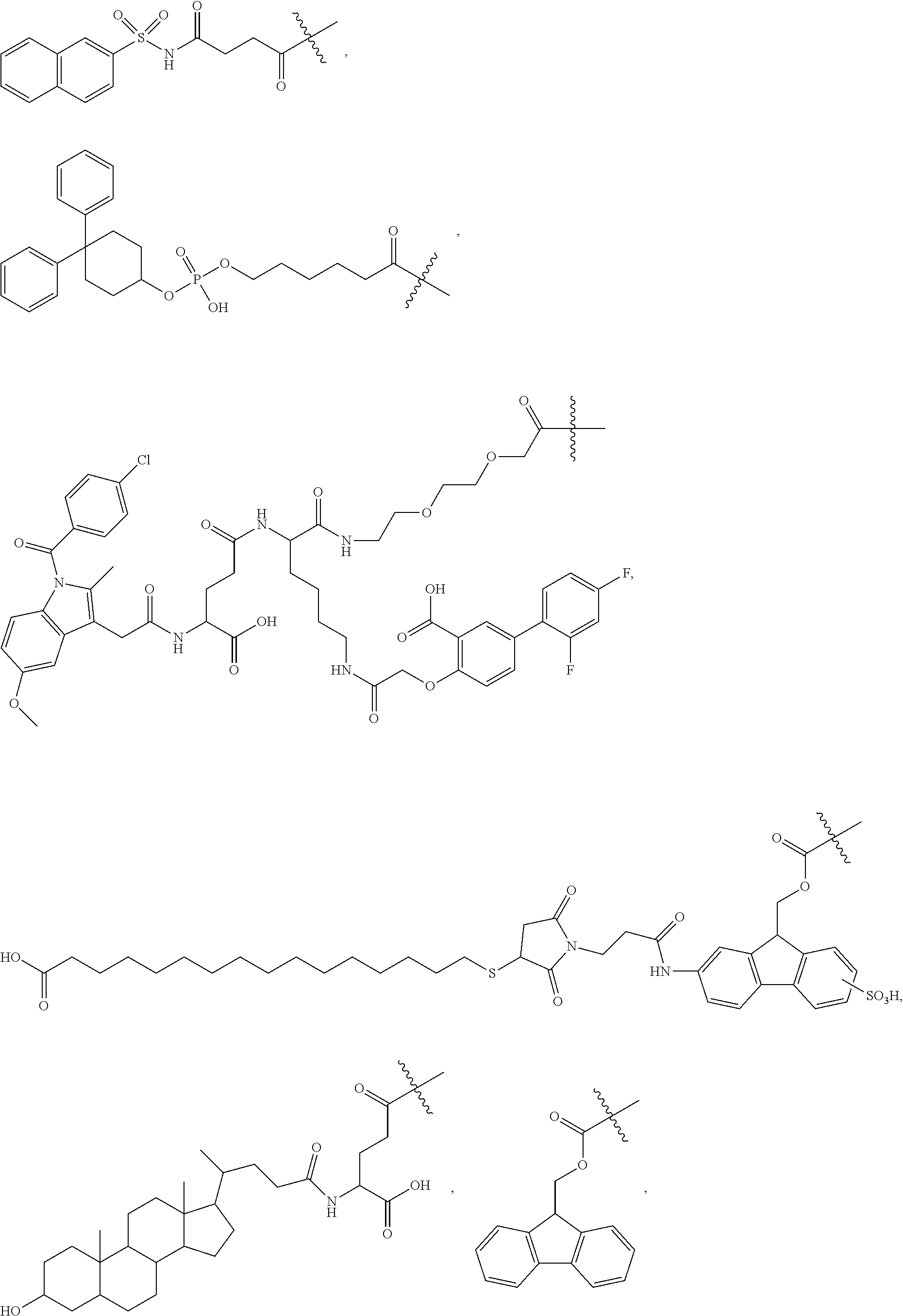

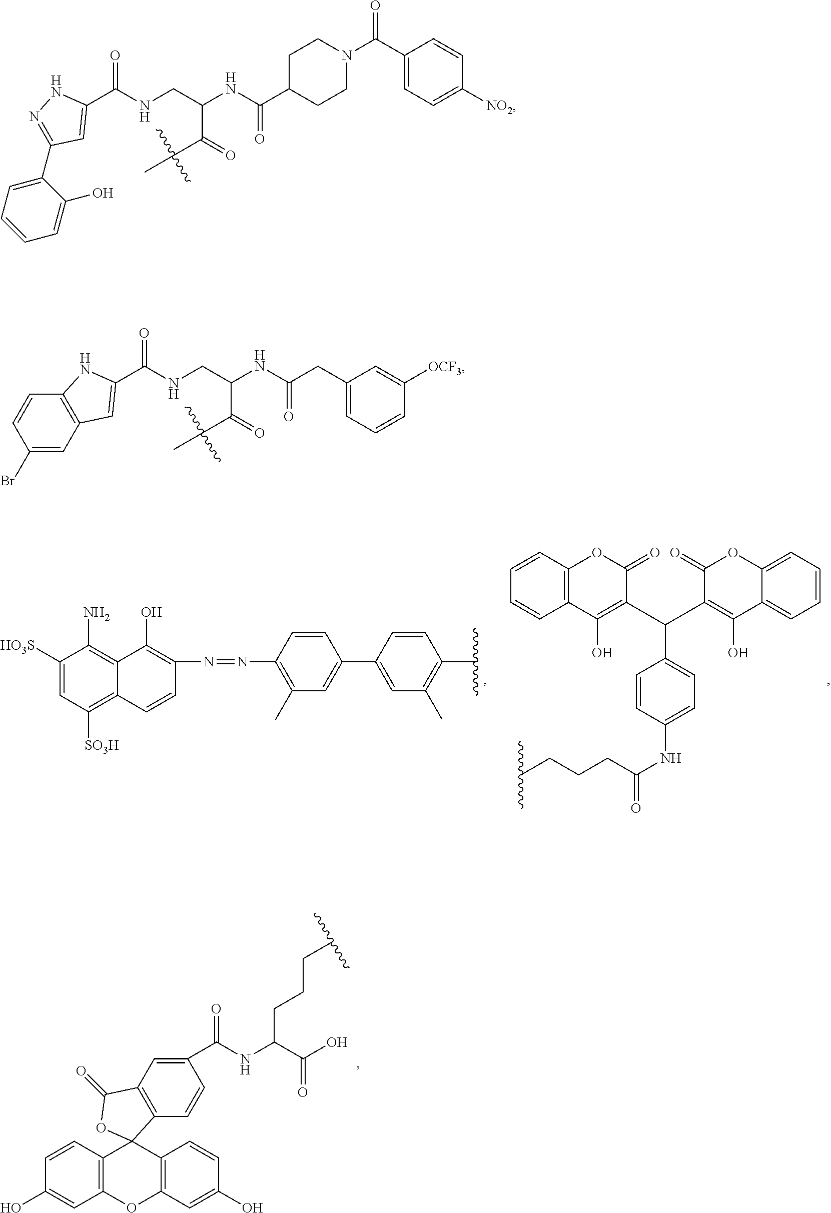 [SMILES: *C(=O)C(CNC(=O)c1cc(-c2ccccc2O)n[nH]1)NC(=O)C1CCN(C(=O)c2ccc([N+](=O)[O-])cc2)CC1 ; *C(=O)C(CNC(=O)c1cc2cc(Br)ccc2[nH]1)NC(=O)Cc1cccc(OC(F)(F)F)c1 ; *c1ccc(-c2ccc(N=Nc3ccc4c(S(=O)(=O)O)cc(S(=O)(=O)O)c(N)c4c3O)c(C)c2)cc1C ; *CCCC(=O)Nc1ccc(C(c2c(O)c3ccccc3oc2=O)c2c(O)c3ccccc3oc2=O)cc1 ; *CCCC(NC(=O)c1ccc2c(c1)C(=O)OC21c2ccc(O)cc2Oc2cc(O)ccc21)C(=O)O]

and structures derived from any one thereof.

In some embodiments, the linker comprises a click chemistry residue. In some embodiments, the linker is attached to the peptide, to the payload molecule, or both via click chemistry, thereby forming a click chemistry residue. For example, the peptide can comprise an azide group (at N- or C-terminus or at a non-terminal amino acid) that reacts with an alkyne moiety of the linker. For another example, the peptide can comprise an alkyne group (at N- or C-terminus or at a non-terminal amino acid) that reacts with an azide of the linker. The payload molecule and the linker can be attached similarly. In some embodiments, the linker comprises an azide moiety, an alkyne moiety, or both. In some embodiments, the linker comprises a triazole. In some embodiments, the click chemistry residue is

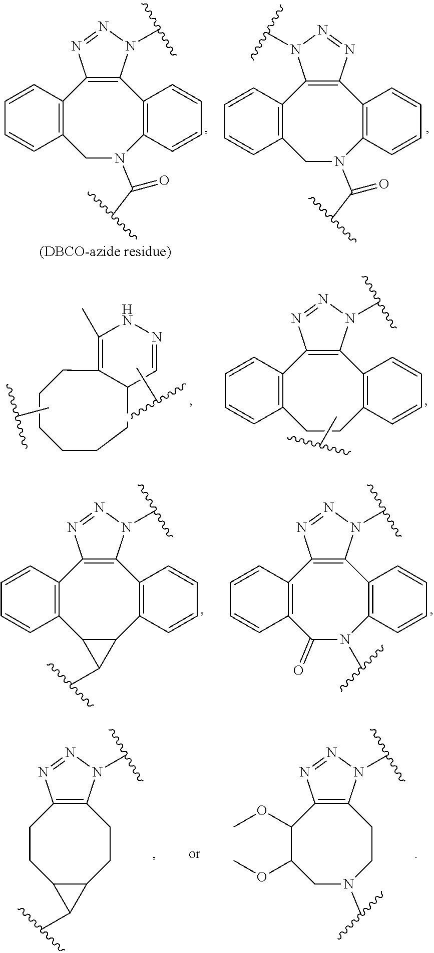

In some embodiments, the click chemistry residue is a DIBO-azide residue, BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry, A Powerful Tool for Pharmaceutical Sciences,” Pharmaceutical Research volume 25, pages 2216-2230 (2008); Thirumurugan et al, “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

In some embodiments, a linker described herein comprises two or more motifs. In some embodiments, one or more of the motifs are connected via click chemistry such that they can be clicked in/out of the linker. Each of the motifs in a linker can have independent functions. For example, a linker can comprise a motif that functions to adjust plasma half-life and/or a motif that functions as a spacer between the peptide and payload molecule.

In some embodiments, the linker has a structure of

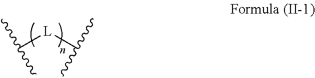

wherein each L is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}, —NR^{L}C(═O)O—, —NR^{L}C(═O)NR^{L}—, —NR^{L}C(═S)NR^{L}—, —CR^{L}═N—, —N═CR^{L}, —NR^{L}—S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂_{⁺}—, —N(R^{L})₂_{⁺}—(C₁-C₃₀ alkylene)-, or a click chemistry residue; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20 (e.g., 1, 2, 3, 4, 5, 6, 7, 8, 9, 10, 11, 12, 13, 14, 15, 16, 17, 18, 19, or 20).
   In some embodiments, the linker has a structure of

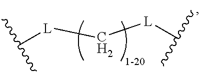

wherein each L is independently —O—, —NR^{L}—, —N(R^{L})₂_{⁺}, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}—C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

In some embodiments, the linker of Formula (II-1) has a structure of Formula (II-1a),

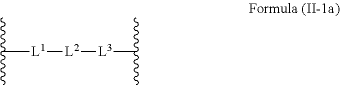

wherein
   each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—; and
   L² is absent, substituted or unsubstituted C₁-C₃₀ alkylene, or substituted or unsubstituted C₁-C₃₀ heteroalkylene.

In some embodiments, the linker comprises a structure of Formula (II-1b),

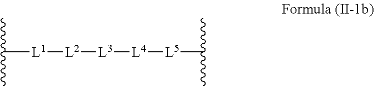

wherein
   each of L¹ and L⁵ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂, —S(═O)₂NR^{L}C(═O)—, substituted or unsubstituted 5-6 membered cycloalkyl, or substituted or unsubstituted 5-6 membered heterocycloalkyl; and
   L², L³ and L⁴ are each independently absent, substituted or unsubstituted 5-6 membered cycloalkyl, substituted or unsubstituted 5-6 membered heterocycloalkyl, substituted or unsubstituted C₁-C₃₀ alkylene, or substituted or unsubstituted C₁-C₃ heteroalkylene.

In some embodiments, L¹ is —NH—.

In some embodiments, L² is absent. In some embodiments, L² is substituted or unsubstituted C₁-C₃₀ alkylene, or substituted or unsubstituted C₁-C₃ heteroalkylene. In some embodiments, L² is substituted or unsubstituted C₁-C₃₀ alkylene. In some embodiments, L² is substituted or unsubstituted C₁-C₃₀ heteroalkylene. In some embodiments, L² is substituted or unsubstituted C₁-C₁₈ alkylene, or substituted or unsubstituted C₁-C₁₈ heteroalkylene. In some embodiments, L² is optionally substituted. In some embodiments, L² is optionally substituted with one or more substituents selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}In some embodiments, L² is C₁-C₃₀ heteroalkylene that is optionally substituted with one or more substituents selected from —OH, —SH, oxo, amino, C₁-C₆ alkyl, C₁-C₆ hydroxyalkyl, C₁-C₆ haloalkyl, and C₁-C₆ aminoalkyl. In some embodiments, L² is optionally substituted with C₁-C₆ alkyl which is further optionally substituted with one or more substituents chosen from —OH, —SH, oxo, amino, C₅-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

In some embodiments, L³ is —NH—. In some embodiments, L³ is absent.

In some embodiments, L⁴ is absent. In some embodiments, L⁴ is substituted or unsubstituted 5-6 membered cycloalkyl, substituted or unsubstituted 5-6 membered heterocycloalkyl, substituted or unsubstituted C₁-C₃₀ alkylene, or substituted or unsubstituted C₁-C₃₀ heteroalkylene.

In some embodiments, L⁵ is —NH—. In some embodiments, L⁵ is absent.

In some embodiments for Formula (II-1b), L¹ is —O—, —N(methyl)-, —NH— or —C(═O)—; L⁵ is —O—, —N(methyl)-, —NH— or —C(═O)—; L², L³ and L⁴ are each independently absent, substituted or unsubstituted 5-6 membered cycloalkyl, substituted or unsubstituted 5-6 membered heterocycloalkyl, substituted or unsubstituted C₁-C₁₂ alkylene, or substituted or unsubstituted C₁-C₃₀ heteroalkylene, wherein L¹ is connected to the payload molecule and L⁵ is connected to the EphA2 binding peptide.

In some embodiments for Formula (II-1b), L² is unsubstituted C₁-C₁₂ alkylene, and L³ and L⁴ are absent.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, each of L¹ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L¹ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L¹ is —O—, —NH—, —S(═O)—, —S(═O)₂—, or —C(═O)—. In some embodiments, L¹ is —C(═O)NH— or —NHC(═O)—. In some embodiments, L¹ is substituted or unsubstituted C₃-C₁₅ cycloalkyl, or substituted or unsubstituted C₁-C₁₂ heterocycloalkyl. In some embodiments, L¹ is substituted or unsubstituted aryl or substituted or unsubstituted heteroaryl. In some embodiments, L¹ is substituted or unsubstituted C₁-C₃₀ alkylene. In some embodiments, L¹ is substituted or unsubstituted C₂-C₃₀ alkenylene. In some embodiments, L¹ is substituted or unsubstituted C₁-C₃₀ heteroalkylene. In some embodiments, L¹ is substituted or unsubstituted C₅-C₂₅ heteroalkylene. In some embodiments, L¹ is substituted or unsubstituted C₅-C₁₂ heteroalkylene.

In some embodiments, each of L² is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L² is —O—, —NR^{L}—, —OP(═O)(OR^{L})O—, —S—, —S(═O)—, —S(═O)₂—, —C(═O)₂—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L² is —O—, —NH—, —S(═O)—, —S(═O)₂—, or —C(═O)—. In some embodiments, L² is —C(═O)NH— or —NHC(═O)—. In some embodiments, L² is substituted or unsubstituted C₃-C₁₅ cycloalkyl, or substituted or unsubstituted C₁-C₁₂ heterocycloalkyl. In some embodiments, L² is substituted or unsubstituted aryl or substituted or unsubstituted heteroaryl. In some embodiments, L² is substituted or unsubstituted C₁-C₃₀ alkylene. In some embodiments, L² is substituted or unsubstituted C₂-C₃₀ alkenylene. In some embodiments, L² is substituted or unsubstituted C₁-C₃₀ heteroalkylene. In some embodiments, L² is substituted or unsubstituted C₅-C₂₅ heteroalkylene. In some embodiments, L² is substituted or unsubstituted C₅-C₁₂ heteroalkylene.

In some embodiments, each of L³ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}L=N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene. In some embodiments, L³ is —O—, —NR^{L}—, —OP(═O)(OR^{L}O—, —S—, —S(═O)—, —S(═O)₂, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L³ is —O—, —NH—, —S(═O)—, —S(═O)₂—, or —C(═O)—. In some embodiments, L³ is —C(═O)NH— or —NHC(═O)—. In some embodiments, L³ is substituted or unsubstituted C₃-C₁₅ cycloalkyl, or substituted or unsubstituted C₁-C₁₂ heterocycloalkyl. In some embodiments, L³ is substituted or unsubstituted aryl or substituted or unsubstituted heteroaryl. In some embodiments, L³ is substituted or unsubstituted C₁-C₃₀ alkylene. In some embodiments, L³ is substituted or unsubstituted C₂-C₃₀ alkenylene. In some embodiments, L³ is substituted or unsubstituted C₁-C₃₀ heteroalkylene. In some embodiments, L³ is substituted or unsubstituted C₅-C₂₅ heteroalkylene. In some embodiments, L³ is substituted or unsubstituted C₅-C₁₂ heteroalkylene. In some embodiments, L³ is absent.

In some embodiments, each of L⁴ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁴ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁴ is —O—, —NH—, —S(═O)—, —S(═O)₂—, or —C(═O)—. In some embodiments, L⁴ is —C(═O)NH— or —NHC(═O)—. In some embodiments, L⁴ is substituted or unsubstituted C₃-C₁₅ cycloalkyl, or substituted or unsubstituted C₁-C₁₂ heterocycloalkyl. In some embodiments, L⁴ is substituted or unsubstituted aryl or substituted or unsubstituted heteroaryl. In some embodiments, L⁴ is substituted or unsubstituted C₁-C₃₀ alkylene. In some embodiments, L⁴ is substituted or unsubstituted C₂-Cas alkenylene. In some embodiments, L⁴ is substituted or unsubstituted C₁-C₃₀ heteroalkylene. In some embodiments, L⁴ is substituted or unsubstituted C₅-C₂₅ heteroalkylene. In some embodiments, L⁴ is substituted or unsubstituted C₅-C₁₂ heteroalkylene. In some embodiments, L⁴ is absent.

In some embodiments, each of L⁵ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁵ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁵ is —O—, —NH—, —S(═O)—, —S(═O)₂—, or —C(═O)—. In some embodiments, L⁵ is —C(═O)NH— or —NHC(═O)—. In some embodiments, L⁵ is substituted or unsubstituted C₃-C₅ cycloalkyl, or substituted or unsubstituted C₁-C₁₂ heterocycloalkyl. In some embodiments, L⁵ is substituted or unsubstituted aryl or substituted or unsubstituted heteroaryl. In some embodiments, L⁵ is substituted or unsubstituted C₁-C₃₀ alkylene. In some embodiments, L⁵ is substituted or unsubstituted C₂-C₃₀ alkenylene. In some embodiments, L⁵ is substituted or unsubstituted C₁-C₃₀ heteroalkylene. In some embodiments, L⁵ is substituted or unsubstituted C₅-C₂₅ heteroalkylene. In some embodiments, L⁵ is substituted or unsubstituted C₅-C₁₂ heteroalkylene. In some embodiments, L⁵ is absent.

In some embodiments, the linker has a structure of

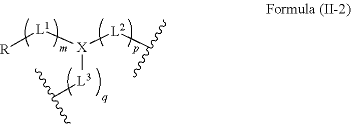

wherein
   each L¹, L², and L³ is independently —O—, —NR^{L}—, —N(R^{L})₂_{⁺}—, —OP(═O)(OR^{L})O—, —S—, —S(═O)—, —S(═O)₂—, ═CH—, —C(═O)—, —C(═O)O—, —OC(═O)—, —OC(═O)—, —C(═O)NR^{L}—, —NR^{L}C(═O)—, —OC(═O)NR^{L}—, —NR^{L}C(═O)O—, —NR^{L}C(═O)NR^{L}, —NR^{L}C(═S)NR^{L}—, —CR^{L}═N—, —N═CR^{L}, —NR^{L}S(═O)₂—, —S(═O)₂NR^{L}, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted C₁-C₁₅ arylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂_{⁺}—, —N(R^{L})₂_{⁺}—(C₁-C₃₀ alkylene)-, or a click chemistry residue; and
   R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
   R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
   X is N or CRY; and
   each of m, p, and q is independently 0, 1, 2, 3, 4, 5, 6, 7, 8, 9, 10, 11, 12, 13, 14, or 15.

In some embodiments, the linker has a structure of

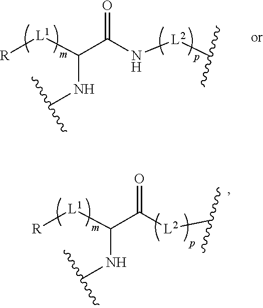

wherein
   each L¹ and L² is independently —O—, —NR^{L}—, —N(R^{L})₂_{⁺}—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CHR^{L}—CHR^{L}—O)₁₋₁₀—;
   R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, or substituted or unsubstituted C₂-C₇ heterocycloalkyl;
   R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;
   m is 1 to 10; and p is 0-3.

In some embodiments, L² is —O—, —NR^{L}—, —N(R^{L})₂_{⁺}—, —OP(═O)(OR^{L})O—, —S—, —S(═O)—, —S(═O)—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂—O)₁₋₆—;
L¹ is —O—, —NR^{L}—, —N(R^{L})₂_{⁺}—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CH₂—CH₂—O)₁₋₆—;
R^{L} is hydrogen or substituted or unsubstituted C₁-C₄ alkyl;
R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
m is 1 to 10; and p is 0-3.

In some embodiments, the linker has a structure of

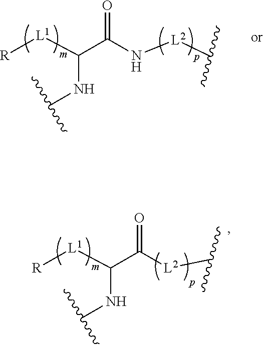

wherein
L² is —O—, —NR^{L}—, —N(R^{L})₂_{⁺}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂—O)₁₋₆—;
L¹ is —O—, —NR^{L}—, —N(R^{L})₂_{⁺}—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}S(═O)₂—, —S(═O)—NR^{L}—, —C(═O)NR^{L}S(═O)₂—, —S(═O)—NR^{L}C(═O)—, substituted or unsubstituted C₁-C₂₀ alkylene, or —(CH₂—CH₂—O)₁₋₆;
R^{L} is hydrogen or substituted or unsubstituted C₁-C₄ alkyl;
R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted C₆-C₁₀ aryl, or substituted or unsubstituted C₅-C₉ heteroaryl;
m is 1 to 4; and p is 0-3.

In some embodiments, R is hydrogen, substituted or unsubstituted C₆-C₁₀ aryl, substituted or unsubstituted C₅-C₉ heteroaryl, or a sterol.

In some embodiments, at least one L¹ is unsubstituted C₃-C₂₀ alkylene.

In some embodiments, the linker comprises one or more of a substituted or unsubstituted C₆-C₁₀ aryl, substituted or unsubstituted C₅-C₉ heteroaryl, a sterol, sulfonamide, phosphate ester, polyethylene glycol, or C₃-C₂₀ alkylene, or amino acid residues.

In some embodiments, the linker comprises one or more selected from AEEA, AEEP, AEEEP, and AEEEEP groups. In some embodiments, the linker comprises

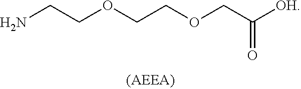 [SMILES: NCCOCCOCC(=O)O]

In some embodiments, the linker comprises

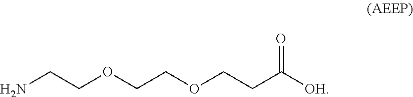 [SMILES: NCCOCCOCCC(=O)O]

In some embodiments, the linker comprises

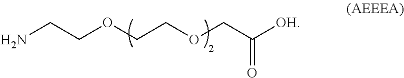

In some embodiments, the linker comprises

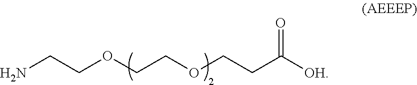

In some embodiments, the linker comprises

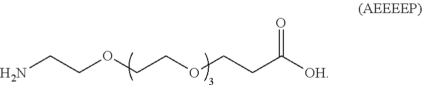

In some embodiments, the linker is

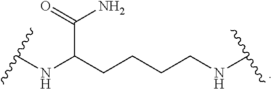 [SMILES: *NCCCCC(N*)C(N)=O]

In some embodiments, the linker is or comprises lysine. In some embodiments, the linker comprises C₁-C₁₂ alkylene. In some embodiments, the linker comprises C₃-C₉ alkylene. In some embodiments, the linker comprises C₂-C₈ alkylene. In some embodiments, the linker comprises 1 to 10 repeating ethylene glycol units. In some embodiments, the linker comprises 2 to 4 repeating ethylene glycol units. In some embodiments, the linker comprises 5 to 8 repeating ethylene glycol units. In some embodiments, the linker comprises NH₂—(CH₂)ₙ—COOH, wherein n is 1 to 12. In some embodiments, the linker comprises NH₂—(CH₂)₂—COOH. In some embodiments, the linker comprises NH₂—(CH₂)₃—COOH. In some embodiments, the linker comprises NH₂—(CH₂)₄—COOH. In some embodiments, the linker comprises NH₂—(CH₂)₅—COOH. In some embodiments, the linker comprises NH₂—(CH₂)₆—COOH. In some embodiments, the linker comprises NH₂—(CH₂)₇—COOH. In some embodiments, the linker comprises NH₂—(CH₂)₈—COOH. In some embodiments, the linker comprises NH₂—(CH₂)₁₀—COOH. In some embodiments, the linker is absent.

In some embodiments, a linker of the present disclosure (e.g., a linker of Formula (II-1), (II-1a) or (II-1b)) comprises a structure of

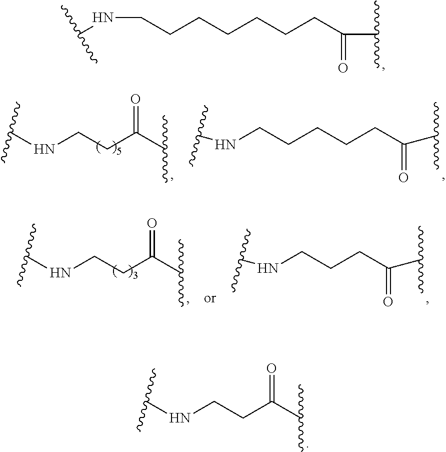

In some embodiments, a linker of the present disclosure comprises a structure of

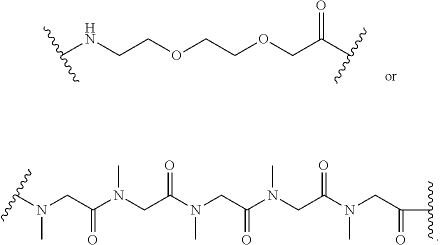 [SMILES: *NCCOCCOCC(*)=O ; *C(=O)CN(C)C(=O)CN(C)C(=O)CN(C)C(=O)CN(C)C(=O)CN(*)C]

In some embodiments, a linker of the present disclosure (e.g., a linker of Formula (II-1), (II-1a) or (II-1b)) comprises a structure of

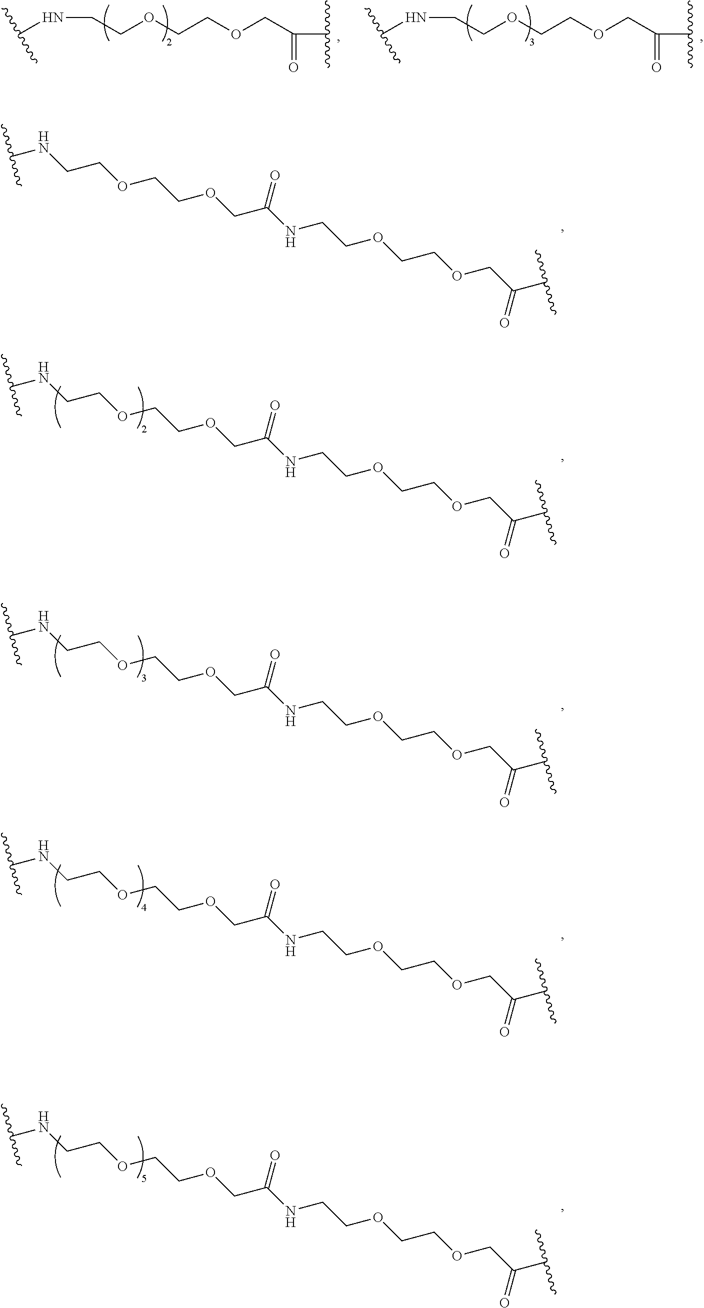

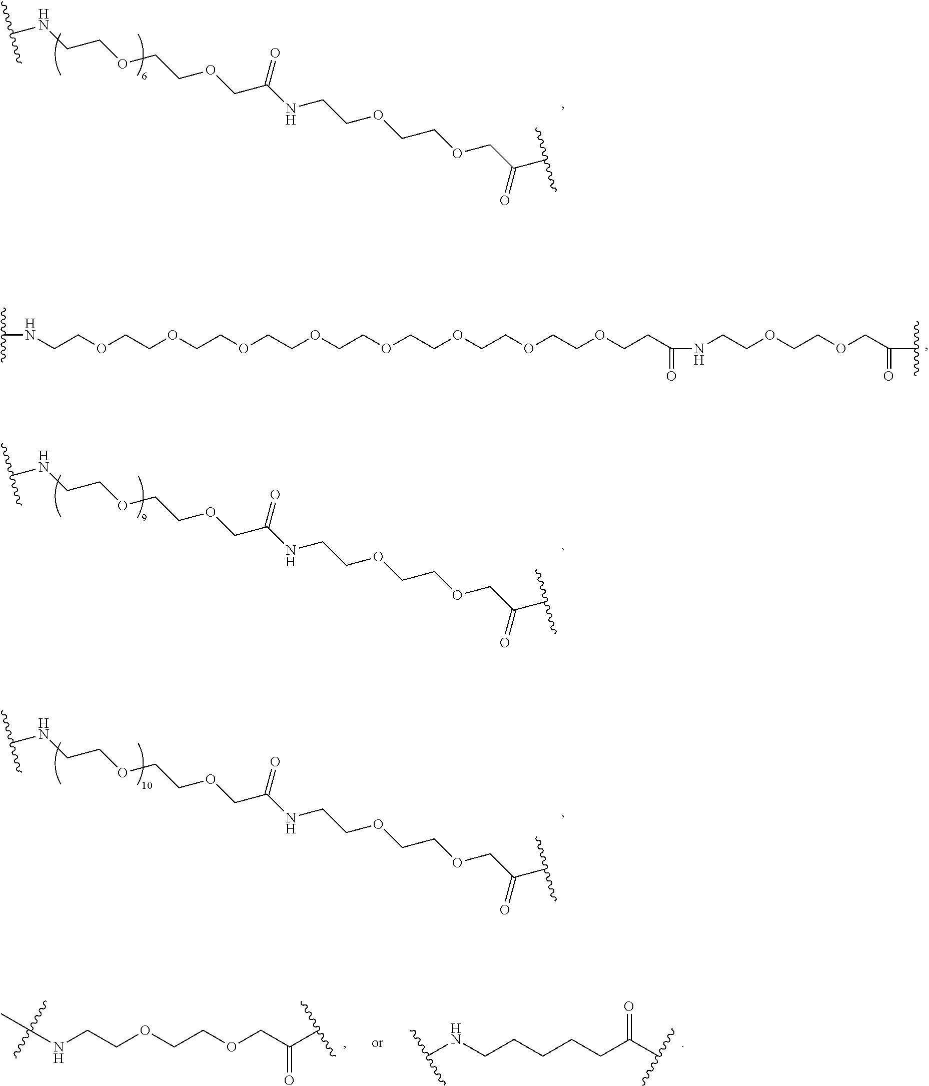

In some embodiments, the linker comprises a structure selected from:

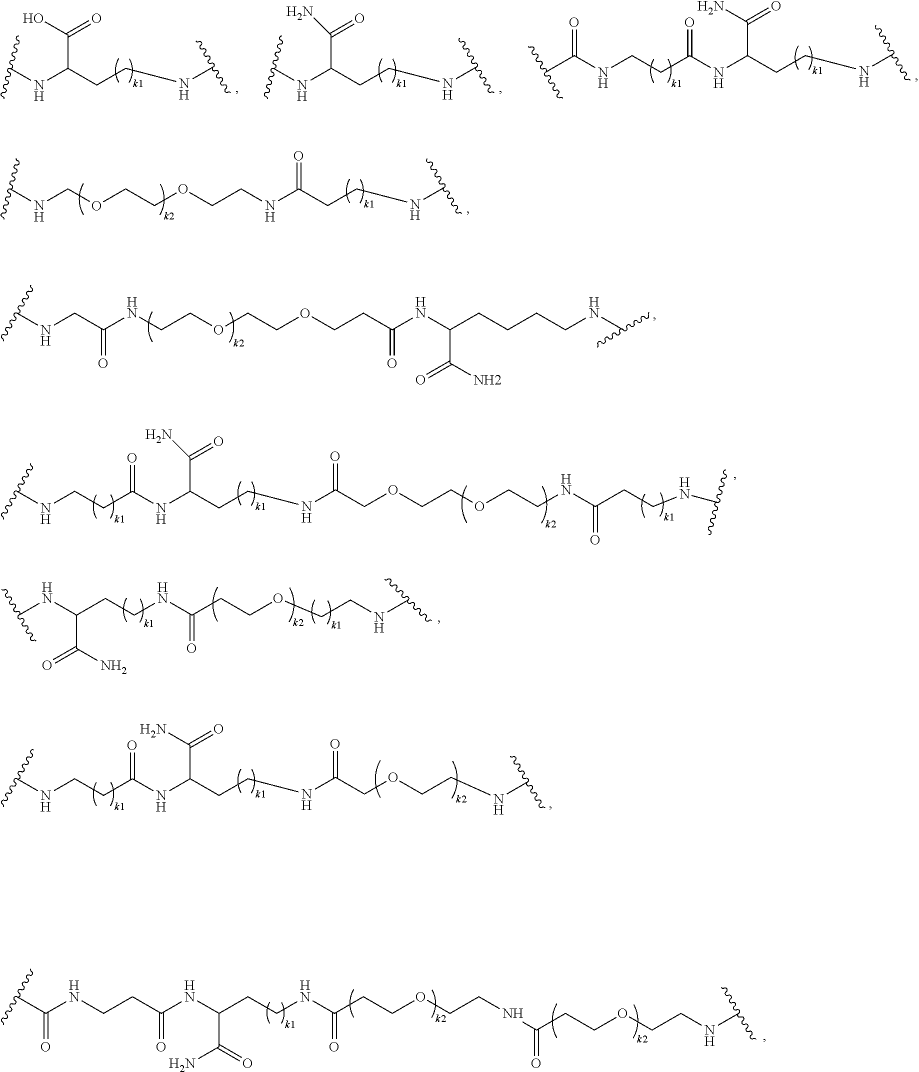

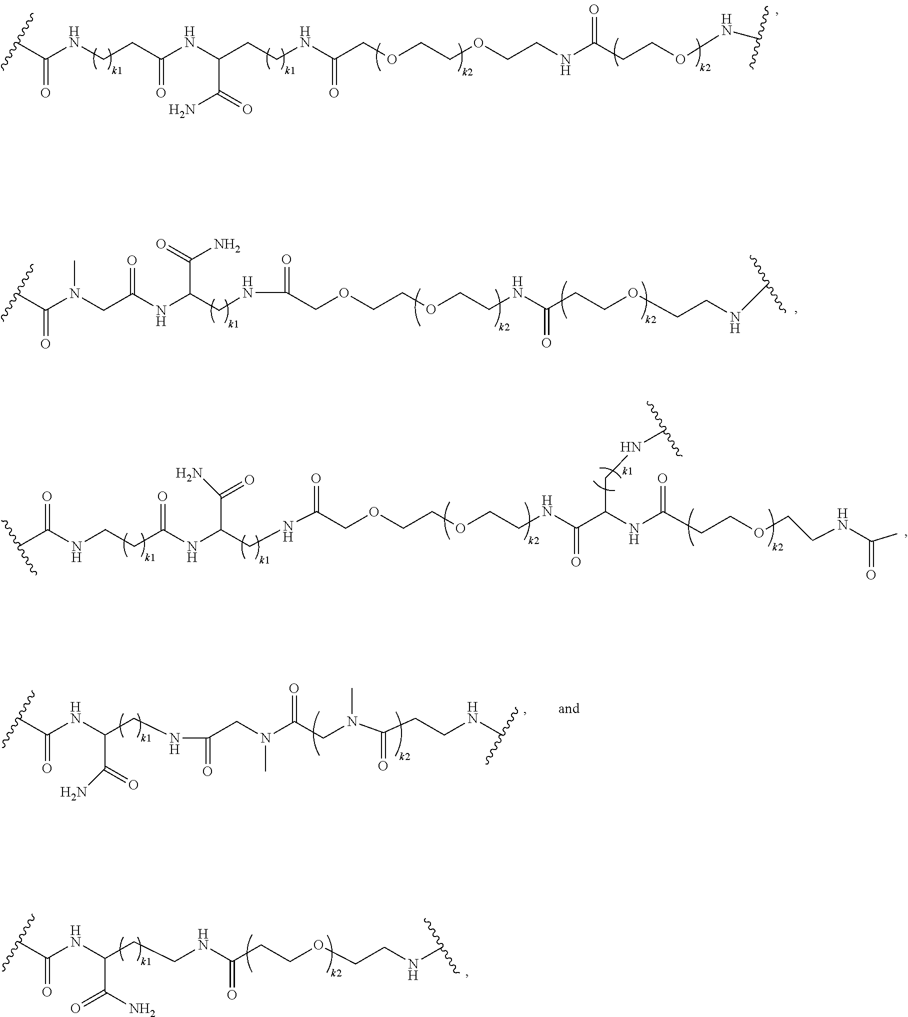

wherein each k1 is independently 0 or an integer from 1 to 20; and each k2 is independently 0 or an integer from 1 to 15. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments, k1 is 11. In some embodiments, k1 is 12. In some embodiments, k1 is 13. In some embodiments, k1 is 14. In some embodiments, k1 is 15. In some embodiments, k1 is 16. In some embodiments, k1 is 17. In some embodiments, k1 is 18. In some embodiments, k1 is 19. In some embodiments, k1 is 20. In some embodiments, k2 is 0. In some embodiments, k2 is 1. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10. In some embodiments, k2 is 11. In some embodiments, k2 is 12. In some embodiments, k2 is 13. In some embodiments, k2 is 14. In some embodiments, k2 is 15.

In some embodiments, the linker comprises a structure selected from:

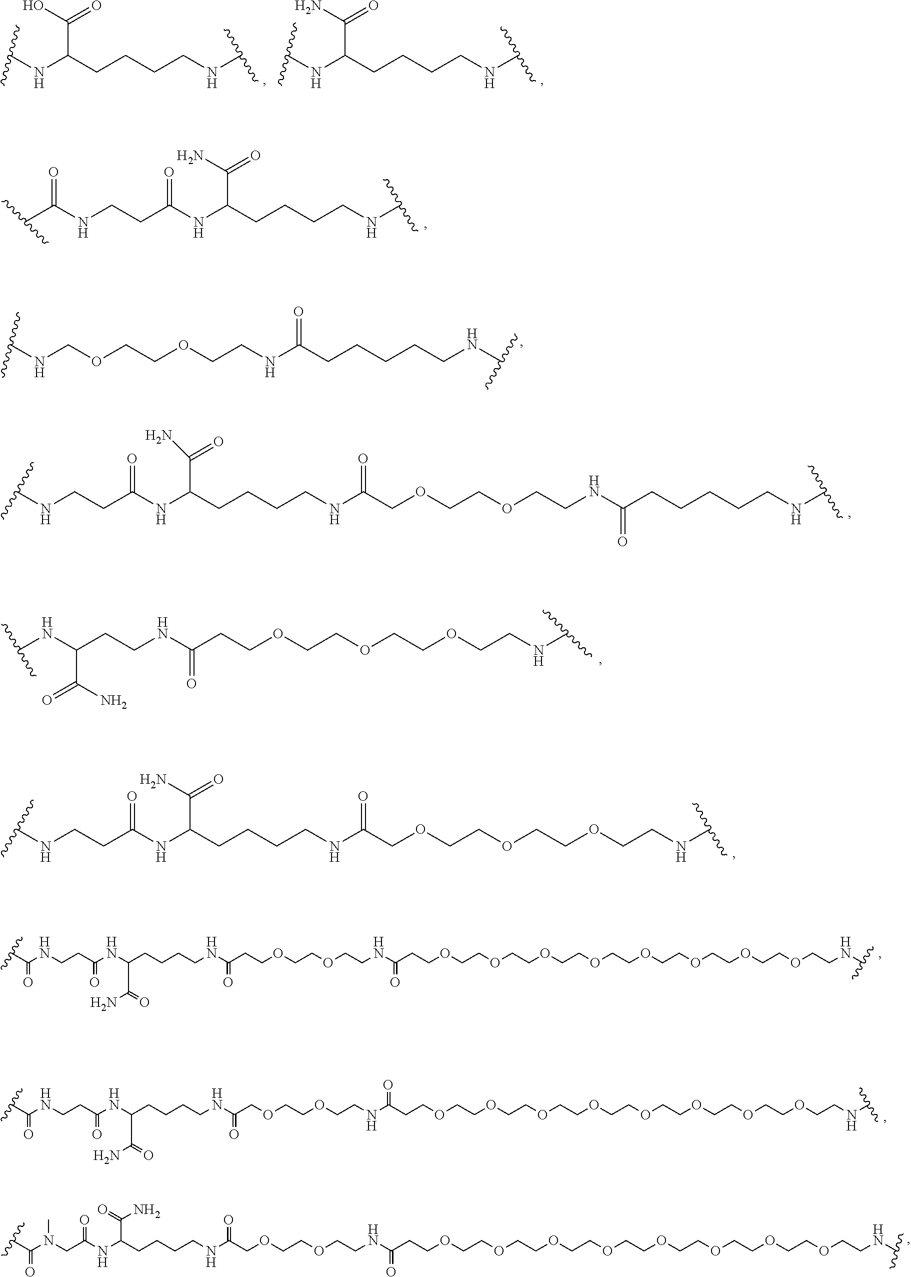

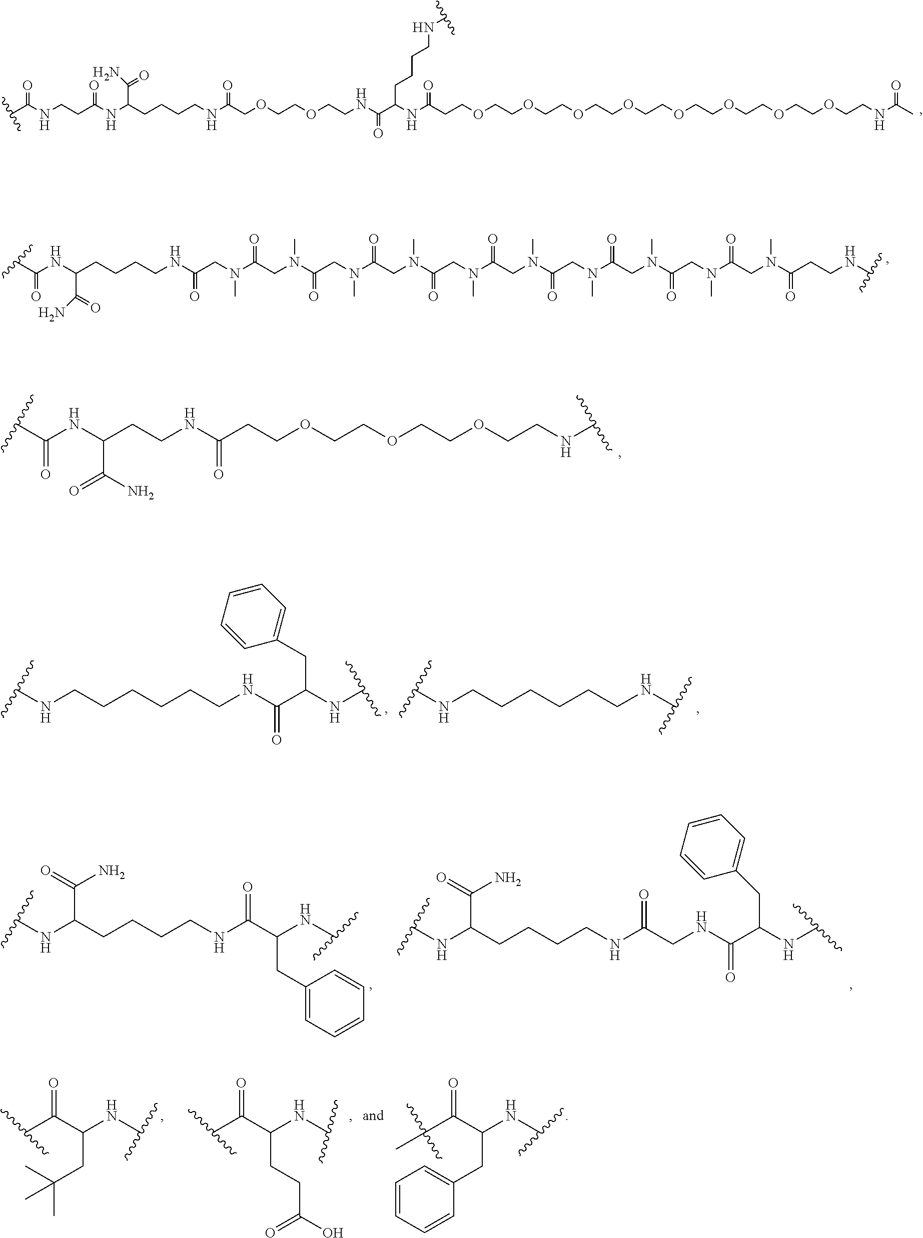

In some embodiments, the linker comprises a structure selected from:

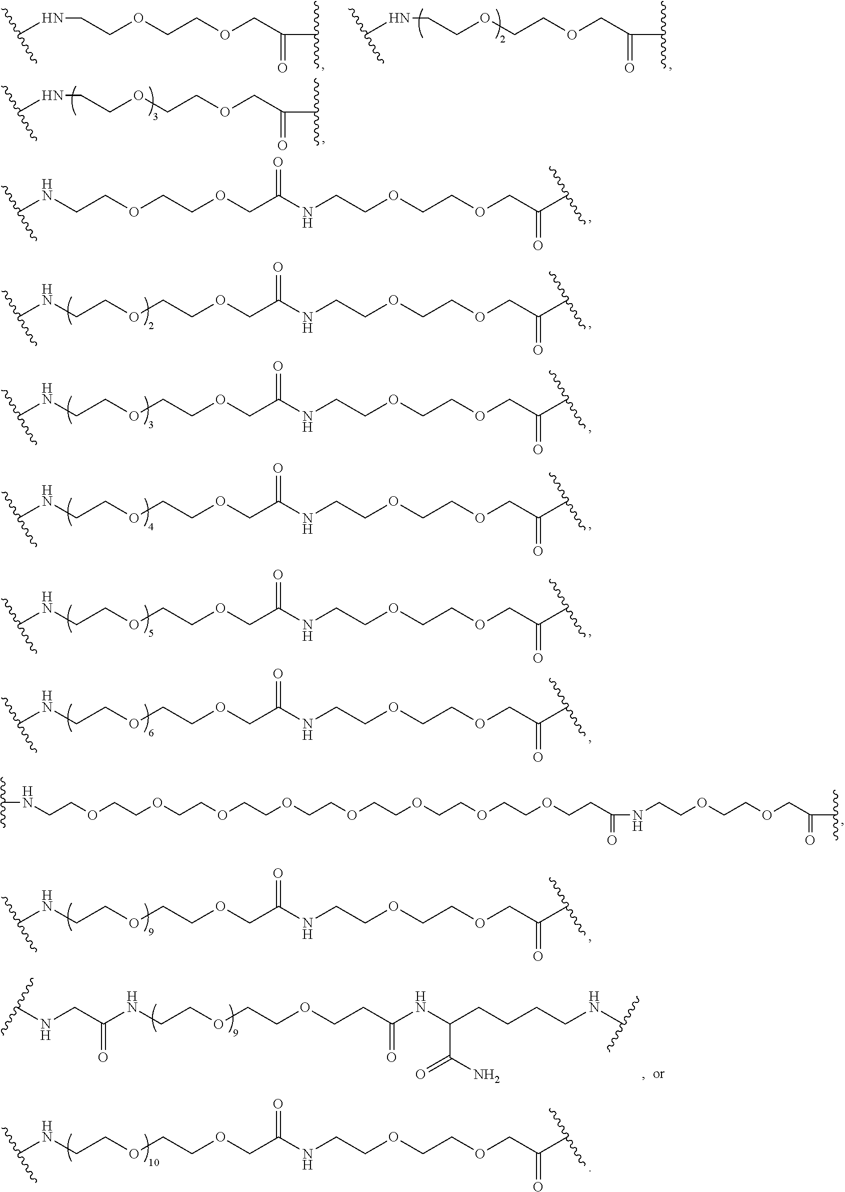

In some embodiments, the linker is configured to reversibly bind to a plasma protein such as albumin. In some embodiments, a dissociation constant (Kd) between the linker and human serum albumin is at most 15 μM, as determined at room temperature in human serum condition. In some embodiments, the Kd is from about 0.1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

In some embodiments, the linker is a bond.

### Payload-Bound PDC (Peptide-Drug Conjugate, or “Conjugate”)

In one embodiment, the present technology relates to a complex or conjugate. This complex/conjugate comprises any of the peptides described herein, any of the linkers described herein bound to the peptide, and/or a substance (e.g., a payload molecule) bound to this linker (PDC). Since the peptide is capable of binding to the EphA2, the complex/conjugate can transport the substance/payload molecule to the EphA2.

In one aspect, described herein is a PDC with structure of FIG. 1, wherein the [symbol] represents the linker connected to the peptide.

In some embodiments, the linker is connected to the peptide at the side chain of any amino acid included in the peptide of the invention, such as the amino acid at the C-terminus of the peptide. For example, but not limited to, the linker bound to the substance is connected to the Cys at the C-terminus of the peptide, or any amino acid within the peptide, preferably amino acid at X5, X8 or X11.

The substance or payload molecule may be any substance desired by a person having ordinary skill in the art, as long as it is a substance the skilled person desires to be delivered to the EphA2. Examples of the substance are not limited, but include the following:
A compound: Including low molecular weight compounds, middle molecular weight compounds, and examples include known low/middle molecular weight drugs. Examples includes any ligand for a receptor, antagonist, agonist or like. Examples also includes any compound that is not used as drugs such as fluorescent molecules or tags. Further to examples, any chemical compound that is used for connecting or conjugating to a substance (e.g., biotin/avidin, click chemistry compounds) or conjugations thereof is included.
A peptide: May be a peptide that binds to a target in the body and exhibits some kind of effect, for example, a cyclic peptide.
A protein: May be any protein that exhibits a useful function in the body, such as an antibody or an enzyme. Examples include enzymes used in enzyme replacement therapy.
A nucleic acid: Any substance having a base sequence, such as DNA and RNA, and combination thereof (e.g. heteroduplex oligonucleotide). Examples include nucleic acid medicines.
Any molecule used in a drug delivery system (DDS): May be a known molecule used in DDS, such as a liposome or a micelle. The DDS molecule may further comprise a compound therein such as a pharmaceutical.

In all aspects of this disclosure, however, the substance or payload molecule excludes any radioactive materials. Examples of the substance that are excluded are: radioisotope, radiopharmaceutical, or any compound having radioactive component. In all aspects of this disclosure, the substance further excludes any chelators for radioisotope conjugation, regardless of whether the chelator is connected to the peptide directly or via a linker. Accordingly, a complex, conjugate or PDC described herein does not encompass any compound containing a chelator for radioisotope conjugation, and does not encompass a radioisotope.

Moreover, the DDS molecule may be a complex in which several of the examples given above are combined.

### Peptide Production

The peptide of the present technology may be produced by, for example, any known method for producing a peptide, such as the following:
a chemical synthesis method such as a liquid phase method, a solid phase method, a hybrid method combining a liquid phase method and a solid phase method, or the like;
a genetic recombination method; or the like.

In some of the instances where the peptide of the present technology is produced by a chemical synthesis method, it can be said that the peptide of the present technology is a synthetic peptide.

In the solid phase method, for example, a hydroxy group of a resin having a hydroxy group and a carboxy group of a first amino acid (normally a C-terminal amino acid of a target peptide) in which an α-amino group is protected by a protecting group are subjected to an esterification reaction. For the esterification catalyst, a known dehydrating and condensing agent such as 1-mesitylenesulfonyl-3-nitro-1,2,4-triazole (MSNT), dicyclohexylcarbodiimide (DCC), and diisopropylcarbodiimide (DIPCDI) may be used.

Next, the protecting group of the α-amino group of the first amino acid is removed, a second amino acid in which all functional groups except the carboxy group of the main chain are protected is added, and the carboxy group is activated, binding the first and second amino acids. Furthermore, the α-amino group of the second amino acid is deprotected, a third amino acid in which all functional groups except the carboxy group of the main chain are protected is added, the carboxy group is activated, binding the second and third amino acids. This is repeated, and after a peptide having a target length is synthesized, all of the functional groups are deprotected.

Examples of the resin for solid-phase synthesis include Merrifield resin, MBHA resin, Cl-Trt resin, SASRIN resin, Wang resin, Rink amide resin, HMFS resin, Amino-PEGA resin (Merck KGaA), HMPA-PEGA resin (Merck KGaA), and the like. These resins may be used after being washed using a solvent (dimethylformamide (DMF), 2-propanol, methylene chloride, and the like). A peptide chain can be cleaved from the resin by treating it with an acid such as TFA or hydrogen fluoride (HF).

Examples of the protecting group of the α-amino group include a benzyloxycarbonyl (Cbz or Z) group, tert-butoxycarbonyl (Boc) group, fluorenylmethoxycarbonyl (Fmoc) group, benzyl group, allyl group, allyloxycarbonyl (Alloc) group, and the like. The Cbz group may be deprotected by a treatment using hydrofluoric acid, hydrogenation, or the like, the Boc group may be deprotected by a treatment using trifluoroacetic acid (TFA), and the Fmoc group may be deprotected by a treatment using pipericine or pyrrolysine.

Examples, such as methyl ester, ethyl ester, allyl ester, benzyl ester, tert-butyl ester, cyclohexyl ester, and the like may be used to protect the α-carboxy group.

As other functional groups of an amino acid, the hydroxyl group of serine or threonine can be protected with a benzyl group or a tert-butyl group and the hydroxyl group of tyrosine can be protected with a 2-bromobenzyloxycarbonyl group or a tert-butyl group. The amino group of a lysine side chain or the carboxyl group of glutamic acid or aspartic acid can be protected in a manner similar to the α-amino group or a-carboxyl group.

Activation of the carboxy group may be performed using a condensing agent. Examples of the condensing agent include dicyclohexylcarbodiimide (DCC), diisopropylcarbodiimide (DIPCDI), 1-ethyl-3-(3-dimethylaminopropyl)carbodiimide (EDC or WSC), (1H-benzotriazole-1-yloxy)tris(dimethylamino)phosphonium hexafluorophosphate (BOP), 1-[bis(dimethylamino)methyl]-1H-benzotriazolium-3-oxide hexafluorophosphate (HBTU), and the like.

Cleavage of the peptide chain from the resin may be performed by treating the peptide chain using an acid such as TFA, hydrogen fluoride (HF), or the like.

Production of a peptide by a gene recombination method (translation/synthesis system) may be performed using a nucleic acid encoding the peptide. The nucleic acid encoding the peptide may be DNA or RNA.

The nucleic acid encoding the peptide may be prepared by a known method or a method equivalent thereto. For example, the peptide may be synthesized by an automated synthesizer. A restriction enzyme recognition site may be added to insert the obtained DNA into a vector. Alternatively, a base sequence encoding an amino acid sequence for splicing a formed peptide chain using an enzyme or the like may be incorporated. The peptide obtained may be converted from a free peptide to a salt thereof or from a salt thereof to a free peptide by a known method or a method based thereon.

As described above, when the peptide is fused to a cell-penetrating peptide or the like, the nucleic acid also includes a nucleic acid encoding the cell-penetrating peptide.

A chimeric protein expression method for expressing the target peptide as a chimeric peptide of another peptide may also be used to suppress degradation by a host-derived protease. In this case, a nucleic acid encoding the target peptide and the peptide bound thereto may be used as the nucleic acid.

Subsequently, an expression vector is prepared using the nucleic acid encoding the peptide. The nucleic acid may be digested as is or by a restriction enzyme, and alternatively, the nucleic acid may be inserted downstream of a promotor of the expression vector by adding a linker, or the like. Examples of the vector include an Escherichia coli-derived plasmid (pBR322, pBR325, pUC12, pUC13, pUC18, pUC19, pUC118, pBluescript II, and the like), a Bacillus subtilis-derived plasmid (pUB110, pTP5, pC1912, pTP4, pE194, pC194, and the like), a yeast-derived plasmid (pSH19, pSH15, YEp, YRp, YIp, YAC, and the like), a bacteriophage (e phage, M13 phage, and the like), a virus (retrovirus, vaccinia virus, adenovirus, adeno-associated virus (AAV), cauliflower mosaic virus, tobacco mosaic virus, baculovirus, and the like), a cosmid, and the like.

The promoter may be selected appropriately according to the type of host. When the host is an animal cell, for example, a promoter derived from SV40 (simian virus 40) or a promoter derived from CMV (cytomegalovirus) may be used. When the host is Escherichia coli, a trp promoter, a T7 promoter, a lac promoter, or the like may be used.

The expression vector may incorporate, for example, a DNA replication starting point (ori), a selective marker (antibiotic resistance, auxotrophy, or the like), an enhancer, a splicing signal, a poly-A addition signal, a nucleic acid encoding a tag (FLAG, HA, GST, GFP, or the like), or the like.

Next, an appropriate host cell is transformed by the expression vector. The host may be appropriately selected in relation to the vector. Examples such as Escherichia coli, Bacillus subtilis (Bacillus), yeast, insects or insect cells, animal cells, or the like may be used as the host. As the animal cells, for example, HEK293T cells, CHO cells, COS cells, myeloma cells, Hela cells, and Vero cells may be used. Transformation may be carried out according to a known method, such as a lipofection method, a calcium phosphate method, an electroporation method, a microinjection method, a gene gun method, or the like depending on the type of host. The target peptide is expressed by culturing a transformant according to a conventional method.

As for purification of the peptide from the transformant culture, cultured cells are recovered and then suspended in an appropriate buffer solution, followed by disruption of cells by a method such as sonication, freeze-thawing, or the like, and then a crude extract is obtained by centrifugation or filtration. When the peptide is secreted into the culture solution, a supernatant is recovered.

Purification of the crude extract or the culture supernatant may also be performed by a known method or a method equivalent thereto (for example, salting-out, dialysis, an ultrafiltration method, gel filtration method, SDS-PAGE method, ion exchange chromatography, affinity chromatography, reversed-phase high-performance liquid chromatography, and the like).

The obtained peptide may be converted from a free body to a salt or from a salt to a free body by a known method or a method equivalent thereto.

In one aspect, the translation/synthesis system may be a cell-free translation system. According to the cell-free translation system, a highly pure form of an expression product can generally be obtained without purification. The cell-free translation system includes, for example, a ribosome protein, an aminoacyl-tRNA synthase (ARS), a ribosome RNA, an amino acid, rRNA, GTP, ATP, a translation initiation factor (IF), an elongation factor (EF), a release factor (RF), and a ribosome regeneration factor (RRF), or another factor required for translation. An Escherichia coli extract or a wheat embryo extract may be added to increase expression efficiency. In addition, a rabbit red blood cell extract or an insect cell extract may be added.

By continuously supplying energy to a system including these using dialysis, a protein of several hundred μg to several mg/ml may be produced in a non-limiting manner. The system may include an RNA polymerase to concurrently perform transcription of genomic DNA. Examples of commercially available cell-free translation systems that may be used include RTS-100 (registered trademark) by Roche Diagnostics K.K., PURE System by GeneFrontier Corporation, PURExpress In vitro Protein Synthesis Kit by New England Biolabs Inc., and the like for a system derived from Escherichia coli, and a system by ZOIGENE, CellFree Sciences Co., Ltd., or the like for a system using wheat embryo extract.

In the cell translation system, artificial aminoacyl-tRNA may be used and a desired amino acid or hydroxy acid may be linked (acylated) to a tRNA in place of an aminoacyl-tRNA synthesized by a natural aminoacyl-tRNA synthase. The aminoacyl-tRNA may be synthesized using an artificial ribozyme.

An example of the ribozyme includes a flexizyme (flexizyme) (H. Murakami, H. Saito, and H. Suga, (2003), Chemistry & Biology, Vol. 10, 655-662; and WO 2007/066627 and the like), all incorporated herein by reference. Flexizymes are also known under the names of prototype flexizyme (Fx), newly modified dinitrobenzyl flexizyme (dFx), enhanced flexizyme (eFx), aminoflexizyme (aFx), and the like.

A desired codon may be translated in association with the desired amino acid or hydroxy acid by using the tRNA produced by flexizyme and to which the desired amino acid or hydroxy acid is linked. A specialty amino acid may be used as the desired amino acid. For example, an unnatural amino acid required for the above circularization may also be introduced into the binding peptide by this method.

Various methods commonly used in the technical field may be used for chemical synthesis of the peptide, including, for example, stepwise solid-phase synthesis, semisynthesis of peptide fragments undergoing conformationally supported religation, and chemical ligation. Synthesis of the peptide is chemical synthesis using various solid phase technologies described in, for example, K. J. Jensen, P. T. Shelton, S. L. Pedersen, Peptide Synthesis and Applications, 2nd Edition, Springer, 2013, and the like. A preferable strategy is based on a combination of an Fmoc group capable of temporarily protecting the α-amino group and being selectively removed using a base, and a protecting group that temporarily protects a side chain functional group and is stable under Fmoc deprotection conditions. Selection of this kind of general peptide side chain is known according to the aforementioned Peptide Synthesis and Applications, 2nd Edition; G. B. Fields, R. L. Noble, Solid Phase Peptide Synthesis Utilizing 9-Fluorenylmethoxycarbonyl Amino Acids, Int. J. Peptide Protein Res. 35, 1990, 161-214, and the like; however, preferable peptide side chain protecting groups include, for example, a benzyl group or a tert-butyl group and a trityl (Trt) group for the hydroxy group of serine or threonine; a 2-bromobenzyloxycarbonyl group or a tert-butyl group for the hydroxy group of tyrosine; a Boc group, a methyltetrazole thiol (Mtt) group, an Alloc group, and an ivDde group for the amino group of the lysine side chain; a Trt group or a Boc group for the imidazole group of histidine; a 2,2,4,6,7-pentamethyldihydrobenzofuran-5-sulfonyl group (Pbf) group for the guanidyl group of arginine; a tert-butyl group, an allyl group, and a 3-methylpentane (Mpe) group for carboxyl groups, such as glutamic acid and aspartic acid; a Trt group for the carboxamide group of glutamine or asparagine; or a Trt group and a monomethoxytrityl (Mmt) group for the thiol group of cysteine.

The peptide may be synthesized by a stepwise method on the solid-phase resin described above. The C-terminal amino acid to be used and all of the amino acids or peptides to be used for synthesis must be selectively removed during the process of synthesizing the α-amino protecting group. Preferably, the solid-phase resin described above is used, and once a C-terminal carboxyl group of a peptide having its N-terminal properly protected by Fmoc or the like or a C-terminal carboxyl group of an amino acid having its N-terminal protected by Fmoc is made into an activated ester by an appropriate reagent, this is then added to the amino group on the solid-phase resin to start. Subsequent elongation of the peptide chain may be achieved by removing the N-terminal protecting group (Fmoc group) then successively repeating condensation of the protected amino acid derivative according to the amino acid sequence of the target peptide. Note that these may release the target peptide in a final stage. Examples of releasing conditions are given in Teixeira, W. E. Benckhuijsen, P. E. de Koning, A. R. P. M. Valentijn, J. W. Drijfhout, Protein Pept. Lett., 2002, 9, 379-385, and the like, and the peptide may be released in a TFA solution containing water/silyl hydride/thiol as a scavenger in TFA. Typical examples include TFA/Water/TIS/DODT (volume ratio 92.5:2.5:2.5:2.5).

Synthesis of the peptide described in the present specification may be carried out using a single or multi-channel peptide synthesizer, for example, a Liberty Blue synthesizer from CEM Corporation, a Syro I synthesizer or a successor machine thereof from Biotage Japan, Ltd., or the like.

Activation of the carboxy group may be performed using a condensing agent. Examples of the condensing agent include dicyclohexylcarbodiimide (DCC), diisopropylcarbodiimide (DIPCDI), 1-ethyl-3-(3-dimethylaminopropyl) carbodiimide (EDC or WSC), (1H-benzotriazole-1-yloxy)tris(dimethylamino)phosphonium hexafluorophosphate (BOP), 1-[bis(dimethylamino)methyl]-1H-benzotriazolium-3-oxide hexafluorophosphate (HBTU), and the like.

Cyclization of the peptide may be carried out according to a known method. In a non-limiting manner, by designing the peptide to comprise two or more cysteine residues, for example, a cyclic structure may be formed by a disulfide bond after translation. Furthermore, according to the method of Goto et al. (Y. Goto, et al. ACS Chem. Biol. 3 120-129 (2008)), a peptide having a chloroacetyl group at its N-terminal may be synthesized by genetic code reprogramming technology and may also be circularized by disposing a cysteine residue containing a sulfur molecule in the peptide. Thus, a mercapto group spontaneously performs a nucleophilic attack on the chloroacetyl group after translation, and the peptide is circularized by thioether binding. Other amino acid combinations that bind to form a ring may be disposed within the peptide and circularized by genetic code reprogramming technology. Alternatively, circularization may be carried out by disposing an L-2-aminoadipic acid residue in the peptide and binding it to the main chain amino group of the N-terminal. In this manner, a known circularization method may be used without any particular limitation.

### Pharmaceutical Composition and Uses Thereof

The present technology also relates to a pharmaceutical composition that comprises the peptide of the present technology. Diseases targeted by the pharmaceutical composition of the present technology are those associated with overexpression or decreased expression of EphA2, such as certain cancer.

As used herein, the expression “a disease or disorder characterized by overexpression or decreased expression of EphA2” refers to a disease that exhibits various symptoms mainly caused by excessive or decreased EphA2 expression.

In some aspect, the diseases targeted by the pharmaceutical composition of the present technology are those associated with overexpression of EphA2.

In some embodiments, the pharmaceutical composition of the present technology may comprise the peptide itself or may comprise a pharmaceutically acceptable salt of the peptide. The “peptide” in the present specification may comprise a pharmaceutically acceptable salt of the peptide unless otherwise specified. The pharmaceutical composition preferably comprises the peptide as an active ingredient in an effective amount.

The salt of the peptide (pharmaceutically acceptable salt) is preferably an acid addition salt. For example, a salt of an inorganic acid (such as hydrochloric acid, phosphoric acid, hydrobromic acid, sulfuric acid), a salt of an organic acid (such as acetic acid, formic acid, propionic acid, fumaric acid, maleic acid, succinic acid, tartaric acid, citric acid, malic acid, oxalic acid, benzoic acid, methanesulfonic acid, benzenesulfonic acid), or the like is used as this salt. The peptide or a salt thereof also comprises a solvate such as a hydrate.

In the present specification, the form of administration of the pharmaceutical composition is not particularly limited and may be oral or parenteral. Examples of parenteral administration include injection, such as intramuscular injection, intravenous injection, or subcutaneous injection; transdermal administration; transmucosal administration (transnasal, transoral, transocular, transpulmonary, transvaginal, or transrectal); or the like.

The pharmaceutical composition may be modified in various ways, considering a property where a polypeptide is easily metabolized and excreted. For example, polyethylene glycol (PEG) or a sugar chain may be added to the polypeptide to extend its retention time in the blood to reduce antigenicity. Furthermore, an emulsion, nanoparticles, nanospheres, or the like prepared in a biodegradable polymerized compound such as polylactic acid/glycol (PLGA), porous hydroxyapatite, liposomes, surface-modified liposomes, and unsaturated fatty acids are used as a controlled-release base, and the polypeptide may be present in the base. In the case of transdermal administration, a weak current is allowed to pass through the skin surface and penetrate the stratum corneum (iontophoresis).

As for the pharmaceutical composition, the active ingredient may be used as is, or the pharmaceutical composition may comprise a pharmaceutically acceptable carrier, an excipient, an additive, or the like, or may be formulated. Examples of the dosage form include a liquid agent (for example, an injection), a dispersant, a suspension, a tablet, a pill, a powder, a suppository, a powdered drug, a fine granule, a granule, a capsule, a syrup, a lozenge, an inhalant, an ointment, an eye drop, a nasal drop, an ear drop, a patch, or the like. The formulation may be carried out by a common method using, for example, an excipient, a binder, a disintegrant, a lubricant, a dissolving agent, a solubilizing agent, a colorant, a flavoring agent, a stabilizer, an emulsifier, an absorption promoter, a surfactant, a pH regulator, a preservative, an antioxidant, or the like as appropriate.

Examples of ingredients used for formulation include but are not limited to purified water, saline, phosphate buffer solution, dextrose, glycerol, a pharmaceutically acceptable organic solvent such as ethanol, animal and vegetable oil, lactose, mannitol, glucose, sorbitol, crystalline cellulose, hydroxypropyl cellulose, starch, corn starch, anhydrous silicio acid, magnesium aluminum silicate, collagen, polyvinyl alcohol, polyvinyl pyrrolidone, carboxyvinyl polymer, sodium carboxymethylcellulose, sodium polyacrylate, sodium alginate, water-soluble dextran, sodium carboxymethyl starch, pectin, methyl cellulose, ethyl cellulose, xanthan gum, gum arabic, tragacanth, casein, agar, polyethylene glycol, diglycerin, glycerin, propylene glycol, vaseline, paraffin, octyldodecyl myristate, isopropyl myristate, higher alcohol, stearyl alcohol, stearic acid, human serum albumin, or the like.

The pharmaceutical composition may comprise an absorption promoter for improving absorption of a poorly absorbable drug, in consideration of the fact that it is generally difficult for peptides to be absorbed through mucous membranes. The following may be used as the absorption promoter: a surfactant such as polyoxyethylene lauryl ether, sodium lauryl sulfate, and saponin; a bile salt such as glycocholic acid, deoxycholic acid, and taurocholic acid; a chelating agent such as EDTA and salicylic acid; a fatty acid such as caproic acid, capric acid, lauric acid, oleic acid, linoleic acid, a mixed micelle; an enamine derivative, an N-acyl collagen peptide, an N-acyl amino acid, a cyclodextrin, chitosan, a nitric oxide donor, or the like.

When the pharmaceutical composition is a pill or tablet, it may be coated using a sugar coating, or a gastric-soluble or enteric-coated substance.

When the pharmaceutical composition is an injection, it may comprise distilled water for injection, physiological saline, propylene glycol, polyethylene glycol, vegetable oil, alcohol, or the like. Additionally, a humectant, an emulsifier, a dispersant, a stabilizer, a dissolving agent, a solubilizing agent, a preservative, or the like may be added.

Furthermore, the pharmaceutical composition may be targeted not only to humans but also to non-human mammals or birds. Examples of non-human mammals include primates other than humans (monkeys, chimpanzees, gorillas, and the like), livestock animals (pigs, cows, horses, sheep, and the like), dogs, cats, rats, mice, guinea pigs, rabbits, and the like.

In particular, the dosage in the case of administering to a human changes depending on symptoms, age, sex, and weight of the patient, sensitivity difference, administration method, administration interval, type of active ingredient, and type of formulation, and it may be administered in a non-limiting manner: for example, by administering between about 30 μg to about 100 g, between about 1 μg to about 10 g, between about 1 μg to about 1 g, between about 10 μg to about 1 g, between about 10 μg to about 500 mg, between about 100 μg to about 10 g, between about 100 μg to about 1 g, between about 10 μg to about to about 500 mg, between about 100 μg to about 500 mg, or between about 100 μg to about 100 mg once or divided into several doses. In the case of injection, between about 1 μg/kg and about 3,000 μg/kg or between about 3 μg/kg and about 1,000 μg/kg according to the bodyweight of the patient may be administered once or divided into several doses.

The present technology also relates to a method for treating a disease or disorder characterized by overexpression or decreased expression of EphA2 by administering the peptide of the present technology to a subject.

The present technology also relates to a use of the peptide of the present technology for the treatment of a disease or disorder characterized by overexpression or decreased expression of EphA2.

The present technology also relates to a use of the peptide for manufacturing a pharmaceutical composition for the treatment of a disease or disorder characterized by overexpression or decreased expression of EphA2.

The present technology also relates to the peptide of the present technology for use in a method for treating a disease or disorder characterized by overexpression or decreased expression of EphA2.

The present technology also relates to a kit for use in a method of diagnosing disease or disorder characterized by overexpression or decreased expression of EphA2 by determination of the expression level of EphA2.

The present technology also relates to a composition for use in a method of diagnosing disease or disorder characterized by overexpression or decreased expression of EphA2.

The present technology also relates to use of a peptide or a salt thereof for use in a method of diagnosing disease or disorder characterized by overexpression or decreased expression of EphA2.

The present technology also provides methods of treating a disease or condition in a subject in need thereof. In some embodiments, the disease or disorder is characterized by overexpression or decreased expression of EphA2 in diseased tissue. In some embodiments, the disease or disorder is characterized by overexpression of EphaA2 in diseased tissue. In some embodiments, the disease or disorder is characterized by decreased expression of EphaA2 in diseased tissue. In some embodiments, the methods comprise administering a peptide described herein, a conjugate described herein, or a pharmaceutically acceptable salt or solvate thereof described herein, or a pharmaceutical composition comprising the same, to the subject in need thereof. In some embodiments, provided herein is a method of providing a therapeutic and/or prophylactic benefit to a subject in need thereof comprising administering a peptide, a conjugate, or a pharmaceutical composition described herein.

In some embodiments, the methods comprise administering to a subject a therapeutically effective amount of a peptide, a conjugate, or a pharmaceutically acceptable salt or solvate thereof. In some embodiments, the peptide, conjugate or pharmaceutically acceptable salt or solvate thereof is administered in a pharmaceutical composition. In some embodiments, the subject has cancer. In some embodiments, the cancer is a solid tumor or hematological cancer.

In one aspect, provided herein is a method of treating cancer by administering a herein described peptide, conjugate or a pharmaceutically acceptable salt or solvate thereof to a subject in need thereof. According to a further aspect of the disclosure, there is provided a peptide, or a drug conjugate thereof as defined herein, for use in preventing, suppressing or treating a disease or disorder characterized by overexpression or decreased expression of EphA2 in diseased tissue. In some embodiments, the disease or disorder is characterized by overexpression of EphaA2 in diseased tissue (such as a tumor). In one embodiment, the EphA2 is mammalian EphA2. In a further embodiment, the mammalian EphA2 is human EphA2.

In one aspect, provided herein is a method of preventing, suppressing or treating a disease or disorder characterized by overexpression or decreased expression of EphA2 in diseased tissue. In some embodiments, the disease or disorder is characterized by overexpression of EphaA2 in diseased tissue (such as a tumor), which comprises administering to a patient in need thereof a peptide or a conjugate described herein (e.g., including a peptide, a payload molecule, and optionally a linker). In some embodiments, the disease or disorder characterized by overexpression of EphA2 in diseased tissue is a cancer.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”). Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, bladder cancer, colorectal cancer, gastric adenocarcinoma, ovarian cancer, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer. In some embodiments, a cancer to be treated by the methods of the present disclosure is non-small cell lung cancer, ovarian cancer, or bladder cancer. In some embodiments, a cancer to be treated by the methods of the present disclosure is non-small cell lung cancer. In some embodiments, a cancer to be treated by the methods of the present disclosure is bladder cancer. In some embodiments, a cancer to be treated by the methods of the present disclosure is ovarian cancer.

Further examples of cancers (and their benign counterparts) which may be treated include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); hematological malignancies (i.e. leukemias, lymphomas) and premalignant hematological disorders and disorders of borderline malignancy including hematological malignancies and related conditions of lymphoid lineage (for example acute lymphocytic leukemia (ALL), chronic lymphocytic leukemia, B-cell lymphomas such as diffuse large B-cell lymphoma, follicular lymphoma, Burkitt's lymphoma, mantle cell lymphoma, T-cell lymphomas and leukemias, natural killer cell lymphomas, Hodgkin's lymphomas, hairy cell leukemia, monoclonal gammopathy of uncertain significance, plasmacytoma, multiple myeloma, and post-transplant lymphoproliferative disorders), and hematological malignancies and related conditions of myeloid lineage (for example acute myelogenous leukemia, chronic myelogenous leukemia, chronic myelomonocytic leukemia, hypereosinophilic syndrome, myeloproliferative disorders such as polycythemia vera, essential thrombocythemia and primary myelofibrosis, myeloproliferative syndrome, myelodysplastic syndrome, and promyelocytic leukemia); tumors of mesenchymal origin, for example sarcomas of soft tissue, bone or cartilage such as osteosarcomas, fibrosarcoma's, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcoma protuberans; tumors of the central or peripheral nervous system (for example astrocytoma's, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In some embodiments, the cancer is selected from glioblastoma, prostate cancer, lung cancer, breast cancer, gastric cancer, ovarian cancer, gladder cancer, colon cancer, esophageal cancer, multiple myeloma and fibrosarcoma. In some embodiments, the cancer is selected from: breast cancer, lung cancer, gastric cancer, pancreatic cancer, prostate cancer, liver cancer, glioblastoma and angiogenesis. In some embodiments, the cancer is selected from: prostate cancer, lung cancer (such as non-small cell lung carcinomas (NSCLC)), breast cancer (such as triple negative breast cancer), gastric cancer, ovarian cancer, esophageal cancer, multiple myeloma and fibrosarcoma. In some embodiments, the cancer is prostate cancer. In some embodiments, the conjugate is useful for preventing, suppressing or treating solid tumors such as fibrosarcoma's and breast, and non-small cell lung carcinomas. In some embodiments, the cancer is selected from lung cancer, such as non-small cell lung carcinomas (NSCLC). In some embodiments, the cancer is breast cancer. In some embodiments, the breast cancer is triple negative breast cancer. In some embodiments, the breast cancer is Herceptin resistant breast cancer. In some embodiments, the subject has failed to respond to Herceptin. In some embodiments, the cancer is gastric cancer. In some embodiments, the cancer is ovarian cancer. In some embodiments, the cancer is esophageal cancer. In some embodiments, the cancer is multiple myeloma. In some embodiments, the cancer is fibrosarcoma.

In some embodiments, provided herein are methods for killing a cell comprising contacting the cell with a peptide, a conjugate, or a pharmaceutically acceptable salt or solvate thereof. In some embodiments, the cell expresses EphA2. In some embodiments, the cell over-expresses EphA2. In some embodiments, the conjugate or pharmaceutically acceptable salt or solvate thereof binds to a structure on the cell, wherein the structure is an EphA2.

After contacting a cell, the described peptide or conjugate can be internalized by the cell. The internalization can be mediated by cell receptors, cell membrane endocytosis, etc. In some embodiments, the described peptide or conjugate is internalized by a cell through EphA2. In some embodiments, rapid internalization rate into cancer cells accompanied by a slow externalization rate can offer therapeutic benefit.

In one aspect, the disclosed peptide, conjugate or a pharmaceutically acceptable salt or solvate thereof is configured to treat cancer by ablating tumor cells. In some embodiments, the peptide, conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immunological cascade.

In addition to the methods of treatment described above, the peptide, conjugates and compositions described herein can be used to image, and/or as part of a treatment for diseases. Suitable imaging applications include single-photon emission computed tomography (SPECT) and positron emission tomography (PET).

In one aspect, described herein is a method of diagnosing or imaging a cancer in a subject in need thereof, comprising administering to the subject a peptide, a conjugate or a pharmaceutical composition described herein.

In some embodiments, the subject is 1 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

### Combination Therapy

In some embodiments, a peptide or conjugate described herein can be administered alone or in combination with one or more additional therapeutic agents. For example, the combination therapy can include a composition comprising a peptide or a conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the peptide or conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the herein-described peptide or conjugate is used in combination with a chemotherapeutic agent, e.g., a DNA damaging chemotherapeutic agent, a platinum based agent, a topoisomerase inhibitor, a taxane, an antimetabolite, a vinca alkaloid, or an anthracycline. In some embodiments, the herein-described peptide or conjugate is used in combination with a radiation sensitizer, which makes tumor cells more sensitive to radiation therapy. In some embodiments, the herein-described peptide or conjugate is used in combination with a DNA damage repair inhibitor (or DNA damage response (DDR) inhibitor). In some embodiments, the DNA damage repair inhibitor or DDR inhibitor is a poly (ADP-ribose) polymerase (PARP) inhibitor. In some embodiments, the herein-described peptide or conjugate is used in combination with an immune checkpoint inhibitor. In some embodiments, the immune checkpoint inhibitor is a PD-L1 inhibitor, a PD-1 inhibitor, or a CTLA-4 inhibitor. In some embodiments, the herein-described peptide or conjugate is used in combination with a chemotherapeutic agent, a PARP inhibitor, and/or an immune checkpoint inhibitor. In some embodiments, the herein-described peptide or conjugate is used in combination with a chemotherapeutic agent, a PARP inhibitor, and an immune checkpoint inhibitor.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

The present technology is described in detail below based on examples, but the present technology is not limited to these examples. A person having ordinary skill in the art is capable of easily adding modifications and changes to the present technology based on the description of the present specification, and these are included in the technical scope of the present technology.

### Example 1—Chemical Synthesis

### Analytical Methods, Materials, and Instrumentation

Unless otherwise noted, reagents and solvents were used as received from commercial suppliers. If not otherwise specified, purity and low-resolution mass spectral data were measured using a Shimadzu LC/MS system or Waters ACQUITY LC/MS system (ESI). Methods are specified below.

### General Analytical Methods

Method A-A (HPLC-MS): Shimadzu LC/MS system, Kinetex EVO C18 2.6 um, 2.1 ID×150 mm, 100 Å (with a guard cartridge 2.1 mmID); 60° C.; 0.5 mL/min; (A) H2O+0.025% TFA/(B) MeCN+0.025% TFA; Gradient: from 5 to 45% B in 7.2 min. Electrospray mass spectra (+), PDA-UV chromatogram TIC, 225 nm.
Method A-B (HPLC-MS): Shimadzu LC/MS system, Kinetex EVO C18 2.6 um, 2.1 ID×150 mm, 100 Å (with a guard cartridge 2.1 mmID); 60° C.; 0.5 mL/min; (A) H2O+0.025% TFA/(B) MeCN+0.025% TFA; Gradient: from 20 to 60% B in 7.2 min. Electrospray mass spectra (+), PDA-UV chromatogram TIC, 225 nm.
Method A-C (HPLC-MS): Shimadzu LC/MS system, Kinetex EVO C18 2.6 um, 2.1 ID×150 mm, 100 Å (with a guard cartridge 2.1 mmID); 60° C.; 0.5 mL/min; (A) H2O+0.025% TFA/(B) MeCN+0.025% TFA; Gradient: from 40 to 80% B in 7.2 min. Electrospray mass spectra (+), PDA-UV chromatogram TIC, 225 nm.

### General Preparative HPLC Purification Procedure

The crude peptides were purified by preparative reverse phase C18-HPLC, using columns of different sizes and with varying flow rates, depending on the amount of crude peptide to be purified. Usually, 0.1% TFA in H2O (A) and Acetonitrile (B) were employed as eluents. Product-containing fractions were collected and lyophilized to obtain the purified product.
Method P-A: XBridge C18 5 um 50×150 mm; (20 ml/min-20 mL/min)/1 min, (20 mL/min-120 mL/min)/1 min, 120 mL/min for the rest; (A) H2O+0.1% TFA/(B) MeCN+0.1% TFA; varying gradients; Detection: DAD-UV chromatogram TIC, 220 nm
Method P—B: XBridge C18 5 um 50×150 mm; (20 mL/min-20 mL/min)/1 min, (20 mL/min-120 mL/min)/2 min, 120 mL/min for the rest; (A) H2O+0.1% TFA/(B) MeCN+0.1% TFA; varying gradients; Detection: DAD. UV chromatogram TIC, 220 nm
Method P—C: XBridge C18 5 um 50×150 mm; (120 mL/min-120 mL/min)/5 min, (20 mL/min-20 mL/min)/1 min, (20 mL/min-120 mL/min)/1 min, 120 mL/min for the rest; (A) H2O+0.1% TFA/(B) MeCN+0.1% TFA; varying gradients; Detection: DAD-UV chromatogram TIC, 220 nm
Method P-D: XBridge C18 5 um 50×150 mm; 120 ml/min; (A) H2O+0.1% TFA/(B) MeCN+0.1% TFA; varying gradients; Detection: DAD-UV chromatogram TIC, 220 nm
Method P-E: XBridge C18 5 um 50×250 mm; (118 mL/min-18 mL/min)/0.1 min. (18 mL/min-18 mL/min)/4.9 min, (18 mL/min-118 mL/min)/2 min, 118 mL/min for the rest; (A) H2O+0.1% TFA/(B) MeCN+0.1% TFA; varying gradients; Detection: DAD-UV chromatogram TIC, 220 nm

### General Scheme A

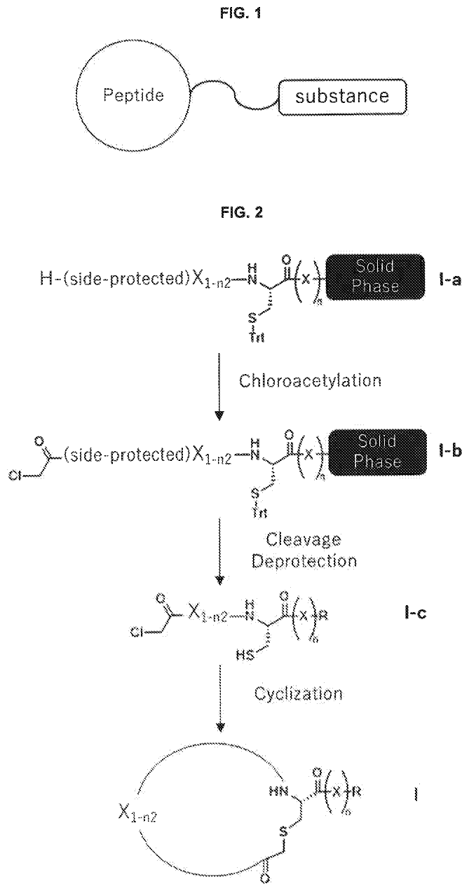

Macrocyclic peptides I in the present invention can be synthesized by the general method outlined in scheme A, shown in FIG. 2.

### Step 1: Solid Phase Peptide Synthesis (SPPS) Method A:

The peptide synthesis in this invention was performed on the Liberty BLUE HT 12TM (CEM. Inc.) according to the manufacturer's instruction. In fact, Fmoc-Sieber amide Resin was suspended in the solvent (e.g., DMF or DCM) and then loaded onto the peptide synthesizer. After the Fmoc removal of Fmoc-Sieber amide Resin, the coupling steps and Fmoc removal steps were repeatedly continued until the desired linear polypeptide of Formula I-a was obtained. AA coupling and Fmoc deprotection conditions for method A were listed in the table below.

### General Method for AA Coupling

TABLE 1-1

| AA | Reagents | coupling conditions |
|---|---|---|
| Fmoc-Cys(Trt)-OH | AA/DIC/Oxyma | 50° C., 1-2x 15-20 min |
| Other than listed | Pure ® in DMF | 90° C., 1-2x 3-10 min |
| above | | or 75° C., 2x 20-30 min |

The AAs listed below were introduced by employing DIC/Oxyma or HATU/DIEA.

TABLE 1-2

| AA | HATU | | DIEA |
|---|---|---|---|
| Fmoc-Me3Py-OH 4.2 eq. | 4-5 | eq. | 8-12 eq. |
| Fmoc-W1Me7Cl—OH 2.5-4.2 eq. | 2.5-5 | eq. | 5-10 eq. |
| Fmoc-df3CON—OH 4.2-5.3 eq. | 4-5 | eq. | 8-10 eq. |

### General Method for Fmoc Deprotection

TABLE 1-3

| AA | Reagents | deprotection conditions |
|---|---|---|
| AA next to N-Me AA | pyrrolidine/DMF (1/9) | 25° C., 1-2x 1-5 min |
| Other than listed | or piperidine/DMF | 90° C., 1 min or |
| above | (1/4) | 75° C., 3 min |

### Step 1: Solid Phase Peptide Synthesis (SPPS) Method B:

The peptide synthesis in this invention was performed on the Liberty BLUE HT 12™ (CEM. Inc.) according to the manufacturer's instruction. In fact, Fmoc-Sieber amide Resin was suspended in the solvent (e.g., DMF or DCM) and then loaded onto the peptide synthesizer. After the Fmoc removal of Fmoc-Sieber amide Resin, the coupling steps and Fmoc removal steps were repeatedly continued until the desired linear polypeptide of Formula I-a was obtained. AA coupling and Fmoc deprotection conditions for method B were listed in the table below.

### General Method for AA Coupling

TABLE 2-1

| AA | Reagents | coupling conditions |
|---|---|---|
| Fmoc-Cys(Trt)-OH | AA/DIC/Oxyma | 50° C., 1-2x 15-20 min |
| Other than listed | Pure ® in DMF | 105° C., 1-2x 2 min |
| above | | or 90° C., 1-2x 3-10 min |
| | | or 75° C., 2x 20-30 min |

### General Method for Fmoc Deprotection

TABLE 2-2

| | | deprotection |
|---|---|---|
| AA | Reagents | conditions |
| AA next to N-Me AA | pyrrolidine/DMF (1/9) | 25° C., 1-2x 1-5 min |
| Other than listed | 83 mM Oxyma pure in | 110° C., 1-1.5 min |
| above | pyrrolidine/DMF (4/96) | |

### Step 2: The Introduction of Chloroacetyl Group:

The obtained resin in the step-1 on the resin was transferred in a syringe with a flit. The resin was shaken in one of the reagents listed below at room temperature for 0.5-2 h. The solution was then drained through the frit. The resin was washed successively a few times with DMF, CH2Cl2, and Et2O to afford the linear peptides of Formula I-b on the resin.

Chloroacetylation conditions were listed in the table below.

TABLE 3-1

| | |
|---|---|
| ClAc-1 | resin/ClAcOH in DMF(0.1-0.2M)/HATU in DMF(0.1-0.5M)/ |
| | DIPEA in DMF (0.2-1M) (1 eq/4.2-5 eq/4-5 eq/4.2-10 eq) |
| ClAc-2 | ClAcOSu NMP/DCM (0.1-0.2M) prepared by ClAcOH/DIC/ |
| | HOSu (4-10 eq/4-10 eq/4-10 eq to 1 eq of resin) |
| ClAc-3 | ClAcOSu in DMF/DCM (0.1-0.25M) prepared by ClAcOH/DIC/ |
| | HOSu (4-10 eq/4-10 eq/4-10 eq to 1 eq of resin) |
| ClAc-4 | ClAcOSu in DMF (4-10 eq to 1 eq of resin) |

Step 3: Cleavage from Resin and Global Deprotection of Side Chain Protecting Groups (PGs)

The intermediate I-b was shaken at rt for 20-90 min with the cleavage cocktail solution listed below. The resin was filtered and washed with the cleavage solution. The resin was then filtered, and the combined filtrates were poured into cold diethyl ether. The resulting suspension was centrifuged (9000 rpm, 1 min at 0° C.) and the supernatant was decanted out. The precipitate was suspended in cold ether, vortexed briefly, and then centrifuged. This process was repeated when appropriate. The crude containing peptide I-c was dried under reduced pressure.

Cleavage conditions were listed in the table below.

TABLE 4

| | |
|---|---|
| Cocktail-A | TFA/TIS/DODT/H₂O = 92.5/2.5/2.5/2.5 |
| Cocktail-B | TFA/TIS/DODT/H₂O = 90/2.5/5.0/2.5 |

### Step 4: Peptide Cyclization

The crude containing polypeptide I-c was dissolved in one of solvents listed below, and subsequently added TEA (5-15 eq). The mixture was then shaken at room temperature for 1 h to ca. 16 h until completion of the reaction. The resulting reaction mixture was concentrated by Genevac E-2 Elite or lyophilization. The resulting residue containing the desired polypeptide was purified by preparative reverse phase C18-HPLC to afford polypeptides of formula I.

Peptide cyclization conditions were listed in the table below.

TABLE 5

| | Solvent | Reagent | Time |
|---|---|---|---|
| Cy-1 | DMSO/H₂O (95/5) | TEA (5-15 eq) | 1-16 h |
| Cy-2 | MeCN/H₂O (1/1) | | |
| Cy-3 | DMSO/H₂O (9/1) | | |
| Cy-4 | DMSO | | |
| Cy-5 | DMSO/H₂O/IPA (90/5/5) | | |
| Cy-6 | DMSO/MeCN/H2O (1/1/1) | | |

### General Scheme B

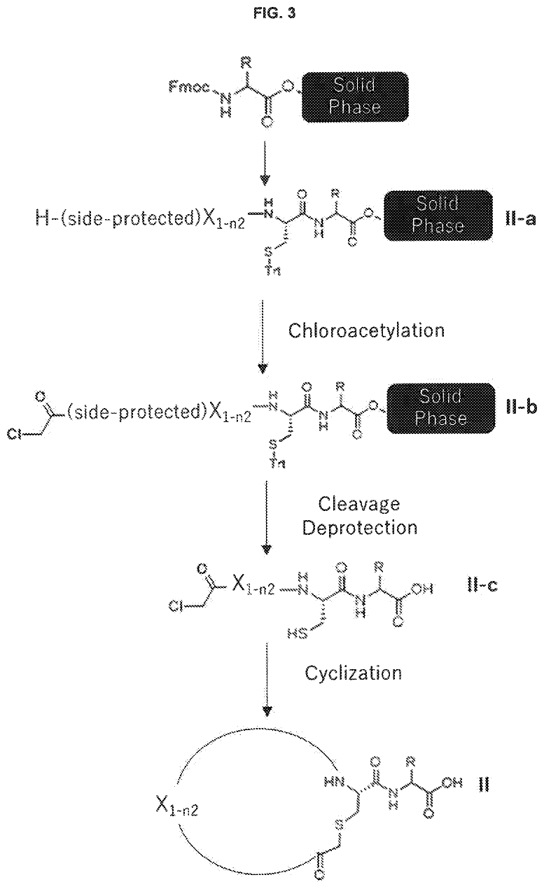

Macrocyclic peptides II in the present invention can be synthesized on the pre-loaded resin in the analogous manner to the scheme A. The general synthetic scheme is described in Scheme B shown in FIG. 3.

### General Scheme C

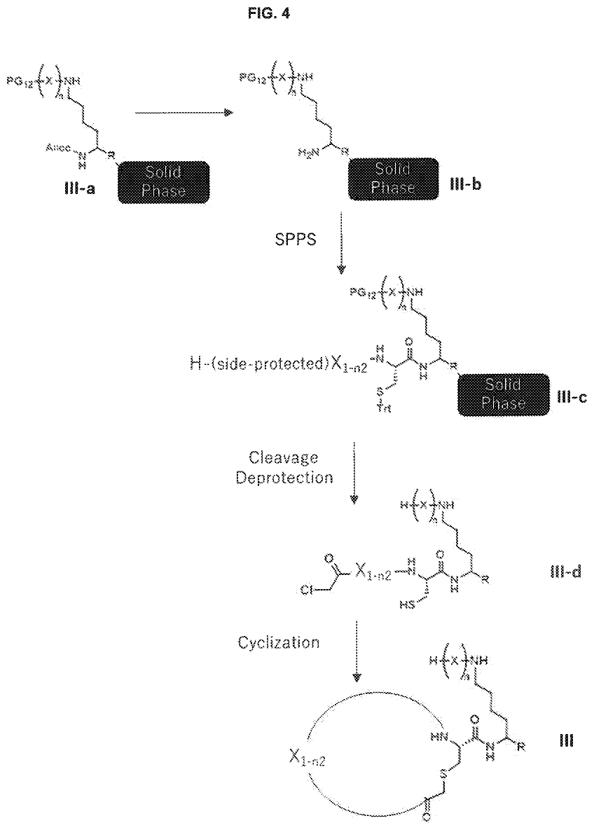

Macrocyclic peptides III in the present invention can be synthesized by following the steps outlined in scheme C shown in FIG. 4.

### Step 1: Peptide Synthesis

Intermediates III-a on-resin were synthesized in the analogous manner to the step 1 of the scheme A.

### Step 2: Deprotection of Alloc Protecting Group

The polypeptides III-a on-resin were suspended in DCM. The resin was shaken with Pd(PPh3)4 (0.2-0.25 eq) and PhSiH3 (10-15 eq) at room temperature for 1 h. The resin was washed with DMF. Then the resin was washed by DCM followed by DMF to provide the intermediate polypeptides III-b on the resin.

### Step 3-5:

Polypeptides of formula III were synthesized from the intermediates III-b on-resin in the analogous manner to scheme A.

### Example-1: PDC_EphA2_00007196-C002 (SEQ ID NO: 200)

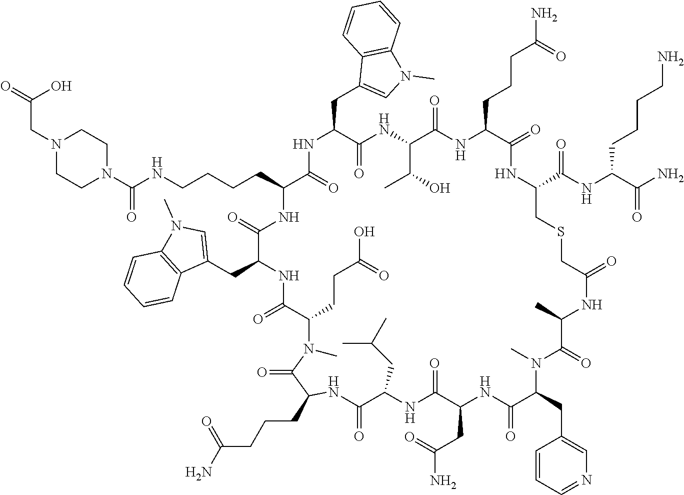 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O]

The peptide sequence was synthesized on Fmoc-Sieber amide Resin (0.375 mmol) following general peptide synthesis method A. The obtained peptide on the resin was subjected to the general method CIAc-1. The polypeptide on the resin was treated with Cleavage Cocktail-A (18 mL) to furnish the linear peptide. The crude containing the linear peptide (0.375 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.16 min, ESI-MS m/z: 988.9 [M+2H]2+, AUC (UV 225 nm): 94%.

### Example-2: PDC_EphA2-00008093-C002 (SEQ ID NO: 199)

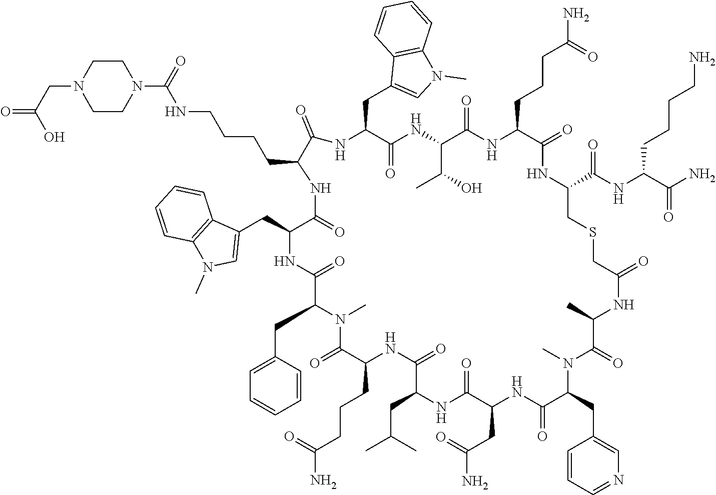 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC1=O]

The peptide sequence was synthesized on Fmoc-Sieber amide Resin (0.375 mmol) following general peptide synthesis method A. The obtained peptide on the resin was subjected to the general method CIAc-1. The polypeptide on the resin was treated with Cleavage Cocktail-A (18 mL) to furnish the linear peptide. The crude containing the linear peptide (0.375 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-B): 3.51 min, ESI-MS m/z: 998.0 [M+2H]2+, AUC (UV 225 nm): 97%.

### Example-3: PDC_EphA2-00019437-C002 (SEQ ID NO: 204)

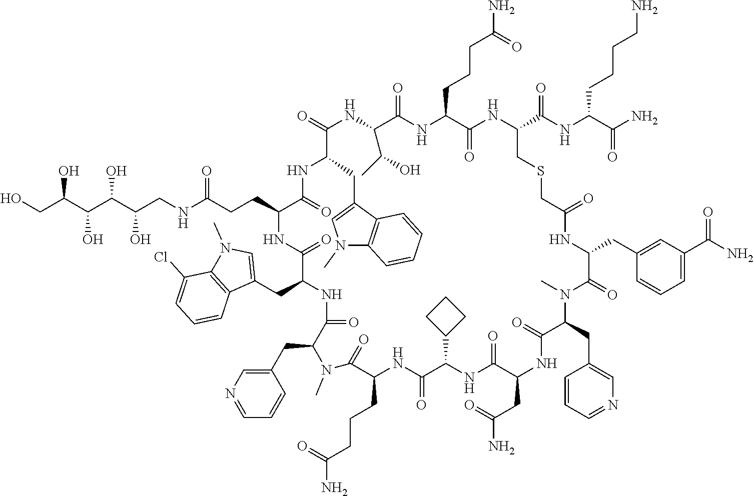 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@H](CCCCN)C(N)=O)NC(=O)[C@H](CCCC(N)=O)NC1=O]

The peptide sequence was synthesized on Fmoc-Sieber amide Resin (0.57 mmol/g, 0.25 mmol) following general peptide synthesis method A. The obtained peptide on the resin was subjected to the general method CIAc-2. The polypeptide on the resin was treated with Cleavage Cocktail-A (15 mL) to furnish the linear peptide. The crude containing the linear peptide (0.25 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.07 min, ESI-MS m/z: 1071.6 [M+2H]2+, AUC (UV 225 nm): 98%.

### Example-4: PDC_EphA2-00019440-C002 (SEQ ID NO: 208)

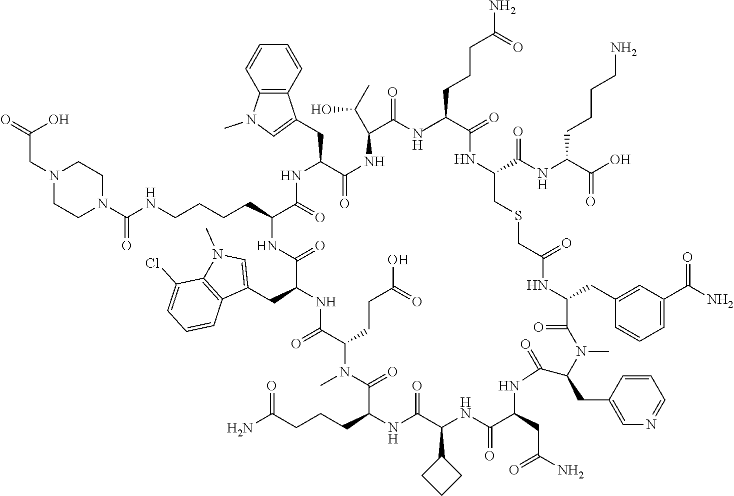 [SMILES: CNC(=O)[C@@H](NC(=O)[C@H](Cc1cn(C)c2ccccc12)NC(=O)[C@H](CCCCNC(=O)N1CCN(CC(=O)O)CC1)NC(=O)[C@H](Cc1cn(C)c2c(Cl)cccc12)NC(=O)[C@H](CCC(=O)O)N(C)C(=O)[C@H](CCCC(N)=O)NC(=O)[C@@H](NC(=O)[C@H](CC(N)=O)NC(=O)[C@H](Cc1cccnc1)N(C)C(=O)[C@@H](Cc1cccc(C(N)=O)c1)NC(=O)CSC[C@H](NC(=O)[C@@H](C)CCCC(N)=O)C(=O)N[C@H](CCCCN)C(=O)O)C1CCC1)[C@@H](C)O]

The peptide sequence was synthesized on Fmoc-Sieber amide Resin (0.25 mmol) following general peptide synthesis method A. The obtained peptide on the resin was subjected to the general method CIAc-3. The polypeptide on the resin was treated with Cleavage Cocktail-B (15 mL) to furnish the linear peptide. The crude containing the linear peptide (0.25 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.44 min, ESI-MS m/z: 1065.1 [M+2H]2+, AUC (UV 225 nm): 99%.

### Example 5

The following Examples were prepared by following similar methods described in the examples above. The amino acid sequence for each peptide was shown in Tables 6A and/or 6B. The term “Term” means the functional group at C-terminus of the peptide.

TABLE 6A

| SEQ | | | | | | | | | | | | |
|---|---|---|---|---|---|---|---|---|---|---|---|---|
| ID | Sequence | | | | | | | | | | | |
| NO: | 1 | 2 | 3 | 4 | 5 | 6 | 7 | 8 | 9 | 10 | 11 | 12 |
| 1 | da | MeF | N | L | Hgl | MeF | W1Me | V | W1Me | T | E | C |
| 2 | da | Me3Py | N | L | Hgn | MeF | W1Me | KCOpipzaa | W1Me | T | Hgn | C |
| 3 | da | Me3Py | N | Cbg | Hgn | MeF | W1Me | KCOpipzaa | W1Me | T | Hgn | C |
| 4 | da | Me3Py | N | Cbg | Hgn | N | W1Me | KCOpipzaa | W1Me | T | Hgn | C |
| 5 | da | Me3Py | N | Cbg | Hgn | MeN | W1Me | KCOpipzaa | W1Me | T | Hgn | C |
| 6 | da | Me3Py | N | Cbg | Hgn | MeF | W1Me7N | KCOpipzaa | W1Me | T | Hgn | C |
| 7 | da | Me3Py | N | Cbg | Hgn | MeF | W1Me | KCOpipzaa | F23dMe | T | Hgn | C |
| 8 | da | Me3Py | N | Cbg | Hgn | MeF | W1Me | KCOpipzaa | W1Me | T | N | C |
| 9 | da | Me3Py | N | Chg | Hgn | MeF | W1Me | KCOpipzaa | W1Me | T | Hgn | C |
| 10 | da | Me3Py | N | L | Hgl | MeE | W1Me | KCOpipzaa | W1Me | T | Hgn | C |
| 11 | da | Me3Py | N | Cbg | Hgl | MeE | W1Me | KCOpipzaa | W1Me | T | Hgn | C |
| 12 | da | Me3Py | N | L | Hgn | MeE | W1Me | KCOpipzaa | W1Me | T | Hgn | C |
| 13 | da | Me3Py | N | Cbg | Hgn | MeE | W1Me | KCOpipzaa | W1Me | T | Hgn | C |
| 14 | da | Me3Py | N | Cbg | Hgn | N | W1Me | V | W1Me | T | Hgn | C |
| 15 | da | Me3Py | N | Cbg | Hgn | MeN | W1Me | V | W1Me | T | Hgn | C |
| 16 | da | Me3Py | N | Cbg | Hgn | Me3Py | W1Me | V | W1Me | T | Hgn | C |
| 17 | da | MeF | N | Cbg | Hgn | MeN | W1Me | V | W1Me | T | Hgn | C |
| 18 | da | MeF | N | Cbg | Hgn | MeN | W1Me | Qglucamine | W1Me | T | Hgn | C |
| 19 | da | MeF | N | Cbg | Hgn | Me3Py | W1Me | Qglucamine | W1Me | T | Hgn | C |
| 20 | da | Me3Py | N | Cbg | Hgn | Me3Py | W1Me | Qglucamine | W1Me | T | Hgn | C |
| 21 | da | MeF | N | Cbg | Hgn | MeF | W1Me | QGlucamine | W1Me | T | Hgn | C |
| 22 | dahp | Me3Py | N | L | Hgl | MeF | W1Me | KCOpipzaa | W1Me | T | Hgn | C |
| 23 | da | Me3Py | N | L | Hgl | MeF4C | W1Me | KCOpipzaa | W1Me | T | Hgn | C |
| 24 | da | Me3Py | N | L | Hgl | MeF | W1Me | KCOpipzaa | W1Me | T | Nmm | C |
| 25 | da | Me3Py | N | L | Hgl | MeF | W1Me | KCOpipzaa | W1Me | T | Ndm | C |
| 26 | dahp | Me3Py | N | L | Hgn | MeE | W1Me | KCOpipzaa | W1Me | T | Hgn | C |
| 27 | da | Me3Py | N | L | Nmm | MeE | W1Me | KCOpipzaa | W1Me | T | Hgn | C |
| 28 | da | Me3Py | N | L | Ndm | MeE | W1Me | KCOpipzaa | W1Me | T | Hgn | C |
| 29 | da | Me3Py | N | L | Hgn | MeE | W1Me | KCOpipzaa | W1Me | T | Nmm | C |
| 30 | da | Me3Py | N | L | Hgn | MeE | W1Me | KCOpipzaa | W1Me | T | Ndm | C |
| 31 | dahp | Me3Py | N | Chg | Hgn | MeF | W1Me | KCOpipzaa | W1Me | T | Hgn | C |
| 32 | da | Me3Py | N | Chg | Nmm | MeF | W1Me | KCOpipzaa | W1Me | T | Hgn | C |
| 33 | da | Me3Py | N | Chg | Ndm | MeF | W1Me | KCOpipzaa | W1Me | T | Hgn | C |
| 34 | da | Me3Py | N | Chg | Hgn | MeF4C | W1Me | KCOpipzaa | W1Me | T | Hgn | C |
| 35 | da | Me3Py | N | Chg | Hgn | MeF | W1Me | KCOpipzaa | W1Me | T | Nmm | C |
| 36 | da | Me3Py | N | Chg | Hgn | MeF | W1Me | KCOpipzaa | W1Me | T | Ndm | C |
| 37 | df3CON | MeF | N | Cbg | Hgn | MeN | W1Me7Cl | V | W1Me | T | Hgn | C |
| 38 | df3CON | MeF | N | L | Hgn | MeN | W1Me7Cl | V | W1Me | T | Hgn | C |
| 39 | df3CON | MeF | N | L | Hgn | MeN | W1Me | V | W1Me | T | Hgn | C |
| 40 | df3CON | Me3Py | N | Cbg | Hgn | MeN | W1Me7Cl | V | W1Me | T | Hgn | C |
| 41 | df3CON | MeF | N | Cbg | Hgn | MeN | W1Me7Cl | KCOpipzaa | W1Me | T | Hgn | C |
| 42 | df3CON | MeF | N | Cbg | Hgn | MeN | W1Me7Cl | Hcit | W1Me | T | Hgn | C |
| 43 | df3CON | MeF | N | Cbg | Hgn | MeE | W1Me7Cl | Hcit | W1Me | T | Har | C |
| 44 | df3CON | Me3Py | N | Cbg | Hgn | MeN | W1Me7Cl | Hcit | W1Me | T | R | C |
| 45 | df3CON | Me3Py | N | Cbg | Hgn | MeN | W1Me7Cl | Hcit | W1Me | T | Har | C |
| 46 | df3CON | Me3Py | N | Cbg | Hgn | MeN | W1Me7Cl | KCOpipzaa | W1Me | T | Har | C |
| 47 | df3CON | MeF | N | Cbg | Hgn | MeF | W1Me7Cl | Qglucamine | W1Me | T | Hgn | C |
| 48 | df3CON | MeF | N | Cbg | Hgn | MeF | W1Me7Cl | KCOpipzaa | W1Me | T | Hgn | C |
| 49 | df3CON | Me3Py | N | Cbg | Hgn | MeF | W1Me7Cl | Qglucamine | W1Me | T | Hgn | C |
| 50 | df3CON | Me3Py | N | Cbg | Hgn | MeF | W1Me7Cl | KCOpipzaa | W1Me | T | Hgn | C |
| 51 | df3CON | MeF | N | Cbg | Hgn | Me3Py | W1Me7Cl | Qglucamine | W1Me | T | Hgn | C |
| 52 | df3CON | MeF | N | Cbg | Hgn | Me3Py | W1Me7Cl | KCOpipzaa | W1Me | T | Hgn | C |
| 53 | df3CON | Me3Py | N | Cbg | Hgn | Me3Py | W1Me7Cl | Qglucamine | W1Me | T | Hgn | C |
| 54 | df3CON | Me3Py | N | Cbg | Hgn | Me3Py | W1Me7Cl | KCOpipzaa | W1Me | T | Hgn | C |
| 55 | df3CON | Me3Py | N | Cbg | Hgn | MeE | W1Me7Cl | Qglucamine | W1Me | T | Hgn | C |
| 56 | df3CON | Me3Py | N | Cbg | Hgn | MeE | W1Me7Cl | KCOpipzaa | W1Me | T | Hgn | C |
| 57 | df3CON | Me3Py | N | Cbg | KCOpipzaa | MeF | W1Me7Cl | KCOpipzaa | W1Me | T | Hgn | C |
| 58 | df3CON | Me3Py | N | Cbg | KCOpipzaa | MeF | W1Me7Cl | Qglucamine | W1Me | T | Hgn | C |
| 59 | df3CON | Me3Py | N | L | N | Me3Py | W1Me7Cl | Qglucamine | W1Me | T | N | C |
| 60 | df3CON | Me3Py | N | L | N | MeN | W1Me7Cl | Qglucamine | W1Me | T | N | C |
| 61 | df3CON | Me3Py | N | L | N | MeN | W1Me7Cl | Cit | W1Me | T | N | C |
| 62 | da | Me3Py | N | L | K | MeE | W1Me7Cl | KCOpipzaa | W1Me | T | Hgn | C |
| 63 | da | Me3Py | N | L | Hgn | MeE | W1Me7Cl | K | W1Me | T | Hgn | C |
| 64 | da | Me3Py | N | L | Hgn | MeE | W1Me7Cl | KCOpipzaa | W1Me | T | K | C |
| 65 | df3CON | Me3Py | N | L | K | MeN | W1Me7Cl | Qglucamine | W1Me | T | Hgn | C |
| 66 | df3CON | Me3Py | N | L | Hgn | MeN | W1Me7Cl | K | W1Me | T | Hgn | C |
| 67 | df3CON | Me3Py | N | L | Hgn | MeN | W1Me7Cl | Qglucamine | W1Me | T | K | C |
| 68 | df3CON | Me3Py | N | L | K | MeE | W1Me7Cl | KCOpipzaa | W1Me | T | Hgn | C |
| 69 | df3CON | Me3Py | N | L | Hgn | MeE | W1Me7Cl | K | W1Me | T | Hgn | C |
| 70 | df3CON | Me3Py | N | L | Hgn | MeE | W1Me7Cl | KCOpipzaa | W1Me | T | K | C |
| 71 | da | Me3Py | N | Cbg | Hgn | MeN | W1Me | V | W1Me | T | Hgn | C |
| 72 | da | Me3Py | N | Cbg | Hgn | Me3Py | W1Me | V | W1Me | T | Hgn | C |
| 73 | da | MeF | N | Cbg | Hgn | MeN | W1Me | V | W1Me | T | Hgn | C |
| 74 | da | MeF | N | L | Hgl | MeF | W1Me | V | W1Me | T | E | C |
| 75 | da | Me3Py | N | L | Hgl | MeF | W1Me | V | W1Me | T | E | C |
| 76 | dkCOpipzaa | Me3Py | N | L | Hgl | MeF | W1Me | V | W1Me | T | E | C |
| 77 | dkCOpipzaa | Me3Py | N | L | KCOpipzaa | MeF | W1Me | V | W1Me | T | E | C |
| 78 | dkCOpipzaa | Me3Py | N | L | KCOpipzaa | MeF | W1Me | V | W1Me | T | Hgn | C |
| 79 | da | Me3Py | N | L | Hgl | MeF | W1Me | KCOpipzaa | W1Me | T | E | C |
| 80 | da | Me3Py | N | L | KCOpipzaa | MeF | W1Me | KCOpipzaa | W1Me | T | E | C |
| 81 | da | Me3Py | N | L | KCOpipzaa | MeF | W1Me | KCOpipzaa | W1Me | T | E | C |
| 82 | da | Me3Py | N | L | KCOpipzaa | MeF | W1Me | KCOpipzaa | W1Me | T | E | C |
| 83 | da | Me3Py | N | L | Hgl | MeF | W1Me | KCOpipzaa | W1Me | T | Hgn | C |
| 84 | da | Me3Py | N | L | KCOpipzaa | MeF | W1Me | KCOpipzaa | W1Me | T | Hgn | C |
| 85 | da | Me3Py | N | L | Hgn | MeF | W1Me | KCOpipzaa | W1Me | T | Hgn | C |
| 86 | da | Me3Py | N | L | Hgl | MeE | W1Me | KCOpipzaa | W1Me | T | Hgn | C |
| 87 | da | Me3Py | N | L | KCOpipzaa | MeE | W1Me | KCOpipzaa | W1Me | T | Hgn | C |
| 88 | da | Me3Py | N | L | Hgn | MeE | W1Me | KCOpipzaa | W1Me | T | Hgn | C |
| 89 | A | Me3Py | N | L | Hgl | MeF | W1Me | KCOpipzaa | W1Me | T | E | C |
| 90 | da | Me3Py | N | L | Hgn | MeF | W1Me | KCOpipzaa | W1Me | T | Hgn | C |
| 91 | da | Me3Py | N | L | Hgn | MeE | W1Me | KCOpipzaa | W1Me | T | Hgn | C |
| 92 | da | Me3Py | N | Cbg | Hgn | MeN | W1Me | V | W1Me | T | Hgn | C |
| 93 | da | Me3Py | N | Cbg | Hgn | Me3Py | W1Me | V | W1Me | T | Hgn | C |
| 94 | da | MeF | N | Cbg | Hgn | MeN | W1Me | V | W1Me | T | Hgn | C |
| 95 | df3CON | Me3Py | N | Cbg | Hgn | Me3Py | W1Me7Cl | Qglucamine | W1Me | T | Hgn | C |
| 96 | df3CON | Me3Py | N | Cbg | Hgn | MeN | W1Me7Cl | Qglucamine | W1Me | T | Hgn | C |
| 97 | df3CON | Me3Py | N | Cbg | Qglucamine | MeN | W1Me7Cl | Qglucamine | W1Me | T | Hgn | C |
| 98 | df3CON | Me3Py | N | Cbg | Hgn | MeN | W1Me | Qglucamine | W1Me | T | Hgn | C |
| 99 | df3CON | Me3Py | N | Cbg | Hgn | MeE | W1Me7Cl | KCOpipzaa | W1Me | T | Hgn | C |
| 100 | df3CON | MeF3CN | N | Cbg | Hgn | MeE | W1Me7Cl | KCOpipzaa | W1Me | T | Hgn | C |
| 101 | df3CON | MeF3H | N | Cbg | Hgn | MeE | W1Me7Cl | KCOpipzaa | W1Me | T | Hgn | C |
| 102 | df3CON | MeF3CN | N | Cbg | Hgn | Me3Py | W1Me7Cl | Qglucamine | W1Me | T | Hgn | C |
| 103 | df3CON | MeF3CN | N | Cbg | Hgn | MeN | W1Me7Cl | Qglucamine | W1Me | T | Hgn | C |
| 104 | da | MeF | N | Cbg | Hgn | MeN | W1Me | Qglucamine | W1Me | T | Hgn | C |
| 105 | da | MeF | N | Cbg | Hgn | Me3Py | W1Me | Qglucamine | W1Me | T | Hgn | C |
| 106 | da | Me3Py | V | Cbg | Hgn | Me3Py | W1Me | Qglucamine | W1Me | T | Hgn | C |
| 107 | df3CON | Me3Py | N | L | N | Me3Py | W1Me7Cl | Qglucamine | W1Me | T | N | C |
| 108 | df3CON | Me3Py | N | L | N | MeN | W1Me7Cl | Qglucamine | W1Me | T | N | C |
| 109 | df3CON | Me3Py | N | L | N | MeN | W1Me7Cl | Cit | W1Me | T | N | C |
| 110 | da | Me3Py | N | L | Hgn | MeE | W1Me | KCOpipzaa | W1Me | T | Hgn | C |
| 111 | da | MeF | N | Cbg | Hgn | MeN | W1Me | V | W1Me | T | Hgn | C |
| 112 | df3CON | Me3Py | N | Cbg | Hgn | Me3Py | W1Me7Cl | Qglucamine | W1Me | T | Hgn | C |
| 113 | df3CON | MeF3H | N | Cbg | Hgn | MeE | W1Me7Cl | KCOpipzaa | W1Me | T | Hgn | C |
| 114 | da | MeF | N | L | Hgl | MeF | W1Me | V | W1Me | T | E | C |
| 115 | da | Me3Py | N | L | Hgn | MeE | W1Me | KCOpipzaa | W1Me | T | Hgn | C |
| 116 | da | Me3Py | N | L | Hgn | MeF | W1Me | KCOpipzaa | W1Me | T | Hgn | C |
| 117 | da | Me3Py | N | L | Hgl | MeE | W1Me | KCOpipzaa | W1Me | T | Hgn | C |
| 118 | da | Me3Py | N | Cbg | Hgn | MeN | W1Me | V | W1Me | T | Hgn | C |
| 119 | da | Me3Py | N | Cbg | Hgn | Me3Py | W1Me | V | W1Me | T | Hgn | C |
| 120 | da | MeF | N | Cbg | Hgn | MeN | W1Me | V | W1Me | T | Hgn | C |
| 121 | da | Me3Py | N | L | Hgn | MeF | W1Me | KCOpipzaa | W1Me | T | Hgn | C |
| 122 | da | Me3Py | N | L | Hgn | MeE | W1Me | KCOpipzaa | W1Me | T | Hgn | C |
| 123 | F | H | N | L | S | MeF | W1Me | Hcit | W1Me | C | | |
| 124 | df3CON | Me3Py | N | L | S | MeF | W1Me7Cl | Hcit | W1Me | C | | |
| 125 | df3CON | Me3Py | N | L | S | Me3Py | W1Me7Cl | Hcit | W1Me | C | | |
| 126 | df3CON | Me3Py | N | Cbg | S | Me3Py | W1Me7Cl | Hcit | W1Me | C | | |
| 127 | df3CON | Me3Py | N | Cbg | Qglucamine | Me3Py | W1Me7Cl | KCOpipzaa | W1Me | C | | |
| 128 | df3CON | Me3Py | N | Cbg | Qglucamine | Me3Py | W1Me7Cl | Qglucamine | W1Me | C | | |
| 129 | df3CON | Me4Py2NH2 | N | Cbg | Qglucamine | Me3Py | W1Me7Cl | KCOpipzaa | W1Me | C | | |
| 130 | df3CON | Me3Py | N | Cbg | Hgn | MeE | W1Me7Cl | KCOpipzaa | W1Me | C | | |
| 131 | df3CON | Me3Py | N | Cbg | Qglucamine | MeE | W1Me7Cl | Hcit | W1Me | C | | |
| 132 | df3CON | MeF3H | N | Cbg | Hgn | MeE | W1Me7Cl | KCOpipzaa | W1Me | C | | |
| 133 | df3CON | Me3Py | N | Cbg | Hgn | MeE | W1Me7Cl | KCOpipzaa | W1Me | CdMe | | |
| 134 | df3CON | Me3Py | N | Cbg | Hgn | MeE | W1Me7Cl | KCOpipzaa | W1Me | C3SMe | | |
| 135 | df3CON | Me3Py | N | Cbg | Hgn | MeE | W1Me7Cl | KCOpipzaa | W1Me | C3RMe | | |
| 136 | F | H | N | L | S | MeF | W1Me | Hcit | W1Me | C | | |
| 137 | F | H | N | L | S | MeF | W1Me | Hcit | W1Me | C | | |
| 138 | F | H | N | L | S | MeF | W1Me | Hcit | W1Me | C | | |
| 139 | da | MeF | N | Cba | T | N | W1Me | Hcit | W1Me | C | | |
| 140 | F | 4Py | N | L | E | MeF | W1Me | 3Py6-NH2 | W1Me | C | | |
| 141 | F | H | N | L | S | MeF | W1Me | Hcit | W1Me | C | | |
| 142 | df3CON | Me3Py | N | L | S | MeF | W1Me7Cl | Hcit | W1Me | C | | |
| 143 | df3CON | Me3Py | N | L | S | Me3Py | W1Me7Cl | Hcit | W1Me | C | | |
| 144 | df3CON | Me3Py | N | Cbg | S | Me3Py | W1Me7Cl | Hcit | W1Me | C | | |
| 145 | df3CON | Me3Py | N | Cbg | Qglucamine | Me3Py | W1Me7Cl | KCOpipzaa | W1Me | C | | |
| 146 | df3CON | Me3Py | N | Cbg | Qglucamine | Me3Py | W1Me7Cl | Qglucamine | W1Me | C | | |
| 147 | df3CON | Me3Py | N | Cbg | Hgn | MeE | W1Me7Cl | KCOpipzaa | W1Me | C | | |
| 148 | df3CON | Me3Py | N | Cbg | Qglucamine | MeE | W1Me7Cl | Hcit | W1Me | C | | |
| 149 | df3CON | MeF3H | N | Cbg | Hgn | MeE | W1Me7Cl | KCOpipzaa | W1Me | C | | |
| 150 | F | 4Py2NH2 | L | Hcit | W1Me | MeF | W1Me | C | | | | |
| 151 | F | 4Py2NH2 | L | MeA | W1Me | MeF | W1Me | C | | | | |
| 152 | F | 4Py2NH2 | L | MeHcit | W1Me | MeF | W1Me | C | | | | |
| 153 | F | 4Py2NH2 | L | Hcit | W1Me | MeF | W1Me | C | | | | |
| 154 | F | 4Py2NH2 | L | Hcit | W1Me | MeF | W1Me | C | | | | |
| 155 | F | 4Py2NH2 | L | Hcit | W1Me | MeF | W1Me | C | | | | |
| 156 | F | 4Py | L | G | W1Me | MeF | W1Me | C | | | | |
| 157 | F | 4Py2NH2 | L | Hcit | W1Me | MeF | W1Me | C | | | | |
| 158 | da | 4Py2NH2 | MeF | W1Me | MeF | W1Me | C | | | | | |
| 159 | df3CON | Me3Py | N | Cbg | Hgn | MeE | W1Me7N | KCOpipzaa | W1Me | T | Hgn | C |
| 160 | da | Me3Py | N | Cbg | Hgn | MeE | W1Me7N | KCOpipzaa | W1Me | T | Hgn | C |
| 161 | da | Me3Py | N | Cbg | K | MeE | W1Me7Cl | KCOpipzaa | W1Me | T | Hgn | C |
| 162 | df3CON | Me3Py | N | Cbg | K | MeE | W1Me7Cl | KCOpipzaa | W1Me | T | Hgn | C |
| 163 | da | Me3Py | N | Cbg | K | MeE | W1Me7Cl | KCOpipzaa | W1Me | T | Hgn | C |
| 164 | df3CON | Me3Py | N | Cbg | K | MeE | W1Me | T | Hgn | C | | |
| 165 | da | Me3Py | N | Cbg | Hgn | MeE | W1Me7Cl | KCOpipzaa | W1Me | T | K | C |
| 166 | da | Me3Py | N | Cbg | K | MeE | W1Me7Cl | KCOpipzaa | W1Me | T | Hgn | C |
| 167 | da | Me3Py | N | L | Hgn | MeE | W1Me | KCOpipzaa | W1Me | T | E | C |
| 168 | da | Me3Py | N | L | Hgn | MeE | W1Me | KCOpipzaa | W1Me | T | hArg | C |
| 169 | da | Me3Py | N | L | Hgn | MeE | W1Me | KCOpipzaa | W1Me | T | hCit | C |
| 170 | da | Me3Py | N | L | Hgn | MeN | W1Me | KCOpipzaa | W1Me | T | hArg | C |
| 171 | df3CON | Me3Py | N | Cbg | Hgn | MeE | W1Me7Cl | KCOpipzaa | W1Me | T | hArg | C |

TABLE 6B
Example peptides in Table 6B include the indicated SEQ ID NO corresponding
to Table 6A and, optionally, an additional linker structure. The term
“Term” means the functional group at C-terminus of the peptide.

| | | | | | SEQ ID NO: |
|---|---|---|---|---|---|
| | SEQ | C-Term | | | (including |
| Example No. | ID NO | Ext | Linker | Term | linker) |
| PDC_EphA2-00001417 | 1 | | | —NH₂ | |
| PDC_EphA2-00009998 | 2 | | | —NH₂ | |
| PDC_EphA2-00009999 | 3 | | | —NH₂ | |
| PDC_EphA2-00010000 | 4 | | | —NH₂ | |
| PDC_EphA2-00010001 | 5 | | | —NH₂ | |
| PDC_EphA2-00010002 | 6 | | | —NH₂ | |
| PDC_EphA2-00010003 | 7 | | | —NH₂ | |
| PDC_EphA2-00010004 | 8 | | | —NH₂ | |
| PDC_EphA2-00010005 | 9 | | | —NH₂ | |
| PDC_EphA2-00010006 | 10 | | | —NH₂ | |
| PDC_EphA2-00010007 | 11 | | | —NH₂ | |
| PDC_EphA2-00010008 | 12 | | | —NH₂ | |
| PDC_EphA2-00010009 | 13 | | | —NH₂ | |
| PDC_EphA2-00010010 | 14 | | | —NH₂ | |
| PDC_EphA2-00010011 | 15 | | | —NH₂ | |
| PDC_EphA2-00010012 | 16 | | | —NH₂ | |
| PDC_EphA2-00013693 | 17 | | | —NH₂ | |
| PDC_EphA2-00013703 | 18 | | | —NH₂ | |
| PDC_EphA2-00013704 | 19 | | | —NH₂ | |
| PDC_EphA2-00013705 | 20 | | | —NH₂ | |
| PDC_EphA2-00013917 | 21 | | | —NH₂ | |
| PDC_EphA2-00018526 | 22 | | | —NH₂ | |
| PDC_EphA2-00018527 | 23 | | | —NH₂ | |
| PDC_EphA2-00018528 | 24 | | | —NH₂ | |
| PDC_EphA2-00018529 | 25 | | | —NH₂ | |
| PDC_EphA2-00018530 | 26 | | | —NH₂ | |
| PDC_EphA2-00018531 | 27 | | | —NH₂ | |
| PDC_EphA2-00018532 | 28 | | | —NH₂ | |
| PDC_EphA2-00018533 | 29 | | | —NH₂ | |
| PDC_EphA2-00018534 | 30 | | | —NH₂ | |
| PDC_EphA2-00018535 | 31 | | | —NH₂ | |
| PDC_EphA2-00018536 | 32 | | | —NH₂ | |
| PDC_EphA2-00018537 | 33 | | | —NH₂ | |
| PDC_EphA2-00018538 | 34 | | | —NH₂ | |
| PDC_EphA2-00018539 | 35 | | | —NH₂ | |
| PDC_EphA2-00018540 | 36 | | | —NH₂ | |
| PDC_EphA2-00019421 | 37 | | | —NH₂ | |
| PDC_EphA2-00019422 | 38 | | | —NH₂ | |
| PDC_EphA2-00019423 | 39 | | | —NH₂ | |
| PDC_EphA2-00019424 | 40 | | | —NH₂ | |
| PDC_EphA2-00019425 | 41 | | | —NH₂ | |
| PDC_EphA2-00019426 | 42 | | | —NH₂ | |
| PDC_EphA2-00019427 | 43 | | | —NH₂ | |
| PDC_EphA2-00019428 | 44 | | | —NH₂ | |
| PDC_EphA2-00019429 | 45 | | | —NH₂ | |
| PDC_EphA2-00019430 | 46 | | | —NH₂ | |
| PDC_EphA2-00019431 | 47 | | | —NH₂ | |
| PDC_EphA2-00019432 | 48 | | | —NH₂ | |
| PDC_EphA2-00019433 | 49 | | | —NH₂ | |
| PDC_EphA2-00019434 | 50 | | | —NH₂ | |
| PDC_EphA2-00019435 | 51 | | | —NH₂ | |
| PDC_EphA2-00019436 | 52 | | | —NH₂ | |
| PDC_EphA2-00019437 | 53 | | | —NH₂ | |
| PDC_EphA2-00019438 | 54 | | | —NH₂ | |
| PDC_EphA2-00019439 | 55 | | | —NH₂ | |
| PDC_EphA2-00019440 | 56 | | | —NH₂ | |
| PDC_EphA2-00019441 | 57 | | | —NH₂ | |
| PDC_EphA2-00019442 | 58 | | | —NH₂ | |
| PDC_EphA2-00026424 | 59 | | | —NH₂ | |
| PDC_EphA2-00026425 | 60 | | | —NH₂ | |
| PDC_EphA2-00026426 | 61 | | | —NH₂ | |
| PDC_EphA2-00026521 | 62 | | | —NH₂ | |
| PDC_EphA2-00026522 | 63 | | | —NH₂ | |
| PDC_EphA2-00026523 | 64 | | | —NH₂ | |
| PDC_EphA2-00026524 | 65 | | | —NH₂ | |
| PDC_EphA2-00026525 | 66 | | | —NH₂ | |
| PDC_EphA2-00026526 | 67 | | | —NH₂ | |
| PDC_EphA2-00026527 | 68 | | | —NH₂ | |
| PDC_EphA2-00026528 | 69 | | | —NH₂ | |
| PDC_EphA2-00026529 | 70 | | | —NH₂ | |
| PDC_EphA2-00010011-C003 | 71 | | bA dk | —NH₂ | 180 |
| PDC_EphA2-00010012-C003 | 72 | | bA dk | —NH₂ | 181 |
| PDC_EphA2-00013693-C003 | 73 | | bA dk | —NH₂ | 182 |
| PDC_EphA2-00008082 | 74 | | bA dkAc | —NH₂ | 183 |
| PDC_EphA2-00008083 | 75 | | bA dkAc | —NH₂ | 184 |
| PDC_EphA2-00008084 | 76 | | bA dkAc | —NH₂ | 185 |
| PDC_EphA2-00008085 | 77 | | bA dkAc | —NH₂ | 186 |
| PDC_EphA2-00008086 | 78 | | bA dkAc | —NH₂ | 187 |
| PDC_EphA2-00008087 | 79 | | bA dkAc | —NH₂ | 188 |
| PDC_EphA2-00008088 | 80 | | bA dkAc | —NH₂ | 189 |
| PDC_EphA2-00008089 | 81 | | KAc | —NH₂ | 190 |
| PDC_EphA2-00008090 | 82 | | dkAc | —NH₂ | 191 |
| PDC_EphA2-00008091 | 83 | | bA dkAc | —NH₂ | 192 |
| PDC_EphA2-00008092 | 84 | | bA dkAc | —NH₂ | 193 |
| PDC_EphA2-00008093 | 85 | | bA dkAc | —NH₂ | 194 |
| PDC_EphA2-00008094 | 86 | | bA dkAc | —NH₂ | 195 |
| PDC_EphA2-00008095 | 87 | | bA dkAc | —NH₂ | 196 |
| PDC_EphA2-00007196 | 88 | | bA dkAc | —NH₂ | 197 |
| PDC_EphA2-00008097 | 89 | | bA dkAc | —NH₂ | 198 |
| PDC_EphA2-00008093-C002 | 90 | | dk | —NH₂ | 199 |
| PDC_EphA2_00007196-C002 | 91 | | dk | —NH₂ | 200 |
| PDC_EphA2-00010011-C002 | 92 | | dk | —NH₂ | 201 |
| PDC_EphA2-00010012-C002 | 93 | | dk | —NH₂ | 202 |
| PDC_EphA2-00013693-C002 | 94 | | dk | —NH₂ | 203 |
| PDC_EphA2-00019437-C002 | 95 | | dk | —NH₂ | 204 |
| PDC_EphA2-00022593-C002 | 96 | | dk | —NH₂ | 205 |
| PDC_EphA2-00022594-C002 | 97 | | dk | —NH₂ | 206 |
| PDC_EphA2-00022595-C002 | 98 | | dk | —NH₂ | 207 |
| PDC_EphA2-00019440-C002 | 99 | | dk | —NH₂ | 208 |
| PDC_EphA2-00022612-C002 | 100 | | dk | —NH₂ | 209 |
| PDC_EphA2-00022613-C002 | 101 | | dk | —NH₂ | 210 |
| PDC_EphA2-00022614-C002 | 102 | | dk | —NH₂ | 211 |
| PDC_EphA2-00022615-C002 | 103 | | dk | —NH₂ | 212 |
| PDC_EphA2-00013703-C002 | 104 | | dk | —NH₂ | 213 |
| PDC_EphA2-00013704-C002 | 105 | | dk | —NH₂ | 214 |
| PDC_EphA2-00013705-C002 | 106 | | dk | —NH₂ | 215 |
| PDC_EphA2-00026424-C002 | 107 | | dk | —NH₂ | 216 |
| PDC_EphA2-00026425-C002 | 108 | | dk | —NH₂ | 217 |
| PDC_EphA2-00026426-C002 | 109 | | dk | —NH₂ | 218 |
| PDC_EphA2-00026624 | 110 | | dk | —OH | 219 |
| PDC_EphA2-00026625 | 111 | | dk | —OH | 220 |
| PDC_EphA2-00026626 | 112 | | dk | —OH | 221 |
| PDC_EphA2-00026627 | 113 | | dk | —OH | 222 |
| PDC_EphA2-00001417-C004 | 114 | | bA dk(PEG8c-PEG2c) | —NH₂ | 223 |
| PDC_EphA2_00007196-C004 | 115 | | bA dk(PEG80-PEG2c) | —NH₂ | 224 |
| PDC_EphA2-00008093-C004 | 116 | | bA dk(PEG80-PEG2c) | —NH₂ | 225 |
| PDC_EphA2-00008094-C004 | 117 | | bA dk(PEG8c-PEG2c) | —NH₂ | 226 |
| PDC_EphA2-00010011-C004 | 118 | | bA dk(PEG8c-PEG20) | —NH₂ | 227 |
| PDC_EphA2-00010012-C004 | 119 | | bA dk(PEG8c-PEG20) | —NH₂ | 228 |
| PDC_EphA2-00013693-C004 | 120 | | bA dk(PEG80-PEG2c) | —NH₂ | 229 |
| PDC_EphA2-00008093-C010 | 121 | | dk(bA-MeG-MeG-MeG-MeG- | —NH₂ | 230 |
| | | | MeG-MeG-MeG-MeG-MeG- | | |
| | | | MeG (SEQ ID NO: 279)) | | |
| PDC_EphA2_00007196-C010 | 122 | | Dk(bA-MeG-MeG-MeG-MeG- | —NH₂ | 231 |
| | | | MeG-MeG-MeG-MeG-MeG- | | |
| | | | MeG (SEQ ID NO: 279)) | | |
| PDC_EphA2-00022417 | 123 | | | —NH₂ | |
| PDC_EphA2-00026600 | 124 | | | —NH₂ | |
| PDC_EphA2-00026601 | 125 | | | —NH₂ | |
| PDC_EphA2-00026602 | 126 | | | —NH₂ | |
| PDC_EphA2-00026603 | 127 | | | —NH₂ | |
| PDC_EphA2-00026604 | 128 | | | —NH₂ | |
| PDC_EphA2-00026605 | 129 | | | —NH₂ | |
| PDC_EphA2-00026606 | 130 | | | —NH₂ | |
| PDC_EphA2-00026607 | 131 | | | —NH₂ | |
| PDC_EphA2-00026608 | 132 | | | —NH₂ | |
| PDC_EphA2-00026609 | 133 | | | —NH₂ | |
| PDC_EphA2-00026610 | 134 | | | —NH₂ | |
| PDC_EphA2-00026611 | 135 | | | —NH₂ | |
| PDC_EphA2-00022606 | 136 | | G | —NH₂ | 232 |
| PDC_EphA2-00022607 | 137 | | KAc | —NH₂ | 233 |
| PDC_EphA2-00022608 | 138 | | dkAc | —NH₂ | 234 |
| PDC_EphA2-00021407-L092 | 139 | | G | —NH₂ | 235 |
| PDC_EphA2-00022057-L092 | 140 | | G | —NH₂ | 236 |
| PDC_EphA2-00022417-C002 | 141 | | dk | —NH₂ | 237 |
| PDC_EphA2-00026600-C001 | 142 | | K | —NH₂ | 238 |
| PDC_EphA2-00026601-C001 | 143 | | K | —NH₂ | 239 |
| PDC_EphA2-00026602-C001 | 144 | | K | —NH₂ | 240 |
| PDC_EphA2-00026603-C001 | 145 | | K | —NH₂ | 241 |
| PDC_EphA2-00026604-C001 | 146 | | K | —NH₂ | 242 |
| PDC_EphA2-00026606-C001 | 147 | | K | —NH₂ | 243 |
| PDC_EphA2-00026607-C001 | 148 | | K | —NH₂ | 244 |
| PDC_EphA2-00026608-C001 | 149 | | K | —NH₂ | 245 |
| PDC_EphA2-00020708 | 150 | | | —NH₂ | |
| PDC_EphA2-00026479 | 151 | | | —NH₂ | |
| PDC_EphA2-00026481 | 152 | | | —NH₂ | |
| PDC_EphA2-00022601 | 153 | | G | —NH₂ | 246 |
| PDC_EphA2-00022602 | 154 | | KAc | —NH₂ | 247 |
| PDC_EphA2-00022603 | 155 | | dkAc | —NH₂ | 248 |
| PDC_EphA2-00020295-L092 | 156 | | G | —NH₂ | 249 |
| PDC_EphA2-00020708-C002 | 157 | | dk | —NH₂ | 250 |
| PDC_EphA2-00019498-L092 | 158 | | G | —NH₂ | 251 |
| PDC_EphA2-00027094-C002 | 159 | | dk | —NH₂ | 252 |
| PDC_EphA2-00027095-C002 | 160 | | dk | —NH₂ | 253 |
| PDC_EphA2-00027090 | 161 | de | | —OH | 254 |
| PDC_EphA2-00027091 | 162 | de | | —OH | 255 |
| PDC_EphA2-00027092 | 163 | de | | —NH₂ | 256 |
| PDC_EphA2-00027106 | 164 | de | | —NH₂ | 257 |
| PDC_EphA2-0007196-C008 | 88 | | dk df | —NH₂ | 258 |
| PDC_EphA2-0007196-C009 | 88 | | dk(G-df) | —NH₂ | 259 |
| PDC_EphA2-0027128 | 165 | | de | —NH₂ | 260 |
| PDC_EphA2-0027129 | 165 | | df | —NH₂ | 261 |
| PDC_EphA2-0027131 | 165 | | tma | —NH₂ | 262 |
| PDC_EphA2-0027126 | 166 | de | de | —NH₂ | 263 |
| PDC_EphA2-0027127 | 166 | de | df | —NH₂ | 264 |
| PDC_EphA2-0027130 | 166 | de | tma | —NH₂ | 265 |
| PDC_EphA2-0007199-C002 | 167 | | dk | —NH₂ | 266 |
| PDC_EphA2-0008010-C002 | 168 | | dk | —NH₂ | 267 |
| PDC_EphA2-0007180-C002 | 169 | | dk | —NH₂ | 268 |
| PDC_EphA2-0007198-C002 | 170 | | dk | —NH₂ | 269 |
| PDC_EphA2-0019443-C002 | 171 | | dk | —NH₂ | 270 |
| PDC_EphA2-0019444-C002 | 171 | | dk | —OH | 271 |

Note that in Tables 6A and 6B above, each SEQ ID NO includes the corresponding peptide sequence in Table 6A, and any applicable amino acid sequence in the linker (if any).

### Example 6: PDC_EphA2-00026626 (SEQ ID NO: 221)

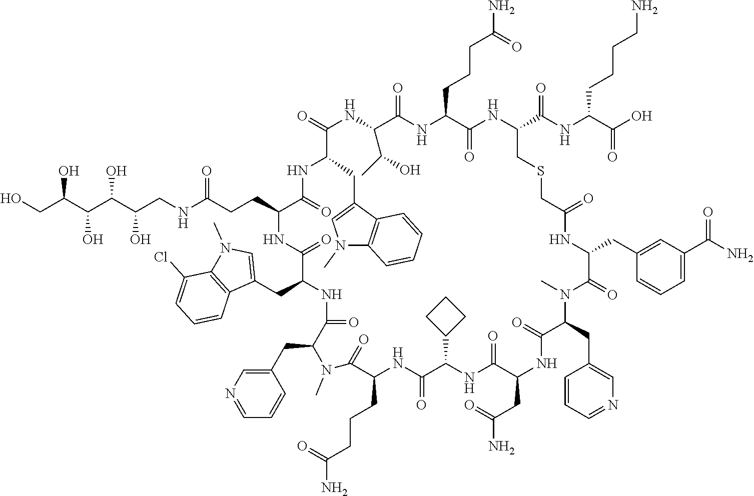 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@H](CCCCN)C(=O)O)NC(=O)[C@H](CCCC(N)=O)NC1=O]

The peptide sequence was synthesized on Fmoc-dk(Boc)-Alko Resin (0.7 mmol/g, 0.250 mmol) following general peptide synthesis method A. The obtained peptide on the resin was subjected to the general method CIAc-4. The polypeptide on the resin was treated with Cleavage Cocktail-A (15 mL) to furnish the linear peptide. The crude containing the linear peptide (0.250 mmol as theoretical based on the resin used) was subjected to the peptide cyclization condition-2. The resulting residue was purified by preparative reversed-phase HPLC (Method P-A). Pure fractions were combined and lyophilized to afford the title macrocyclic peptide. HPLC-MS (Method A-A): 5.32 min, ESI-MS m/z: 1071.8 [M+2H]2+, AUC (UV 225 nm): 93%.

The following Examples were prepared by following similar methods described in the examples above. The amino acid sequence for each peptide was shown in Table 6A.

TABLE 7

| Example | | |
|---|---|---|
| No. | Structure | Analytics |
| PDC_EphA2- 00001417 (SEQ ID NO: 1) | 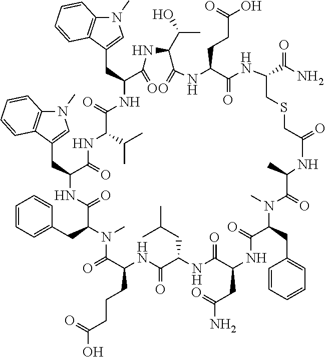 [SMILES: CC(C)C[C@@H]1NC(=O)[C@H](CC(N)=O)NC(=O)[C@H](Cc2ccccc2)N(C)C(=O)[C@@H](C)NC(=O)CSC[C@@H](C(N)=O)NC(=O)[C@H](CCC(=O)O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3ccccc23)NC(=O)[C@H](Cc2ccccc2)N(C)C(=O)[C@H](CCCC(=O)O)NC1=O] | HPLC-MS (Method A-C): 3.56 min ESI-MS m/z: 827.7 [M + 2H]2+ |
| PDC_EphA2- 00008082 (SEQ ID NO: 183) | 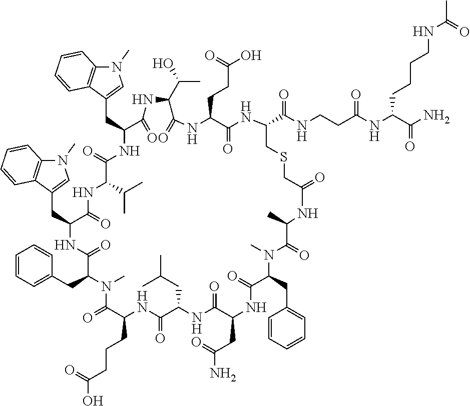 [SMILES: CC(=O)NCCCC[C@@H](NC(=O)CCNC(=O)[C@@H]1CSCC(=O)N[C@H](C)C(=O)N(C)[C@@H](Cc2ccccc2)C(=O)N[C@@H](CC(N)=O)C(=O)N[C@@H](CC(C)C)C(=O)N[C@@H](CCCC(=O)O)C(=O)N(C)[C@@H](Cc2ccccc2)C(=O)N[C@@H](Cc2cn(C)c3ccccc23)C(=O)N[C@@H](C(C)C)C(=O)N[C@@H](Cc2cn(C)c3ccccc23)C(=O)N[C@@H]([C@@H](C)O)C(=O)N[C@@H](CCC(=O)O)C(=O)N1)C(N)=O] | HPLC-MS (Method A-E): 3.14 min ESI-MS m/z: 948.4 [M + 2H]2+ |
| PDC_EphA2- 00008083 (SEQ ID NO: 184) | 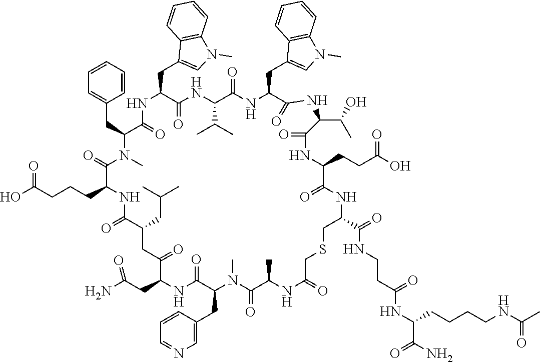 | HPLC-MS (Method A-B): 4.98 min ESI-MS m/z: 949.2 [M + 2H]2+ |
| PDC_EphA2- 00008084 (SEQ ID NO: 185) | 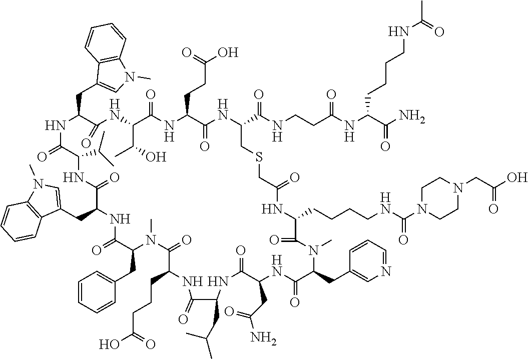 | HPLC-MS (Method A-B): 4.64 min ESI-MS m/z: 1062.8 [M + 2H]2+ |
| PDC_EphA2- 00008085 (SEQ ID NO: 186) | 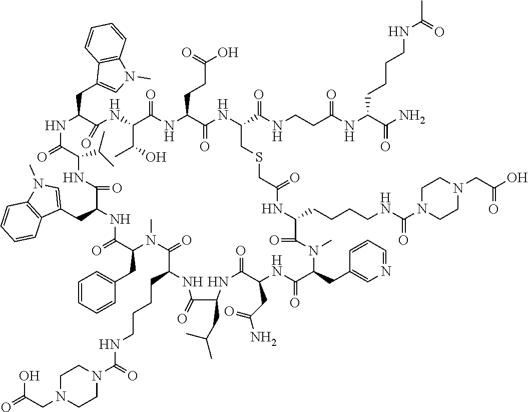 | HPLC-MS (Method A-B): 4.16 min ESI-MS m/z: 1140.3 [M + 2H]2+ |
| PDC_EphA2- 00008086 (SEQ ID NO: 187) | 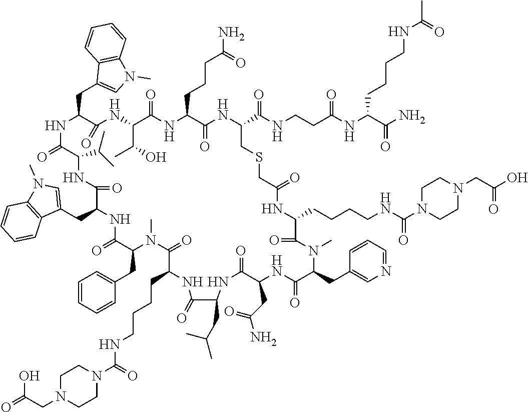 [SMILES: CC(=O)NCCCC[C@@H](NC(=O)CCNC(=O)[C@@H]1CSCC(=O)N[C@H](CCCCNC(=O)N2CCN(CC(=O)O)CC2)C(=O)N(C)[C@@H](Cc2cccnc2)C(=O)N[C@@H](CC(N)=O)C(=O)N[C@@H](CC(C)C)C(=O)N[C@@H](CCCCNC(=O)N2CCN(CC(=O)O)CC2)C(=O)N(C)[C@@H](Cc2ccccc2)C(=O)N[C@@H](Cc2cn(C)c3ccccc23)C(=O)N[C@@H](C(C)C)C(=O)N[C@@H](Cc2cn(C)c3ccccc23)C(=O)N[C@@H]([C@@H](C)O)C(=O)N[C@@H](CCCC(N)=O)C(=O)N1)C(N)=O] | HPLC-MS (Method A-B): 4.43 min ESI-MS m/z: 1146.7 [M + 2H]2+ |
| PDC_EphA2- 00008087 (SEQ ID NO: 188) | 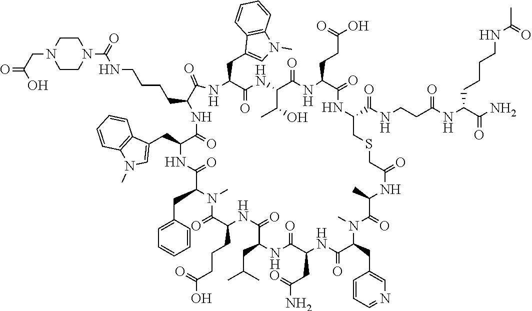 [SMILES: CC(=O)NCCCC[C@@H](NC(=O)CCNC(=O)[C@@H]1CSCC(=O)N[C@H](C)C(=O)N(C)[C@@H](Cc2cccnc2)C(=O)N[C@@H](CC(N)=O)C(=O)N[C@@H](CC(C)C)C(=O)N[C@@H](CCCC(=O)O)C(=O)N(C)[C@@H](Cc2ccccc2)C(=O)N[C@@H](Cc2cn(C)c3ccccc23)C(=O)N[C@@H](CCCCNC(=O)N2CCN(CC(=O)O)CC2)C(=O)N[C@@H](Cc2cn(C)c3ccccc23)C(=O)N[C@@H]([C@@H](C)O)C(=O)N[C@@H](CCC(=O)O)C(=O)N1)C(N)=O] | HPLC-MS (Method A-B): 4.33 min ESI-MS m/z: 1048.6 [M + 2H]2+ |
| PDC_EphA2- 00008088 (SEQ ID NO: 189) | 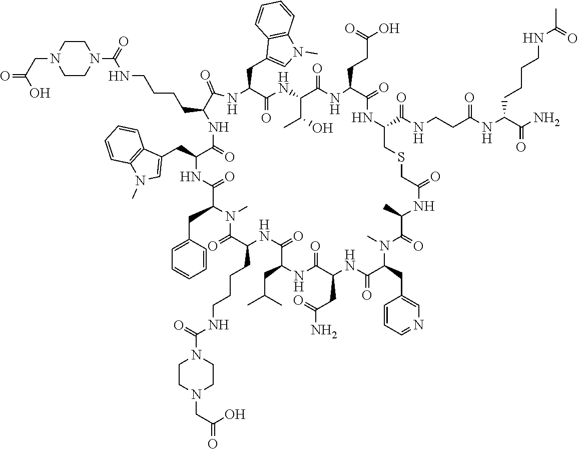 | HPLC-MS (Method A-B): 3.87 min ESI-MS m/z: 751.2 [M + 3H]3+ |
| PDC_EphA2- 00008089 (SEQ ID NO: 190) | 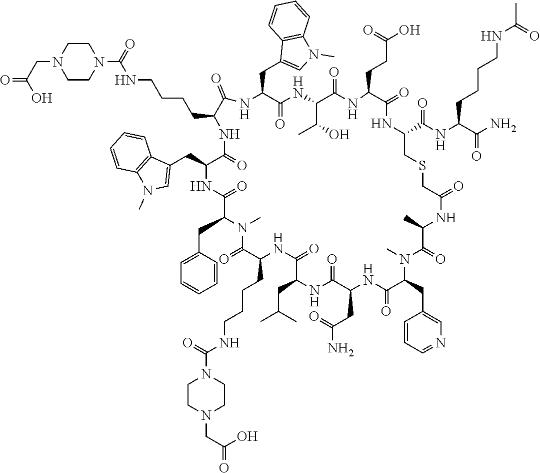 | HPLC-MS (Method A-B): 3.45 min ESI-MS m/z: 757.5 [M + 3H]3+ |
| PDC_EphA2- 00008090 (SEQ ID NO: 191) | 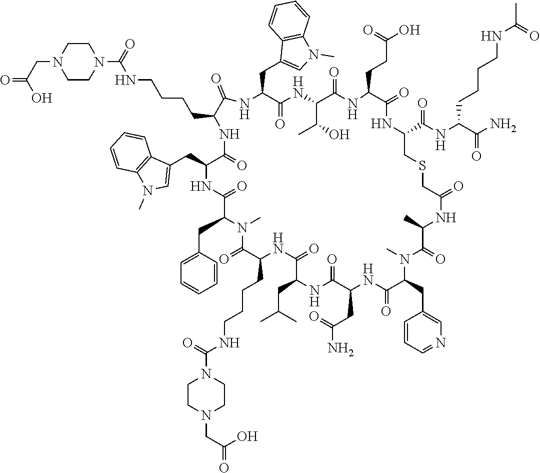 | HPLC-MS (Method A-B): 3.91 min ESI-MS m/z: 727.5 [M + 3H]3+ |
| PDC_EphA2- 00008091 (SEQ ID NO: 192) | 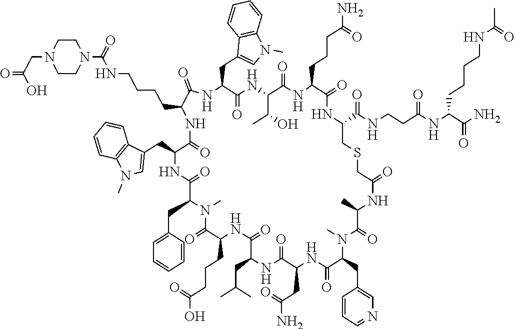 | HPLC-MS (Method A-B): 3.91 min ESI-MS m/z: 704.0 [M + 3H]3+ |
| PDC_EphA2- 00008092 (SEQ ID NO: 193) | 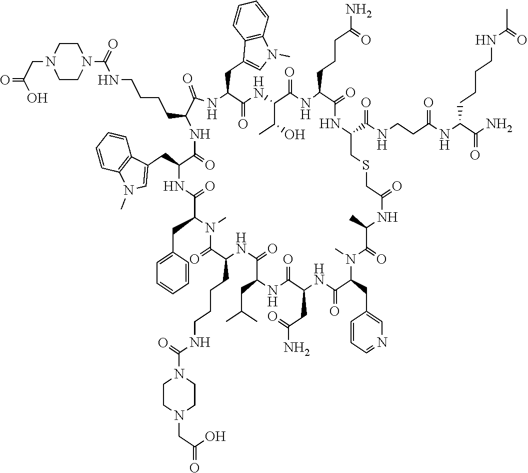 | HPLC-MS (Method A-B): 3.42 min ESI-MS m/z: 755.6 [M + 3H]3+ |
| PDC_EphA2- 00008093 (SEQ ID NO: 194) | 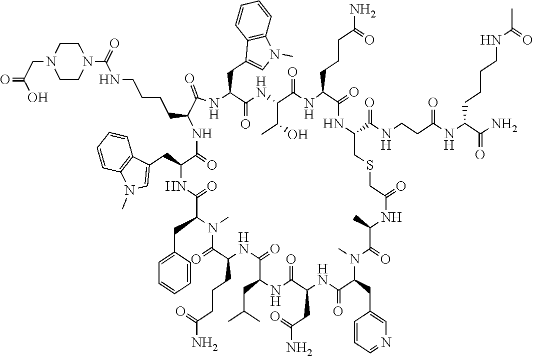 | HPLC-MS (Method A-B): 3.72 min ESI-MS m/z: 703.6 [M + 3H]3+ |
| PDC_EphA2- 00008094 (SEQ ID NO: 195) | 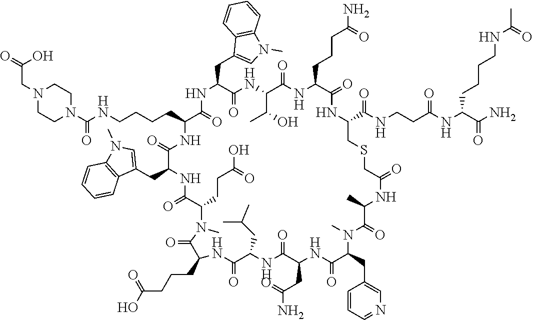 | HPLC-MS (Method A-B): 3.11 min ESI-MS m/z: 697.9 [M + 3H]3+ |
| PDC_EphA2- 00008095 (SEQ ID NO: 196) | 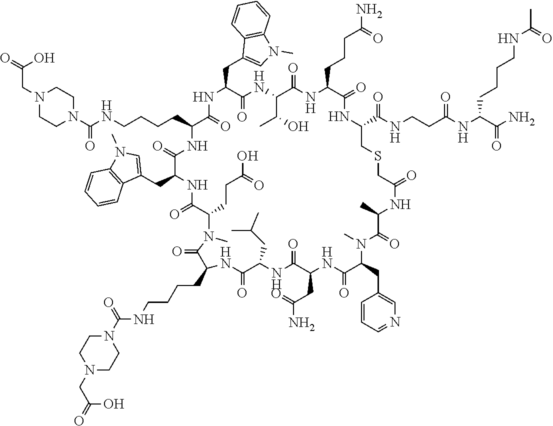 | HPLC-MS (Method A-A): 5.43 min ESI-MS m/z: 749.6 [M + 3H]3+ |
| PDC_EphA2- 00007196 (SEQ ID NO: 197) | 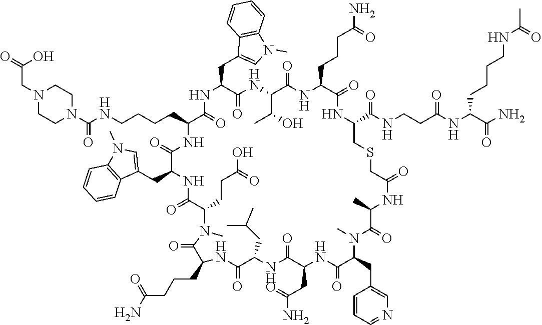 | HPLC-MS (Method A-A): 5.66 min ESI-MS m/z: 697.6 [M + 3H]3+ |
| PDC_EphA2- 00008097 (SEQ ID NO: 198) | 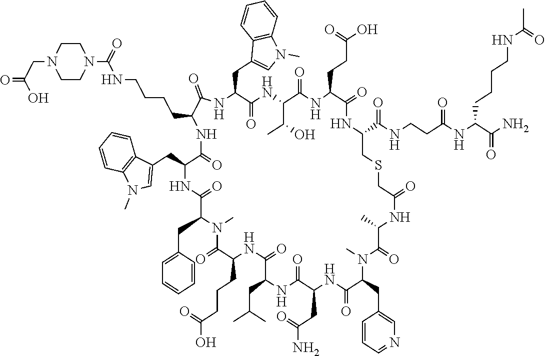 | HPLC-MS (Method A-A): 3.96 min ESI-MS m/z: 699.7 [M + 3H]3+ |
| PDC_EphA2- 00009998 (SEQ ID NO: 2) | 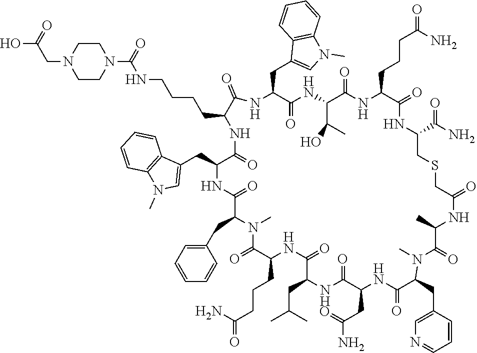 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC1=O] | HPLC-MS (Method A-B): 3.82 min ESI-MS m/z: 934.0 [M + 2H]2+ |
| PDC_EphA2- 00009999 (SEQ ID NO: 3) | 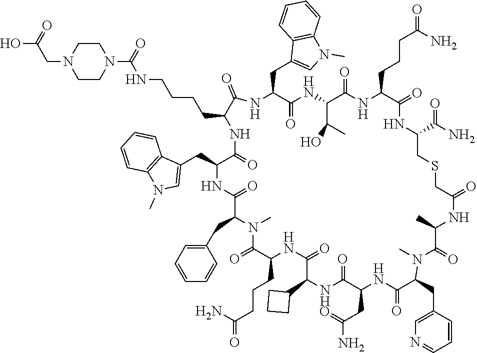 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-B): 3.72 min ESI-MS m/z: 933.0 [M + 2H]2+ |
| PDC_EphA2- 00010000 (SEQ ID NO: 4) | 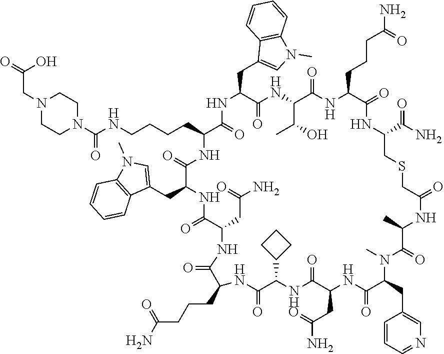 | HPLC-MS (Method A-A): 5.02 min ESI-MS m/z: 909.7 [M + 2H]2+ |
| PDC_EphA2- 00010001 (SEQ ID NO: 5) | 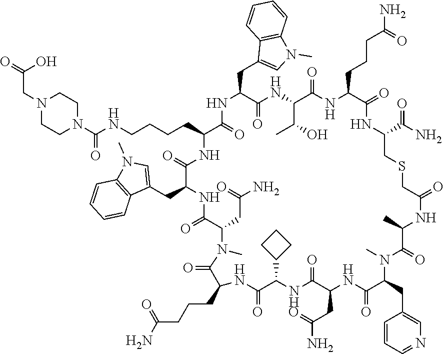 | HPLC-MS (Method A-A): 5.20 min ESI-MS m/z: 916.7 [M + 2H]2+ |
| PDC_EphA2- 00010002 (SEQ ID NO: 6) | 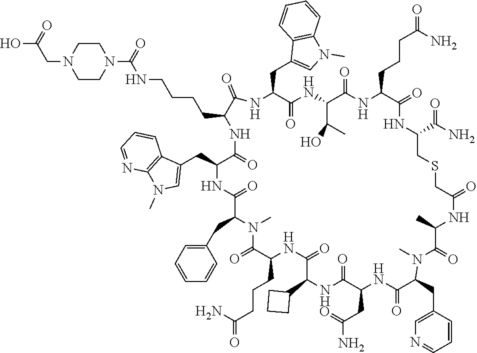 | HPLC-MS (Method A-A): 5.16 min ESI-MS m/z: 933.8 [M + 2H]2+ |
| PDC_EphA2- 00010003 (SEQ ID NO: 7) | 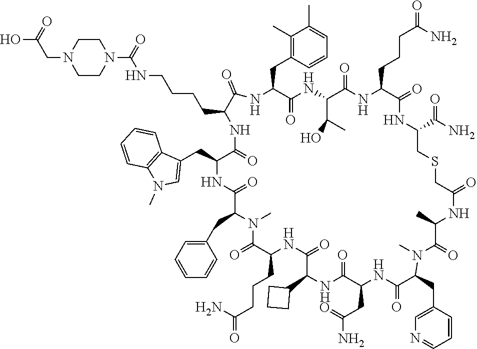 [SMILES: Cc1cccc(C[C@@H]2NC(=O)[C@H](CCCCNC(=O)N3CCN(CC(=O)O)CC3)NC(=O)[C@H](Cc3cn(C)c4ccccc34)NC(=O)[C@H](Cc3ccccc3)N(C)C(=O)[C@H](CCCC(N)=O)NC(=O)[C@H](C3CCC3)NC(=O)[C@H](CC(N)=O)NC(=O)[C@H](Cc3cccnc3)N(C)C(=O)[C@@H](C)NC(=O)CSC[C@@H](C(N)=O)NC(=O)[C@H](CCCC(N)=O)NC(=O)[C@H]([C@@H](C)O)NC2=O)c1C] | HPLC-MS (Method A-B): 3.75 min ESI-MS m/z: 920.5 [M + 2H]2+ |
| PDC_EphA2- 00010004 (SEQ ID NO: 8) | 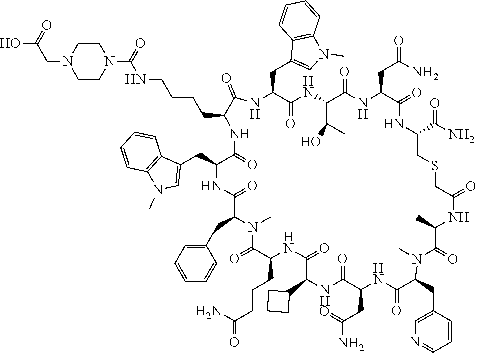 [SMILES: C[C@H]1NC(=O)CSC[C@@H](C(N)=O)NC(=O)[C@H](CC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-B): 3.71 min ESI-MS m/z: 919.0 [M + 2H]2+ |
| PDC_EphA2- 00010005 (SEQ ID NO: 9) | 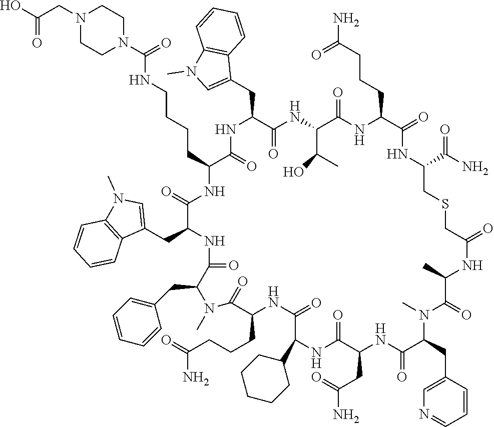 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCCCC2)NC(=O)[C@H](CC(N)=O)NC(=O)[C@H](Cc2cccnc2)N(C)C1=O] | HPLC-MS (Method A-B): 4.09 min ESI-MS m/z: 947.0 [M + 2H]2+ |
| PDC_EphA2- 00010006 (SEQ ID NO: 10) | 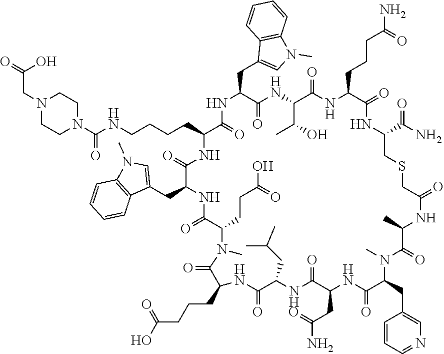 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(=O)O)NC1=O] | HPLC-MS (Method A-B): 3.08 min ESI-MS m/z: 925.4 [M + 2H]2+ |
| PDC_EphA2- 00010007 (SEQ ID NO: 11) | 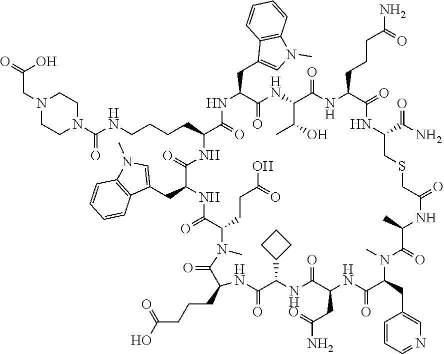 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(=O)O)NC(=O)[C@H](C2CCC2)NC(=O)[C@H](CC(N)=O)NC(=O)[C@H](Cc2cccnc2)N(C)C1=O] | HPLC-MS (Method A-B): 2.94 min ESI-MS m/z: 924.4 [M + 2H]2+ |
| PDC_EphA2- 00010008 (SEQ ID NO: 12) | 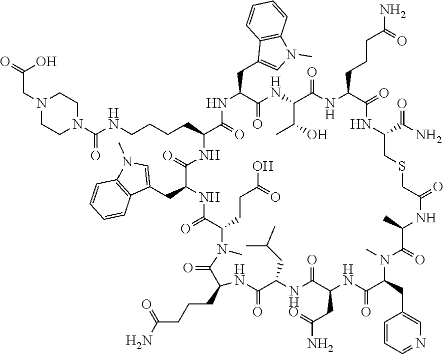 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O] | HPLC-MS (Method A-B): 2.95 min ESI-MS m/z: 924.9 [M + 2H]2+ |
| PDC_EphA2- 00010009 (SEQ ID NO: 13) | 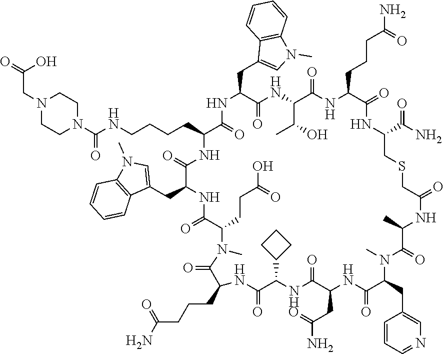 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-A): 5.54 min ESI-MS m/z: 923.9 [M + 2H]2+ |
| PDC_EphA2- 00010010 (SEQ ID NO: 14) | 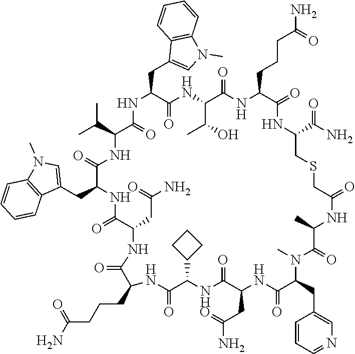 | HPLC-MS (Method A-A): 3.28 min ESI-MS m/z: 810.1 [M + 2H]2+ |
| PDC_EphA2- 00010011 (SEQ ID NO: 15) | 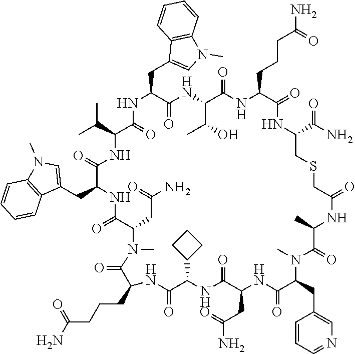 | HPLC-MS (Method A-B): 3.24 min ESI-MS m/z: 817.2 [M + 2H]2+ |
| PDC_EphA2- 00010012 (SEQ ID NO: 16) | 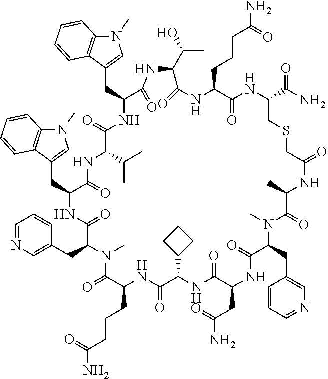 [SMILES: CC(C)[C@@H]1NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-A): 5.56 min ESI-MS m/z: 834.1 [M + 2H]2+ |
| PDC_EphA2- 00013693 (SEQ ID NO: 17) | 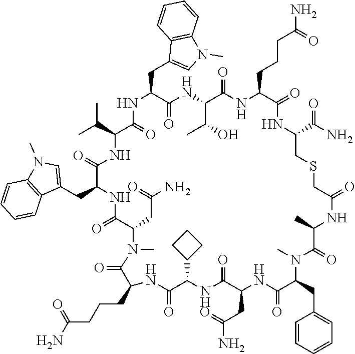 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 5.34 min ESI-MS m/z: 816.4 [M + 2H]2+ |
| PDC_EphA2- 00013703 (SEQ ID NO: 18) | 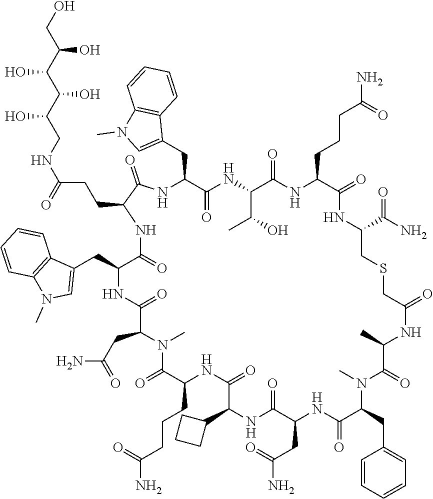 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 4.66 min ESI-MS m/z: 912.7 [M + 2H]2+ |
| PDC_EphA2- 00013704 (SEQ ID NO: 19) | 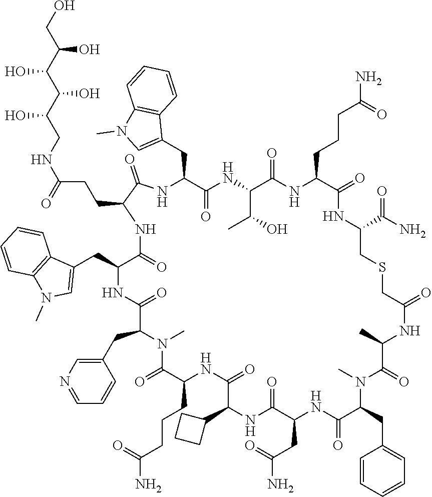 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 4.08 min ESI-MS m/z: 929.9 [M + 2H]2+ |
| PDC_EphA2- 00013705 (SEQ ID NO: 20) | 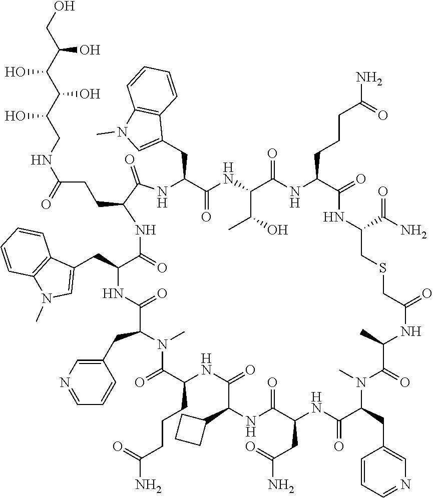 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-A): 5.04 min ESI-MS m/z: 930.4 [M + 2H]2+ |
| PDC_EphA2- 00013917 (SEQ ID NO: 21) | 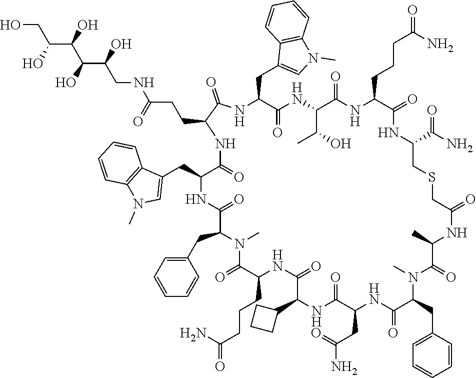 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 5.46 min ESI-MS m/z: 929.3 [M + 2H]2+ |
| PDC_EphA2- 00018526 (SEQ ID NO: 22) | 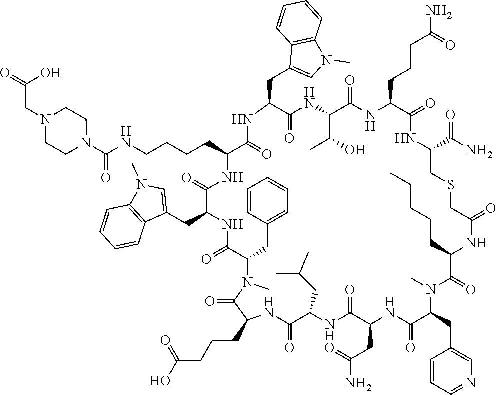 | HPLC-MS (Method A-B): 4.39 min ESI-MS m/z: 962.8 [M + 2H]2+ |
| PDC_EphA2- 00018527 (SEQ ID NO: 23) | 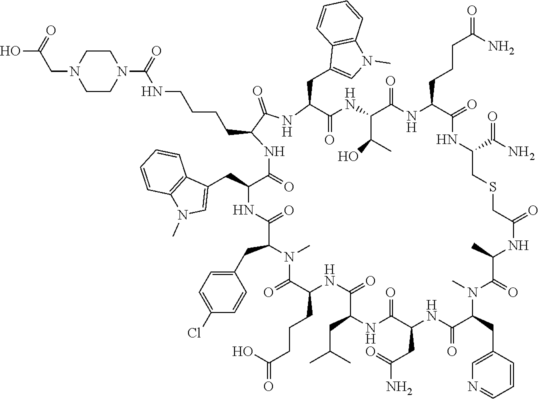 | HPLC-MS (Method A-B): 4.22 min ESI-MS m/z: 952.0 [M + 2H]2+ |
| PDC_EphA2- 00018528 (SEQ ID NO: 24) | 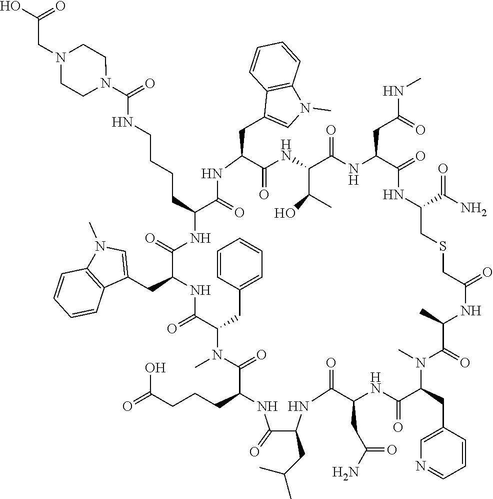 | HPLC-MS (Method A-B): 3.98 min ESI-MS m/z: 927.8 [M + 2H]2+ |
| PDC_EphA2- 00018529 (SEQ ID NO: 25) | 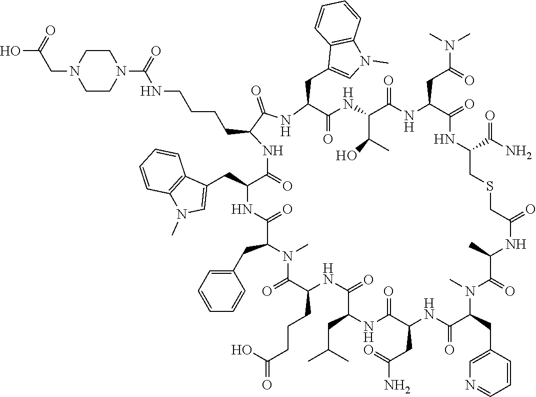 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CC(=O)N(C)C)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(=O)O)NC1=O] | HPLC-MS (Method A-B): 4.13 min ESI-MS m/z: 934.4 [M + 2H]2+ |
| PDC_EphA2- 00018530 (SEQ ID NO: 26) | 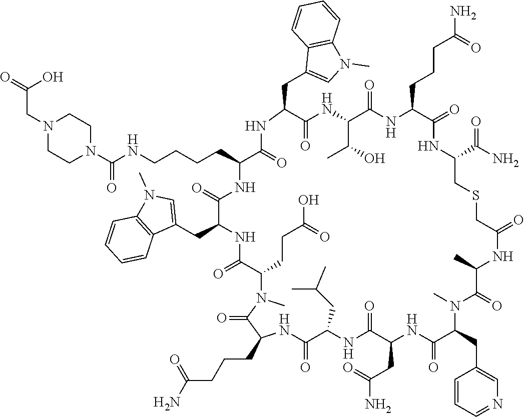 | HPLC-MS (Method A-B): 3.65 min ESI-MS m/z: 952.9 [M + 2H]2+ |
| PDC_EphA2- 00018531 (SEQ ID NO: 27) | 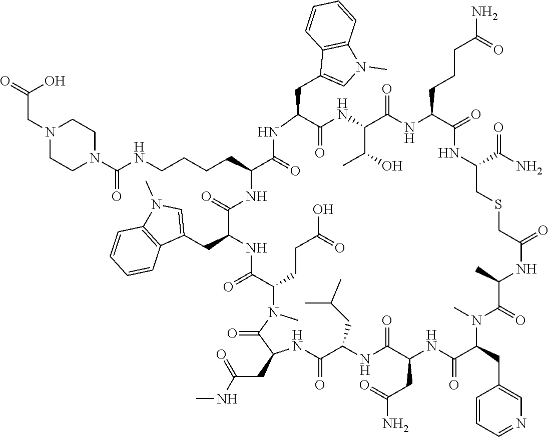 [SMILES: CNC(=O)C[C@@H]1NC(=O)[C@H](CC(C)C)NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C1=O] | HPLC-MS (Method A-B): 3.06 min ESI-MS m/z: 917.8 [M + 2H]2+ |
| PDC_EphA2- 00018532 (SEQ ID NO: 28) | 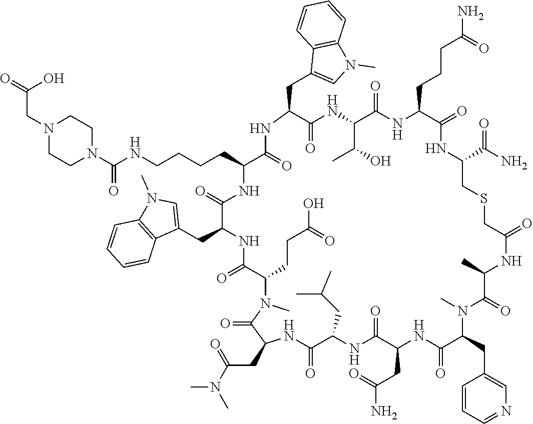 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CC(=O)N(C)C)NC1=O] | HPLC-MS (Method A-B): 3.30 min ESI-MS m/z: 924.9 [M + 2H]2+ |
| PDC_EphA2- 00018533 (SEQ ID NO: 29) | 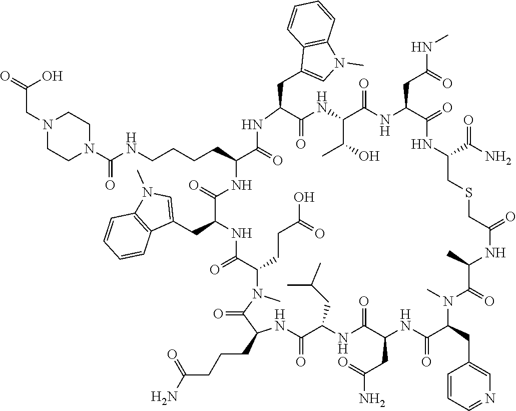 [SMILES: CNC(=O)C[C@@H]1NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC(=O)[C@H](CC(C)C)NC(=O)[C@H](CC(N)=O)NC(=O)[C@H](Cc2cccnc2)N(C)C(=O)[C@@H](C)NC(=O)CSC[C@@H](C(N)=O)NC1=O] | HPLC-MS (Method A-B): 3.09 min ESI-MS m/z: 917.8 [M + 2H]2+ |
| PDC_EphA2- 00018534 (SEQ ID NO: 30) | 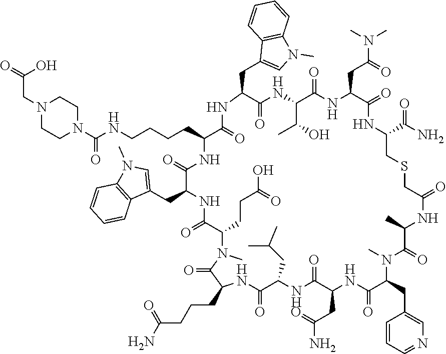 | HPLC-MS (Method A-B): 3.10 min ESI-MS m/z: 925.3 [M + 2H]2+ |
| PDC_EphA2- 00018535 (SEQ ID NO: 31) | 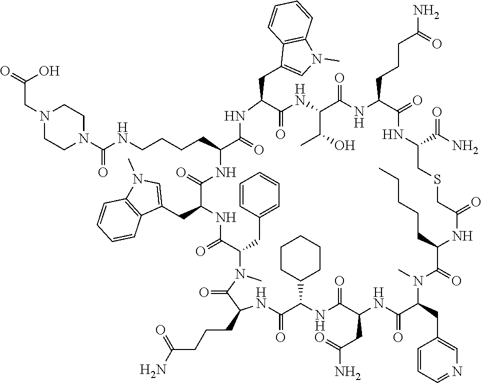 | HPLC-MS (Method A-B): 4.51 min ESI-MS m/z: 975.3 [M + 2H]2+ |
| PDC_EphA2- 00018536 (SEQ ID NO: 32) | 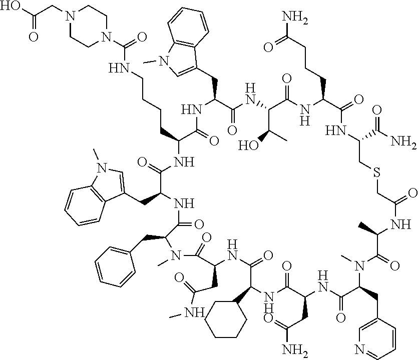 | HPLC-MS (Method A-B): 4.14 min ESI-MS m/z: 940.2 [M + 2H]2+ |
| PDC_EphA2- 00018537 (SEQ ID NO: 33) | 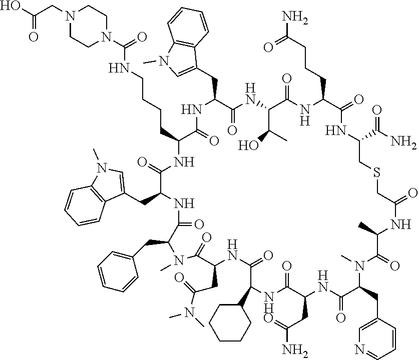 | HPLC-MS (Method A-B): 4.36 min ESI-MS m/z: 947.3 [M + 2H]2+ |
| PDC_EphA2- 00018538 (SEQ ID NO: 34) | 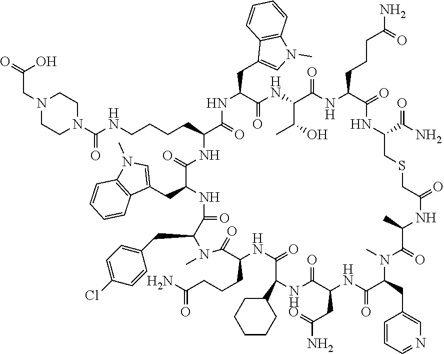 | HPLC-MS (Method A-B): 4.25 min ESI-MS m/z: 964.5 [M + 2H]2+ |
| PDC_EphA2- 00018539 (SEQ ID NO: 35) | 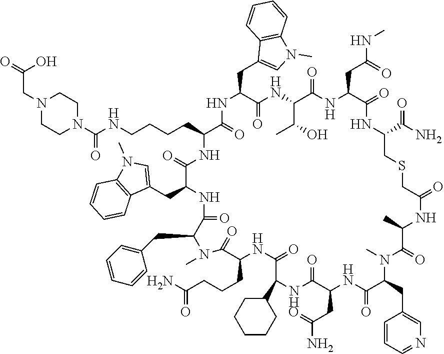 | HPLC-MS (Method A-B): 4.04 min ESI-MS m/z: 940.2 [M + 2H]2+ |
| PDC_EphA2- 00018540 (SEQ ID NO: 36) | 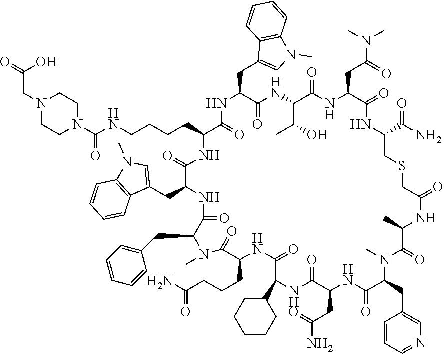 | HPLC-MS (Method A-B): 4.14 min ESI-MS m/z: 947.3 [M + 2H]2+ |
| PDC_EphA2- 00019421 (SEQ ID NO: 37) | 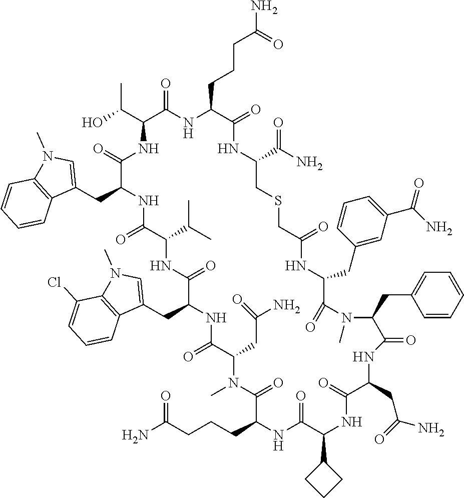 [SMILES: CC(C)[C@@H]1NC(=O)[C@H](Cc2cn(C)c3c(Cl)cccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 5.91 min ESI-MS m/z: 893.0 [M + 2H]2+ |
| PDC_EphA2- 00019422 (SEQ ID NO: 38) | 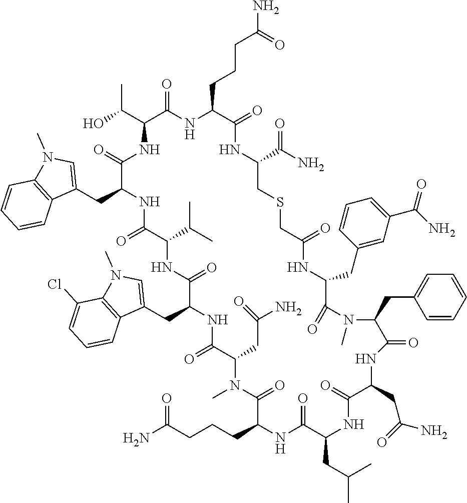 [SMILES: CC(C)C[C@@H]1NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3c(Cl)cccc23)NC(=O)[C@H](CC(N)=O)N(C)C(=O)[C@H](CCCC(N)=O)NC1=O] | HPLC-MS (Method A-B): 6.00 min ESI-MS m/z: 894.1 [M + 2H]2+ |
| PDC_EphA2- 00019423 (SEQ ID NO: 39) | 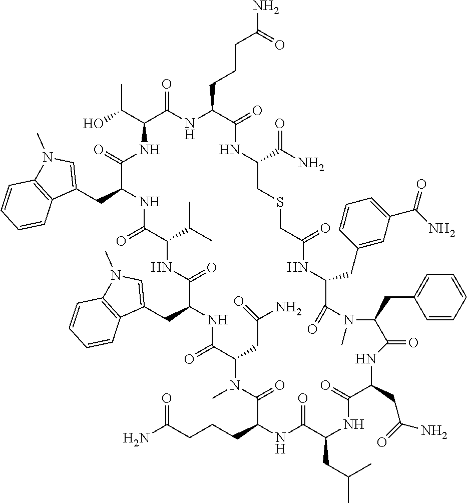 [SMILES: CC(C)C[C@@H]1NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3ccccc23)NC(=O)[C@H](CC(N)=O)N(C)C(=O)[C@H](CCCC(N)=O)NC1=O] | HPLC-MS (Method A-B): 5.46 min ESI-MS m/z: 876.9 [M + 2H]2+ |
| PDC_EphA2- 00019424 (SEQ ID NO: 40) | 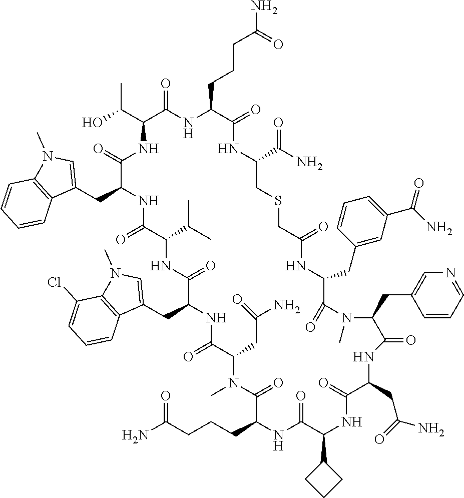 | HPLC-MS (Method A-B): 3.93 min ESI-MS m/z: 893.6 [M + 2H]2+ |
| PDC_EphA2- 00019425 (SEQ ID NO: 41) | 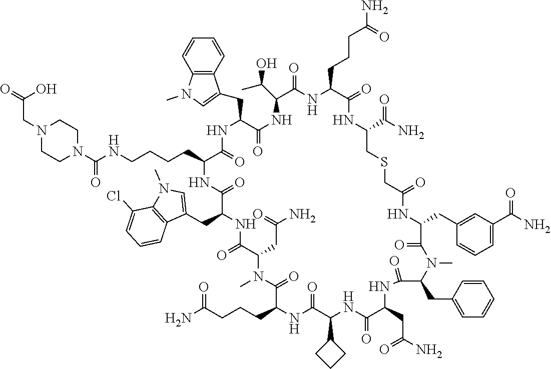 | HPLC-MS (Method A-B): 4.96 min ESI-MS m/z: 992.8 [M + 2H]2+ |
| PDC_EphA2- 00019426 (SEQ ID NO: 42) | 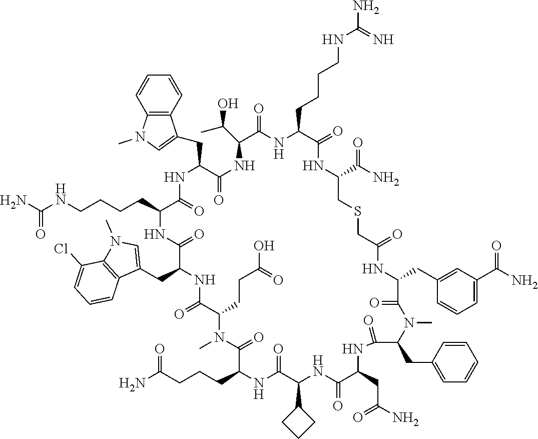 | HPLC-MS (Method A-B): 5.52 min ESI-MS m/z: 929.1 [M + 2H]2+ |
| PDC_EphA2- 00019427 (SEQ ID NO: 43) | 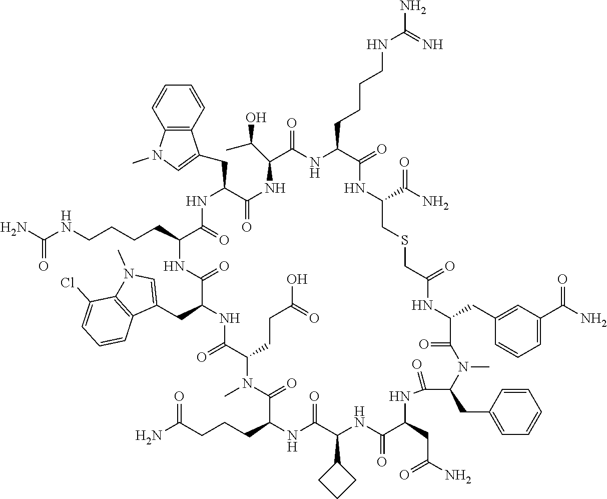 [SMILES: C[C@@H](O)[C@@H]1NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](CCC(=O)O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCCNC(=N)N)NC1=O] | HPLC-MS (Method A-B): 4.89 min ESI-MS m/z: 950.6 [M + 2H]2+ |
| PDC_EphA2- 00019428 (SEQ ID NO: 44) | 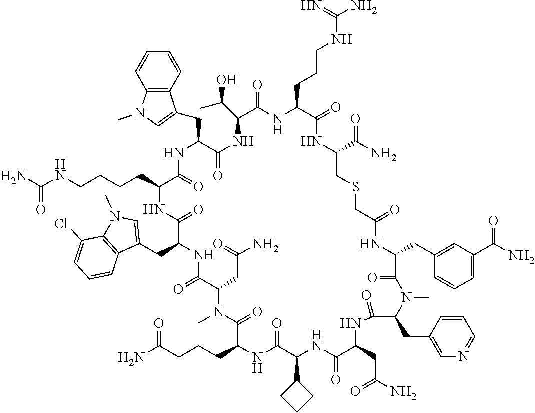 [SMILES: C[C@@H](O)[C@@H]1NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCNC(=N)N)NC1=O] | HPLC-MS (Method A-B): 3.17 min ESI-MS m/z: 936.6 [M + 2H]2+ |
| PDC_EphA2- 00019429 (SEQ ID NO: 45) | 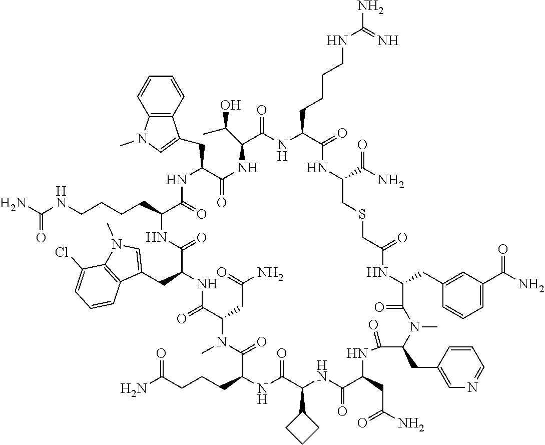 | HPLC-MS (Method A-B): 3.18 min ESI-MS m/z: 943.7 [M + 2H]2+ |
| PDC_EphA2- 00019430 (SEQ ID NO: 46) | 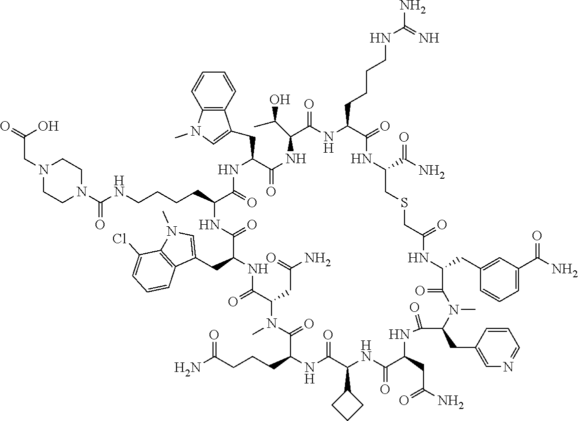 | HPLC-MS (Method A-B): 2.83 min ESI-MS m/z: 1007.2 [M + 2H]2+ |
| PDC_EphA2- 00019431 (SEQ ID NO: 47) | 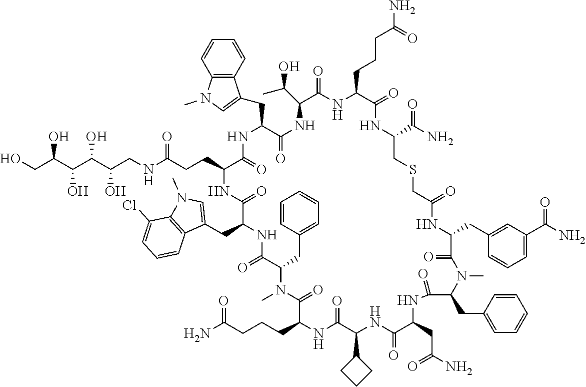 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 6.04 min ESI-MS m/z: 1006.1 [M + 2H]2+ |
| PDC_EphA2- 00019432 (SEQ ID NO: 48) | 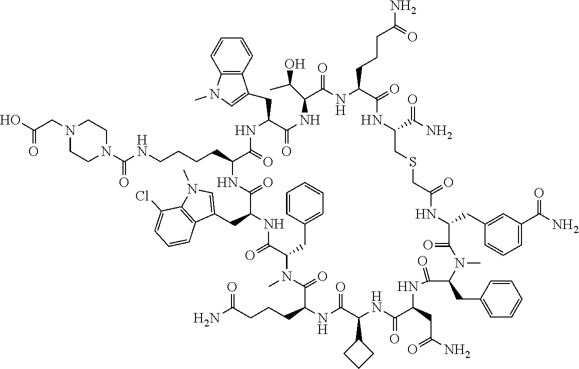 [SMILES: C[C@@H](O)[C@@H]1NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 5.74 min ESI-MS m/z: 1009.1 [M + 2H]2+ |
| PDC_EphA2- 00019433 (SEQ ID NO: 49) | 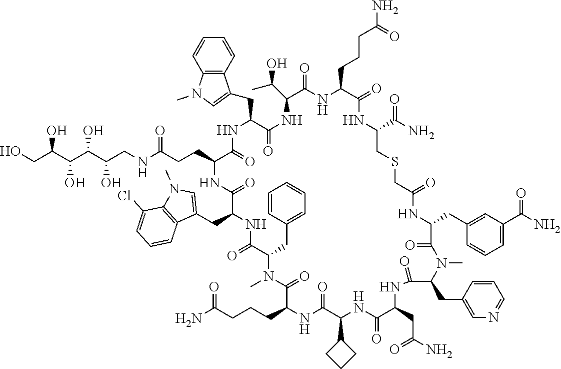 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 4.17 min ESI-MS m/z: 1006.6 [M + 2H]2+ |
| PDC_EphA2- 00019434 (SEQ ID NO: 50) | 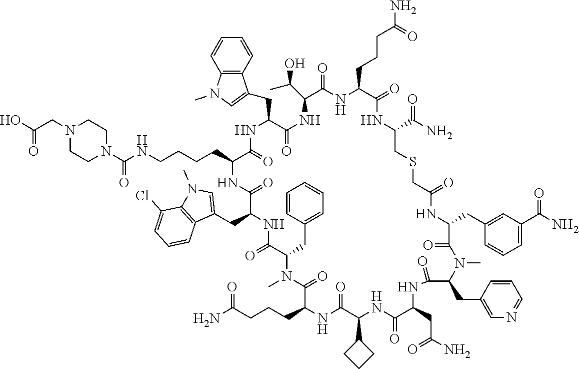 [SMILES: C[C@@H](O)[C@@H]1NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 4.10 min ESI-MS m/z: 1009.6 [M + 2H]2+ |
| PDC_EphA2- 00019435 (SEQ ID NO: 51) | 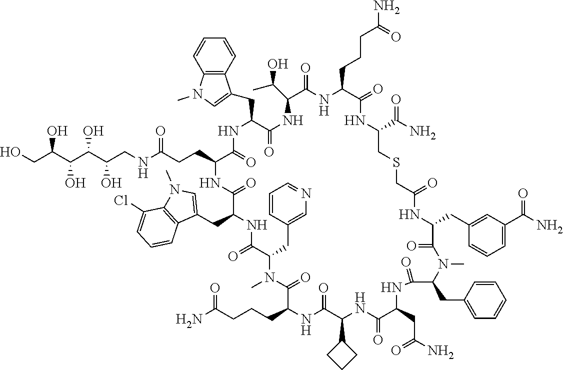 | HPLC-MS (Method A-B): 4.76 min ESI-MS m/z: 1006.7 [M + 2H]2+ |
| PDC_EphA2- 00019436 (SEQ ID NO: 52) | 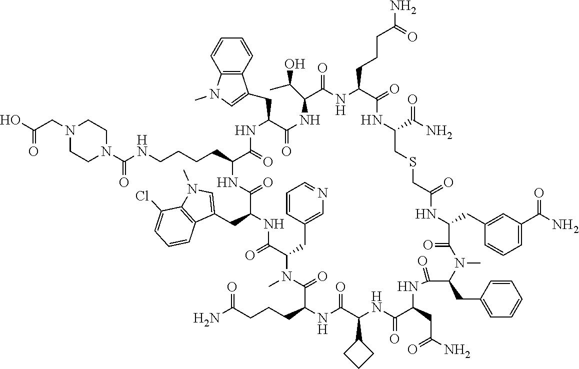 | HPLC-MS (Method A-B): 4.38 min ESI-MS m/z: 1010.0 [M + 2H]2+ |
| PDC_EphA2- 00019437 (SEQ ID NO: 53) | 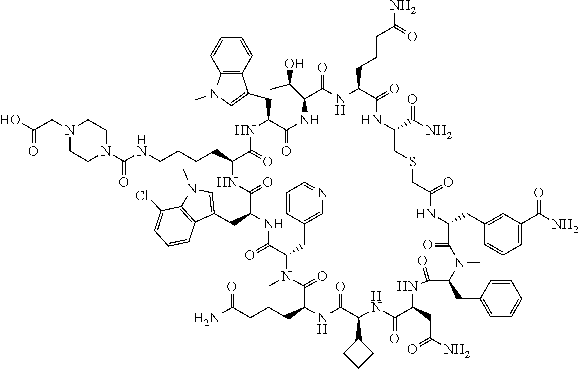 | HPLC-MS (Method A-B): 2.96 min ESI-MS m/z: 1007.1 [M + 2H]2+ |
| PDC_EphA2- 00019438 (SEQ ID NO: 54) | 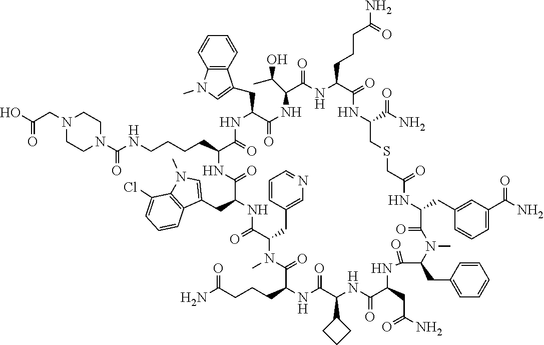 | HPLC-MS (Method A-B): 2.91 min ESI-MS m/z: 1010.1 [M + 2H]2+ |
| PDC_EphA2- 00019439 (SEQ ID NO: 55) | 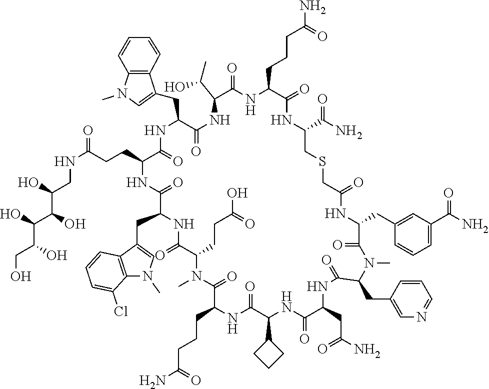 | HPLC-MS (Method A-B): 3.30 min ESI-MS m/z: 998.0 [M + 2H]2+ |
| PDC_EphA2- 00019440 (SEQ ID NO: 56) | 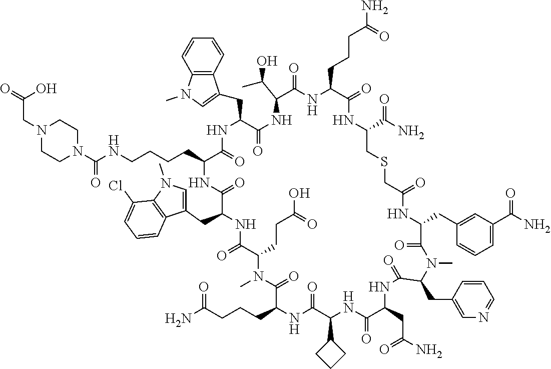 | HPLC-MS (Method A-B): 3.26 min ESI-MS m/z: 1001.0 [M + 2H]2+ |
| PDC_EphA2- 00019441 (SEQ ID NO: 57) | 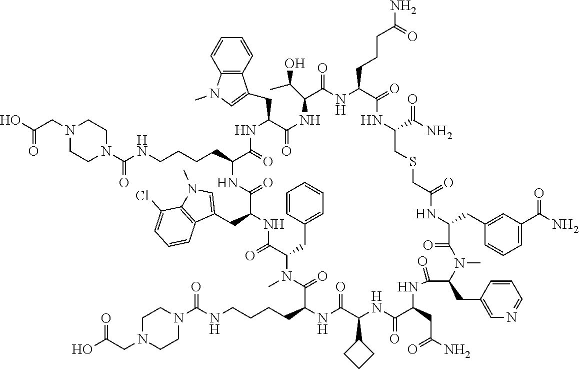 | HPLC-MS (Method A-B): 3.70 min ESI-MS m/z: 1001.0 [M + 3H]3+ |
| PDC_EphA2- 00019442 (SEQ ID NO: 58) | 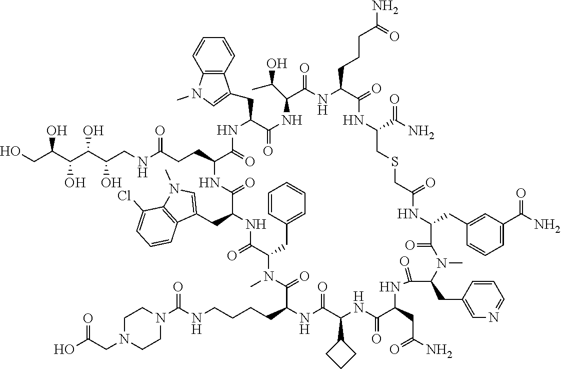 | HPLC-MS (Method A-B): 3.86 min ESI-MS m/z: 1084.7 [M + 2H]2+ |
| PDC_EphA2- 00026424 (SEQ ID NO: 59) | 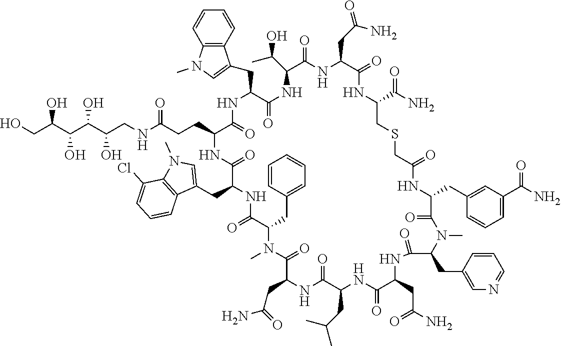 | HPLC-MS (Method A-B): 2.86 min ESI-MS m/z: 980.4 [M + 2H]2+ |
| PDC_EphA2- 00026425 (SEQ ID NO: 60) | 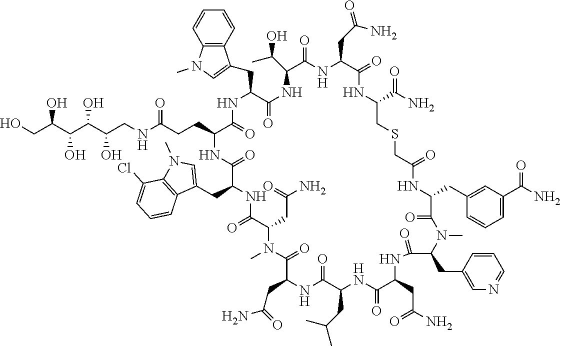 | HPLC-MS (Method A-B): 3.25 min ESI-MS m/z: 963.4 [M + 2H]2+ |
| PDC_EphA2- 00026426 (SEQ ID NO: 61) | 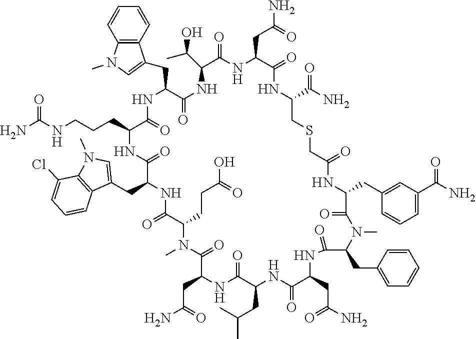 | HPLC-MS (Method A-B): 3.47 min ESI-MS m/z: 896.0 [M + 2H]2+ |
| PDC_EphA2- 00010011-C002 (SEQ ID NO: 201) | 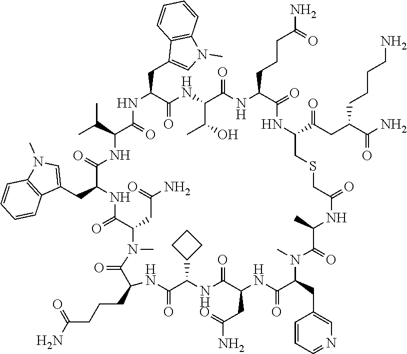 | HPLC-MS (Method A-A): 5.50 min ESI-MS m/z: 880.9 [M + 2H]2+ |
| PDC_EphA2- 00010012-C002 (SEQ ID NO: 202) | 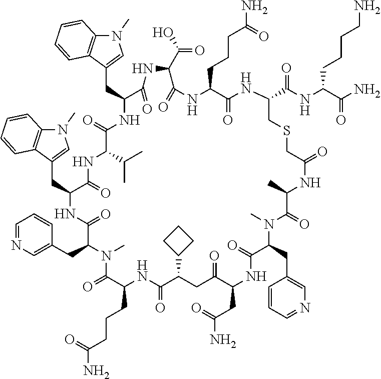 | HPLC-MS (Method A-A): 5.24 min ESI-MS m/z: 898.0 [M + 2H]2+ |
| PDC_EphA2- 00013693-C002 (SEQ ID NO: 203) | 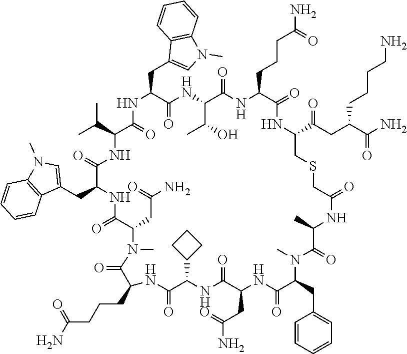 | HPLC-MS (Method A-B): 4.34 min ESI-MS m/z: 880.3 [M + 2H]2+ |
| PDC_EphA2- 00022593-C002 (SEQ ID NO: 205) | 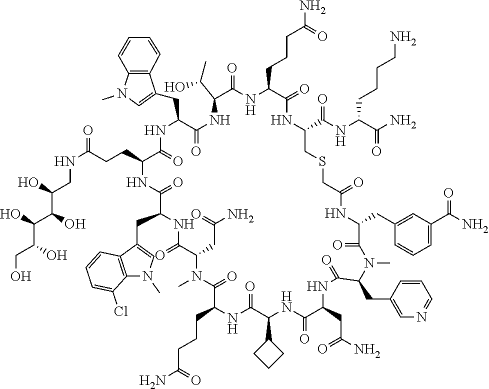 | HPLC-MS (Method A-A): 5.34 min ESI-MS m/z: 703.5 [M + 3H]3+ |
| PDC_EphA2- 00022594-C002 (SEQ ID NO: 206) | 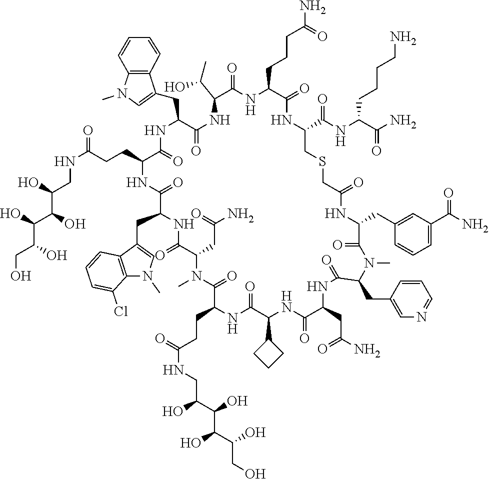 | HPLC-MS (Method A-A): 5.19 min ESI-MS m/z: 753.6 [M + 3H]3+ |
| PDC_EphA2- 00022595-C002 (SEQ ID NO: 207) | 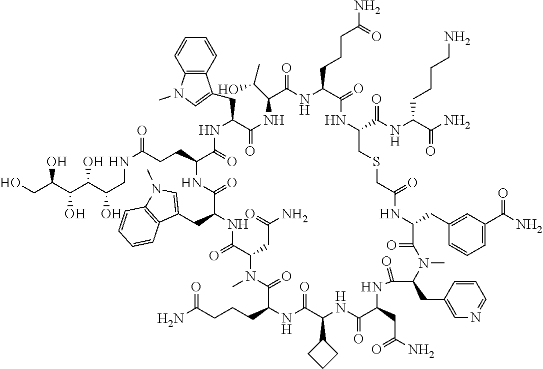 | HPLC-MS (Method A-A): 4.84 min ESI-MS m/z: 1037.2 [M + 2H]2+ |
| PDC_EphA2- 00022612-C002 (SEQ ID NO: 209) | 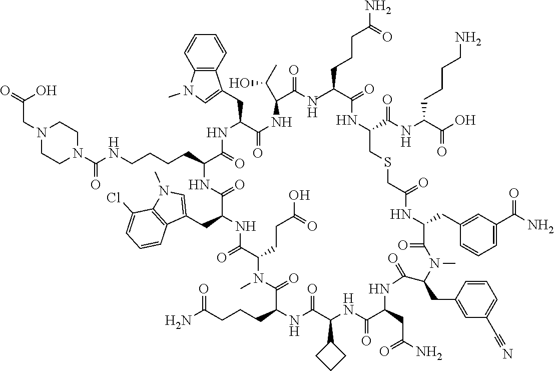 | HPLC-MS (Method A-B): 4.14 min ESI-MS m/z: 718.6 [M + 3H]3+ |
| PDC_EphA2- 00022613-C002 (SEQ ID NO: 210) | 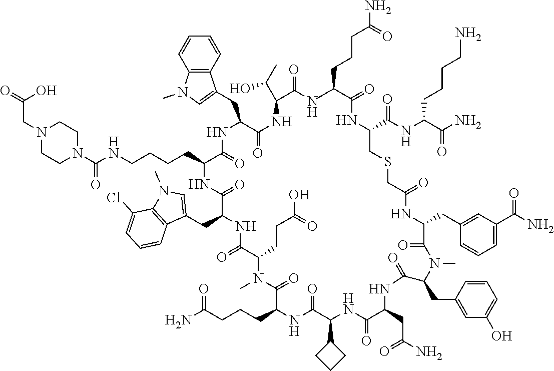 | HPLC-MS (Method A-B): 3.90 min ESI-MS m/z: 1072.2 [M + 2H]2+ |
| PDC_EphA2- 00022614-C002 (SEQ ID NO: 211) | 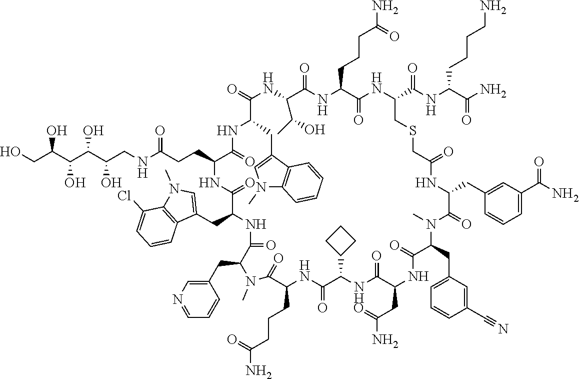 | HPLC-MS (Method A-B): 3.82 min ESI-MS m/z: 722.9 [M + 3H]3+ |
| PDC_EphA2- 00022615-C002 (SEQ ID NO: 212) | 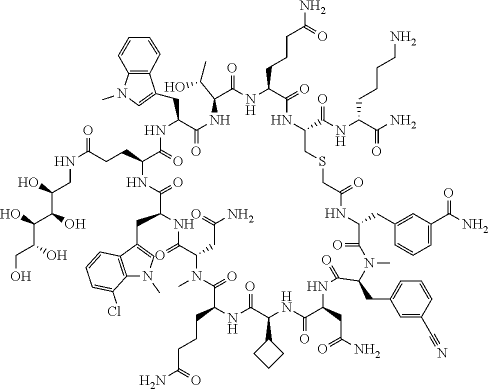 | HPLC-MS (Method A-B): 4.44 min ESI-MS m/z: 1066.2 [M + 2H]2+ |
| PDC_EphA2- 00013703-C002 (SEQ ID NO: 213) | 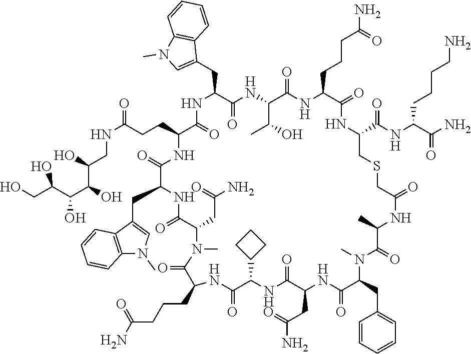 [SMILES: C[C@H]1NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 3.88 min ESI-MS m/z: 976.9 [M + 2H]2+ |
| PDC_EphA2- 00013704-C002 (SEQ ID NO: 214) | 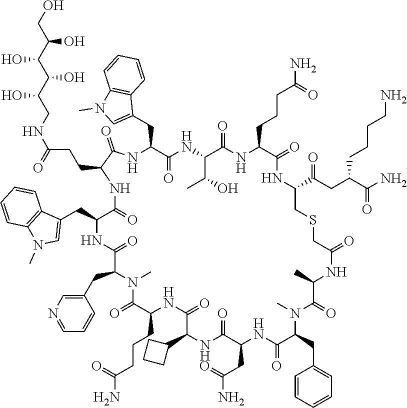 | HPLC-MS (Method A-B): 3.39 min ESI-MS m/z: 993.9 [M + 2H]2+ |
| PDC_EphA2- 00013705-C002 (SEQ ID NO: 215) | 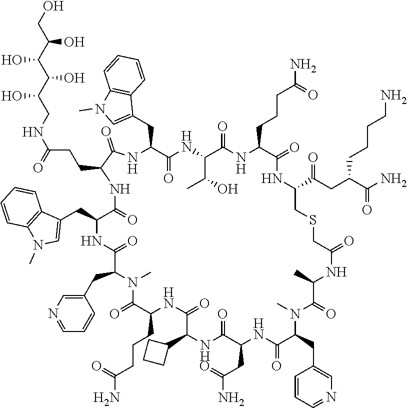 | HPLC-MS (Method A-A): 4.45 min ESI-MS m/z: 994.8 [M + 2H]2+ |
| PDC_EphA2- 00026424-C002 (SEQ ID NO: 216) | 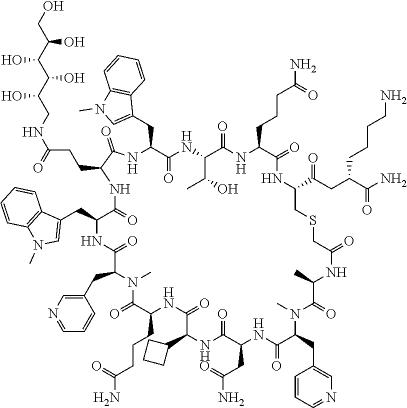 | HPLC-MS (Method A-A): 5.14 min ESI-MS m/z: 696.8 [M + 3H]3+ |
| PDC_EphA2- 00026425-C002 (SEQ ID NO: 217) | 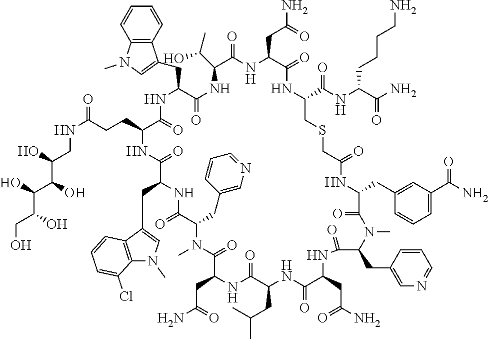 | HPLC-MS (Method A-B): 3.01 min ESI-MS m/z: 1027.2 [M + 2H]2+ |
| PDC_EphA2- 00026426-C002 (SEQ ID NO: 218) | 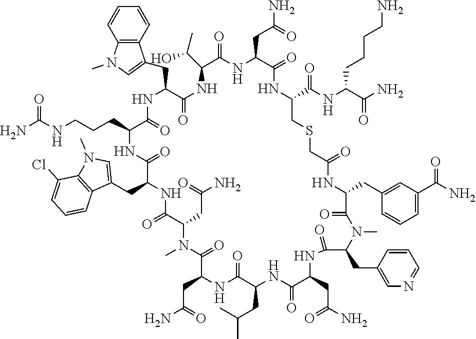 | HPLC-MS (Method A-B): 3.19 min ESI-MS m/z: 959.7 [M + 2H]2+ |
| PDC_EphA2- 00010011-C003 (SEQ ID NO: 180) | 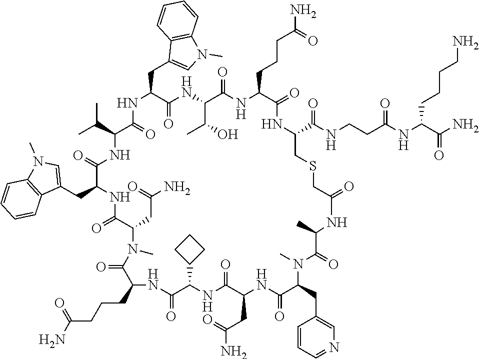 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(=O)NCCC(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 3.10 min ESI-MS m/z: 916.5 [M + 2H]2+ |
| PDC_EphA2- 00010012-C003 (SEQ ID NO: 181) | 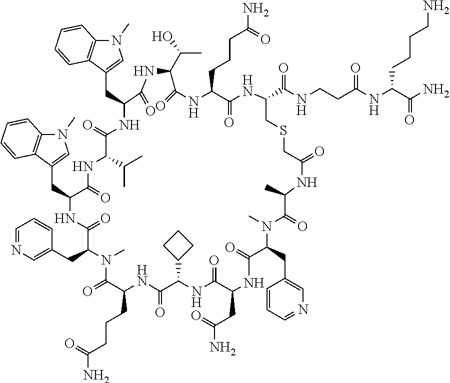 | HPLC-MS (Method A-A): 5.17 min ESI-MS m/z: 622.6 [M + 3H]3+ |
| PDC_EphA2- 00013693-C003 (SEQ ID NO: 182 | 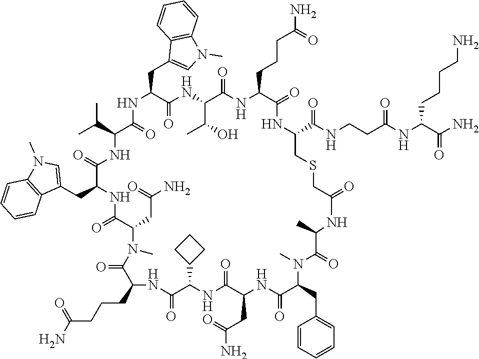 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(=O)NCCC(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 4.27 min ESI-MS m/z: 915.9 [M + 2H]2+ |
| PDC_EphA2- 00026521 (SEQ ID NO: 62) | 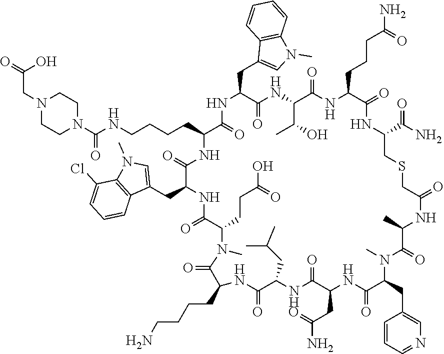 | HPLC-MS (Method A-A): 5.80 min ESI-MS m/z: 623.8 [M + 3H]3+ |
| PDC_EphA2- 00026522 (SEQ ID NO: 63) | 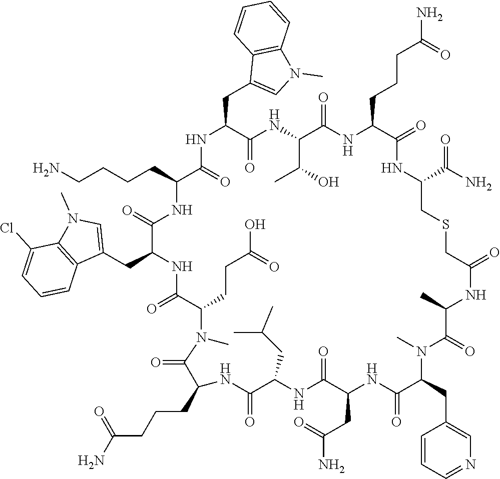 | HPLC-MS (Method A-A): 6.04 min ESI-MS m/z: 857.1 [M + 2H]2+ |
| PDC_EphA2- 00026523 (SEQ ID NO: 64) | 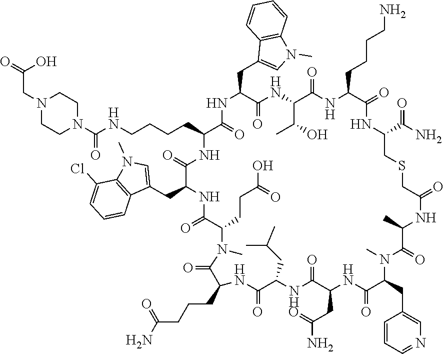 | HPLC-MS (Method A-A): 5.83 min ESI-MS m/z: 623.8 [M + 3H]3+ |
| PDC_EphA2- 00026524 (SEQ ID NO: 65) | 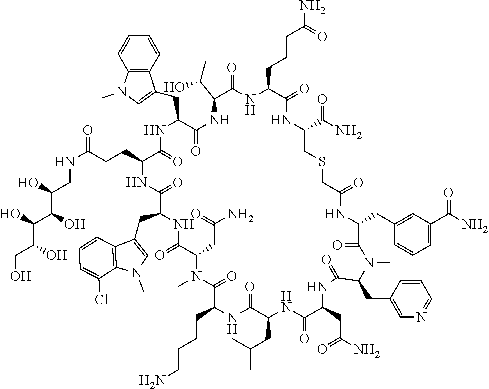 | HPLC-MS (Method A-A): 5.83 min ESI-MS m/z: 984.2 [M + 2H]2+ |
| PDC_EphA2- 00026525 (SEQ ID NO: 66) | 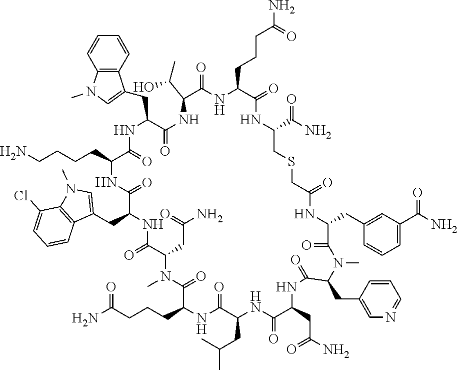 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCN)NC(=O)[C@H](Cc2cn(C)c3c(Cl)cccc23)NC(=O)[C@H](CC(N)=O)N(C)C(=O)[C@H](CCCC(N)=O)NC1=O] | HPLC-MS (Method A-B): 3.28 min ESI-MS m/z: 909.1 [M + 2H]2+ |
| PDC_EphA2- 00026526 (SEQ ID NO: 67) | 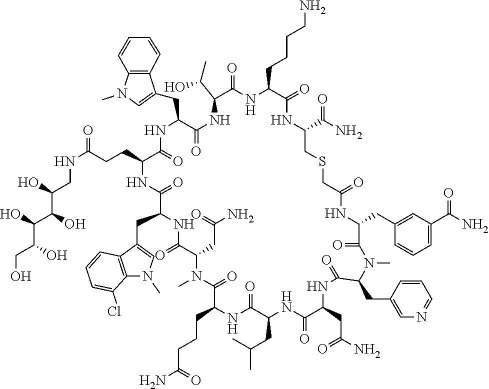 | HPLC-MS (Method A-A): 5.84 min ESI-MS m/z: 984.2 [M + 2H]2+ |
| PDC_EphA2- 00026527 (SEQ ID NO: 68) | 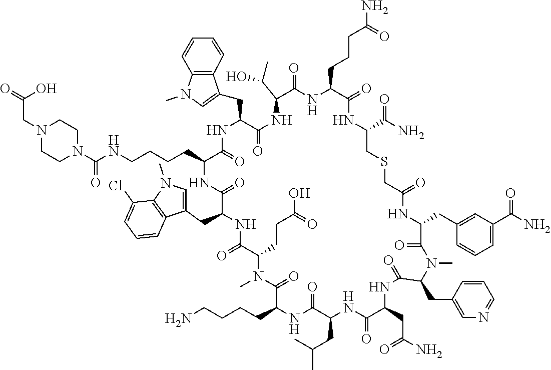 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](CCC(=O)O)N(C)C(=O)[C@H](CCCCN)NC1=O] | HPLC-MS (Method A-B): 3.13 min ESI-MS m/z: 994.6 [M + 2H]2+ |
| PDC_EphA2- 00026528 (SEQ ID NO: 69) | 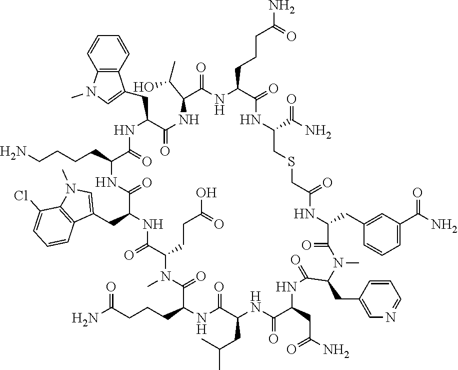 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCN)NC(=O)[C@H](Cc2cn(C)c3c(Cl)cccc23)NC(=O)[C@H](CCC(=O)O)N(C)C(=O)[C@H](CCCC(N)=O)NC1=O] | HPLC-MS (Method A-B): 3.40 min ESI-MS m/z: 916.5 [M + 2H]2+ |
| PDC_EphA2- 00026529 (SEQ ID NO: 70) | 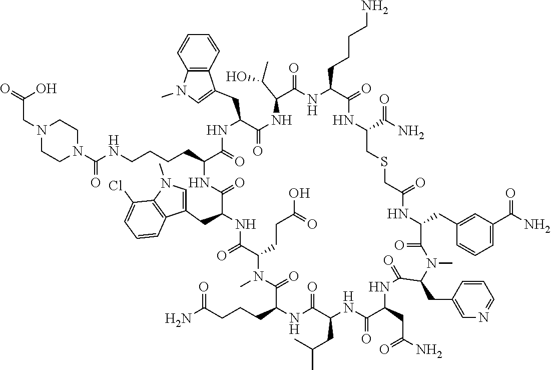 | HPLC-MS (Method A-B): 3.17 min ESI-MS m/z: 663.5 [M + 3H]3+ |
| PDC_EphA2- 00027094-C002 (SEQ ID NO: 252) | 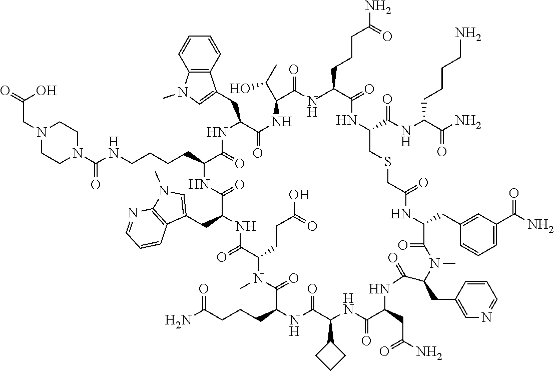 | HPLC-MS (Method A-A): 4.31 min ESI-MS m/z: 699.0 [M + 3H]3+ |
| PDC_EphA2- 00027095-C002 (SEQ ID NO: 253) | 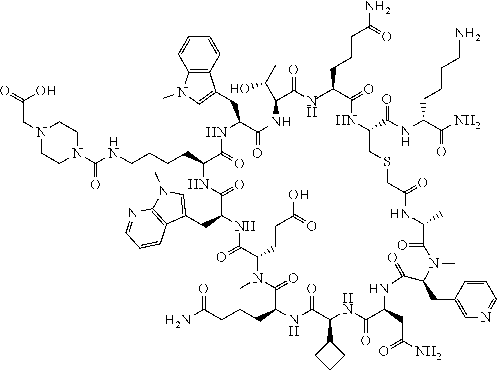 | HPLC-MS (Method A-A): 4.18 min ESI-MS m/z: 659.3 [M + 3H]3+ |
| PDC_EphA2- 00027092 (SEQ ID NO: 256) | 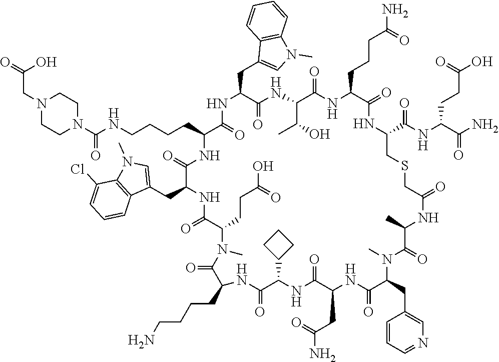 | HPLC-MS (Method A-A): 5.59 min ESI-MS m/z: 666.1 [M + 3H]3+ |

### Example 7

The following Examples were prepared by following similar methods described in the examples above. The amino acid sequence for each peptide was shown in Tables 6A and/or 6B.

TABLE 8

| Example | | |
|---|---|---|
| No. | Structure | Analytics |
| PDC_ EphA2- 00026624 (SEQ ID NO: 219) | 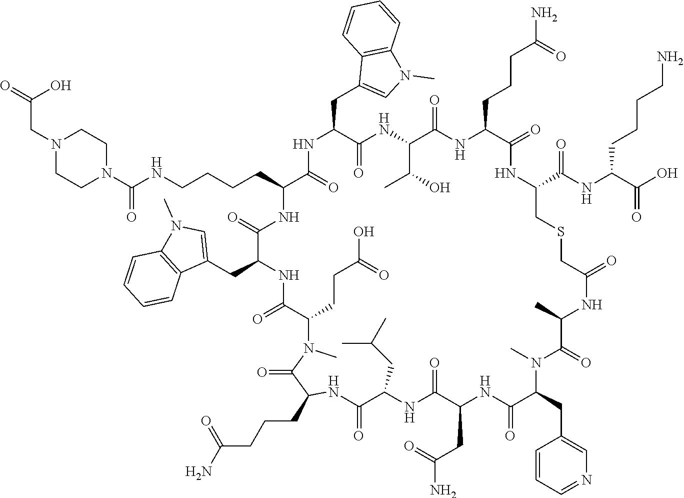 | HPLC-MS (Method A-A): 5.23 min ESI-MS m/z: 660.0 [M + 3H]3+ |
| PDC_ EphA2- 00026625 (SEQ ID NO: 220) | 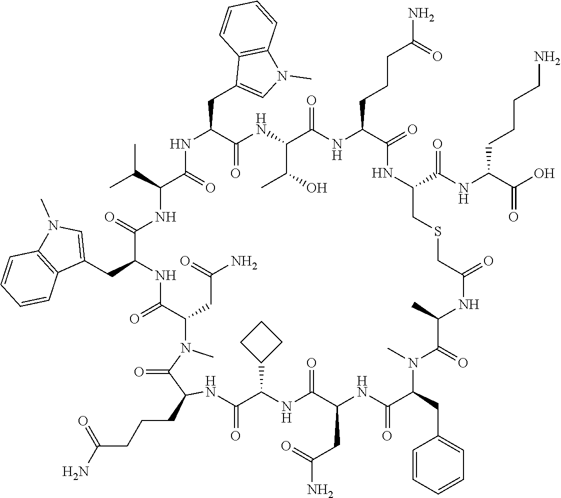 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(=O)N[C@H](CCCCN)C(=O)O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 4.56 min ESI-MS m/z: 880.9 [M + 2H]2+ |
| PDC_ EphA2- 00026627 (SEQ ID NO: 222) | 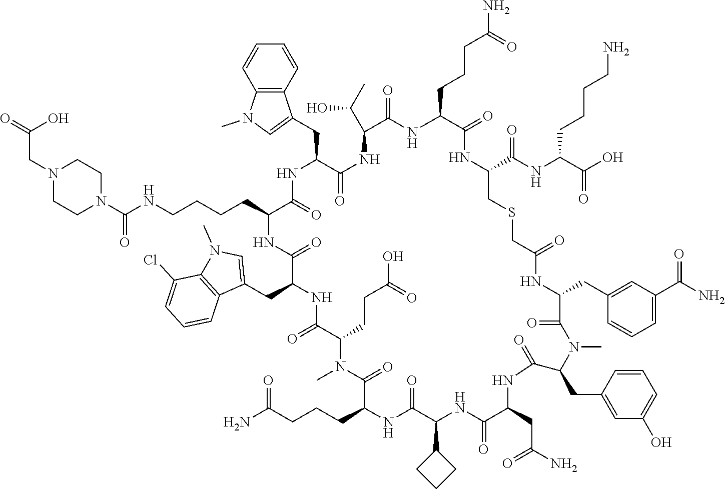 | HPLC-MS (Method A-B): 3.96 min ESI-MS m/z: 1072.8 [M + 2H]2+ |
| PDC_ EphA2- 00027090 (SEQ ID NO: 254) | 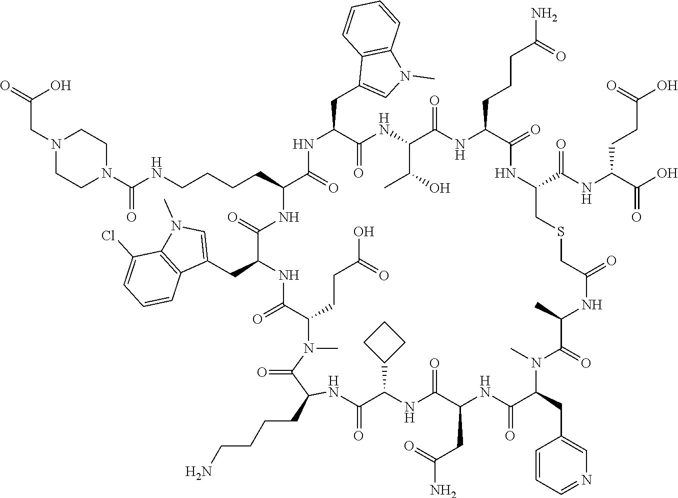 | HPLC-MS (Method A-A): 5.65 min ESI-MS m/z: 666.5 [M + 3H]3+ |
| PDC_ EphA2- 00027091 (SEQ ID NO: 255) | 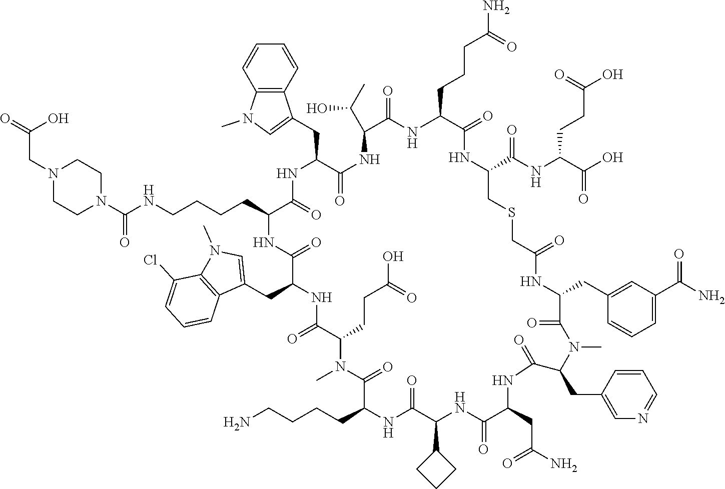 | HPLC-MS (Method A-A): 5.71 min ESI-MS m/z: 706.2 [M + 3H]3+ |

### Example 8: PDC_EphA2-00007196-C004 (SEQ ID NO: 224)

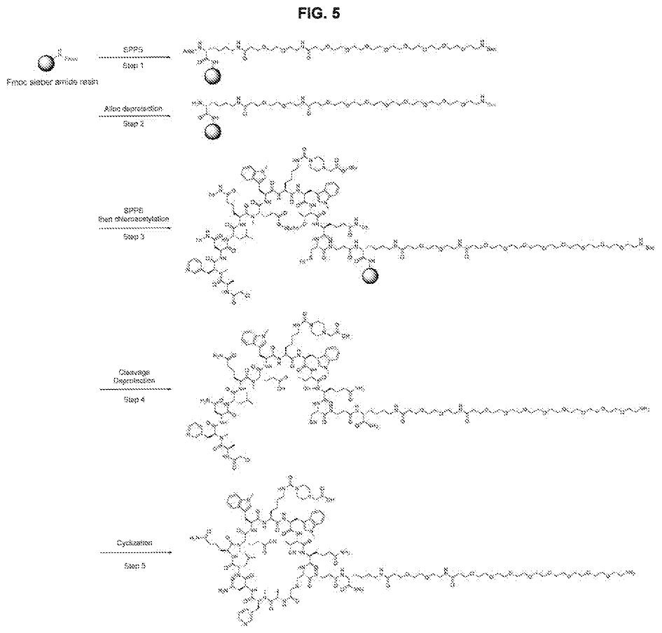

Intermediates on-resin were synthesized in the analogous manner to the step 1 of the scheme A. The intermediate peptide on-resin was synthesized on Fmoc-Sieber amide Resin (0.25 mmol) in the analogous manner to the step 1 of the scheme A. The resin was shaken with Pd(PPh3)4 (0.2 eq) and PhSiH3 (10 eq) at room temperature for 1 h. The peptide sequence was then synthesized on the intermediate following general peptide synthesis method A. The obtained linear peptide on the resin was subjected to the general method CIAc-3. Polypeptide on the resin was treated with Cleavage Cocktail-A (25 mL) to furnish the linear peptide. The crude containing the linear peptide (0.375 mmol as theoretical based on the resin used) was subjected to the peptide cyclization condition-2. The resulting residue was purified by preparative reversed-phase HPLC (Method P—C). Pure fractions were combined and lyophilized to afford the title macrocyclic peptide. HPLC-MS (Method A-A): 5.64 min, ESI-MS m/z: 877.6 [M+3H]3+, AUC (UV 225 nm): 92%. See FIG. 5.

### Example 9: PDC_EphA2-00007196-C010 (SEQ ID NO: 231)

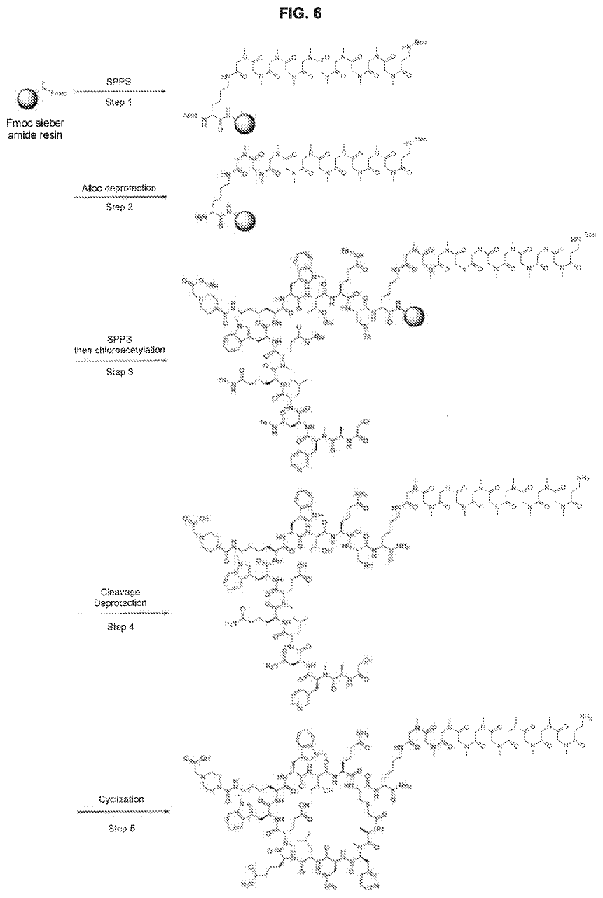

Intermediates on-resin were synthesized in the analogous manner to the step 1 of the scheme A. The intermediate peptide on-resin was synthesized on Fmoc-Sieber amide Resin (0.25 mmol) in the analogous manner to the step 1 of the scheme A. The resin was shaken with Pd(PPh3)4 (0.2 eq) and PhSiH3 (10 eq) at room temperature for 1 h. The peptide sequence was then synthesized on the intermediate following general peptide synthesis method A. The obtained linear peptide on the resin was subjected to the general method CIAc-3. Polypeptide on the resin was treated with Cleavage Cocktail-A (18 mL) to furnish the linear peptide. The crude containing the linear peptide (0.375 mmol as theoretical based on the resin used) was subjected to the peptide cyclization condition-2. The resulting residue was purified by preparative reversed-phase HPLC (Method P—C). Pure fractions were combined and lyophilized to afford the title macrocyclic peptide. HPLC-MS (Method A-A): 5.58 min, ESI-MS m/z: 920.3 [M+3H]3+, AUC (UV 225 nm): 96%. See FIG. 6.

### Example 10

The following Examples were prepared by following similar methods described in the examples above. The amino acid sequence for each peptide was shown in Tables 6A and/or 6B.

TABLE 9

| Example | | Ana- |
|---|---|---|
| No. | Structure | lytics |
| PDC_ EphA2- 00001417- C004 (SEQ ID NO: 223) | 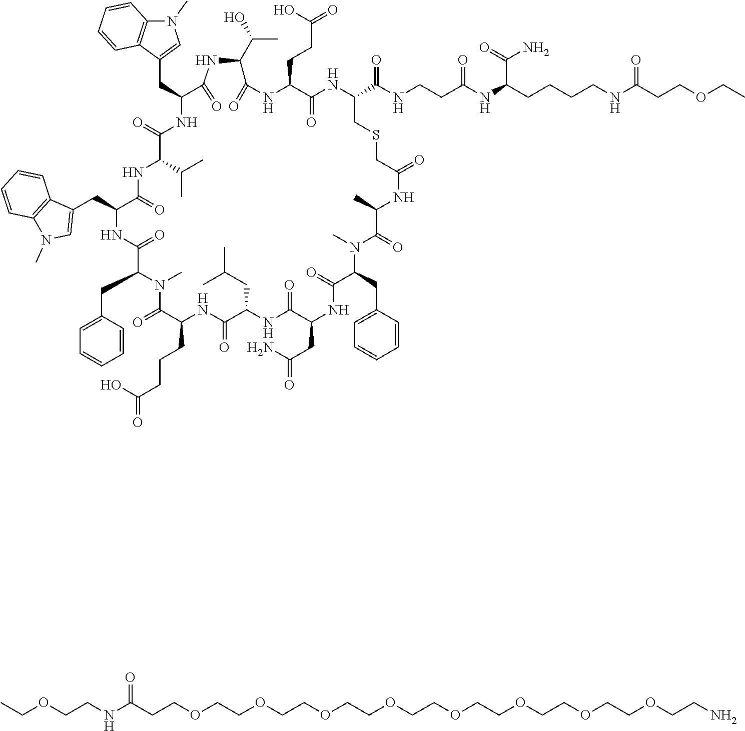 | HPLC- MS (Method A-B): 5.90 min ESI-MS m/z: 812.9 [M + 3H]3+ |
| PDC_ EphA2- 00008093- C004 (SEQ ID NO: 225) | 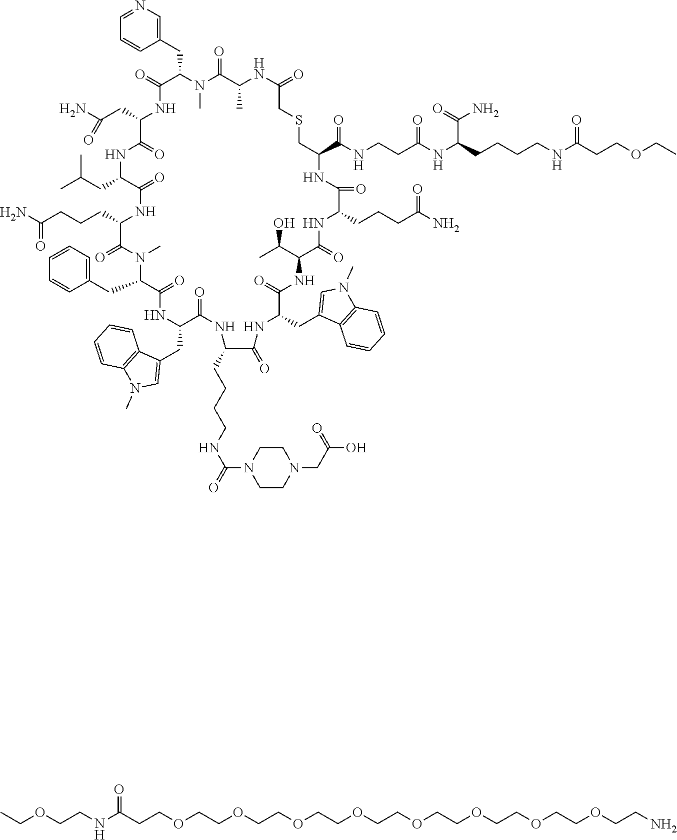 | HPLC- MS (Method A-B): 3.39 min ESI-MS m/z: 883.6 [M + 3H]3+ |
| PDC_ EphA2- 00008094- C004 (SEQ ID NO: 226) | 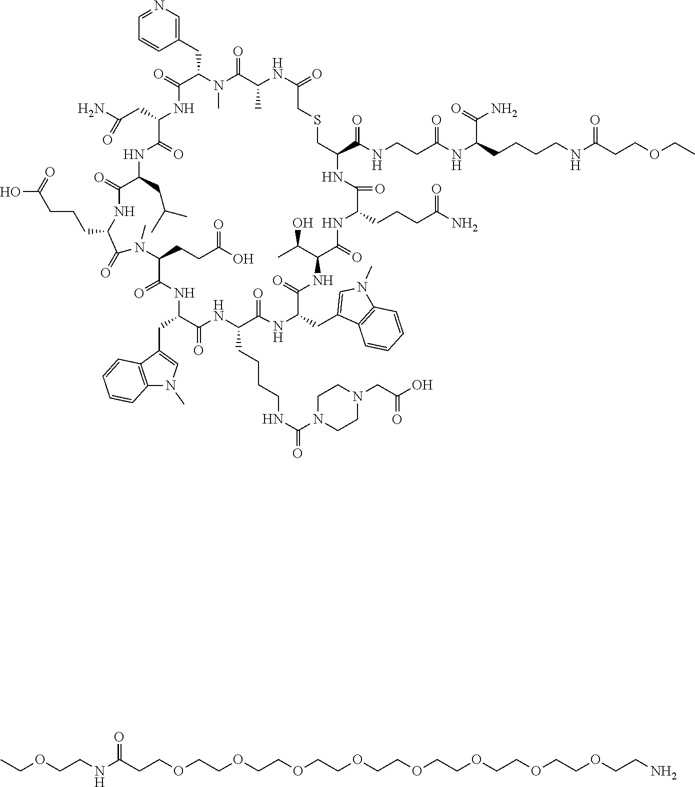 | HPLC- MS (Method A-A): 5.56 min ESI-MS m/z: 878.0 [M + 3H]3+ |
| PDC_ EphA2- 00010011- C004 (SEQ ID NO: 227) | 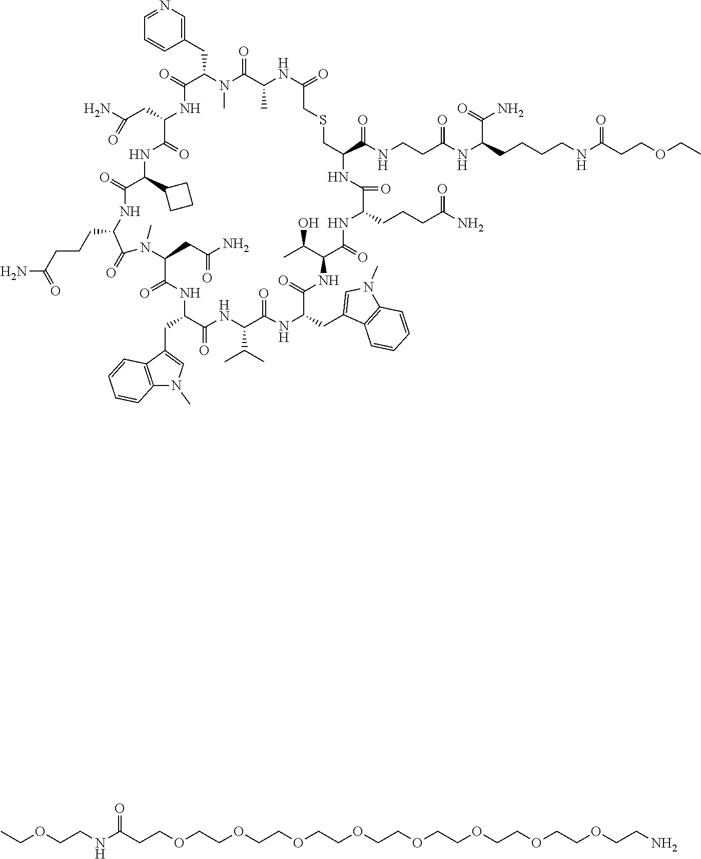 | HPLC- MS (Method A-B): 3.04 min ESI-MS m/z: 805.7 [M + 3H]3+ |
| PDC_ EphA2- 00010012- C004 (SEQ ID NO: 228) | 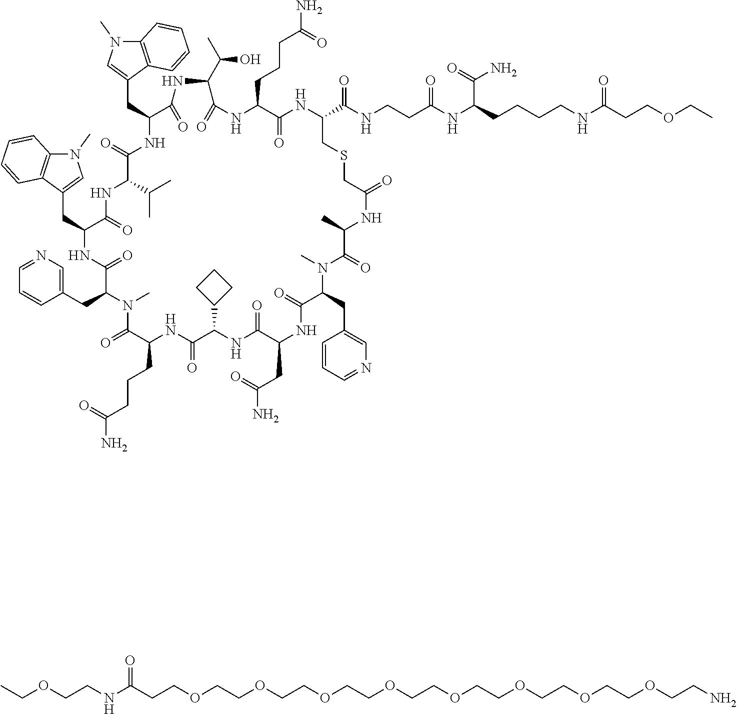 | HPLC- MS (Method A-B): 2.79 min ESI-MS m/z: 816.9 [M + 3H]3+ |
| PDC_ EphA2- 00013693- C004 (SEQ ID NO: 229) | 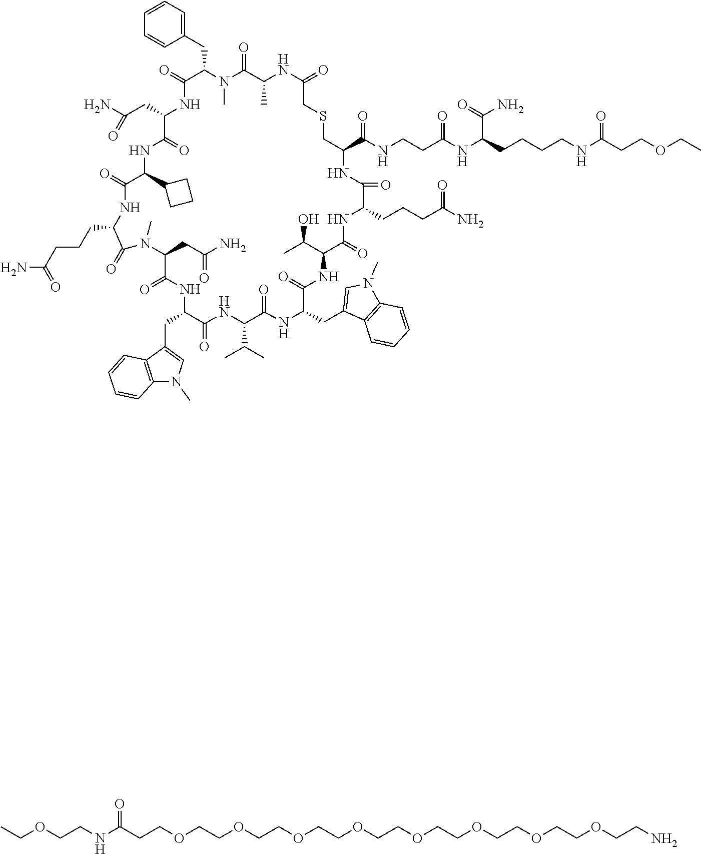 | HPLC- MS (Method A-B): 4.47 min ESI-MS m/z: 805.1 [M + 3H]3+ |
| PDC_ EphA2- 00008093- C010 (SEQ ID NO: 230) | 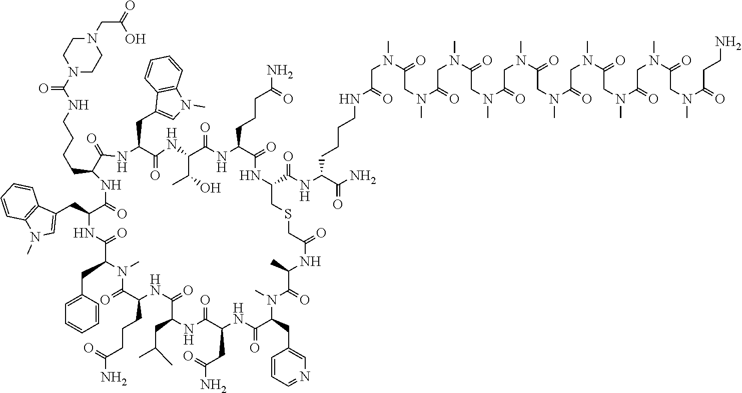 | HPLC- MS (Method A-B): 3.12 min ESI-MS m/z: 926.3 [M + 3H]3+ |

### Example 11 PDC_EphA2-00026603-C001 (SEQ ID NO: 241)

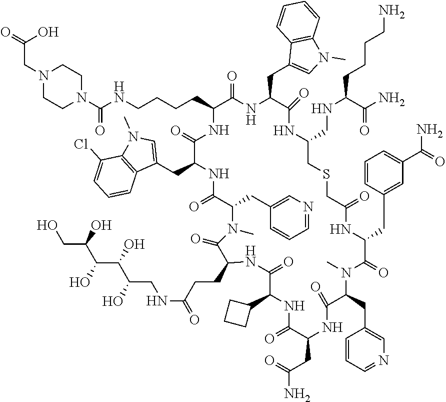 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](CN[C@@H](CCCCN)C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1]

The peptide sequence was synthesized on Fmoc-Sieber amide Resin (0.250 mmol) following general peptide synthesis method A. The obtained peptide on the resin was subjected to the general method CIAc-3. The polypeptide on the resin was treated with Cleavage Cocktail-A (10 mL) to furnish the linear peptide. The crude containing the linear peptide (0.250 mmol as theoretical based on the resin used) was subjected to the peptide cyclization condition-2. The resulting residue was purified by preparative reversed-phase HPLC (Method P-A). Pure fractions were combined and lyophilized to afford the title macrocyclic peptide. HPLC-MS (Method A-A): 5.13 min, ESI-MS m/z: 685.5 [M+3H]3+, AUC (UV 225 nm): 98%.

### Example 12

The following Examples were prepared by following similar methods described in the examples above. The amino acid sequence for each peptide was shown in Tables 6A and/or 6B.

TABLE 10

| Example | | |
|---|---|---|
| No. | Structure | Analytics |
| PDC_EphA2- 00026600- C001 (SEQ ID NO: 238) | 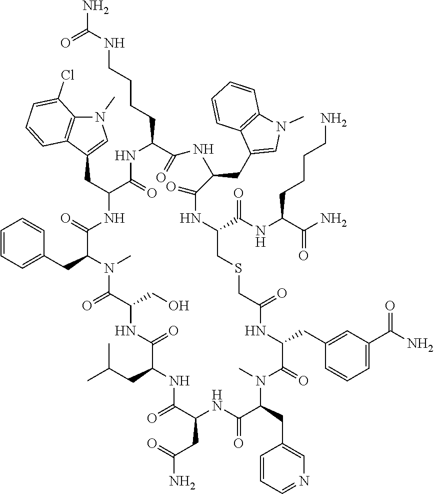 | HPLC-MS (Method A-B): 3.84 min ESI-MS m/z: 862.4 [M + 2H]2+ |
| PDC_EphA2- 00026601- C001 (SEQ ID NO: 239) | 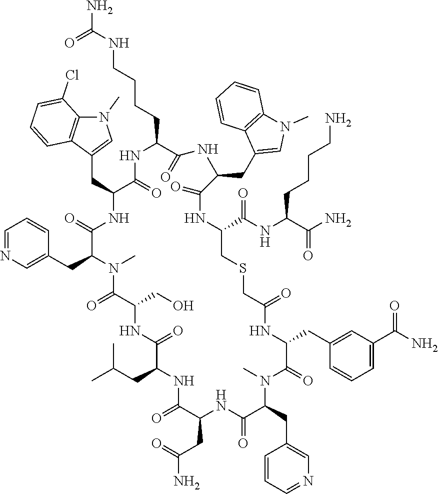 | HPLC-MS (Method A-A): 5.27 min ESI-MS m/z: 863.0 [M + 2H]2+ |
| PDC_EphA2- 00026602- C001 (SEQ ID NO: 240) | 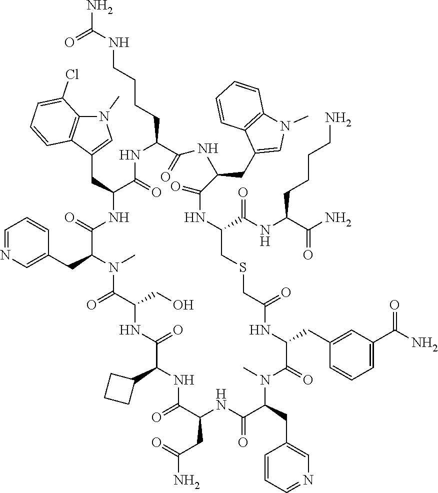 [SMILES: CN1C(=O)[C@H](CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@@H](CCCCN)C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1Cc1cccnc1] | HPLC-MS (Method A-A): 5.54 min ESI-MS m/z: 861.5 [M + 2H]2+ |
| PDC_EphA2- 00026604- C001 (SEQ ID NO: 242) | 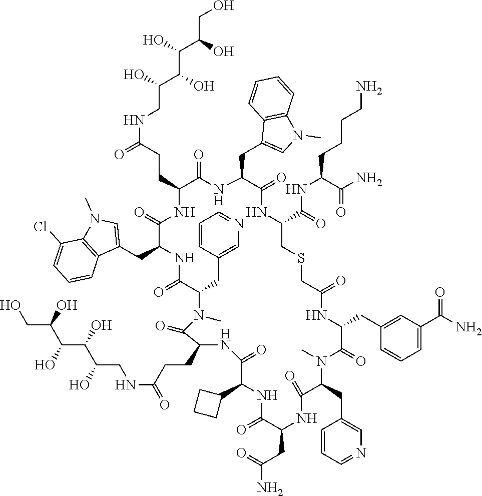 | HPLC-MS (Method A-A): 5.19 min ESI-MS m/z: 683.5 [M + 3H]3+ |
| PDC_EphA2- 00026606- C001 (SEQ ID NO: 243) | 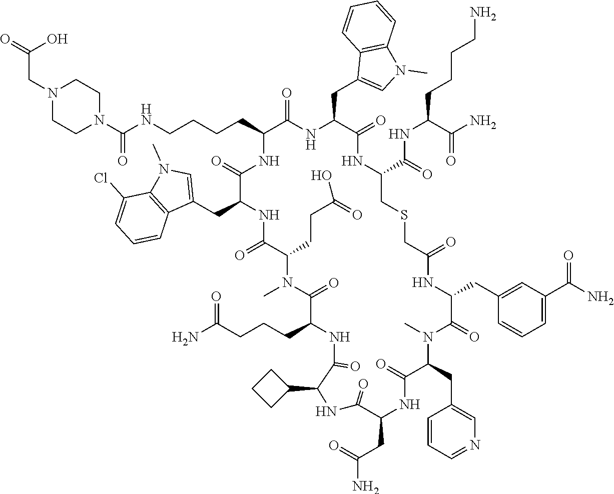 | HPLC-MS (Method A-A): 5.59 min ESI-MS m/z: 629.1 [M + 3H]3+ |
| PDC_EphA2- 00026607- C001 (SEQ ID NO: 244) | 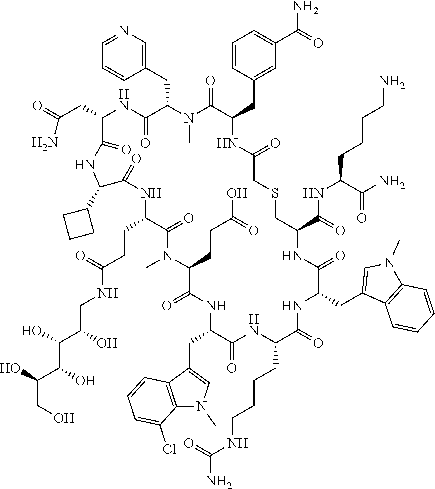 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@@H](CCCCN)C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1CCC(=O)O] | HPLC-MS (Method A-A): 5.66 min ESI-MS m/z: 954.6 [M + 2H]2+ |
| PDC_EphA2- 00026608- C001 (SEQ ID NO: 245) | 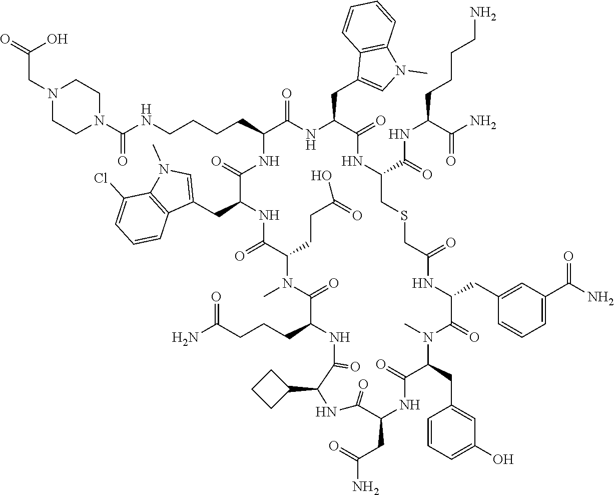 | HPLC-MS (Method A-B): 4.00 min ESI-MS m/z: 950.7 [M + 2H]2+ |

### Example 13

The following Examples were prepared by following similar methods described in the examples above. The amino acid sequence for each peptide was shown in Tables 6A and/or 6B.

TABLE 11

| Example | | |
|---|---|---|
| No. | Structure | Analytics |
| PDC_EphA2- 00020708 (SEQ ID NO: 150) | 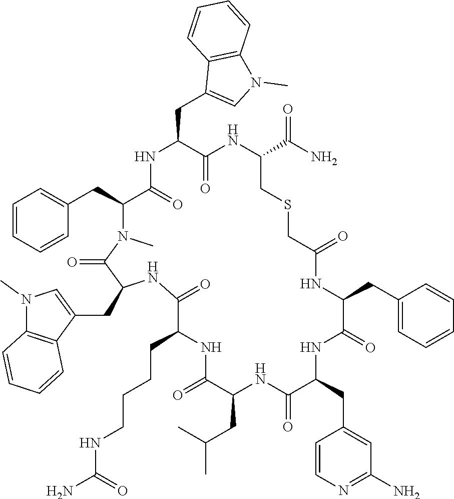 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)NC1=O] | HPLC-MS (Method A-B): 5.05 min ESI-MS m/z: 659.1 [M + 2H]2+ |
| PDC_EphA2- 00022601 (SEQ ID NO: 246) | 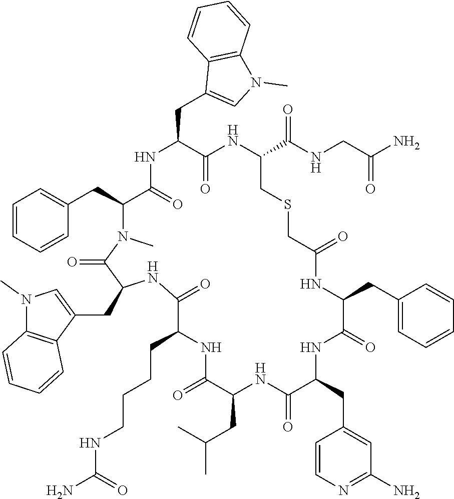 [SMILES: CC(C)C[C@@H]1NC(=O)[C@H](Cc2ccnc(N)c2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)NC1=O] | HPLC-MS (Method A-B): 4.85 min ESI-MS m/z: 687.7 [M + 2H]2+ |
| PDC_EphA2- 00022602 (SEQ ID NO: 247) | 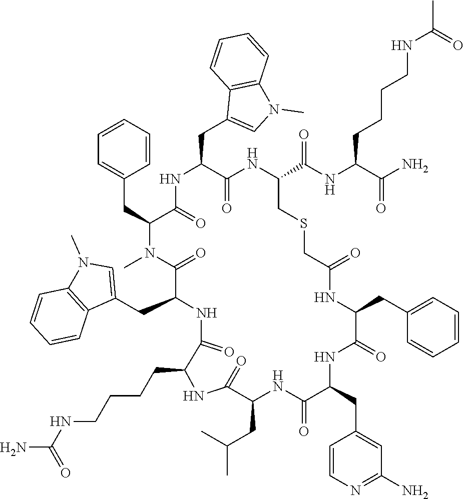 [SMILES: CC(=O)NCCCC[C@H](NC(=O)[C@@H]1CSCC(=O)N[C@@H](Cc2ccccc2)C(=O)N[C@@H](Cc2ccnc(N)c2)C(=O)N[C@@H](CC(C)C)C(=O)N[C@@H](CCCCNC(N)=O)C(=O)N[C@@H](Cc2cn(C)c3ccccc23)C(=O)N(C)[C@@H](Cc2ccccc2)C(=O)N[C@@H](Cc2cn(C)c3ccccc23)C(=O)N1)C(N)=O] | HPLC-MS (Method A-B): 4.85 min ESI-MS m/z: 744.2 [M + 2H]2+ |
| PDC_EphA2- 00022603 (SEQ ID NO: 248) | 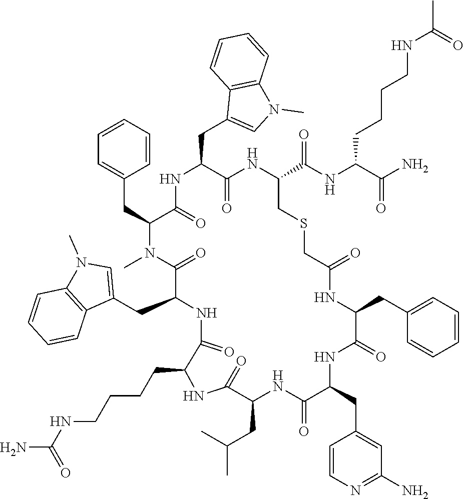 [SMILES: CC(=O)NCCCC[C@@H](NC(=O)[C@@H]1CSCC(=O)N[C@@H](Cc2ccccc2)C(=O)N[C@@H](Cc2ccnc(N)c2)C(=O)N[C@@H](CC(C)C)C(=O)N[C@@H](CCCCNC(N)=O)C(=O)N[C@@H](Cc2cn(C)c3ccccc23)C(=O)N(C)[C@@H](Cc2ccccc2)C(=O)N[C@@H](Cc2cn(C)c3ccccc23)C(=O)N1)C(N)=O] | HPLC-MS (Method A-B): 4.84 min ESI-MS m/z: 744.2 [M + 2H]2+ |
| PDC_EphA2- 00019498- L092 (SEQ ID NO: 251) | 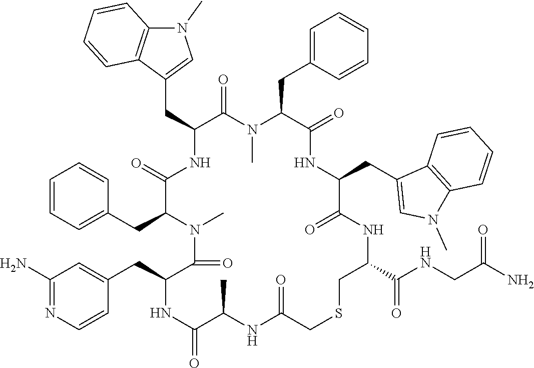 [SMILES: C[C@H]1NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](Cc2ccccc2)N(C)C(=O)[C@H](Cc2ccnc(N)c2)NC1=O] | HPLC-MS (Method A-B): 5.46 min ESI-MS m/z: 588.1 [M + 2H]2+ |
| PDC_EphA2- 00020295- L092 (SEQ ID NO: 249) | 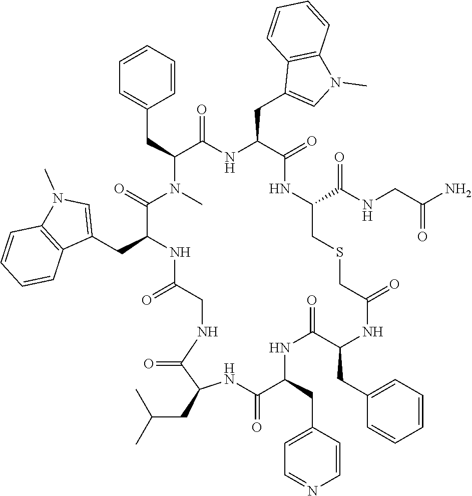 [SMILES: CC(C)C[C@@H]1NC(=O)[C@H](Cc2ccncc2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)CNC1=O] | HPLC-MS (Method A-B): 5.31 min ESI-MS m/z: 623.1 [M + 2H]2+ |
| PDC_EphA2- 00026479 (SEQ ID NO: 151) | 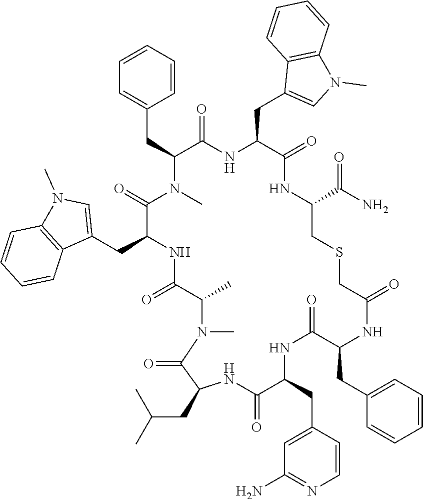 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](C)N(C)C1=O] | HPLC-MS (Method A-B): 6.04 min ESI-MS m/z: 616.2 [M + 2H]2+ |
| PDC_EphA2- 00026481 (SEQ ID NO: 152) | 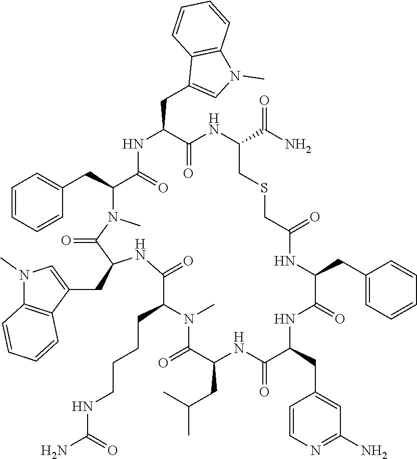 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)N(C)C1=O] | HPLC-MS (Method A-B): 5.32 min ESI-MS m/z: 666.2 [M + 2H]2+ |
| PDC_EphA2- 00021407- L092 (SEQ ID NO: 235) | 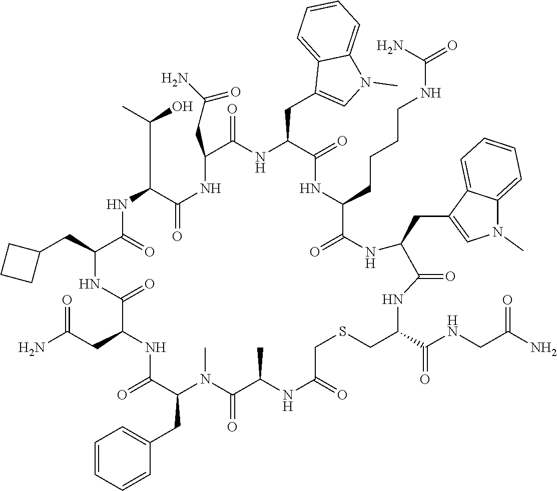 [SMILES: C[C@H]1NC(=O)CSC[C@@H](C(=O)NCC(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](CC(N)=O)NC(=O)[C@H]([C@@H](C)O)NC(=O)[C@H](CC2CCC2)NC(=O)[C@H](CC(N)=O)NC(=O)[C@H](Cc2ccccc2)N(C)C1=O] | |
| PDC_EphA2- 00022057- L092 (SEQ ID NO: 236) | 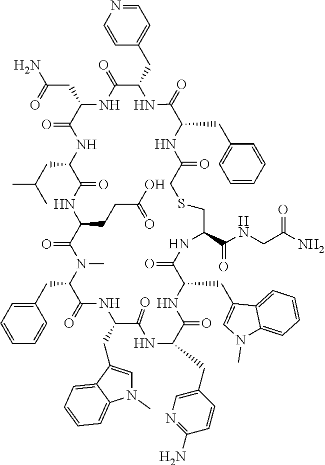 [SMILES: CC(C)C[C@@H]1NC(=O)[C@H](CC(N)=O)NC(=O)[C@H](Cc2ccncc2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccc(N)nc2)NC(=O)[C@H](Cc2cn(C)c3ccccc23)NC(=O)[C@H](Cc2ccccc2)N(C)C(=O)[C@H](CCC(=O)O)NC1=O] | |
| PDC_EphA2- 00022417 (SEQ ID NO: 123) | 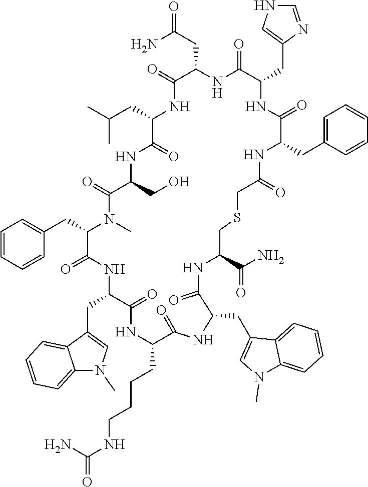 [SMILES: CC(C)C[C@@H]1NC(=O)[C@H](CC(N)=O)NC(=O)[C@H](Cc2c[nH]cn2)NC(=O)[C@H](Cc2ccccc2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](Cc2ccccc2)N(C)C(=O)[C@H](CO)NC1=O] | |
| PDC_EphA2- 00022606 (SEQ ID NO: 232) | 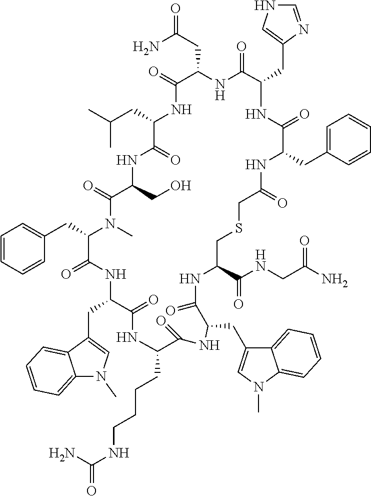 [SMILES: CC(C)C[C@@H]1NC(=O)[C@H](CC(N)=O)NC(=O)[C@H](Cc2c[nH]cn2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](Cc2ccccc2)N(C)C(=O)[C@H](CO)NC1=O] | |
| PDC_EphA2- 00022607 (SEQ ID NO: 233) | 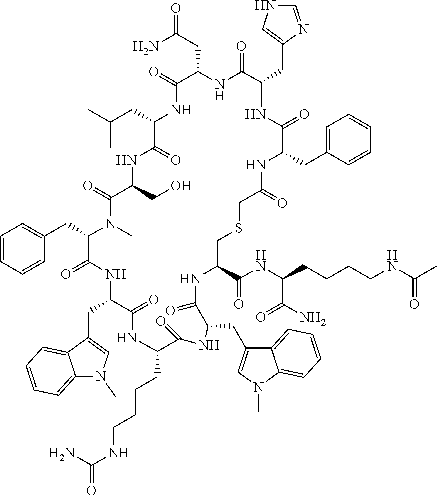 [SMILES: CC(=O)NCCCC[C@H](NC(=O)[C@@H]1CSCC(=O)N[C@@H](Cc2ccccc2)C(=O)N[C@@H](Cc2c[nH]cn2)C(=O)N[C@@H](CC(N)=O)C(=O)N[C@@H](CC(C)C)C(=O)N[C@@H](CO)C(=O)N(C)[C@@H](Cc2ccccc2)C(=O)N[C@@H](Cc2cn(C)c3ccccc23)C(=O)N[C@@H](CCCCNC(N)=O)C(=O)N[C@@H](Cc2cn(C)c3ccccc23)C(=O)N1)C(N)=O] | |
| PDC_EphA2- 00022608 SEQ ID NO: 234) | 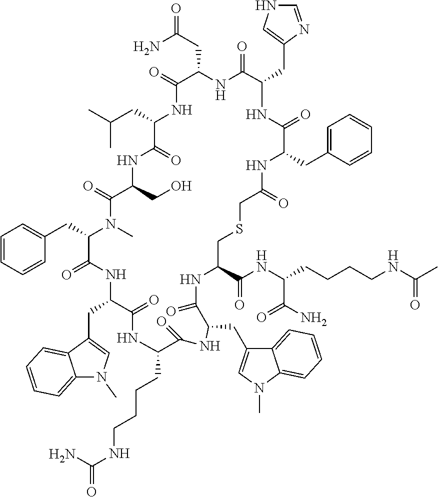 [SMILES: CC(=O)NCCCC[C@@H](NC(=O)[C@@H]1CSCC(=O)N[C@@H](Cc2ccccc2)C(=O)N[C@@H](Cc2c[nH]cn2)C(=O)N[C@@H](CC(N)=O)C(=O)N[C@@H](CC(C)C)C(=O)N[C@@H](CO)C(=O)N(C)[C@@H](Cc2ccccc2)C(=O)N[C@@H](Cc2cn(C)c3ccccc23)C(=O)N[C@@H](CCCCNC(N)=O)C(=O)N[C@@H](Cc2cn(C)c3ccccc23)C(=O)N1)C(N)=O] | |
| PDC_EphA2- 00026600 (SEQ ID NO: 124) | 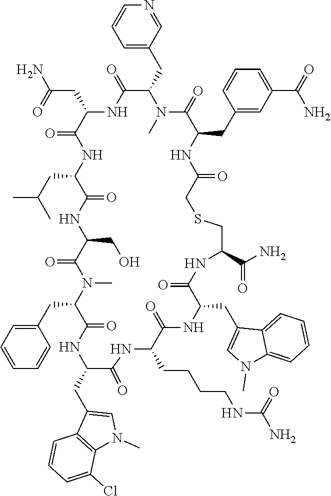 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](Cc2ccccc2)N(C)C(=O)[C@H](CO)NC1=O] | |
| PDC_EphA2- 00026601 (SEQ ID NO: 125) | 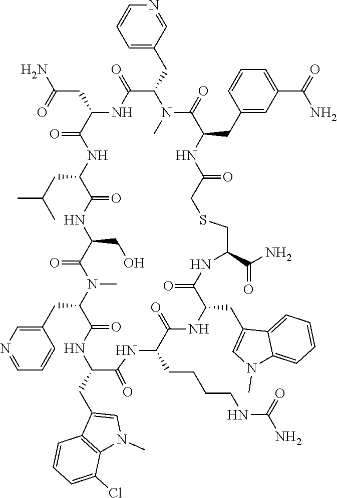 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](Cc2cccnc2)N(C)C(=O)[C@H](CO)NC1=O] | |
| PDC_EphA2- 00026602 (SEQ ID NO: 126) | 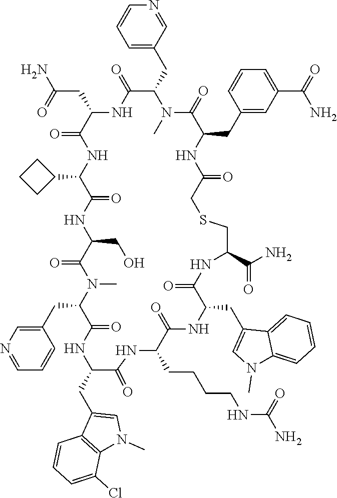 [SMILES: CN1C(=O)[C@H](CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1Cc1cccnc1] | |
| PDC_EphA2- 00026603 (SEQ ID NO: 127) | 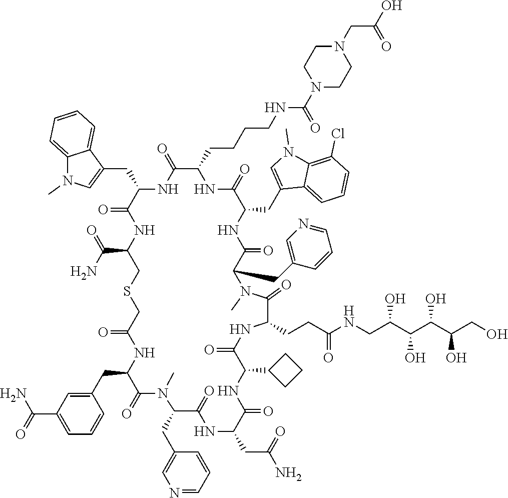 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1] | |
| PDC_EphA2- 00026604 (SEQ ID NO: 128) | 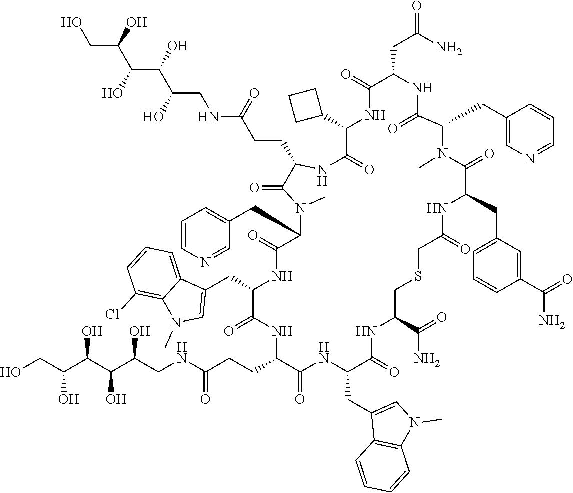 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@@H]1Cc1cccnc1] | |
| PDC_EphA2- 00026605 (SEQ ID NO: 129) | 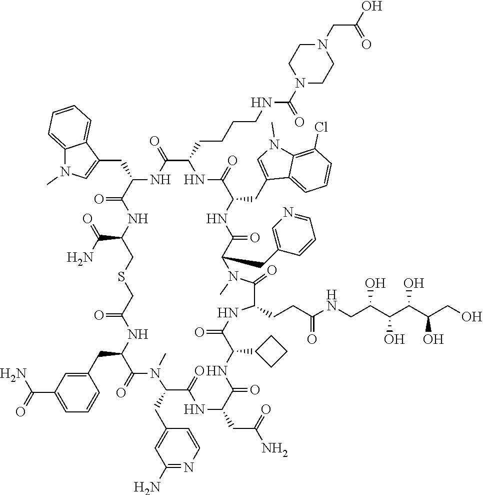 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2ccnc(N)c2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1] | |
| PDC_EphA2- 00026606 (SEQ ID NO: 130) | 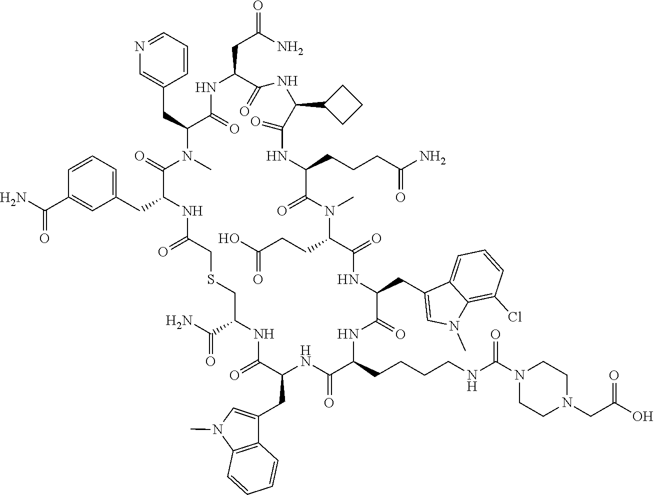 [SMILES: CN1C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026607 (SEQ ID NO: 131) | 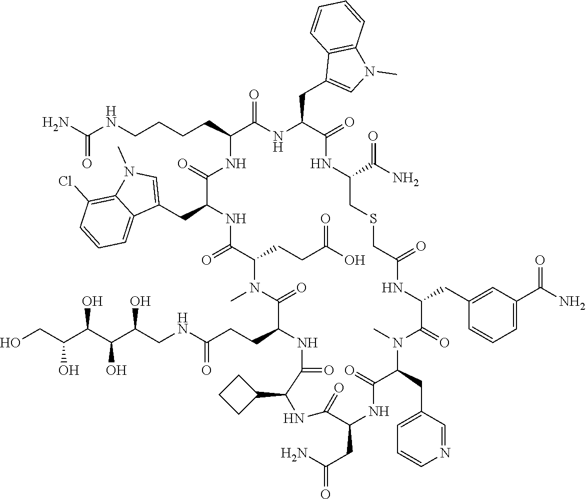 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1CCC(=O)O] | |
| PDC_EphA2- 00026608 (SEQ ID NO: 132) | 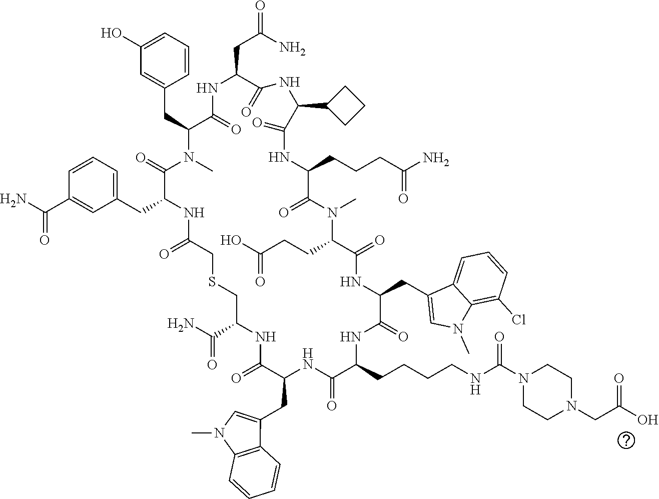 [SMILES: CN1C(=O)[C@H](CCCC(N)=O)NC(=O)[C@H](C2CCC2)NC(=O)[C@H](CC(N)=O)NC(=O)[C@H](Cc2cccc(O)c2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026609 (SEQ ID NO: 133) | 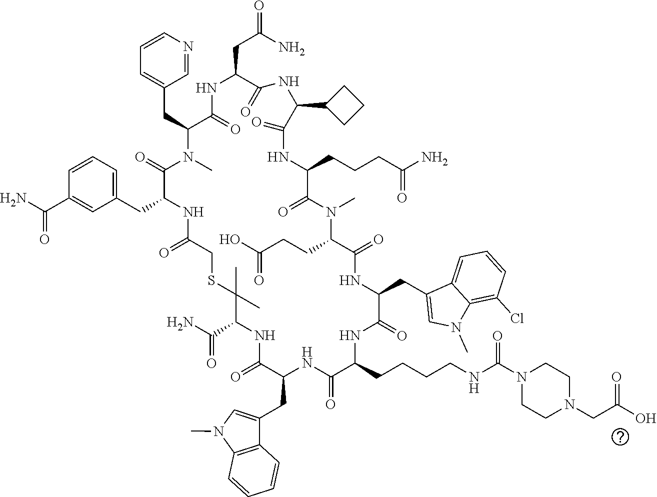 [SMILES: CN1C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC(C)(C)[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026610 (SEQ ID NO: 134) | 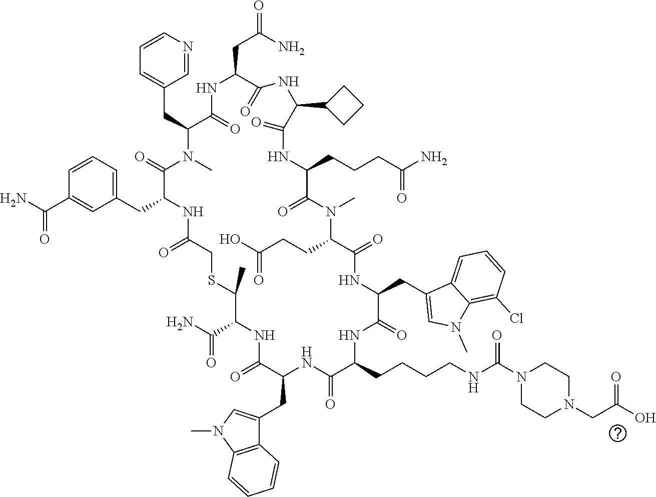 [SMILES: C[C@@H]1SCC(=O)N[C@H](Cc2cccc(C(N)=O)c2)C(=O)N(C)[C@@H](Cc2cccnc2)C(=O)N[C@@H](CC(N)=O)C(=O)N[C@@H](C2CCC2)C(=O)N[C@@H](CCCC(N)=O)C(=O)N(C)[C@@H](CCC(=O)O)C(=O)N[C@@H](Cc2cn(C)c3c(Cl)cccc23)C(=O)N[C@@H](CCCCNC(=O)N2CCN(CC(=O)O)CC2)C(=O)N[C@@H](Cc2cn(C)c3ccccc23)C(=O)N[C@@H]1C(N)=O] | |
| PDC_EphA2- 00026611 (SEQ ID NO: 135) | 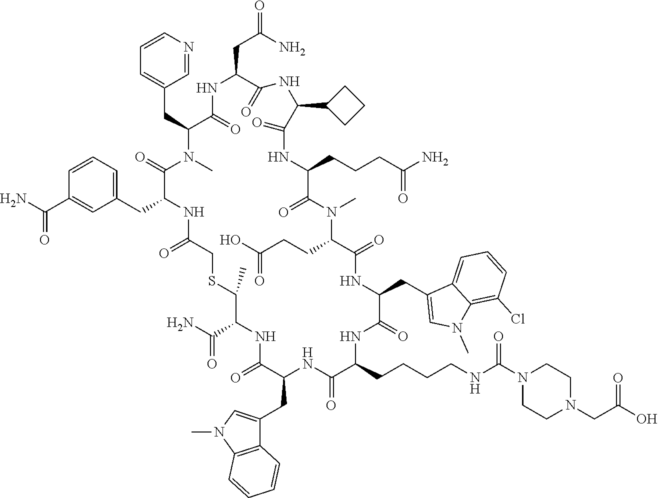 [SMILES: C[C@H]1SCC(=O)N[C@H](Cc2cccc(C(N)=O)c2)C(=O)N(C)[C@@H](Cc2cccnc2)C(=O)N[C@@H](CC(N)=O)C(=O)N[C@@H](C2CCC2)C(=O)N[C@@H](CCCC(N)=O)C(=O)N(C)[C@@H](CCC(=O)O)C(=O)N[C@@H](Cc2cn(C)c3c(Cl)cccc23)C(=O)N[C@@H](CCCCNC(=O)N2CCN(CC(=O)O)CC2)C(=O)N[C@@H](Cc2cn(C)c3ccccc23)C(=O)N[C@@H]1C(N)=O] | |

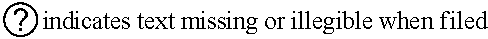

### Example 14 Interaction Analysis by SPR was Performed Using Biacore (Cytiva)

SPR assay was performed using Biacore T200 (Cytiva).

The HBS-P+ buffer (10 mM HEPES (pH7.4), 150 mM NaCl, 0.05% (v/V) Surfactant P20) containing 1% DMSO was used as running buffer. Recombinant Human EphA2 Fc Protein (Fc tag, Elabscience) was captured by Human Antibody Capture kit (Cytiva). Peptide samples in DMSO were diluted with running buffer, and prepared five serial dilutions. Using these serial dilutions, kinetics of peptides against EphA2 was measured at a flow rate of 30 mL/min at 25° C. The method adopted for sample measurement was a single-cycle kinetics method. The analysis was conducted using the evaluation software 3.0 provided with Biacore T200. Kinetics fitting was done on the difference data obtained by subtracting the baseline data from sample measurement data. KD values were calculated based on the association rate constant (ka) and dissociation rate constant (kd).

TABLE 12

| SPR | | | SPR |
|---|---|---|---|
| Example | | SEQ | Kd |
| No. | Peptide ID | ID NO: | (nM) |
| 1 | PDC_EphA2-00001417 | 1 | B |
| 2 | PDC_EphA2-00008082 | 74, 183 | A |
| 3 | PDC_EphA2-00008083 | 75, 184 | A |
| 4 | PDC_EphA2-00008084 | 76, 185 | B |
| 5 | PDC_EphA2-00008085 | 77, 186 | B |
| 6 | PDC_EphA2-00008086 | 78, 187 | A |
| 7 | PDC_EphA2-00008087 | 79, 188 | A |
| 8 | PDC_EphA2-00008088 | 80, 189 | A |
| 9 | PDC_EphA2-00008089 | 81, 190 | A |
| 10 | PDC_EphA2-00008090 | 82, 191 | A |
| 11 | PDC_EphA2-00008091 | 83, 192 | A |
| 12 | PDC_EphA2-00008092 | 84, 193 | A |
| 13 | PDC_EphA2-00008093 | 85, 194 | A |
| 14 | PDC_EphA2-00008094 | 86, 195 | A |
| 15 | PDC_EphA2-00008095 | 87, 196 | A |
| 16 | PDC_EphA2-00007196 | 88, 197 | A |
| 17 | PDC_EphA2-00008097 | 89, 198 | B |
| 18 | PDC_EphA2-00009998 | 2 | A |
| 19 | PDC_EphA2-00009999 | 3 | A |
| 20 | PDC_EphA2-00010000 | 4 | A |
| 21 | PDC_EphA2-00010001 | 5 | A |
| 22 | PDC_EphA2-00010002 | 6 | A |
| 23 | PDC_EphA2-00010003 | 7 | A |
| 24 | PDC_EphA2-00010004 | 8 | A |
| 25 | PDC_EphA2-00010005 | 9 | A |
| 26 | PDC_EphA2-00010006 | 10 | A |
| 27 | PDC_EphA2-00010007 | 11 | A |
| 28 | PDC_EphA2-00010008 | 12 | A |
| 29 | PDC_EphA2-00010009 | 13 | A |
| 30 | PDC_EphA2-00010010 | 14 | A |
| 31 | PDC_EphA2-00010011 | 15 | A |
| 32 | PDC_EphA2-00010012 | 16 | A |
| 33 | PDC_EphA2-00013693 | 17 | A |
| 34 | PDC_EphA2-00013703 | 18 | A |
| 35 | PDC_EphA2-00013704 | 19 | A |
| 36 | PDC_EphA2-00013705 | 20 | A |
| 37 | PDC_EphA2-00018526 | 22 | A |
| 38 | PDC_EphA2-00018527 | 23 | A |
| 39 | PDC_EphA2-00018528 | 24 | A |
| 40 | PDC_EphA2-00018529 | 25 | A |
| 41 | PDC_EphA2-00018530 | 26 | A |
| 42 | PDC_EphA2-00018531 | 27 | A |
| 43 | PDC_EphA2-00018532 | 28 | A |
| 44 | PDC_EphA2-00018533 | 29 | A |
| 45 | PDC_EphA2-00018534 | 30 | A |
| 46 | PDC_EphA2-00018535 | 31 | A |
| 47 | PDC_EphA2-00018536 | 32 | A |
| 48 | PDC_EphA2-00018537 | 33 | A |
| 49 | PDC_EphA2-00018538 | 34 | A |
| 50 | PDC_EphA2-00018539 | 35 | A |
| 51 | PDC_EphA2-00018540 | 36 | A |
| 52 | PDC_EphA2-00019421 | 37 | A |
| 53 | PDC_EphA2-00019422 | 38 | A |
| 54 | PDC_EphA2-00019423 | 39 | A |
| 55 | PDC_EphA2-00019424 | 40 | A |
| 56 | PDC_EphA2-00019425 | 41 | A |
| 57 | PDC_EphA2-00019426 | 42 | A |
| 58 | PDC_EphA2-00019427 | 43 | A |
| 59 | PDC_EphA2-00019428 | 44 | A |
| 60 | PDC_EphA2-00019429 | 45 | A |
| 61 | PDC_EphA2-00019430 | 46 | A |
| 62 | PDC_EphA2-00019431 | 47 | A |
| 63 | PDC_EphA2-00019432 | 48 | A |
| 64 | PDC_EphA2-00019433 | 49 | A |
| 65 | PDC_EphA2-00019434 | 50 | A |
| 66 | PDC_EphA2-00019435 | 51 | A |
| 67 | PDC_EphA2-00019436 | 52 | A |
| 68 | PDC_EphA2-00019437 | 53 | A |
| 69 | PDC_EphA2-00019438 | 54 | A |
| 70 | PDC_EphA2-00019439 | 55 | A |
| 71 | PDC_EphA2-00019440 | 56 | A |
| 72 | PDC_EphA2-00019441 | 57 | A |
| 73 | PDC_EphA2-00019442 | 58 | A |
| 74 | PDC_EphA2-00026424 | 59 | A |
| 75 | PDC_EphA2-00026425 | 60 | A |
| 76 | PDC_EphA2-00026426 | 61 | A |
| 77 | PDC_EphA2-00022417 | 123 | C |
| 78 | PDC_EphA2-00022606 | 136, 232 | C |
| 79 | PDC_EphA2-00022607 | 137, 233 | C |
| 80 | PDC_EphA2-00022608 | 138, 234 | C |
| 81 | PDC_EphA2-00021407-L092 | 139, 235 | D |
| 82 | PDC_EphA2-00022057-L092 | 140, 236 | C |
| 83 | PDC_EphA2-00026600 | 124 | B |
| 84 | PDC_EphA2-00026601 | 125 | B |
| 85 | PDC_EphA2-00026602 | 126 | A |
| 86 | PDC_EphA2-00026603 | 127 | A |
| 87 | PDC_EphA2-00026604 | 128 | A |
| 88 | PDC_EphA2-00026605 | 129 | B |
| 89 | PDC_EphA2-00026606 | 130 | A |
| 90 | PDC_EphA2-00026607 | 131 | A |
| 91 | PDC_EphA2-00026608 | 132 | A |
| 92 | PDC_EphA2-00026609 | 133 | B |
| 93 | PDC_EphA2-00026610 | 134 | B |
| 94 | PDC_EphA2-00026611 | 135 | A |
| 95 | PDC_EphA2-00020708 | 150 | D |
| 96 | PDC_EphA2-00022601 | 153, 246 | D |
| 97 | PDC_EphA2-00022602 | 154, 247 | D |
| 98 | PDC_EphA2-00022603 | 155, 248 | D |
| 99 | PDC_EphA2-00020295-L092 | 156, 249 | D |
| 100 | PDC_EphA2-00026479 | 151 | D |
| 101 | PDC_EphA2-00026481 | 152 | D |
| 102 | PDC_EphA2-00013917 | 21 | A |

SPR (K_{d} (nM)): 0 < A ≤ 1; 1 < B ≤ 10; 10 < C ≤ 100; 100 < D ≤ 400

### Example 15 PDC_EphA2-00007196-L039 (SEQ ID NO: 293)

We synthesized of the conjugated form consisting of the peptide of this invention and a payload; fluorescent compound Cys5.

### General Preparative HPLC Purification Procedure

Method P—F: XSelect C18 5 μm 19×150 mm; 17 mL/min; (A) H2O+0.1% TFA/(B) MeCN+0.1% TFA; varying gradients; Detection: DAD-UV chromatogram TIC, 220 nm

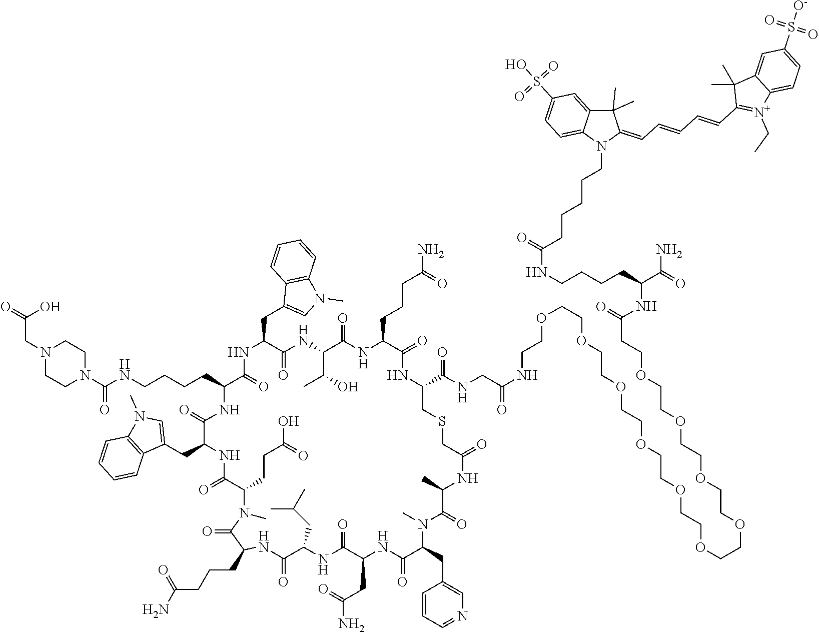 [SMILES: CC[N+]1=C(/C=C/C=C/C=C2/N(CCCCCC(=O)NCCCC[C@H](NC(=O)CCOCCOCCOCCOCCOCCOCCOCCOCCOCCOCCNC(=O)CNC(=O)[C@@H]3CSCC(=O)N[C@H](C)C(=O)N(C)[C@@H](Cc4cccnc4)C(=O)N[C@@H](CC(N)=O)C(=O)N[C@@H](CC(C)C)C(=O)N[C@@H](CCCC(N)=O)C(=O)N(C)[C@@H](CCC(=O)O)C(=O)N[C@@H](Cc4cn(C)c5ccccc45)C(=O)N[C@@H](CCCCNC(=O)N4CCN(CC(=O)O)CC4)C(=O)N[C@@H](Cc4cn(C)c5ccccc45)C(=O)N[C@@H]([C@@H](C)O)C(=O)N[C@@H](CCCC(N)=O)C(=O)N3)C(N)=O)c3ccc(S(=O)(=O)O)cc3C2(C)C)C(C)(C)c2cc(S(=O)(=O)[O-])ccc21]

The peptide sequence; PDC_EphA2-00007196-L039 was synthesized on Fmoc-Sieber amide Resin (0.050 mmol) following general peptide synthesis method A. The obtained peptide on the resin was subjected to the general method CIAc-3. The polypeptide on the resin was treated with Cleavage Cocktail-A (3 mL) to furnish the linear peptide. The crude containing the linear peptide (0.050 mmol as theoretical based on the resin used) was subjected to the peptide cyclization condition-3. After concentration, the obtained cyclic peptide was dissolved in DMSO and subsequently added DIEA (5 eq.) and SulfoCy5-OSu (1.2 eq.). The mixture was then shaken at room temperature for 1 h. The reaction was quenched with AcOH, and the resulting residue was purified by preparative reversed-phase HPLC (Method P-A). Pure fractions were combined and lyophilized to afford the title macrocyclic peptide-SulfoCy5 conjugate. HPLC-MS (Method A-B): 3.94 min, ESI-MS m/z: 1062.1 [M+3H]3+, AUC (UV 225 nm): 96%.

### Example 16 PDC_EphA2-00007196-L026 (SEQ ID NO: 307)

We synthesized of the conjugated form consisting of the peptide of this invention and a payload; biotin.

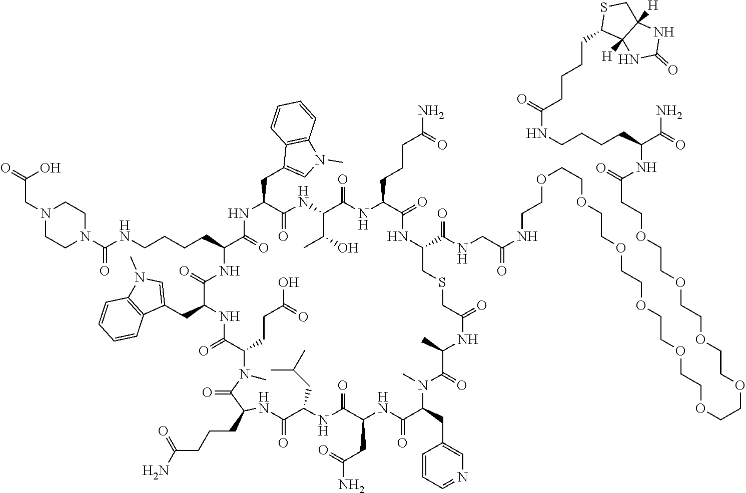 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)NCC(=O)NCCOCCOCCOCCOCCOCCOCCOCCOCCOCCOCCC(=O)N[C@@H](CCCCNC(=O)CCCC[C@@H]2SC[C@@H]3NC(=O)N[C@@H]32)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O]

The peptide sequence was synthesized on Fmoc-Sieber amide Resin (0.050 mmol) following general peptide synthesis method A. The obtained peptide on the resin was subjected to the general method CIAc-3. The polypeptide on the resin was treated with Cleavage Cocktail-A (3 mL) to furnish the linear peptide. The crude containing the linear peptide (0.050 mmol as theoretical based on the resin used) was subjected to the peptide cyclization condition-3. After concentration, the obtained cyclic peptide was dissolved in DMSO and subsequently added DIEA (3 eq.) and Biotin-OSu (1.2 eq.). The mixture was then shaken at room temperature for 1 h. The reaction was quenched with AcOH, and the resulting residue was purified by preparative reversed-phase HPLC (Method P-D). Pure fractions were combined and lyophilized to afford the title macrocyclic peptide-biotin conjugate. HPLC-MS (Method A-B): 3.30 min, ESI-MS m/z: 924.9 [M+3H]3+, AUC (UV 225 nm): 91%.

### Example 17 Other PDCs

We synthesized other PDCs including the peptide of this invention and Cys5 or biotin according to working examples described above. The amino acid sequence for each peptide was shown in Table 13. The term “Term” means the functional group at C-terminus of the peptide.

TABLE 3

| | | SEQ ID | | |
|---|---|---|---|---|
| SEQ | | NO of | | |
| ID | | cyclic | | |
| NO: | Example No. | peptides | Linker/payload | Term |
| 280 | PDC_EphA2-00001417-L026 | 1 | G PEG10c K(Biotin) | -NH₂ |
| 281 | PDC_EphA2-00001417-L039 | 1 | G PEG10c K(SulfoCy5) | -NH₂ |
| 282 | PDC_EphA2-00008083-L039 | 75 | G PEG10c K(SulfoCy5) | -NH₂ |
| 283 | PDC_EphA2-00008084-L039 | 76 | G PEG100 K(SulfoCy5) | -NH₂ |
| 284 | PDC_EphA2-00008085-L039 | 77 | G PEG10c K(SulfoCy5) | -NH₂ |
| 285 | PDC_EphA2-00008086-L039 | 78 | G PEG10c K(SulfoCy5) | -NH₂ |
| 286 | PDC_EphA2-00008087-L039 | 79 | G PEG10c K(SulfoCy5) | -NH₂ |
| 287 | PDC_EphA2-00008088-L039 | 80 | G PEG10c K(SulfoCy5) | -NH₂ |
| 288 | PDC_EphA2-00008091-L039 | 83 | G PEG10c K(SulfoCy5) | -NH₂ |
| 289 | PDC_EphA2-00008092-L039 | 84 | G PEG10c K(SulfoCy5) | -NH₂ |
| 290 | PDC_EphA2-00008093-L039 | 85 | G PEG10c K(SulfoCy5) | -NH₂ |
| 291 | PDC_EphA2-00008094-L039 | 86 | G PEG10c K(SulfoCy5) | -NH₂ |
| 292 | PDC_EphA2-00008095-L039 | 87 | G PEG10c K(SulfoCy5) | -NH₂ |
| 293 | PDC_EphA2-00007196-L039 | 88 | G PEG10c K(SulfoCy5) | -NH₂ |
| 294 | PDC_EphA2-00008097-L039 | 89 | G PEG10c K(SulfoCy5) | -NH₂ |
| 295 | PDC_EphA2-00008083-L026 | 75 | G PEG10c K(Biotin) | -NH₂ |
| 296 | PDC_EphA2-00008084-L026 | 76 | G PEG10c K(Biotin) | -NH₂ |
| 297 | PDC_EphA2-00008085-L026 | 77 | G PEG10c K(Biotin) | -NH₂ |
| 298 | PDC_EphA2-00008086-L026 | 78 | G PEG10c K(Biotin) | -NH₂ |
| 299 | PDC_EphA2-00008087-L026 | 79 | G PEG100 K(Biotin) | -NH₂ |
| 300 | PDC_EphA2-00008088-L026 | 80 | G PEG10c K(Biotin) | -NH₂ |
| 301 | PDC_EphA2-00008091-L026 | 83 | G PEG10c K(Biotin) | -NH₂ |
| 302 | PDC_EphA2-00008092-L026 | 84 | G PEG10c K(Biotin) | -NH- |
| 303 | PDC_EphA2-00008093-L026 | 85 | G PEG10c K(Biotin) | -NH₂ |
| 304 | PDC_EphA2-00008094-L026 | 86 | G PEG10c K(Biotin) | -NH₂ |
| 305 | PDC_EphA2-00008095-L026 | 87 | G PEG10c K(Biotin) | -NH₂ |
| 306 | PDC_EphA2-00007196-L026 | 88 | G PEG10c K(Biotin) | -NH₂ |
| 307 | PDC_EphA2-00008097-L026 | 89 | G PEG10c K(Biotin) | -NH₂ |
| 308 | PDC_EphA2-00010011-L026 | 15 | G PEG10c K(Biotin) | -NH₂ |
| 309 | PDC_EphA2-00010012-L026 | 16 | G PEG100 K(Biotin) | -NH₂ |
| 310 | PDC_EphA2-00013693-L026 | 17 | G PEG10c K(Biotin) | -NH₂ |
| 311 | PDC_EphA2-00020708-L026 | 150 | G PEG10c K(Biotin) | -NH₂ |
| 312 | PDC_EphA2-00022417-L026 | 123 | G PEG10c K(Biotin) | -NH₂ |
| 313 | PDC_EphA2-00019437-L026 | 53 | G PEG10c K(Biotin) | -NH₂ |
| 314 | PDC_EphA2-00022593-L026 | 96 | G PEG10c K(Biotin) | -NH₂ |
| 315 | PDC_EphA2-00022594-L026 | 97 | G PEG10c K(Biotin) | -NH₂ |
| 316 | PDC_EphA2-00022595-L026 | 98 | G PEG10c K(Biotin) | -NH₂ |
| 317 | PDC_EphA2-00022614-L026 | 102 | G PEG10c K(Biotin) | -NHA |
| 318 | PDC_EphA2-00022615-L026 | 103 | G PEG10c K(Biotin) | -NH₂ |
| 319 | PDC_EphA2-00019440-L026 | 99 | G PEG10c K(Biotin) | -NH₂ |
| 320 | PDC_EphA2-00022612-L026 | 100 | G PEG10c K(Biotin) | -NH₂ |
| 321 | PDC_EphA2-00022613-L026 | 101 | G PEG10c K(Biotin) | -NH₂ |
| 322 | PDC_EphA2-00013703-L026 | 104 | G PEG10c K(Biotin) | -NH₂ |
| 323 | PDC_EphA2-00013704-L026 | 105 | G PEG10c K(Biotin) | -NH₂ |
| 324 | PDC_EphA2-00013705-L026 | 106 | G PEG10c K(Biotin) | -NH₂ |
| 325 | PDC_EphA2-00019498-L026 | 158 | G PEG10c K(Biotin) | -NH₂ |
| 326 | PDC_EphA2-00020295-L026 | 156 | G PEG10c K(Biotin) | -NH₂ |
| 327 | PDC_EphA2-00021407-L026 | 139 | G PEG10c K(Biotin) | -NH₂ |
| 328 | PDC_EphA2-00022057-L026 | 140 | G PEG10c K(Biotin) | -NH₂ |
| 329 | PDC_EphA2-00019424-L026 | 40 | G PEG10c K(Biotin) | -NH₂ |
| 330 | PDC_EphA2-00019427-L026 | 43 | G PEG100 K(Biotin) | -NH₂ |
| 331 | PDC_EphA2-00019433-L026 | 49 | G PEG10c K(Biotin) | -NH₂ |
| 332 | PDC_EphA2-00019439-L026 | 55 | G PEG10c K(Biotin) | -NH₂ |
| 333 | PDC_EphA2-00013693-L103 | 17 | bA dk(SulfoCy5-PEG8c-PEG2c) | -NH₂ |
| 334 | PDC_EphA2-00008093-L104 | 85 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 335 | PDC_EphA2_00007196-L104 | 88 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 336 | PDC_EphA2-00013693-L102 | 17 | dk(SulfoCy5) | -NH₂ |
| 337 | PDC_EphA2-00013704-L102 | 19 | dk(SulfoCy5) | -NH₂ |
| 338 | PDC_EphA2-00013705-L102 | 20 | dk(SulfoCy5) | -NH₂ |
| 339 | PDC_EphA2-00020708-L039 | 150 | G PEG10c K(SulfoCy5) | -NH₂ |
| 340 | PDC_EphA2-00026600-L026 | 124 | G PEG10c K(Biotin) | -NH₂ |
| 341 | PDC_EphA2-00026601-L026 | 125 | G PEG10c K(Biotin) | -NH₂ |
| 342 | PDC_EphA2-00026602-L026 | 126 | G PEG10c K(Biotin) | -NHA |
| 343 | PDC_EphA2-00026603-L026 | 127 | G PEG10c K(Biotin) | -NH₂ |
| 344 | PDC_EphA2-00026604-L026 | 128 | G PEG10c K(Biotin) | -NH |
| 345 | PDC_EphA2-00026606-L026 | 130 | G PEG10c K(Biotin) | -NH₂ |
| 346 | PDC_EphA2-00026607-L026 | 131 | G PEG10c K(Biotin) | -NH₂ |
| 347 | PDC_EphA2-00026608-L026 | 132 | G PEG10c K(Biotin) | -NH₂ |
| 348 | PDC_EphA2-00001417-L104 | 1 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 349 | PDC_EphA2-00008083-L104 | 75 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 350 | PDC_EphA2-00008084-L104 | 76 | dk(SulfoCy5-bA-MeG-MeG-MeG- | -NH: |
| | | | MeG-MeG-MeG-MeG-MeG-MeG-MeG | |
| | | | (SEQ ID NO: 361)) | |
| 351 | PDC_EphA2-00008085-L104 | 77 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 352 | PDC_EphA2-00008086-L104 | 78 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 353 | PDC_EphA2-00008087-L104 | 79 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 354 | PDC_EphA2-00008088-L104 | 80 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 355 | PDC_EphA2-00008091-L104 | 83 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 356 | PDC_EphA2-00008092-L104 | 84 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| | PDC_EphA2-00008093-L104 | 85 | dk(SulfoCy5-bA-MeG-MeG-MeG- | -NH₂ |
| 357 | | | MeG-MeG-MeG-MeG-MeG-MeG-MeG | |
| | | | (SEQ ID NO: 361)) | |
| 358 | PDC_EphA2-00008094-L104 | 86 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 359 | PDC_EphA2-00008095-L104 | 87 | dk(SulfoCy5-bA-MeG-MeG-MeG- | -NH₂ |
| | | | MeG-MeG-MeG-MeG-MeG-MeG-MeG | |
| | | | (SEQ ID NO: 361)) | |
| 360 | PDC_EphA2-00008097-L104 | 89 | dk(SulfoCy5-bA-MeG-MeG-MeG- | NH₂ |
| | | | MeG-MeG-MeG-MeG-MeG-MeG-MeG | |
| | | | (SEQ ID NO: 361)) | |

Certain linker-added peptides have identical or substantially the same peptide sequences compared to certain conjugates comprising such linker-added peptides, or “naked” peptides without the linker. Although the EphA2 binding affinity for such linker-added peptides have not been directly tested, they likely bind to EphA2 with similar avidity compared to that of the conjugates comprising such linker-added peptides, or that of the “naked” peptides without the linker portion.

The present technology may be used in bio-related industries and the pharmaceutical industry.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A (cyclic) peptide that has avidity for ephrin type-A receptor 2 (EphA2), wherein the peptide comprises an amino acid sequence including deletion, substitution, and/or addition of one or several (e.g., 1-6) amino acids in the amino acid sequence of SEQ ID NO: 1:
(SEQ ID NO: 1)
da-MeF-N-L-Hgl-MeF-W1Me-V-W1Me-T-E-C
or a pharmaceutically acceptable salt thereof, wherein the (cyclic) peptide consists of 10 to 12 amino acid residues.

2. The (cyclic) peptide of claim 1, wherein 1-5 amino acids selected the group consisting of the 3^{rd} N, 4^{th} L, 6^{th} MeF, 10^{th} T and 11^{th} E of SEQ ID NO: 1, is/are deleted, optionally without additional addition and/or substitution.

3. The (cyclic) peptide of claim 1 or 2, wherein one to several (e.g., 1, 2, 3, 4 or 5) amino acids are added.

4. The (cyclic) peptide of any one of claims 1 to 3, wherein one or more amino acid residues selected from the 2^{nd} MeF, 6^{th} MeF, 8^{th} V and 11^{th} E are substituted.

5. The (cyclic) peptide of any one of claims 1 to 4, wherein the peptide comprises an amino acid sequence with deletion of 2 or less amino acids in the amino acid SEQ ID NO: 1, optionally without additional addition and/or substitution.

6. The (cyclic) peptide of claim 5, wherein 1-2 amino acids selected from the group consisting of the 10^{th} T and 11^{th} E of SEQ ID NO: 1 is/are deleted, optionally without additional addition and/or substitution.

7. A (cyclic) peptide that has avidity for ephrin type-A receptor 2 (EphA2), wherein the peptide comprises an amino acid sequence of Formula (I), or a pharmaceutically acceptable salt thereof, 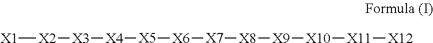
wherein,
X1 is an amino acid;
X2 is an amino acid comprising an aromatic ring, an N-methylated amino acid thereof, or a variant thereof;
X3 is a hydrophilic amino acid (e.g. N, Q, Cit, K or a variant thereof), glycine (G), Alanine (A) or a variant thereof (e.g., da, 2-Aminoisobutyric acid (Aib);
X4 is a hydrophobic amino acid (e.g., leucine (L)), a hydrophilic amino acid (e.g., citrulline (Cit), or a variant thereof;
X5 is a hydrophilic amino acid, or a variant thereof;
X6 is a hydrophilic amino acid, an amino acid comprising an aromatic ring, or an N-methylated amino acid thereof;
X7 is an amino acid comprising an aromatic ring (e.g., W, F, or a variant thereof);
X8 is a hydrophobic amino acid, a hydrophilic amino acid, an N-methylated amino acid, or a variant thereof;
X9 is an amino acid comprising an aromatic ring (e.g., W or a variant thereof);
X10 is absent or a hydrophilic amino acid (e.g., Threonine (T) or a variant thereof);
X11 is absent or a hydrophilic amino acid; and
X12 is cysteine (C) or a variant thereof.

8. The (cyclic) peptide of claim 7, wherein X3 is a hydrophilic amino acid.

9. The (cyclic) peptide of claim 8, wherein X3 is an amino acid comprising an electrically charged side chain (e.g., K or a variant thereof), an amino acid comprising a polar uncharged side chain (e.g., Q, Cit, N, or a variant thereof), or G, A or variant thereof.

10. The (cyclic) peptide of any one of claims 7-9, wherein X4 is a hydrophobic amino acid.

11. The (cyclic) peptide of claim 10, wherein X4 is an amino acid comprising a hydrophobic side chain (e.g., L), an amino acid comprising a polar uncharged side chain (e.g., Cit or a variant thereof).

12. The (cyclic) peptide of any one of claims 7-11, wherein X5 is a hydrophilic amino acid.

13. The (cyclic) peptide of claim 12, wherein X5 is an amino acid comprising an electrically charged side chain (e.g., E, Hgl, D, or a variant thereof), or an amino acid comprising a polar uncharged side chain (e.g., Q, Cit, Hgn, N, or a variant thereof).

14. The (cyclic) peptide of any one of claims 7-13, wherein X6 is a hydrophilic amino acid.

15. The (cyclic) peptide of claim 14, wherein X6 is an amino acid comprising an electrically charged side chain (e.g., E, Hgl, D, or a variant thereof), or an amino acid comprising a polar uncharged side chain (e.g., Q, Cit, Hgn, N, or variant).

16. The (cyclic) peptide of any one of claims 7-15, wherein X11 is a hydrophilic amino acid.

17. The (cyclic) peptide of claim 16, wherein X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof).

18. The (cyclic) peptide of any one of claims 1-17, wherein the peptide has an amino acid sequence of Formula (I), or a pharmaceutically acceptable salt thereof, 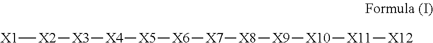
X1 is an amino acid;
X2 is F, or a variant thereof that substitutes the unsubstituted phenyl ring of F with:
(i) a phenyl ring substituted by 1 or 2 substituents each independently selected from —OH, —CN, —C₁₋₃ alkyl (e.g., —CH₃), or
(ii) a 6-membered heteroaryl ring optionally substituted by 1 or 2 substituents each independently selected from —OH, —CN, —C₁₋₃ alkyl (e.g., —CH₃),
wherein the F or the structural variant thereof is optionally N-methylated;
X3 is a hydrophilic amino acid (e.g. N, Q, Cit, K or a variant thereof), G, Alb, Hgn, Ala, or a variant thereof (e.g., da);
X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
X5 is an amino acid (e.g., a hydrophilic amino acid; Dab, Dap, R, E or a variant thereof, or an amino acid with a functional side chain (e.g., not glycine);
X6 is an N-methylated amino acid thereof;
X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F);
X8 is an amino acid with —H on the alpha-amino group;
X9 is W or Y or a variant thereof; (e.g., W or a variant thereof);
X10 is absent, or a polar amino acid (e.g., T or a variant thereof);
X11 is absent, or an amino acid (e.g., a hydrophilic amino acid; Dab, Dap, R, E or a variant thereof; or an amino acid with a functional side chain (e.g., not glycine); and
X12 is C or a variant thereof.

19. The (cyclic) peptide of any one of claims 1 to 18, wherein the peptide has an amino acid sequence of Formula (Ia), or a pharmaceutically acceptable salt thereof, 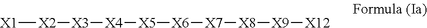
wherein,
X1 is an amino acid (e.g., D-amino acid);
X2 is an amino acid comprising an aromatic ring, an N-methylated amino acid thereof, or a variant thereof;
X3 is a hydrophilic amino acid (e.g., N, Q, Cit, K or a variant thereof), G, A, or a variant thereof (e.g., da, Aib);
X4 is a hydrophobic amino acid, or a hydrophilic amino acid (e.g., Cit or a variant thereof);
X5 is a hydrophilic amino acid (e.g., Dab, Dap, R, E, Q, D, K), or a variant thereof);
X6 is a hydrophilic amino acid, an amino acid comprising an aromatic ring (e.g., W, or F, or a variant thereof), or an N-methylated amino acid thereof;
X7 is an amino acid comprising an aromatic ring (e.g., W, F, or a variant thereof);
X8 is a hydrophobic amino acid, a hydrophilic amino acid, or an N-methylated amino acid;
X9 is an amino acid comprising an aromatic ring (e.g., W, For a variant thereof); and
X12 is C or a variant thereof.

20. The (cyclic) peptide of any one of claims 1 to 18, wherein the peptide has an amino acid sequence according to Formula (I), or a pharmaceutically acceptable salt thereof, 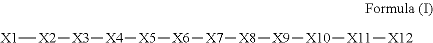
wherein,
X1 is an amino acid (e.g., D-amino acid);
X2 is an amino acid comprising an aromatic ring, an N-methylated amino acid thereof, or a variant thereof;
X3 is a hydrophilic amino acid (e.g., N, Q, Cit, K or a variant thereof), G, A, or a variant thereof (e.g., da, Aib);
X4 is a hydrophobic amino acid, or a hydrophilic amino acid (e.g., Cit or a variant thereof);
X5 is a hydrophilic amino acid (e.g., Dab, Dap, R, E, Q, D, K), or a variant thereof);
X6 is a hydrophilic amino acid, an amino acid comprising an aromatic ring (e.g., W, or F, or a variant thereof), or an N-methylated amino acid thereof;
X7 is an amino acid comprising an aromatic ring (e.g., W, F, or a variant thereof);
X8 is a hydrophobic amino acid, a hydrophilic amino acid, or an N-methylated amino acid;
X9 is an amino acid comprising an aromatic ring (e.g., W, F or a variant thereof);
X10 is a hydrophilic amino acid (e.g., T, S, N, Q, K, Cit, or a variant thereof);
X11 is a hydrophilic amino acid; and
X12 is C or a variant thereof.

21. The (cyclic) peptide of any one of claims 1 to 20, wherein
X1 is an amino acid (e.g., D-amino acid);
X2 is F, Y, W, a variant thereof, or an N-methylated amino acid thereof;
X3 is N, Q, Cit, G, Alb, K, A, or a variant thereof;
X4 is G, A, Cit, or a variant thereof (e.g., G substituted with straight or branched C₁₋₅ alkyl, G substituted with C₃₋₇ cycloalkyl, or A substituted with C₃₋₇ cycloalkyl);
X5 is a hydrophilic L-amino acid, wherein the L-amino acid comprises a functional group selected from —NH₂—C(O)OH, —NHC(NH)NH₂, —NHC(O)NH₂, —C(O)NH₂, and —NHC(O)CH₃;
X6 is a hydrophilic amino acid, F, Y, W, N-methylated amino acid thereof, or a variant thereof, wherein the hydrophilic amino acid comprises a functional group selected from —C(O)OH, C (O) NH₂, and —NHC(O)CH₃;
X7 is F, W, or a variant thereof;
X8 is G substituted with one or two straight or branched C₁₋₅ alkyl, G substituted with C₃₋₇ cycloalkyl, A substituted with C₃₋₇ cycloalkyl, or a hydrophilic L-amino acid wherein the hydrophilic L-amino acid comprises —NH₂, one or more —OH, —C(O)OH, —NHC(NH)NH₂, —NHC(O)NH₂, —C(O)NH₂, or —NHC(O)CH₃; or the hydrophilic amino acid comprises a zwitterion;
X9 is F, W, or a variant thereof;
X10 is absent, Q, S, K, Cit, N, T, or a variant thereof (e.g., Q, S, K, Cit, N, or T optionally substituted with straight or branched C₁₋₅ alkyl) or an L-amino acid comprising —NHC(NH)NH₂, —NHC(O)NH₂, —C(O)NH₂, or —NHC(O)CH₃;
X11 is absent, E, Q, R, Cit, K, D, or N, or a variant thereof; and
X12 is C or a variant thereof.

22. The (cyclic) peptide of any one of claims 1 to 21, wherein the peptide has an amino acid sequence according to Formula (I), or a pharmaceutically acceptable salt thereof, 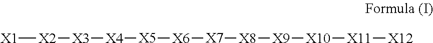
wherein,
X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, d4PyCON, or dhAla;
X2 is MeF, Me3Py, MeF3CON, MeF3F, Me4Py, or MeY(Me);
X3 is absent, N, Q, Cit, G, Aib, Hgn, hCit, norCit, LysAc, OrnAc, Ala, or da;
X4 is L, Cbg, Chg, Cba, Cha, Ahx, Dahp, Cit, I, V, Norleucine, or Norvaline;
X5 is Hgl, Hgn, Dab, Dap, DabAc, DapAc, R, hArg, E, or D;
X6 is absent, MeF, MeE, Me3Py, Me4Py, MeF4F, MeF4F, MeF4C, or MeY;
X7 is W1Me, W1Me7Cl, W1Me7N, W, F, 7-AzaTrp, W7Me, W1Et, W1Me7Br, W1Me7OMe, or W1Me6O7Cl;
X8 is V, KCOpipzaa, N, Cit, Qglucamine, hCit, K, KAc, Aib, Alb, DapAc, OrnAc, A, T, alT, Norleucine, Norvaline, Hgl, E, Hgn, Q, I, or L;
X9 is W1Me, W1Me7Cl, W1Me7N, F23dMe, W1Et, W7Me, W, F, or 7-AzaTrp;
X10 is absent, T, Q, S, Hgn, Alpha-methylserine, hSer, hThr, N, OrAc, LysAc, Cit, or hCit;
X11 is absent, E, Hgn, R, hArg, Cit, hCit, Hgl, Orn, D, N, Q, DapAc, OrnAc, DabAc, norCit; and
X12 is C, hCys, CdMe, C3RMe, C3SMe, Selenocysteine, do, or Penicillamine.

23. The (cyclic) peptide of any one of claims 18-22, wherein
X7 is W1Me or a variant thereof; and
X9 is W1Me or a variant thereof.

24. The (cyclic) peptide of any one of claims 18-23, wherein
X7 is W1Me, W1MeCl, W1MeBr, Nal1, Nal2, W1Et, 3Bzf, 38zt, F23dC, W1Me7N, or F23dMe;
X8 is V, KCOpipzaa, N, Cit, hCit, KAc, DapAc, OrAc, A, T, alT, Aib, Alb, Qglucamine, Hgl, Q, E, Hgn, or K; and
X8 is V, KCOpipzaa, N, Cit, hCit, KAc, DapAc, OrnAc, A, T, alT, Aib, Alb, Qglucamine, Hgl, Q, E, Hgn, or K; and
X9 is W1Me, Nal1, W1Et, Nal21N, 3Bzf, 3Bzt, Nal18N, F23dMe, or F23dC.

25. The (cyclic) peptide of any one of claims 1 to 17, wherein the peptide comprises an amino acid sequence according to Formula (I), or a pharmaceutically acceptable salt thereof, 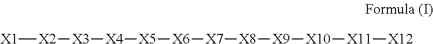
wherein,
X1 is any amino acid,
X2 is an amino acid having an aromatic ring or a variant thereof,
X3 is N,
X4 is a hydrophobic amino acid or a variant thereof;
X5 is a hydrophilic amino acid or a variant thereof;
X6 is a hydrophilic amino acid or amino acid having aromatic ring;
X7 is W or a variant thereof;
X8 is V or hydrophilic amino acid or a variant thereof,
X9 is W or a variant thereof;
X10 is T or a variant thereof;
X11 is a hydrophilic amino acid;
X12 is C or a variant thereof (such as C).

26. The (cyclic) peptide of any one of claims 1 to 17, wherein the peptide has an amino acid sequence according to Formula (Ia), or a pharmaceutically acceptable salt thereof, 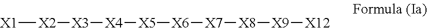
wherein,
X1 is any amino acid;
X2 is an amino acid having an aromatic ring or a variant thereof;
X3 is N or a variant thereof;
X4 is a hydrophobic amino or a variant thereof,
X5 is a hydrophilic amino acid or a variant thereof;
X6 is a hydrophilic amino acid or amino acid having aromatic ring;
X7 is W or a variant thereof;
X8 is a hydrophilic amino acid or a variant thereof,
X9 is W or a variant thereof; and
X12 is C or a variant thereof.

27. A (cyclic) peptide that has avidity for ephrin type-A receptor 2 (EphA2), wherein the peptide consists of a sequence of Formula (I), 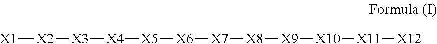
or a pharmaceutically acceptable salt thereof,
wherein
each of X1, X2, X3, X4, X5, X6, and X8 is independently an amino acid;
X7 is W1Me or a variant thereof;
X9 is W1Me or a variant thereof;
each of X10 and X11 is independently absent or an amino acid; and
X12 is cysteine (C) or a variant thereof; and,
optionally, a linker that connects the peptide with a payload molecule.

28. The (cyclic) peptide of any one of claims 7-27, wherein the variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆ alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl.

29. The (cyclic) peptide of claim 28, wherein the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, and C₁-C₆ alkyl.

30. The (cyclic) peptide of any one of claims 7-29, wherein the variant is selected from amino acids that have the similar hydrophilicity or hydrophobicity compared to the reference amino acid.

31. The (cyclic) peptide of any one of claims 7-29, wherein the variant is selected from amino acids that have the same functional group as the reference amino acid, and wherein the variant has a different length of a side chain compared to the reference amino acid.

32. The (cyclic) peptide of any one of claims 7-31, wherein the variant has a molecular weight that does not vary for more than 14, 28, 30, 45 or 60 g/mol compared to the reference amino acid.

33. A (cyclic) peptide having avidity for ephrin type-A receptor 2 (EphA2), wherein the peptide has an amino acid sequence of Formula (I), 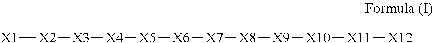
wherein,
X1 is any D- or L-amino acid;
X2 has a structure of 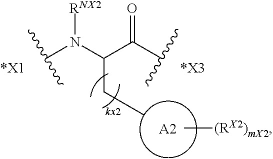
wherein
ring A2 is phenyl or a 6-membered heteroaryl (e.g., heteroaryl having 1 or 2 N);
R^{X2} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)₂R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx2 is 0, 1, 2, or 3;
mx2 is 0, 1, 2, 3 or 4;
R^{NX2} is H, C₁-C₆ alkyl, or C₁-C₆ haloalkyl;
*X1 indicates the point of attachment to X1; and,
*X3 indicates the point of attachment to X3;
X3 has a structure of 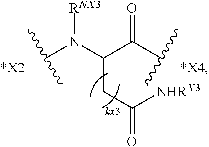
wherein
kx3 is 0, 1, 2, or 3;
R^{NX3} is H, C₁-C₆alkyl, or C₁-C₆haloalkyl;
R^{X3} is H, C₁-C₆alkyl, C₁-C₆haloalkyl, C₁-C₆hydroxyalkyl, C₁-C₆aminoalkyl, or C₁-C₆heteroalkyl;
*X2 indicates the point of attachment to X2; and,
*X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of 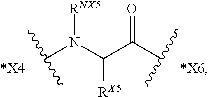
wherein:
R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
R^{XN5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
provided that at least one of R^{NX5} and R^{X5} comprises a moiety selected from —OH, —NH₂, and —NH— (e.g. —NH—C(═NH)—NH₂, —CO—NH₂, —NH₂, —COOH, —C(OH)—C₀₋₆ alkyl, —NH—CO—C₁₋₆ alkyl);
*X4 indicates the point of attachment to X4; and,
*X6 indicates the point of attachment to X6;
X6 is 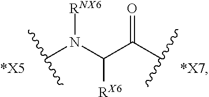
wherein
R^{NX6} is H, C₁-C₆alkyl, or C₁-C₆haloalkyl;
R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{XA};
*X5 indicates the point of attachment to X5; and,
*X7 indicates the point of attachment to X7;
X7 has a structure of 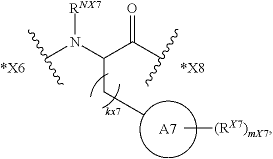
wherein
R^{NX7} is H, C₁-C₆alkyl, or C₁-C₆haloalkyl;
ring A7 is an aryl or heteroaryl;
R^{X7} is each independently halogen, —CN, —NO₂, —OH, —OR^{a}, —OC(═O)R^{a}, —OC(═O)OR^{b}, —OC(═O)NR^{c}R^{d}, —SH, SF₅; —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx7 is 0, 1, 2, or 3;
mx7 is 0, 1, 2, 3, 4 or 5;
*X6 indicates the point of attachment to X6; and,
*X8 indicates the point of attachment to X8;
X8 is an L-amino acid with —H on the alpha-amino group;
X9 has a structure of 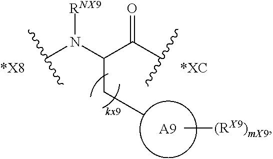
wherein
R^{NX9} is H, C₁-C₆alkyl, or C₁-C₆haloalkyl;
ring A9 is an aryl or heteroaryl;
R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx9 is 0, 1, 2, or 3;
mx9 is 0, 1, 2, 3, 4, or 5;
*X8 indicates the point of attachment to X8; and,
*XC indicates the point of attachment to (i) X10 or (i) when X10 and X11 are absent, X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl) 2, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
optionally, the peptide is linked to a payload molecule via a linker.

34. The (cyclic) peptide of claim 33, wherein ring A7 is a 6-membered aryl or heteroaryl, or a 9- or 10-membered bicyclic aryl or heteroaryl, wherein the 6-, 9- or 10-membered heteroaryl has one heteroatom selected from N, O, and S.

35. The (cyclic) peptide of claim 33 or 34, wherein R^{NX7} is H.

36. The (cyclic) peptide of any one of claims 33 to 35, wherein each R^{X7} is independently selected from —CH₃, -ethyl, —Cl, and —F, and mx7 is 0, 1, or 2.

37. The (cyclic) peptide of claim 33, wherein X7 is W1Me, Nal1, Nal2, W1Et, Nal21N, 3Bzf, 3Bzt, Nal15N, Nal14N, Nal24N, Nal28N, F23dMe, F23dC, W1Me7N, or W1Me7Cl.

38. The (cyclic) peptide of claim 37, wherein X7 is W1Me, F23dMe or W1Me7Cl.

39. The (cyclic) peptide of any one of claims 33 to 38, wherein X9 is 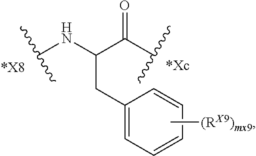
each R^{X9} is independently selected from —OH, CN, NH₂, C₁-C₃alkyl, —Cl, —F, —Br, —CONH₂, and —SO₂F.

40. The (cyclic) peptide of any one of claims 33 to 39, wherein

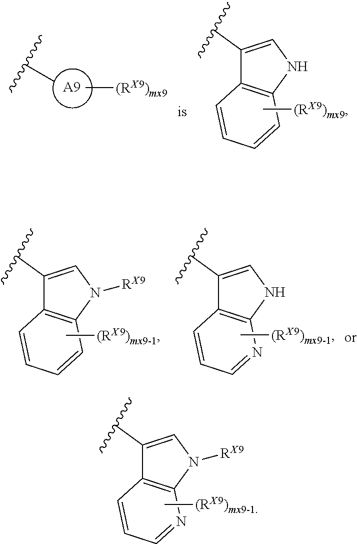

41. The (cyclic) peptide of any one of claims 33 to 40, wherein R^{X9} is each independently halogen, —CN, —NO₂, —OH, —OR^{a}, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

42. The (cyclic) peptide of any one of claims 33 to 38, wherein X9 is W1Me, W, Nal1, W1Et, Nal21N, 3Bzf, 3Bzt, Nal14N, Nal18N, F23dMe, F23dC, or W1Et.

43. The (cyclic) peptide of claim 42, wherein X9 is W1Me or F23dMe.

44. The (cyclic) peptide of any one of claims 33 to 43, wherein ring A2 is a 6-membered heteroaryl containing 1 or 2 N.

45. The (cyclic) peptide of any one of claims 33 to 44, wherein R^{XN5} is C₁-C₆hydroxyalkyl, C₁₋₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

46. The (cyclic) peptide of any one of claims 27-33, wherein
X7 is W1Me, W1MeCl, W1MeBr, Nal1, Nal2, W1Et, 3Bzf, 3Bzt, F23dC, W1Me7N, or F23dMe;
X8 is V, KCOpipzaa, Hse, N, Cit, hCit, KAc, DapAc, OrAc, T, alT, Aib, Alb, Qglucamine, Hgl, E, Hgn, MeF, 3Py6NH2, W1Me, A, Q, or K; and
X9 is W1Me, Nal1, W1Et, Nal21N, 3Bzf, 3Bzt, Nal18N, F23dMe, or F23dC.

47. The (cyclic) peptide of claim 24 or 46, wherein
X7 is W1Me;
X8 is V; and,
X9 is W1Me.

48. The (cyclic) peptide of any one of claims 1-47, wherein the peptide or the pharmaceutically accepted salt thereof has a cyclic structure, wherein the first amino acid (or X1) is covalently linked to the last amino acid (or X12).

49. The (cyclic) peptide of any one of claims 1-48, wherein the peptide or the pharmaceutically accepted salt thereof has a cyclic structure having an amino acid in the first residue X1 and a cysteine residue or a variant thereof, and wherein the amino acid in X1 and the cysteine residue or variant thereof form a covalent bond.

50. The (cyclic) peptide of any one of claims 1-49, wherein the peptide has a monocyclic structure.

51. The (cyclic) peptide of claim 50, wherein the amino acid X1 and a cysteine or a variant thereof form a covalent bond.

52. The (cyclic) peptide of any one of claims 1-51, wherein the peptide has a structure of Formula (I-1), 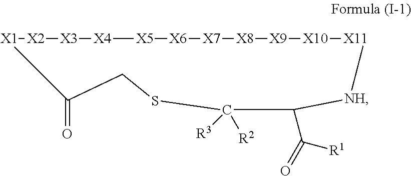
wherein
R¹ is selected from the group consisting of NH₂ and OH;
R² is selected from the group consisting of H or C₁₋₃ alkyl;
R³ is selected from the group consisting of H or C₁₋₃ alkyl;
wherein X1 to X11 have the definitions described in Formula (I).

53. The (cyclic) peptide of claim 52, or a pharmaceutically acceptable salt thereof, wherein the peptide of Formula (I-1) has a structure of Formula (I-2),

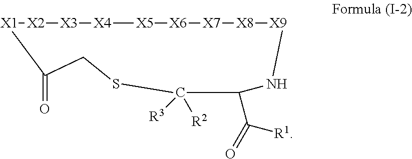

54. The (cyclic) peptide of any one of claims 1-53, wherein the peptide or the salt thereof comprises an amino acid sequence that is at least 95% identical to a sequence selected from SEQ ID NOs: 1-171, or a sequence with up to 1, 2, 3, 4, or 5 substitutions by a conserved variant compared to any one of the sequences selected from SEQ ID NOs: 1-171.

55. The (cyclic) peptide of any one of claims 1-54, wherein the peptide or the salt thereof consists of an amino acid sequence selected from SEQ ID NOs: 1-171.

56. The (cyclic) peptide of claim 55, wherein the peptide consists of an amino acid sequence selected from SEQ ID NOs: 1-122, 159-163, and 165-171, and the peptide has a cyclic structure having a cysteine residue or a variant thereof at the 12^{th} residue, and wherein the amino acid at X1 (e.g., a chloroacetylated amino acid) and the cysteine residue or a variant thereof at the 12^{th} residue form a covalent bond (e.g., by reacting a chloroacetyl group in the amino acid of X1 with the cysteine residue or a variant thereof).

57. The (cyclic) peptide of claim 55, wherein the peptide consists of an amino acid sequence selected from SEQ ID NOs: 123-149 and 164, and the peptide has a cyclic structure having a cysteine residue or a variant thereof at the 10^{th} residue, and wherein the amino acid at X1 (e.g., a chloroacetylated amino acid) and the cysteine residue or a variant thereof at the 10^{th} residue form a covalent bond.

58. The (cyclic) peptide of any one of claims 1-57, wherein the peptide has a binding affinity to a human EphA2 of at most 100 nM as determined by K_{d} in surface plasmon resonance (SPR) analysis.

59. The (cyclic) peptide of claim 58, wherein the peptide has a binding affinity to a human EphA2 of at most 1 nM as determined by K_{d} in surface plasmon resonance (SPR) analysis.

60. The (cyclic) peptide of any one of claims 1-59, wherein the peptide binds to a ligand-binding domain (LBD) domain of the EphA2.

61. The (cyclic) peptide of any one of claims 1-60, wherein the peptide interacts with a human EphA2 at one or more amino acid residues selected from Asp53, Met55, Asn57, Met59, Met66, Thr101, Arg103, Phe156, Glu157, Arg159, Val161, Val189, and Ala190.

62. The (cyclic) peptide of any one of claims 1-61, wherein the peptide interacts with a human EphA2 at Asp53 and Glu157.

63. The (cyclic) peptide of any one of claims 1-62, wherein the peptide has a plasma half-life (Tin) of at least 50, 100, 150, 200, 250, 300, 350, 400, 450, or 500 minutes as determined in vitro in human plasma at 37° C.

64. The (cyclic) peptide of claim 63, wherein the peptide has a plasma half-life (T_{1/2}) of at least 250 minutes as determined in vitro in human plasma at 37° C.

65. A (cyclic) peptide of any one of claims 1-64, covalently linked to a linker that connects the peptide to a payload molecule.

66. The (cyclic) peptide of claim 65, wherein the linker is attached to the peptide via a non-terminal amino acid residue of the peptide.

67. The (cyclic) peptide of claim 66, wherein the linker is attached to the 5^{th} amino acid residue or X5.

68. The (cyclic) peptide of claim 66, wherein the linker is attached to the 8^{th} amino acid residue or X8.

69. The (cyclic) peptide of claim 66, wherein the linker is attached to the 11^{th} amino acid residue or X11.

70. The (cyclic) peptide of any one of claims 65 to 69, wherein linker is attached to a lysine of the peptide.

71. The (cyclic) peptide of any one of claims 65 to 70, wherein the linker is attached to the peptide via the N terminus of the peptide.

72. The (cyclic) peptide of any one of claims 65 to 70, wherein the linker is attached to the peptide via the C terminus of the peptide.

73. The (cyclic) peptide of any one of claims 65 to 72, wherein the linker is a bond.

74. The (cyclic) peptide of any one of claims 65 to 72, wherein the linker comprises 3 to 30 intervening atoms between the payload molecule and the peptide.

75. The (cyclic) peptide of any one of claims 65 to 72, wherein the linker comprises 6 to 18 intervening atoms between the payload molecule and the peptide.

76. The (cyclic) peptide of claim 74 or 75, wherein the intervening atoms comprise 1 to 6 nitrogen and 0 to 4 oxygen.

77. The (cyclic) peptide of any one of claims 65-72 and 74-76, wherein the linker comprises one or more amino acid residues.

78. The (cyclic) peptide of claim 77, wherein the linker comprises one or more amino acids chosen from a lysine residue, an alanine residue, or a phenylalanine residue.

79. The (cyclic) peptide of any one of claims 65-72 and 74-78, wherein the linker comprises one or more structures selected from AEEA, AEEP, AEEEP, and AEEEEP.

80. The (cyclic) peptide of any one of claims 65-72, wherein the linker has a structure of Formula (II-1) 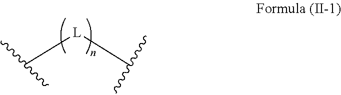
wherein each L is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂, or —N(R^{L})₂—(C₁-C₃₀ alkylene)-; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20.

81. The (cyclic) peptide of claim 80, wherein the linker comprises a structure of Formula (II-1a), 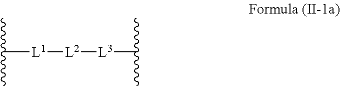
wherein each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—; and
L² is absent, substituted or unsubstituted C₁-C₃₀ alkylene, or substituted or unsubstituted C₁-C₃₀ heteroalkylene.

82. The (cyclic) peptide of claim 81, wherein L¹ is —NH—.

83. The (cyclic) peptide of claim 81 or 82, wherein L² is substituted or unsubstituted C₁-C₃₀ alkylene, or substituted or unsubstituted C₁-C₃₀ heteroalkylene.

84. The (cyclic) peptide of claim 81 or 82, wherein L² is substituted or unsubstituted C₁-C₁₈ alkylene, or substituted or unsubstituted C₁-C₁₈ heteroalkylene.

85. The (cyclic) peptide of any one of claims 81 to 84, wherein L² is optionally substituted with one or more substituents selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}—OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}; and the C₁-C₆ alkyl is further optionally substituted with one or more substituents chosen from —OH, —SH, oxo, amino, C₆-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

86. The (cyclic) peptide of any one of claims 81 to 85, wherein L³ is —NH—.

87. The (cyclic) peptide of claim 81, wherein the linker has a structure of

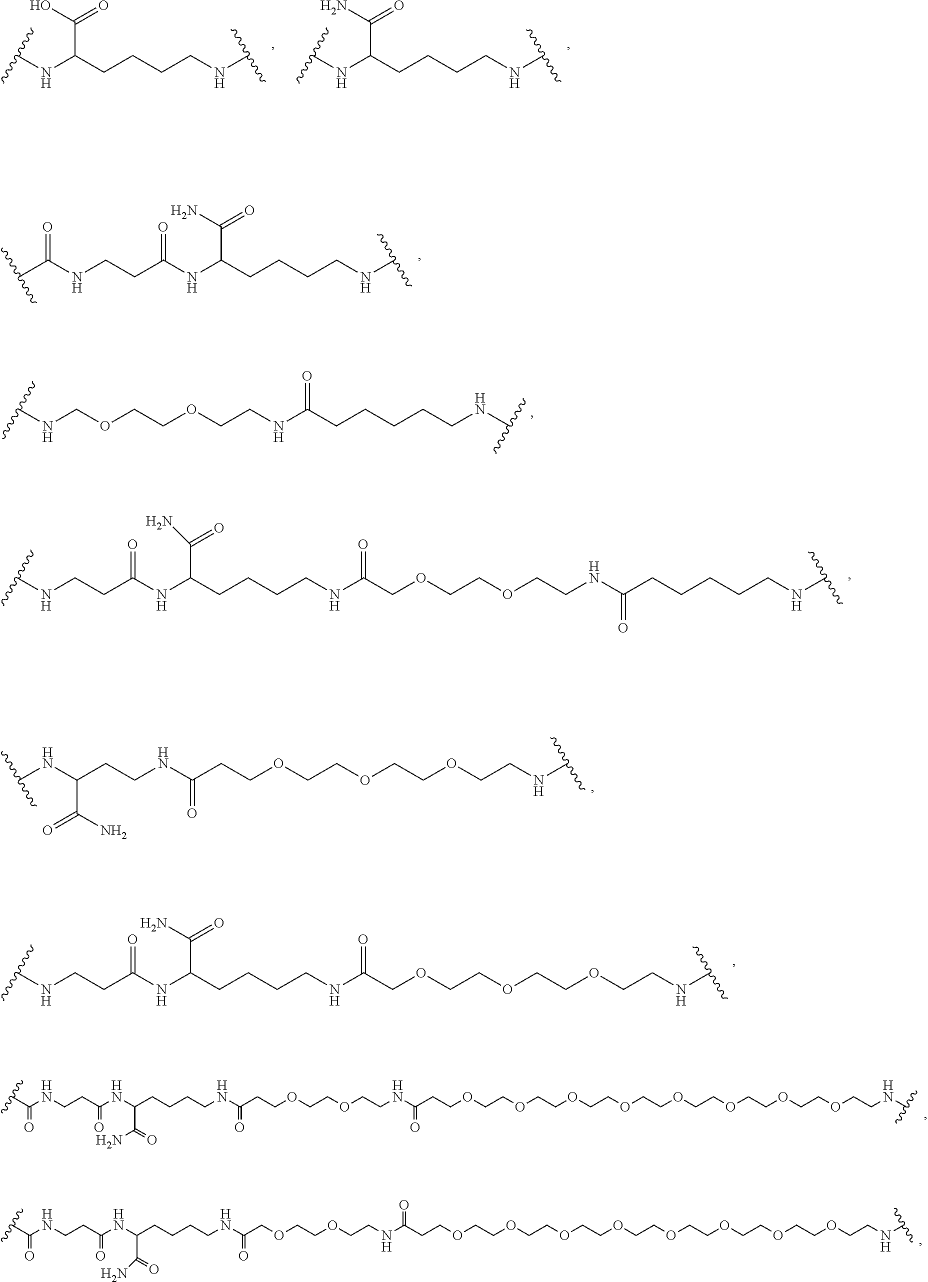

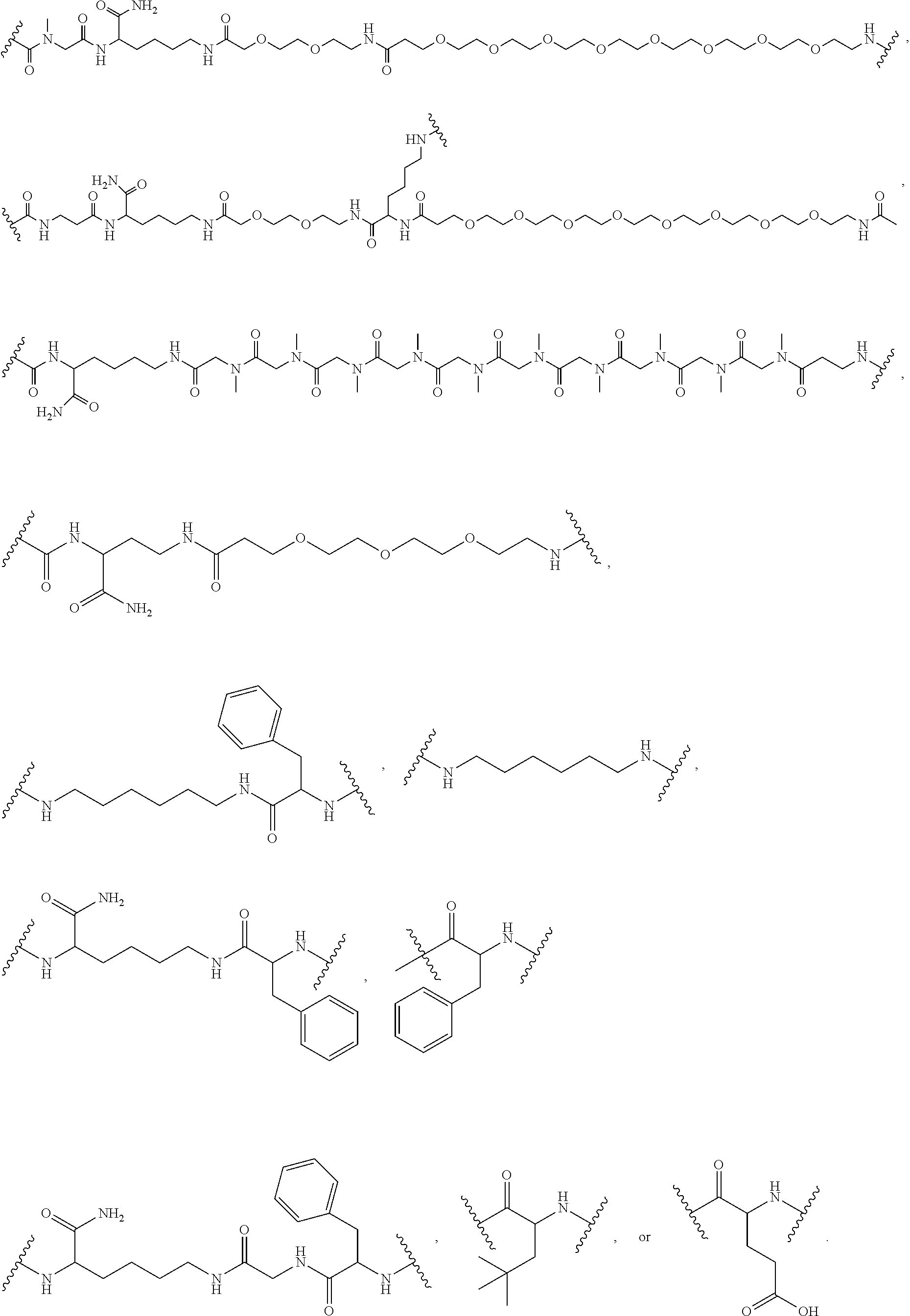

88. The (cyclic) peptide of claim 81, wherein the linker has a structure of

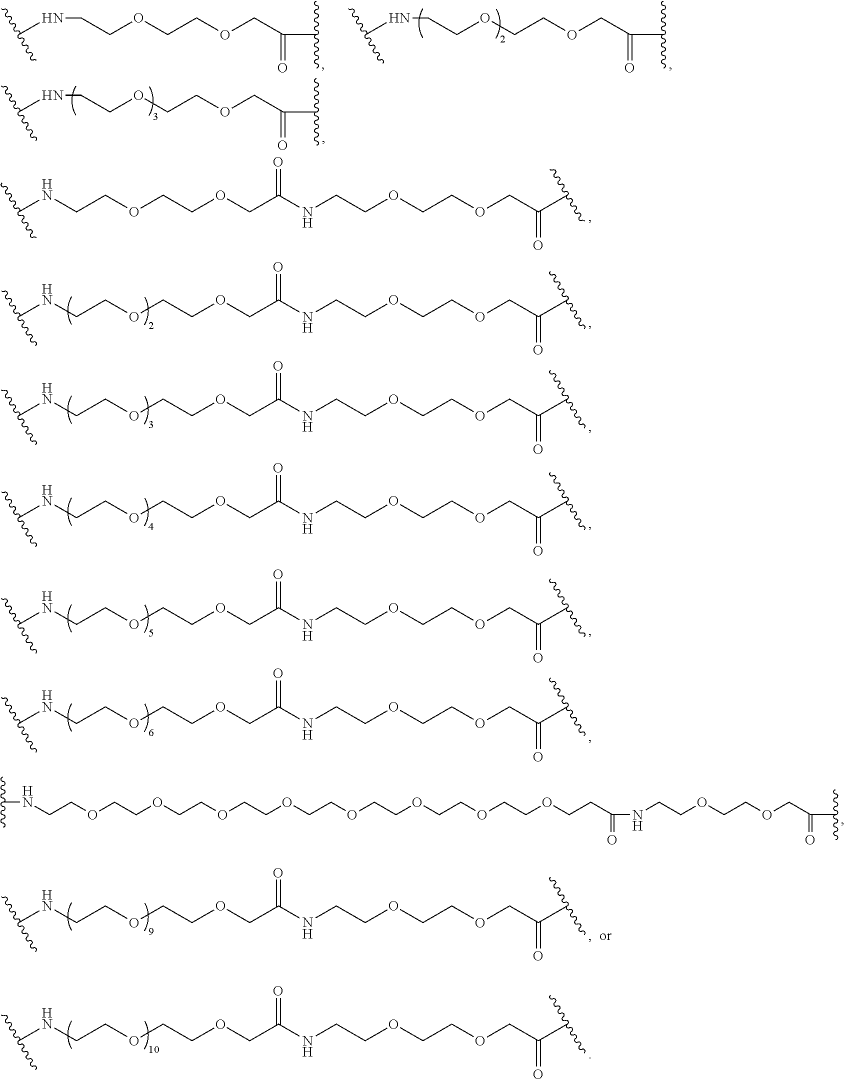

89. The (cyclic) peptide of any one of claims 1-88, wherein the peptide is a peptide of Formula (I) and wherein, when the peptide is bound to the human EphA2, amino acid residue X7 is located less than 10 Å from the Phe156 of the human EphA2.

90. The (cyclic) peptide of claim 89, wherein amino acid residue X7 is located less than 6 Å from the Phe156.

91. The (cyclic) peptide of claim 89, wherein amino acid residue X7 is located less than 4 Å from the Phe156.

92. The (cyclic) peptide of any one of claims 1-91, wherein the peptide is a peptide of Formula (I) and wherein, when the peptide is bound to the human EphA2, amino acid residue X9 is located less than 10 Å from the Phe156 of the human EphA2.

93. The (cyclic) peptide of claim 92, wherein amino acid residue X9 is located less than 6 Å from the Phe156.

94. The (cyclic) peptide of claim 93, wherein amino acid residue X9 is located less than 4 Å from the Phe156.

95. The (cyclic) peptide of any one of claims 1-94, wherein the peptide is a peptide of Formula (I) and wherein, when the peptide is bound to the human EphA2, amino acid residue X8 is located less than 10 Å from the Phe156 of the human EphA2.

96. The (cyclic) peptide of any one of claims 89 to 95, wherein the human EphA2 comprises a sequence of SEQ ID NO: 276 or SEQ ID NO: 277.

97. A (cyclic) peptide that has avidity for ephrin type-A receptor 2 (EphA2), and competes for binding to a human EphA2 with a peptide that has an amino acid sequence including deletion, substitution, or addition of one or several amino acids in the amino acid of SEQ ID NO: 1:
da-MeF—N-L-Hgl-MeF-W1Me-V-W1Me-T-E-C (SEQ ID NO: 1)
or a pharmaceutically acceptable salt thereof.

98. A (cyclic) peptide that has avidity for ephrin type-A receptor 2 (EphA2), wherein the peptide competes for binding to a human EphA2 with a peptide that has a structure of Formula (I), or a pharmaceutically acceptable salt thereof, 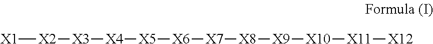
wherein,
X1 is an amino acid;
X2 is an amino acid comprising an aromatic ring, an N-methylated amino acid thereof, or a variant thereof;
X3 is a hydrophilic amino acid (e.g. N, Q, Cit, K or a variant thereof), glycine (G), Alanine (A) or a variant thereof (e.g., da, 2-Aminoisobutyric acid (Aib);
X4 is a hydrophobic amino acid (e.g., leucine (L)), a hydrophilic amino acid (e.g., citrulline (Cit), or a variant thereof;
X5 is a hydrophilic amino acid, or a variant thereof;
X6 is a hydrophilic amino acid, an amino acid comprising an aromatic ring, or an N-methylated amino acid thereof;
X7 is an amino acid comprising an aromatic ring (e.g., W, F, or a variant thereof);
X8 is a hydrophobic amino acid, a hydrophilic amino acid, an N-methylated amino acid, or a variant thereof;
X9 is an amino acid comprising an aromatic ring (e.g., W or a variant thereof);
X10 is absent or a hydrophilic amino acid (e.g., Threonine (T) or a variant thereof);
X11 is absent or a hydrophilic amino acid; and
X12 is cysteine (C) or a variant thereof.

99. A (cyclic) peptide that has avidity for ephrin type-A receptor 2 (EphA2), wherein the peptide consists of a sequence of Formula (I), 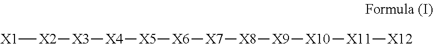
or a pharmaceutically acceptable salt thereof,
wherein
each of X1, X2, X3, X4, X5, X6, and X8 is independently an amino acid;
X7 is W1Me or a variant thereof;
X9 is W1Me or a variant thereof;
each of X10 and X11 is independently absent or an amino acid; and
X12 is cysteine (C) or a variant thereof; and,
wherein the peptide is optionally linked to a payload molecule through a linker.

100. The (cyclic) peptide of any one of claims 97 to 99, wherein the peptide competes for binding to a human EphA2 at one or more amino acid residues selected from Asp53, Met55, Asn57, Met59, Met66, Thr101, Arg103, Phe156, Glu157, Arg159, Val161, Val189, and Ala190.

101. The (cyclic) peptide of claim 100, wherein the peptide competes for binding to human EphA2 at one or more amino acid residues selected from Asp53, Phe156, and Glu157.

102. The (cyclic) peptide of any one of claims 97 to 101, wherein the human EphA2 comprises a sequence of SEQ ID NO: 276 or SEQ ID NO: 277.

103. A pharmaceutical composition comprising the peptide or salt thereof according to any one of claims 1-102, and a pharmaceutically acceptable excipient or carrier.

104. A conjugate comprising the peptide or salt thereof according to any one of the preceding claims and a substance, wherein the substance is selected from the group consisting of: a nucleotide, a small molecule, a medium sized molecule (e.g., with a M.W. of about 1,000-2,500 Da), a large sized molecule (e.g., with a M.W. of >2,500 Da), a polymer compound, a protein, a peptide, a tag, a biological fragment, a carrier including pharmaceutical compound, or a combination thereof.

105. A method of treating a disease or disorder characterized by overexpression of EphA2, comprising administering to the subject the peptide or salt thereof according to any one of claims 1-102, the conjugate of claim 103, or the pharmaceutical composition of claim 104.

106. The method of claim 105, wherein the disease or disorder is cancer.

107. The method of claim 106, wherein the cancer is selected from glioblastoma, prostate cancer, lung cancer, breast cancer, gastric cancer, ovarian cancer, gladder cancer, colon cancer, esophageal cancer, multiple myeloma and fibrosarcoma.

108. The method of claim 106, wherein the cancer is non-small cell lung carcinomas (NSCLC).

109. The method of claim 106, wherein the cancer is triple negative breast cancer.

110. A kit, tester, or composition for determining the expression level of EphA2 in a sample, wherein the kit, tester, or composition comprises the peptide or salt thereof according to any one of claims 1-102, the conjugate of claim 103, or the pharmaceutical composition of claim 104.

111. The kit, tester, or composition of claim 110, adapted for use in a method of diagnosing disease or disorder characterized by an overexpression or a decreased expression of EphA2.

112. The kit, tester, or composition of claim 110 or 111, wherein the sample is from a subject having a disease or disorder characterized by an overexpression or a decreased expression of EphA2.

113. Use of the peptide or salt thereof according to any one of the preceding claims in the manufacture of a medicament for diagnosing and/or treating a disease or disorder characterized by an overexpression or a decreased expression of EphA2.

114. The peptide or salt thereof according to any one of the preceding claims, for use in diagnosing and/or treating a disease or disorder characterized by an overexpression or a decreased expression of EphA2.


SEQUENCE LISTING (361 sequences)

SEQ ID NO: 1 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 2 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 3 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTQC

SEQ ID NO: 4 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWKWTQC

SEQ ID NO: 5 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWKWTQC

SEQ ID NO: 6 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTQC

SEQ ID NO: 7 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(2,3-dimethylphenyl)propanoic acid
  SITE 11: note = 2-amino-5-carbamoylpentanoic acid
  SITE 12: note = Amidated Cysteine
  SITE 1..12: note = Optionally cyclic
AANGQFWKFTQC

SEQ ID NO: 8 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANGQFWKWTNC

SEQ ID NO: 9 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTNC

SEQ ID NO: 10 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 11 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGXEWKWTQC

SEQ ID NO: 12 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (2S)-2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 13 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQEWKWTQC

SEQ ID NO: 14 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 15 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 16 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 17 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 18 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWXWTQC

SEQ ID NO: 19 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQAWXWTQC

SEQ ID NO: 20 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWXWTQC

SEQ ID NO: 21 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQFWXWTQC

SEQ ID NO: 22 - protein, 12 aa, synthetic construct
  SITE 1: note = 2-aminoheptanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLXFWKWTQC

SEQ ID NO: 23 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 3-(4-chlorophenyl)-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXXWKWTQC

SEQ ID NO: 24 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANLXFWKWTNC

SEQ ID NO: 25 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANLXFWKWTNC

SEQ ID NO: 26 - protein, 12 aa, synthetic construct
  SITE 1: note = 2-aminoheptanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQEWKWTQC

SEQ ID NO: 27 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-3-(methylcarbamoyl)propanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLNEWKWTQC

SEQ ID NO: 28 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-3-(dimethylcarbamoyl)propanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLNEWKWTQC

SEQ ID NO: 29 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANLQEWKWTNC

SEQ ID NO: 30 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANLQEWKWTNC

SEQ ID NO: 31 - protein, 12 aa, synthetic construct
  SITE 1: note = 2-aminoheptanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWKWTQC

SEQ ID NO: 32 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-3-(methylcarbamoyl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGNFWKWTQC

SEQ ID NO: 33 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-3-(dimethylcarbamoyl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGNFWKWTQC

SEQ ID NO: 34 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 3-(4-chlorophenyl)-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQXWKWTQC

SEQ ID NO: 35 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANGQFWKWTNC

SEQ ID NO: 36 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANGQFWKWTNC

SEQ ID NO: 37 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWVWTQC

SEQ ID NO: 38 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNLQNWVWTQC

SEQ ID NO: 39 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNLQNWVWTQC

SEQ ID NO: 40 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWVWTQC

SEQ ID NO: 41 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWKWTQC

SEQ ID NO: 42 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWXWTQC

SEQ ID NO: 43 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XFNGQEWXWTRC

SEQ ID NO: 44 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 12: note = Amidated Cysteine
  SITE 1..12: note = Optionally cyclic
XANGQNWXWTRC

SEQ ID NO: 45 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XANGQNWXWTRC

SEQ ID NO: 46 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XANGQNWKWTRC

SEQ ID NO: 47 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQFWXWTQC

SEQ ID NO: 48 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQFWKWTQC

SEQ ID NO: 49 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWXWTQC

SEQ ID NO: 50 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWKWTQC

SEQ ID NO: 51 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWXWTQC

SEQ ID NO: 52 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWKWTQC

SEQ ID NO: 53 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 54 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWKWTQC

SEQ ID NO: 55 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQEWXWTQC

SEQ ID NO: 56 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANNGEWKWTQC

SEQ ID NO: 57 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGKFWKWTQC

SEQ ID NO: 58 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGKFWXWTQC

SEQ ID NO: 59 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNAWXWTNC

SEQ ID NO: 60 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 61 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 62 - protein, 12 aa, synthetic construct
  SITE 1: note = D-2-aminopropanoic acid
  SITE 2: note = 2-(methylamino)-3-(pyridin-3-yl)propanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKEWKWTQC

SEQ ID NO: 63 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 64 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLQEWKWTKC

SEQ ID NO: 65 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLKNWXWTQC

SEQ ID NO: 66 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQNWKWTQC

SEQ ID NO: 67 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLQNWXWTKC

SEQ ID NO: 68 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLKEWKWTQC

SEQ ID NO: 69 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQEWKWTQC

SEQ ID NO: 70 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLQEWKWTKC

SEQ ID NO: 71 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 72 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 73 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 74 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 75 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLXFWVWTEC

SEQ ID NO: 76 - protein, 12 aa, synthetic construct
  SITE 1: note = (2R)-2-amino-6-{[4-(carboxymethyl)piperazine-1-carb onyl]amino}hexanoic acid
  SITE 2: note = (2S)-2-(methylamino)-3-(pyridin-3-yl)propanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
KANLXFWVWTEC

SEQ ID NO: 77 - protein, 12 aa, synthetic construct
  SITE 1: note = (2R)-2-amino-6-{[4-(carboxymethyl)piperazine-1-carb onyl]amino}hexanoic acid
  SITE 2: note = (2S)-2-(methylamino)-3-(pyridin-3-yl)propanoic acid
  SITE 5: note = (2S)-2-amino-6-{[4-(carboxymethyl)piperazine-1-carb onyl]amino}hexanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
KANLKFWVWTEC

SEQ ID NO: 78 - protein, 12 aa, synthetic construct
  SITE 1: note = (2R)-2-amino-6-{[4-(carboxymethyl)piperazine-1-carb onyl]amino}hexanoic acid
  SITE 3: note = (2S)-2-(methylamino)-3-(pyridin-3-yl)propanoic acid
  SITE 5: note = (2S)-2-amino-6-{[4-(carboxymethyl)piperazine-1-carb onyl]amino}hexanoic acid
  SITE 6: note = (2S)-2-(methylamino)-3-phenylpropanoic acid
  SITE 8: note = (2S)-2-amino-3-(1-methyl-1H-indol-3-yl)propanoic acid
  SITE 10: note = (2S)-2-amino-3-(1-methyl-1H-indol-3-yl)propanoic acid
  SITE 12: note = (2S)-2-amino-5-carbamoylpentanoic acid
  SITE 12: note = Amidated Cysteine
  SITE 1..12: note = Optionally cyclic
KANLKFWVWTQC

SEQ ID NO: 79 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLXFWKWTEC

SEQ ID NO: 80 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 81 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 82 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 83 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXFWKWTQC

SEQ ID NO: 84 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKFWKWTQC

SEQ ID NO: 85 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 86 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 87 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKEWKWTQC

SEQ ID NO: 88 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 89 - protein, 12 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLXFWKWTEC

SEQ ID NO: 90 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 91 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 92 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 93 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 94 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 95 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 96 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWXWTQC

SEQ ID NO: 97 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGXNWXWTQC

SEQ ID NO: 98 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWXWTQC

SEQ ID NO: 99 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQEWKWTQC

SEQ ID NO: 100 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 101 - protein, 12 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 102 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWXWTQC

SEQ ID NO: 103 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWXWTQC

SEQ ID NO: 104 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWXWTQC

SEQ ID NO: 105 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQAWXWTQC

SEQ ID NO: 106 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWXWTQC

SEQ ID NO: 107 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNAWXWTNC

SEQ ID NO: 108 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 109 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 110 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 111 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 112 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 113 - protein, 12 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = (2S)-2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 114 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 115 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 116 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 117 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 118 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 119 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 120 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 121 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 122 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 123 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 124 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSFWXWC

SEQ ID NO: 125 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 126 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGSAWXWC

SEQ ID NO: 127 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-(methylamino)-3-(pyridin-3-yl)propanoic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 128 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWXWC

SEQ ID NO: 129 - protein, 10 aa, synthetic construct
  SITE 1: note = 2-amino-3-(3-carbamoylphenyl)propanoic acid
  SITE 2: note = Methyl-(S)-2-amino-3-(2-aminopyridin-4-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 130 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGQEWKWC

SEQ ID NO: 131 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXEWXWC

SEQ ID NO: 132 - protein, 10 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XFNGQEWKWC

SEQ ID NO: 133 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (R)-2-amino-3-mercapto-3-methylbutanoic acid
  SITE 1..10: note = Optionally cyclic
XANGQEWKWC

SEQ ID NO: 134 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (2R,3S)-2-amino-3-mercaptobutanoic acid
  SITE 1..10: note = Optionally cyclic
XANGQEWKWC

SEQ ID NO: 135 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (2R,3R)-2-amino-3-mercaptobutanoic acid
  SITE 1..10: note = Optionally cyclic
XANGQEWKWC

SEQ ID NO: 136 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Glycine
FHNLSFWXWCG

SEQ ID NO: 137 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 138 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 139 - protein, 10 aa, synthetic construct
  SITE 1: note = D-2-aminopropanoic acid
  SITE 2: note = 2-(methylamino)-3-phenylpropanoic acid
  SITE 4: note = 2-amino-3-cyclobutylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
AFNATNWXWC

SEQ ID NO: 140 - protein, 10 aa, synthetic construct
  SITE 2: note = 2-amino-3-(pyridin-4-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 3Py6-NH2
  SITE 9: note = 2-amino-3-(1-methyl-1H-indol-3-yl)propanoic acid
  SITE 1..10: note = Optionally cyclic
FANLEFWXWC

SEQ ID NO: 141 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 142 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSFWXWC

SEQ ID NO: 143 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 144 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 145 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 146 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWXWC

SEQ ID NO: 147 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGQEWKWC

SEQ ID NO: 148 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXGWXWC

SEQ ID NO: 149 - protein, 10 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
XFNGQEWKWC

SEQ ID NO: 150 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = Amidated Cysteine
  SITE 1..8: note = Optionally cyclic
XNLSXXXC

SEQ ID NO: 151 - protein, 8 aa, synthetic construct
  SITE 2: note = 4Py2NH2
  SITE 4: note = MeA
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 8: note = Amidated Cysteine
XXLAXFXC

SEQ ID NO: 152 - protein, 8 aa, synthetic construct
  SITE 2: note = 4Py2NH2
  SITE 4: note = MeHcit
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 8: note = Amidated Cysteine
XXLAXFXC

SEQ ID NO: 153 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 154 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 155 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 156 - protein, 8 aa, synthetic construct
  SITE 2: note = 2-amino-3-(pyridin-4-yl)propanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLGXFXC

SEQ ID NO: 157 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLGXFXC

SEQ ID NO: 158 - protein, 7 aa, synthetic construct
  SITE 1: note = D-2-aminopropanoic acid
  SITE 2: note = 4Py2NH2
  SITE 3: note = 2-(methylamino)-3-phenylpropanoic acid
  SITE 4: note = 2-amino-3-(1-methyl-1H-indol-3-yl)propanoic acid
  SITE 5: note = 2-(methylamino)-3-phenylpropanoic acid
  SITE 6: note = 2-amino-3-(1-methyl-1H-indol-3-yl)propanoic acid
  SITE 1..7: note = Optionally cyclic
AXFWFWC

SEQ ID NO: 159 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
XANGQEWKWTQC

SEQ ID NO: 160 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGQEWKWTQC

SEQ ID NO: 161 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 162 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
XANGKEWKWTQC

SEQ ID NO: 163 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 164 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(1-methyl-1H-indol-3-yl)propanoic acid
  SITE 9: note = 2-amino-5-carbamoylpentanoic acid
  SITE 1..10: note = Optionally cyclic
XANGKEWTQC

SEQ ID NO: 165 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
AANGQEWKWTKC

SEQ ID NO: 166 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 167 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
AANLQEWKWTEC

SEQ ID NO: 168 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
AANLQEWKWTRC

SEQ ID NO: 169 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = D-2-amino-6-(carbamoylamino)hexanoic acid
  SITE 1..12: note = Optionally cyclic
AANLQEWKWTXC

SEQ ID NO: 170 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
AANLQNWKWTRC

SEQ ID NO: 171 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
XANGQEWKWTRC

SEQ ID NO: 172-179 - skipped (8 entries)

SEQ ID NO: 180 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AANGQNWVWTQCAK

SEQ ID NO: 181 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AANGQAWVWTQCAK

SEQ ID NO: 182 - protein, 14 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AFNGQNWVWTQCAK

SEQ ID NO: 183 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AFNLXFWVWTECAK

SEQ ID NO: 184 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLQFWVWTECAK

SEQ ID NO: 185 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
KANLQFWVWTECAK

SEQ ID NO: 186 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
KANLKFWVWTECAK

SEQ ID NO: 187 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
KANLKFWVWTQCAK

SEQ ID NO: 188 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLXFWKWTECAK

SEQ ID NO: 189 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLKFWKWTECAK

SEQ ID NO: 190 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Lysine(Ac)
AANLKFWKWTECK

SEQ ID NO: 191 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine(Ac)
AANLKFWKWTECK

SEQ ID NO: 192 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLXFWKWTQCAK

SEQ ID NO: 193 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLKFWKWTQCAK

SEQ ID NO: 194 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLQFWKWTQCAK

SEQ ID NO: 195 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLXEWKWTQCAK

SEQ ID NO: 196 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLKEWKWTQCAK

SEQ ID NO: 197 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLQEWKWTQCAK

SEQ ID NO: 198 - protein, 14 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLXFWKWTECAK

SEQ ID NO: 199 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANLQFWKWTQCK

SEQ ID NO: 200 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANLQEWKWTQCK

SEQ ID NO: 201 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQNWVWTQCK

SEQ ID NO: 202 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQAWVWTQCK

SEQ ID NO: 203 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQNWVWTQCK

SEQ ID NO: 204 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQAWXWTQCK

SEQ ID NO: 205 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQNWXWTQCK

SEQ ID NO: 206 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 7: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGXNWXWTQCK

SEQ ID NO: 207 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQNWXWTQCK

SEQ ID NO: 208 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQEWKWTQCK

SEQ ID NO: 209 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQEWKWTQCK

SEQ ID NO: 210 - protein, 13 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQEWKWTQCK

SEQ ID NO: 211 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQAWXWTQCK

SEQ ID NO: 212 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQNWXWTQCK

SEQ ID NO: 213 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQNWXWTQCK

SEQ ID NO: 214 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQAWXWTQCK

SEQ ID NO: 215 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQAWXWTQCK

SEQ ID NO: 216 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANLNAWXWTNCK

SEQ ID NO: 217 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANLNNWXWTNCK

SEQ ID NO: 218 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANNLNWXWTNCK

SEQ ID NO: 219 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
AANLQEWKWTQCK

SEQ ID NO: 220 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
AFNGQNWVWTQCK

SEQ ID NO: 221 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
XANGQAWXWTQCK

SEQ ID NO: 222 - protein, 13 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
XFNGQEWKWTQCK

SEQ ID NO: 223 - protein, 14 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AFNLXFXVXTECAK

SEQ ID NO: 224 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXEXXXTXCAK

SEQ ID NO: 225 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXFXXXTXCAK

SEQ ID NO: 226 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXEXXXTXCAK

SEQ ID NO: 227 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNXXXXVXTXCAK

SEQ ID NO: 228 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNXXXXVXTXCAK

SEQ ID NO: 229 - protein, 14 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(PEG8c-PEG2c)
AFNXXXXVXTXCAK

SEQ ID NO: 230 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to bA-MeG-MeG-MeG-MeG-MeG-MeG-MeG-MeG-MeG-MeG via the side chain
AXNLXFXXXTXCK

SEQ ID NO: 231 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to bA-MeG-MeG-MeG-MeG-MeG-MeG-MeG-MeG-MeG-MeG via the side chain
AXNLXEXXXTXCK

SEQ ID NO: 232 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
FHNLSFXXXCG

SEQ ID NO: 233 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine(Acetyl)
FHNLSFWXWCK

SEQ ID NO: 234 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated D-Lysine(Acetyl)
FHNLSFWXWCK

SEQ ID NO: 235 - protein, 11 aa, synthetic construct
  SITE 1: note = D-2-aminopropanoic acid
  SITE 2: note = 2-(methylamino)-3-phenylpropanoic acid
  SITE 4: note = 2-amino-3-cyclobutylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Glycine
AFNATNWXWCG

SEQ ID NO: 236 - protein, 11 aa, synthetic construct
  SITE 2: note = 2-amino-3-(pyridin-4-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-3-(6-aminopyridin-3-yl)propanoic acid
  SITE 9: note = 2-amino-3-(1-methyl-1H-indol-3-yl)propanoic acid
  SITE 1..10: note = Optionally cyclic
  SITE 11: note = Amidated Glycine
FANLEFWXWCG

SEQ ID NO: 237 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated D-Lysine
FHNLSFWXWCK

SEQ ID NO: 238 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANLSFWXWCK

SEQ ID NO: 239 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANLSAWXWCK

SEQ ID NO: 240 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGSAWXWCK

SEQ ID NO: 241 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXAWKWCK

SEQ ID NO: 242 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXAWXWCK

SEQ ID NO: 243 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGQEWXWCK

SEQ ID NO: 244 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXEWXWCK

SEQ ID NO: 245 - protein, 11 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XFNGQEWKWCK

SEQ ID NO: 246 - protein, 9 aa, synthetic construct
  SITE 2: note = 2-amino-3-(2-aminopyridin-4-yl)propanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine
FXLXWFWCG

SEQ ID NO: 247 - protein, 9 aa, synthetic construct
  SITE 2: note = 2-amino-6-(carbamoylamino)hexanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Lysine(Acetyl)
FXLXWFWCK

SEQ ID NO: 248 - protein, 9 aa, synthetic construct
  SITE 2: note = 2-amino-6-(carbamoylamino)hexanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated D-Lysine(Acetyl)
FXLXWFWCK

SEQ ID NO: 249 - protein, 9 aa, synthetic construct
  SITE 2: note = 2-amino-3-(pyridin-4-yl)propanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine
FALGWFWCG

SEQ ID NO: 250 - protein, 9 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated D-Lysine
FXLXWFWCK

SEQ ID NO: 251 - protein, 8 aa, synthetic construct
  SITE 1: note = D-2-aminopropanoic acid
  SITE 2: note = 2-amino-3-(2-aminopyridin-4-yl)propanoic acid
  SITE 3: note = 2-(methylamino)-3-phenylpropanoic acid
  SITE 4: note = 2-amino-3-(1-methyl-1H-indol-3-yl)propanoic acid
  SITE 5: note = 2-(methylamino)-3-phenylpropanoic acid
  SITE 6: note = 2-amino-3-(1-methyl-1H-indol-3-yl)propanoic acid
  SITE 1..7: note = Optionally cyclic
  SITE 8: note = Amidated Glycine
AXFXFXCG

SEQ ID NO: 252 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQEWKWTQCK

SEQ ID NO: 253 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQEWKWTQCK

SEQ ID NO: 254 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = D-Glutamate
AANGKEWKWTQCE

SEQ ID NO: 255 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Glutamate
XANGKEWKWTQCE

SEQ ID NO: 256 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Glutamate
AANGKEWKWTQCE

SEQ ID NO: 257 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(1-methyl-1H-indol-3-yl)propanoic acid
  SITE 9: note = 2-amino-5-carbamoylpentanoic acid
  SITE 1..10: note = Optionally cyclic
  SITE 11: note = Amidated D-Glutamate
XANGKEWTQCE

SEQ ID NO: 258 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = dk
  SITE 14: note = df
AANLQEWKWTQCKF

SEQ ID NO: 259 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lys conjugated to Glycine(D-Phenylalanine) via the side chain
AANLQEWKWTQCK

SEQ ID NO: 260 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = D-Glutamate
AANGQEWKWTKCE

SEQ ID NO: 261 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = D-Phenylalanine
AANGQEWKWTKCF

SEQ ID NO: 262 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 2-amino-4,4-dimethylpentanoic acid
AANGQEWKWTKCX

SEQ ID NO: 263 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = de
  SITE 14: note = Amidated de
AANGKEWKWTQCEE

SEQ ID NO: 264 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = de
  SITE 14: note = Amidated df
AANGKEWKWTQCEF

SEQ ID NO: 265 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = de
  SITE 14: note = Amidated 2-amino-4,4-dimethylpentanoic acid
AANGKEWKWTQCEX

SEQ ID NO: 266 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine
AXNLXEXXXTECK

SEQ ID NO: 267 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQEWKWTRCK

SEQ ID NO: 268 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = D-2-amino-6-(carbamoylamino)hexanoic acid
  SITE 1..12: note = Optionally cyclic
  SITE 13: note = Amidated D-Lysine
AANLQEWKWTRCK

SEQ ID NO: 269 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQEWKWTXCK

SEQ ID NO: 270 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQNWKWTRCK

SEQ ID NO: 271 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = homo-Arginine
  SITE 1..12: note = Optionally cyclic
  SITE 13: note = D-Lysine
XANGQEWKWTRCK

SEQ ID NO: 272 - protein, 10 aa, synthetic construct
  SITE 2..4: note = 2-amino-3-(1-methyl-1H-indol-3-yl)propanoic acid
  SITE 6: note = 2-amino-3-(6-aminopyridin-3-yl)propanoic acid
  SITE 7: note = 2-(methylamino)-3-phenylpropanoic acid
  SITE 1..9: note = Optionally cyclic
  SITE 10: note = Amidated Cysteine
FWWWSXFECG

SEQ ID NO: 273 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = homo-Arginine
  SITE 1..12: note = Optionally cyclic
  SITE 13: note = D-Lysine
XANGQEWKWTRCK

SEQ ID NO: 274 - protein, 13 aa, synthetic construct
KYNDRLPLYISNP

SEQ ID NO: 275 - protein, 30 aa, synthetic construct
  VARIANT 6..30: note = GGGGS repeats may be deleted
GGGGSGGGGSGGGGSGGGGSGGGGSGGGGS

SEQ ID NO: 276 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 277 - protein, 497 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKVTPRGAGLALAGPTAGDRLVT

SEQ ID NO: 278 - protein, 5 aa, synthetic construct
EAAAK

SEQ ID NO: 279 - protein, 11 aa, synthetic construct
  SITE 1: note = 3-aminopropanoic acid
  SITE 2..11: note = Methyl-Glycine
AGGGGGGGGGG

SEQ ID NO: 280 - protein, 13 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AFNLXFWVWTECG

SEQ ID NO: 281 - protein, 13 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AFNLXFWVWTECG

SEQ ID NO: 282 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWVWTECGK

SEQ ID NO: 283 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = (S)-2-aminohexanedioic acid
  SITE 7: note = 2-amino-3-(1-methyl-1H-indol-3-yl)propanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
KANLXFWVWTECG

SEQ ID NO: 284 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
KANLKFWVWTECG

SEQ ID NO: 285 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 12: note = 2-amino-5-carbamoylpentanoic acid
  SITE 1..12: note = Optionally cyclic
  SITE 13: note = Amidated Glycine conjugated to (PEG10c)Lysine(Biotin) via side chain
KANLKFWVWTQCG

SEQ ID NO: 286 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWKWTECG

SEQ ID NO: 287 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLKFWKWTECG

SEQ ID NO: 288 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLXFWKWTQCG

SEQ ID NO: 289 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLKFWKWTQCG

SEQ ID NO: 290 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLQFWKWTQCG

SEQ ID NO: 291 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLXEWKWTQCG

SEQ ID NO: 292 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLKEWKWTQCG

SEQ ID NO: 293 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLQEWKWTQCG

SEQ ID NO: 294 - protein, 13 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWKWTECG

SEQ ID NO: 295 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWVWTECG

SEQ ID NO: 296 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = (S)-2-aminohexanedioic acid
  SITE 7: note = 2-amino-3-(1-methyl-1H-indol-3-yl)propanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
KANLXFWVWTECG

SEQ ID NO: 297 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
KANLKFWVWTECG

SEQ ID NO: 298 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 12: note = 2-amino-5-carbamoylpentanoic acid
  SITE 1..12: note = Optionally cyclic
  SITE 13: note = Amidated Glycine conjugated to (PEG10c)Lysine(Biotin) via side chain
KANLKFWVWTQCG

SEQ ID NO: 299 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWKWTECG

SEQ ID NO: 300 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLKFWKWTECG

SEQ ID NO: 301 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLXFWKWTQCG

SEQ ID NO: 302 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLKFWKWTQCG

SEQ ID NO: 303 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLQFWKWTQCG

SEQ ID NO: 304 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLXEWKWTQCG

SEQ ID NO: 305 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLKEWKWTQCG

SEQ ID NO: 306 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANLQEWKWTQCG

SEQ ID NO: 307 - protein, 13 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Glycine conjugated to (PEG10c)Lysine(Biotin) via side chain
AANLXFWKWTECG

SEQ ID NO: 308 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANGQNWVWTQCG

SEQ ID NO: 309 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANGQAWVWTQCG

SEQ ID NO: 310 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AFNGQNWVWTQCG

SEQ ID NO: 311 - protein, 9 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine conjugated to (PEG10c)Lysine(Biotin) via side chain
XNLSXXXCG

SEQ ID NO: 312 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
FHNLSFWXWCG

SEQ ID NO: 313 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQAWXWTQCG

SEQ ID NO: 314 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQNWXWTQCG

SEQ ID NO: 315 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGXNWXWTQCG

SEQ ID NO: 316 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQNWXWTQCG

SEQ ID NO: 317 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XFNGQAWXWTQCG

SEQ ID NO: 318 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XFNGQNWXWTQCG

SEQ ID NO: 319 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQEWKWTQCG

SEQ ID NO: 320 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XFNGQEWKWTQCG

SEQ ID NO: 321 - protein, 13 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XFNGQEWKWTQCG

SEQ ID NO: 322 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AFNGQNWXWTQCG

SEQ ID NO: 323 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AFNGQAWXWTQCG

SEQ ID NO: 324 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
AANGQAWXWTQCG

SEQ ID NO: 325 - protein, 8 aa, synthetic construct
  SITE 1: note = D-2-aminopropanoic acid
  SITE 2: note = 4Py2NH2
  SITE 3: note = 2-(methylamino)-3-phenylpropanoic acid
  SITE 4: note = 2-amino-3-(1-methyl-1H-indol-3-yl)propanoic acid
  SITE 5: note = 2-(methylamino)-3-phenylpropanoic acid
  SITE 6: note = 2-amino-3-(1-methyl-1H-indol-3-yl)propanoic acid
  SITE 1..7: note = Optionally cyclic
  SITE 8: note = Amidated Glycine conjugated to (PEG10c)Lysine(Biotin) via side chain
AXFWFWCG

SEQ ID NO: 326 - protein, 9 aa, synthetic construct
  SITE 2: note = 2-amino-3-(pyridin-4-yl)propanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine conjugated to (PEG10c)Lysine(Biotin) via side chain
XXLGXFXCG

SEQ ID NO: 327 - protein, 11 aa, synthetic construct
  SITE 1: note = D-2-aminopropanoic acid
  SITE 2: note = 2-(methylamino)-3-phenylpropanoic acid
  SITE 4: note = 2-amino-3-cyclobutylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
AFNATNWXWCG

SEQ ID NO: 328 - protein, 11 aa, synthetic construct
  SITE 2: note = 2-amino-3-(pyridin-4-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 3Py6-NH2
  SITE 9: note = 2-amino-3-(1-methyl-1H-indol-3-yl)propanoic acid
  SITE 1..10: note = Optionally cyclic
  SITE 11: note = Amidated Amidated Glycine conjugated to (PEG10c)Lysine(Biotin) via side chain
FANLEFWXWCG

SEQ ID NO: 329 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQNWVWTQCG

SEQ ID NO: 330 - protein, 13 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = 2-amino-6-carbamimidamidohexanoic acid
  SITE 1..12: note = Optionally cyclic
  SITE 13: note = Amidated Glycine conjugated to (PEG10c)Lysine(Biotin) via side chain
XFNGQEWXWTRCG

SEQ ID NO: 331 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQFWXWTQCG

SEQ ID NO: 332 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated Glycine conjugated to (PEG10c)Lysine(Biotin) via side chain
XANGQEWXWTQCG

SEQ ID NO: 333 - protein, 14 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine conjugated to SulfoCy5-PEG8c-PEG2c via the side chain
AFNGQNWVWTQCAK

SEQ ID NO: 334 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeGvia the side chain
AANLQFWKWTQCK

SEQ ID NO: 335 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeGvia the side chain
AANLQEWKWTQCK

SEQ ID NO: 336 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5 via the side chain
AFNGQNWVWTQCK

SEQ ID NO: 337 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5 via the side chain
AFNGQAWXWTQCK

SEQ ID NO: 338 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5 via the side chain
AANGQAWXWTQCK

SEQ ID NO: 339 - protein, 9 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine conjugated to (PEG10c)Lysine(Biotin) via side chain
XNLSXXXCG

SEQ ID NO: 340 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANLSFWXWCG

SEQ ID NO: 341 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANLSAWXWCG

SEQ ID NO: 342 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANGSAWXWCG

SEQ ID NO: 343 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANGXAWKWCG

SEQ ID NO: 344 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANGXAWXWCG

SEQ ID NO: 345 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANGQEWKWCG

SEQ ID NO: 346 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XANGXEWXWCG

SEQ ID NO: 347 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 9: note = 2-amino-6-(carbamoylamino)hexanoic acid
  SITE 10: note = 2-amino-3-(1-methyl-1H-indol-3-yl)propanoic acid
  SITE 1..10: note = Optionally cyclic
  SITE 11: note = Amidated Glycine conjugated to (PEG10c)Lysine(Biotin) via side chain
XFNGQEWKWCG

SEQ ID NO: 348 - protein, 13 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AFNLXFWVWTECK

SEQ ID NO: 349 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLXFWVWTECK

SEQ ID NO: 350 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = (S)-2-aminohexanedioic acid
  SITE 7: note = 2-amino-3-(1-methyl-1H-indol-3-yl)propanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = D-Lysine conjugated to SulfoCy5-bA-MeG-MeG-MeG-MeG-MeG-MeG-MeG-MeG-MeG-MeG via side chain
KANLXFWVWTECK

SEQ ID NO: 351 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
KANLKFWVWTECK

SEQ ID NO: 352 - protein, 13 aa, synthetic construct
  SITE 2: note = 2-amino-6-{[4-(carboxymethyl)piperazine-1-carbonyl] amino}hexanoic acid
  SITE 3: note = 2-(methylamino)-3-(pyridin-3-yl)propanoic acid
  SITE 6: note = 2-amino-6-{[4-(carboxymethyl)piperazine-1-carbonyl] amino}hexanoic acid
  SITE 7: note = 2-(methylamino)-3-phenylpropanoic acid
  SITE 8: note = 2-amino-3-(1-methyl-1H-indol-3-yl)propanoic acid
  SITE 10: note = 2-amino-3-(1-methyl-1H-indol-3-yl)propanoic acid
  SITE 12: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
KANLKFWVWTQCK

SEQ ID NO: 353 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLXFWKWTECK

SEQ ID NO: 354 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLKFWKWTECK

SEQ ID NO: 355 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLXFWKWTQCK

SEQ ID NO: 356 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLKFWKWTQCK

SEQ ID NO: 357 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-(methylamino)-3-phenylpropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLQFWKWTQCK

SEQ ID NO: 358 - protein, 13 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLXEWKWTQCK

SEQ ID NO: 359 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = 2-(methylamino)pentanedioic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLKEWKWTQCK

SEQ ID NO: 360 - protein, 13 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine conjugated to SulfoCy5-bA-MeG-MeG-MeG-MeG-MeG-MeG-MeG-MeG-MeG-MeG via side chain
AANLXFWKWTECK

SEQ ID NO: 361 - protein, 11 aa, synthetic construct
  SITE 1: note = sulfo Cyanine5-Beta-Alanine
  SITE 2..11: note = Methyl-Glycine
AGGGGGGGGGG